(19) United States
(12) Patent Application Publication
(10) Pub. No.: US 2026/0284016 A1
(43) Pub. Date: Sep. 24, 2026

(54) COMBINATIONS AND METHODS FOR ENHANCING CHECKPOINT INHIBITOR THERAPIES IN CANCER TREATMENT

(71) Applicant: PURDUE RESEARCH FOUNDATION, West Lafayette, IN (US)

(72) Inventors: Philip S. Low, West Lafayette, IN (US); Madduri Srinivasarao, West Lafayette, IN (US); John Victor Napoleon, Tuscaloosa, AL (US)

(21) Appl. No.: 19/168,265

(22) PCT Filed: Jul. 2, 2004
(86) PCT No.: PCT/US2204/021385

§ 371 Date: Sep. 25, 2025

Related U.S. Application Data
(60) Provisional application No. 63/454,306, filed on Mar. 23, 2023.

(51) Int. Cl. A61K 31/437; A61K 31/519; A61K 39/395; A61P 35/00

(52) U.S. Cl. CPC A61K 31/437; A61K 31/519; A61K 39/39558; A61P 35/00

(57) ABSTRACT

Combinations of (i) conjugates comprising a targeted immune modulator and (ii) one or more immune checkpoint inhibitors; compositions; and methods of use to reprogram M2-like macrophages to M1-like macrophages to reverse the proinflammatory response observed in cancer to an anti-inflammatory response.

### PRIORITY

This patent application is related to and claims the priority benefit of U.S. Provisional Patent Application No. 63/454,306 filed Mar. 23, 2023. The content of the foregoing application is hereby incorporated by reference in its entirety into this disclosure.

### TECHNICAL FIELD

This disclosure relates to methods for enhancing the potency of one or more checkpoint inhibitors (as compared to such checkpoint inhibitor(s)' baseline potency) using one or more compounds that comprise a targeting moiety and reprogramming M2-type macrophages to M1-type macrophages.

### BACKGROUND

In a normal healthy system, immune checkpoints are surface proteins that are present to check and thwart any overstimulation of immune response. Their role is to prevent an immune response from being so strong that it destroys healthy cells in the body. In the case of cancer, when the checkpoint protein on a T cell (e.g., programmed cell death protein 1 (PD-1)) engages with its binding protein on a tumor cell (programmed death-ligand 1 (PD-L1)), it sends an “off” signal to the T cells and suppresses the anti-tumor immune response. In this manner, tumor killer cells within the immune system cannot kill the tumor because their method of attack is blocked by those checkpoints on the tumor itself.

Immune checkpoint inhibitors function by inhibiting the checkpoint proteins from binding with their ligands on tumor cells. PD-1, CTLA-4 (cytotoxic T-lymphocyte-associated protein 4), LAG3 (lymphocyte activating 3), TIM3 (T cell immunoglobulin and mucin domain-containing protein 3), TIGIT (T cell immunoreceptor with Ig and ITIM domains), and VISTA (V-domain Ig suppressor of T cell activation) are some of the examples of checkpoint proteins. Checkpoint inhibitor therapy has been approved by the U.S. Food & Drug Administration for several cancer types. Some of the approved checkpoint inhibitors include anti-PD1 antibodies including Pembrolizumab (Keytruda®) and Nivolumab (Opdivo®) and an anti-CTLA4 antibody called Ipilimumab (Yervoy®).

There remain some unmet needs in this area of cancer immunotherapy. For example, ~50% of patients with PD-L1-positive tumors show resistance or relapse post PD-1/PD-L1 checkpoint therapy (Herbst et al., Predictive correlates of response to the anti-PD-L1 antibody MPDL3280A in cancer patients, Nature 515(7528): 563-567 (2014)). One of the critical factors behind the resistance to checkpoint blockade can be the function of myeloid-derived suppressor cells (MDSCs) and tumor-associated macrophages (TAMs) (Bai et al., Regulation of PD-I/PD-L1 pathway and resistance to PD-I/PD-L1 blockade, Oncotarget 8(66): 110693-110707 (2017)). The MDSCs in a tumor microenvironment are known to contribute to the compromise in the efficacy of checkpoint blockade (Meyer et al., Frequencies of circulating MDSC correlate with clinical outcome of melanoma patients treated with ipilimumab, Cancer Immunology Immunotherapy 63: 247-57 (2014)). Similarly, reprogramming of TAMs has been shown to increase the efficacy of checkpoint blockade (Zhu et al., CSF1/CSF1R blockade reprograms tumor-infiltrating macrophages and improves response to T-cell checkpoint immunotherapy in pancreatic cancer models, Cancer Research 74: 5057-69 (2014)).

Despite the clear need for the prevention and treatment of cancers, these conditions remain significant causes of death and suffering worldwide because no effective therapeutic cures presently exist. Further, where drugs or other therapies are available, such treatments typically employ highly potent drugs that risk systemic toxicity in the underlying subject as they are poorly selective for the cancer cells of interest. What is needed is a treatment effective to not only disrupt the pro-growth factor cycle initiated by activated M2-type (alternatively activated) macrophages, but that can do so with very high specificity to the cells at issue.

### SUMMARY

In some instances, activated M2 phenotype macrophages play a role in cancers such as by secreting anti-inflammatory cytokines that activate fibroblasts to synthesize collagen and other extracellular matrix proteins. In certain instances, these macrophages similarly cause the release of growth factors that are problematic in subjects experiencing cancer. In some instances, activated macrophages, which derive from tissue-resident macrophages or peripheral blood monocytes, induce activation of fibroblasts via secretion of chemokine (C-C motif) ligand 18 (CCL18), transforming growth factor-β1 (TGFβ1) and/or platelet derived growth factor (PDGF). This activation, in some instances, promotes the secretion of collagen by the fibroblasts, which can cause cancer associated therewith to advance. In later stages of many cancers, the activated macrophages and myofibroblasts can cross-stimulate each other, resulting in promoted growth of cancerous tumors (e.g., owing to the growth factors secreted by the activated macrophages) and/or collagen formation in cancerous tumors (e.g., through downstream fibrotic collagen production, which can result in a cancerous tumor that is more difficult to treat by blocking drug penetrability thereof).

Provided herein in some embodiments is a compound represented by the formula Q-L-T. In some embodiments, Q is a radical of a folate receptor-binding ligand. In some embodiments, L is a linker. In some embodiments, T is a radical of a toll-like receptor (TLR) agonist. In some embodiments, Q-L-T is a pharmaceutically acceptable salt thereof

In some embodiments, the linker is a non-releasable linker. In some embodiments, the non-releasable linker is represented by the formula:

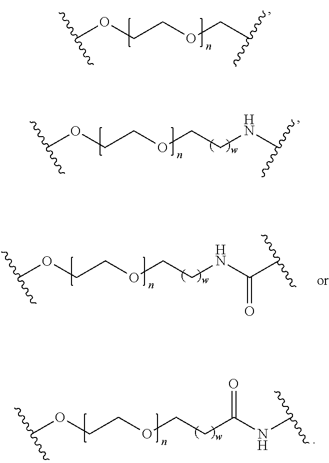

In some embodiments, n is 1-30. In some embodiments, n is 1-24. In some embodiments, n is 1-12. In some embodiments, n is 1-3. In some embodiments, n is 12. In some embodiments, n is 3.

In some embodiments, w is 0-5. In some embodiments, w is 0-2. In some embodiments w is 1.

Methods of treating a subject with cancer are also provided. In at least one embodiment of a method of treating a subject with cancer, the method comprises administering a first therapy to a subject, the first therapy comprising at least one compound or pharmaceutically acceptable salt or hydrate thereof comprising a radical of an immune modulator attached, via a linker, to a folate ligand or functional fragment or analog thereof, or a composition comprising the at least one compound and one or more pharmaceutically acceptable carriers, adjuvants, diluents, excipients, and/or vehicles, or combinations thereof; and administering a second therapy to the subject, the second therapy comprising one or more immune checkpoint inhibitors. The immune modulator (e.g., radical) or pharmaceutically acceptable salt or hydrate thereof can target a pattern recognition receptor or a damage associated molecular pattern (DAMP). In certain embodiments, administering the second therapy to the subject further comprises administering a therapeutically effective amount of the one or more immune checkpoint inhibitors. In certain embodiments, administering the first therapy to the subject comprises administering a therapeutically effective amount of the at least one compound or pharmaceutically acceptable salt or hydrate thereof, or composition comprising the at least one compound or a pharmaceutically acceptable salt or hydrate.

In at least one embodiment, administering the at least one compound or pharmaceutically acceptable salt or hydrate of the first therapy reprograms M2-type macrophages of the subject to M1-type macrophages of the subject and enhances a potency of the one or more checkpoint inhibitors of the second therapy relative to a baseline potency of the one or more checkpoint inhibitors.

In at least one embodiment of the method of treating a subject with cancer, the immune modulator (i.e., radical) is or comprises a toll-like receptor (TLR) 3 agonist, a TLR 7 agonist, a TLR 8 agonist, a TLR9 agonist, or a TLR 7/8 agonist. In certain embodiments of the method of treating a subject with cancer, the radical of the immune modulator is or comprises a TLR7 agonist and the linker is a non-releasable linker.

In at least one embodiment of a method of treating a subject with cancer. the at least one compound is

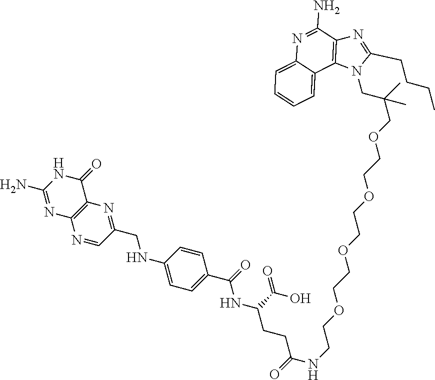

or is a pharmaceutically acceptable salt or hydrate thereof.

In at least one embodiment of a method of treating a subject with cancer, the at least one compound has the following formula:

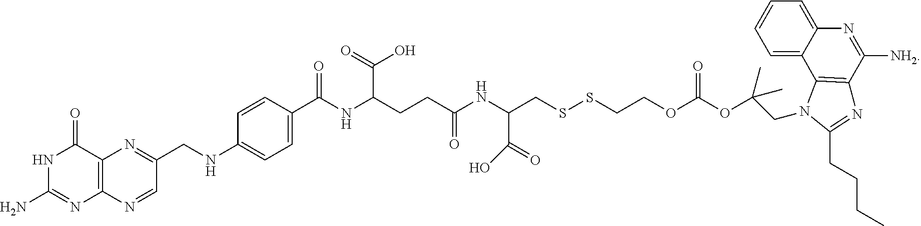 [SMILES: CCCCc1nc2c(N)nc3ccccc3c2n1CC(C)(C)OC(=O)OCCSSCC(NC(=O)CCC(NC(=O)c1ccc(NCc2cnc3nc(N)[nH]c(=O)c3n2)cc1)C(=O)O)C(=O)O]

or is a pharmaceutically acceptable salt or hydrate thereof.

In at least one embodiment of a method for treating a subject with cancer, the immune modulator (i.e., radical) is or comprises a TLR agonist of Formula X or XX, or is a pharmaceutically acceptable salt or hydrate of Formula X or XX:

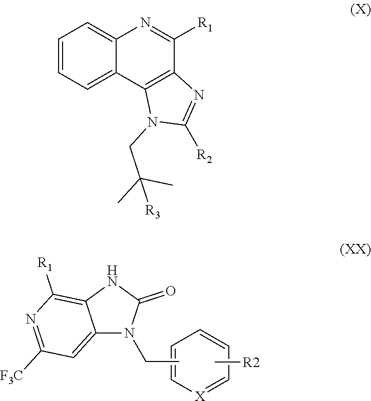

wherein, in Formulas X and XX:
R₁ is —NH₂ or —NH—R_{1X},
R₂ is an H, an alkyl, an alkenyl, an alkynyl, an alicyclic, an aryl, a biaryl, a heteroaryl, —NH—R_{2X}, —O—R_{2X}, —S—R_{2X}, or

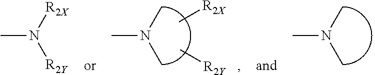

is a 3-10 membered N-containing non-aromatic mono- or bicyclic heterocycle, wherein:
in Formula X, R₃ is —OH, —SH, —NH₂ or —NH—R_{1X};
in Formula XX, X is a CH or an N; and
each of R_{1X}, R_{2X}, and R_{2Y} is independently selected from the group consisting of a hydrogen (H), an alkyl, an alkenyl, an alkynyl, an alicyclic, an aryl, a biaryl, and a heteroaryl.

In at least one embodiment of a method of treating a subject with cancer, the compound comprises:

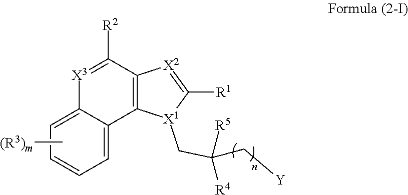

or is a pharmaceutically acceptable salt or hydrate thereof, wherein, in Formula (2-I):
R¹, R³, R⁴, and R⁵ are each independently a H, an alkyl, an alkoxyl, an alkenyl, an alkynyl, an alicyclic, an aryl, a biaryl, a halo, a heteroaryl, —COR^{2x},

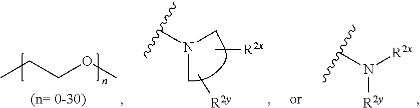

R² is a H, —OH, —NH₂, —NHR^{2x}, N₃, —NH—CH₂—NH₂, —CONH₂, —SO₂NH₂, —NH—CS—NH₂,

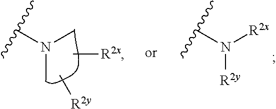

Y is a point of attachment to the linker and/or targeting ligand of the at least one compound and comprises a H, —OH, —NH₂, —NHR^{2x}, —O—R^{2x}, —SO—R^{2x}, —SH, —SO₃H, —N₃, —CHO, —COOH, —CONH₂, —COSH, —COR^{2x}, —SO₂NH₂, alkenyl, alkynyl, alkoxyl, —NH—CH₂—NH₂, —CONH₂, —SO₂NH₂, —NH—CS—NH₂,

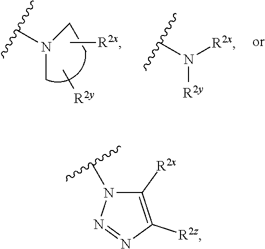

where:
   each of R^{2x} and R^{2y} is independently selected from the group consisting of H, —OH, —CH₂—OH, —NH₂, —CH₂—NH₂, —COOMe, —COOH, —CONH₂, —COCH₃, alkyl, alkenyl, alkynyl, alicyclic, aryl, biaryl, and heteroaryl, and each R² is independently selected from the group consisting of —NH₂, —NR^{2q}R^{2q′}, —O—R^{2q}, —SO—R^{2q}, and —COR^{2q}; wherein each of R^{2q} and R^{2q′} is independently alkyl or H; and

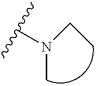 [SMILES: *N(C)CC]

is a 3-10 membered N-containing heterocycle that is non-aromatic, mono- or bicyclic;
wherein, in Formula 2-I, each of X¹, X², and X³ is independently CR^{q} or N, and each R^{q} is independently H, halogen, or an optionally substituted alkyl; and
wherein, in Formula 2-I, n is 0-30, and m is 0-4.

In at least one embodiment of a method of treating a subject with cancer, the subject is experiencing, or at risk for experiencing, a cancer or a relapse of a cancer, and the step of administering the first therapy further comprises administering or applying to the subject a therapeutically effective amount of the at least one compound or pharmaceutically acceptable salt or hydrate thereof.

In at least one embodiment of a method of treating a subject with cancer, the at least one compound or pharmaceutically acceptable salt or hydrate of the first therapy is administered to the subject intravenously, orally, intramuscularly, intraperitoneally, topically or by inhalation.

In at least one embodiment of a method of treating a subject with cancer, the M2-type macrophages of the subject comprise myeloid-derived suppressor cells (MDSCs), tumor-associated macrophages (TAMs), or both MDSCs and TAMs.

In at least one embodiment of the method, the subject has programmed death-ligand 1 (PD-L1), programmed death 1 (PD-1), or cytotoxic T-lymphocyte-associated antigen 4 (CTLA-4) positive tumors.

In at least one embodiment of the method, the at least one compound of the first therapy comprises a composition containing one or more pharmaceutically acceptable carriers, adjuvants, diluents, excipients, and/or vehicles, or combinations thereof.

In at least one embodiment of a method of treating a subject with cancer, the method is performed to treat a relapse of a cancer in the subject or resistance to checkpoint blockade therapy in the subject. The subject can be a human, a mouse, or any other mammal.

In certain embodiments of the method of treating a subject with cancer, the one or more immune checkpoint inhibitors of the second therapy each comprises a small molecule or other agent that disrupts an immune checkpoint of a cell of the subject. In certain embodiments of the method, the one or more immune checkpoint inhibitors of the second therapy are each independently selected from the group consisting of pembrolizumab, nivolumab, ipilimumab, cemiplimab, atezolizumab, avelumab, durvalumab, pidilizumab, monoclonal antibody MEDI-0680, monoclonal antibody REGN2810, or fusion protein AMP-224 that targets PD-1, ociperlimab, islelizumab, a combination of ociperlimab and islelizumab, BMS-936559/MDX-1105, MPDL3280A/RG7446/atezolizumab, MSB0010718C/avelumab, or MED14736/durvalumab, tiragolumab, zimberelimab, tremelimumab, relatlimab, monoclonal antibody IMP321, nivolumab, etigilimab, domvanalimab, tiragolumab (RG6058), vibostolimab, avelumab, and durvalumab. In at least one embodiment of a method of treating a subject with cancer, the one or more checkpoint inhibitors of the second therapy comprise one or more of pembrolizumab, nivolumab, and ipilimumab, cemiplimab, atezolizumab, avelumab, durvalumab, or a small molecule or other agent that disrupts an immune checkpoint of a cell of the subject.

In at least one embodiment of a method of treating a subject with cancer, the method further comprises obtaining a biological sample from the subject and quantifying a level of expression of one or more biomarkers in the biological sample. In at least one embodiment of a method of treating a subject with cancer, the biological sample is obtained from an amount of peripheral blood drawn from the subject. In at least one embodiment a method of treating a subject with cancer, quantifying is performed using a process selected from a group consisting of qPCR, mass spectrometry. ELISA, and another modality that is capable to measure or quantify biomarker expression. The one or more biomarkers can be, for example, selected from a group consisting of CCL18, Arg1, MMP9, TIMP 3, IL-1β, PDGF, TGFβ, FRβ, hydroxyproline, collagen, TNFα, IFN-γ, CD206, CD163, IL-6, CXCL10, IFNα, and CD86.

In at least one embodiment of a method of treating a subject with cancer, the method further comprises comparing a level of expression of each of the one or more biomarkers to an expression level of such biomarker in a control, wherein the control is a healthy individual or an individual that is not experiencing cancer.

In at least one embodiment of a method of treating a subject with cancer, the radical of the immune modulator is or comprises a TLR agonist having the following formula:

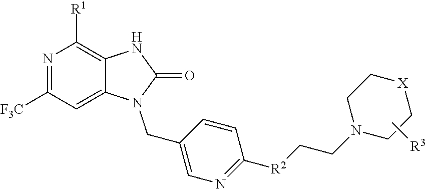

wherein:
R¹ is an amine group:
R² is a single bond —NH—;
R³ is an H, an alkyl, a hydroxy group, or any other substituted group thereof,
X is a CH₂. NH, O. or S; and
the linker is attached at R¹, R² or R.

In at least one embodiment of a method of treating a subject with cancer, the linker of the at least one compound or pharmaceutically acceptable salt or hydrate of the first therapy is a releasable linker. In at least one embodiment of a method of treating a subject with cancer, the linker of the at least one compound or pharmaceutically acceptable salt or hydrate of the first therapy is a non-releasable linker. In at least one embodiment of a method of treating a subject with cancer, the linker of the at least one compound or pharmaceutically acceptable salt or hydrate of the first therapy comprises a polyethylene glycol (PEG) linker or a PEG derivative linker and is a non-releasable linker.

In at least one embodiment of a method of treating a subject with cancer, the one or more immune checkpoint inhibitors of the second therapy inhibit an immune checkpoint of a cell selected from a group consisting of PD-L1, PD-1, CTLA-4, V-domain Ig suppressor of T cell activation (VISTA), lymphocyte activating 3 (LAG3), T cell immunoglobulin and mucin domain-containing protein 3 (TIM3). T cell immunoreceptor with Ig and ITIM domains (TIGIT), programmed death ligand 2 (PD-L2), indoleamine 2,3-deioxygenase (IDO), arginase-1 (AGR1), B7 family inhibitory ligand B7-H3 (B7-H3), B7 family inhibitory ligand B7-H4 (B7-H4), 2B4 (cluster of differentiation 244), B- and T-lymphocyte attenuator (BTLA), adenosine A2A receptor (A2aR), and/or a member of the family of killer cell innunoglobulin-like receptors (KIRs) such as KIRs and C-type lectin receptors, and signal transducer and activator of transcription (STAT3).

In at least one embodiment of a method of treating a subject with cancer, the first and second therapies are administered simultaneously or sequentially (in either order). In certain embodiments, administering the at least one compound or pharmaceutically acceptable salt or hydrate of the first therapy activates anti-tumor cells or an anti-inflammatory signaling cascade in the subject. The anti-tumor cells can be, for example. T cells, macrophages, or both T cells and macrophages.

Additional methods for enhancing a potency of one or more immune checkpoint inhibitors administered to a subject are provided. In certain embodiments, a method of enhancing a potency of one or more immune checkpoint inhibitors administered to a subject comprises: administering, to the subject, one or more compounds or pharmaceutically acceptable salts or hydrates thereof comprising a radical of an immune modulator attached, via a linker, to a folate ligand or functional fragment or analog thereof, wherein the radical of the immune modulator targets a pattern recognition receptor or DAMP, and contacting a targeted cell of the subject with the one or more compounds or pharmaceutically acceptable salts or hydrates to reprogram M2-type macrophages of the subject to M1-type macrophages.

In some instances, activated M2 phenotype macrophages play a role in fibrotic diseases, such as by secreting anti-inflammatory cytokines that activate fibroblasts to synthesize collagen and other extracellular matrix proteins. In certain instances, these macrophages similarly cause the release of growth factors that are problematic in subjects experiencing cancer. For example, such growth factors can promote growth of cancerous tumors. Moreover, in some instances, macrophages (e.g., concurrently) release immune suppression cytokines. As such, macrophages can play an important role in facilitating the establishment and growth of fibrotic disease and/or cancer.

In some instances, activated macrophages, which derive from tissue-resident macrophages or peripheral blood monocytes, induce activation of fibroblasts via secretion of chemokine (C-C motif) ligand 18 (CCL18), transforming growth factor-β1 (TGFβ1) and/or platelet derived growth factor (PDGF). This activation, in some instances, promotes the secretion of collagen by the fibroblasts, which can cause cancer associated therewith to advance. In later stages of many cancers, the activated macrophages and myofibroblasts can cross-stimulate each other, resulting in promoted growth of cancerous tumors (e.g., owing to the growth factors secreted by the activated macrophages) and/or collagen formation in cancerous tumors (e.g., through downstream fibrotic collagen production, which can result in a cancerous tumor that is more difficult to treat by blocking drug penetrability thereof).

In certain embodiments of the method of enhancing a potency of one or more immune checkpoint inhibitors administered to a subject, the radical of the immune modulator is or comprises a TLR 3 agonist, a TLR 7 agonist, a TLR 8 agonist, a TLR9 agonist, or a TLR 7/8 agonist. In certain embodiments of the method of enhancing a potency of one or more immune checkpoint inhibitors administered to a subject, the radical of the immune modulator is or comprises a TLR7 agonist and the linker is a releasable linker. In certain embodiments of the method of enhancing a potency of one or more immune checkpoint inhibitors administered to a subject, the radical of the immune modulator is or comprises a TLR7 agonist and the linker is a non-releasable linker.

In certain embodiments of the method of enhancing a potency of one or more immune checkpoint inhibitors administered to a subject, the one or more compounds is or comprises a structure represented by:

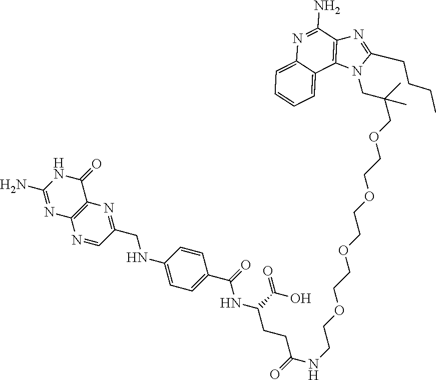

or is a pharmaceutically acceptable salt or hydrate thereof.

The linker can be a non-releasable linker. The linker can be a releasable linker. In certain embodiments, the cancerous disease state comprises a cancer recurrence.

Provided herein in some embodiments is a compound represented by the formula Q-L-T, or a pharmaceutically acceptable salt or hydrate thereof. In some embodiments, Q is a radical of a folate receptor-binding ligand. In some embodiments, L is a linker. In some embodiments, T is a radical of a TLR agonist. In some embodiments, Q-L-T is a pharmaceutically acceptable salt thereof.

In some embodiments, the linker is a non-releasable linker. In some embodiments, the non-releasable linker is represented by the formula:

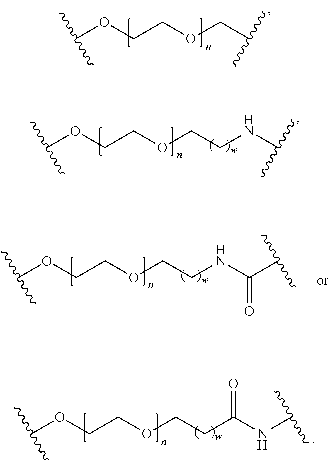

In some embodiments, n is 1-30. In some embodiments, n is 1-24. In some embodiments, n is 1-12. In some embodiments, n is 1-3. In some embodiments, n is 12. In some embodiments, n is 3.

In some embodiments, w is 0-5. In some embodiments, w is 0-2. In some embodiments w is 1.

In some embodiments, the TLR agonist is a TLR7 agonist. In some embodiments, the radical of the TLR agonist has a structure represented by Formula X:

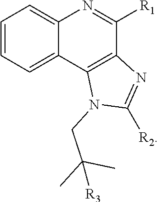

In some embodiments, R₁ is —NH₂ or —NH—R_{1X}. In some embodiments, R₂ is an H, an alkyl, an alkenyl, an alkynyl, an alicyclic, an aryl, a biaryl, a heteroaryl, —NH—R_{2X}, —O—R_{2X}, —S—R_{2X},

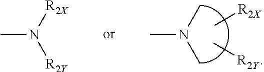

In some embodiments, each of R_{1X}, R_{2X}, and R_{2Y} is independently selected from the group consisting of a hydrogen (H), an alkyl, an alkenyl, an alkynyl, an alicyclic, an aryl, a biaryl, and a heteroaryl. In some embodiments,

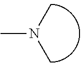

is a 3-10 membered nitrogen (N)-containing non-aromatic mono- or bicyclic heterocycle. In some embodiments, R₃ is —OH, —SH, —NH₂ or —NH—R_{1X}. In some embodiments, R₁ is —NH₂ or —NH—R_{1X}; R₂ is an H, an alkyl, an alkenyl, an alkynyl, an alicyclic, an aryl, a biaryl, a heteroaryl, —NH—R_{2X}, —O—R_{2X}, —S—R_{2X},

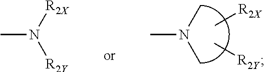

each of R_{1X}, R_{2X}, and R_{2Y} is independently selected from the group consisting of an H, an alkyl, an alkenyl, an alkynyl, an alicyclic, an aryl, a biaryl and a heteroaryl.

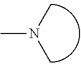

is a 3-10 membered N-containing non-aromatic mono- or bicyclic heterocycle; and R₃ is —OH, —SH, —NH₂ or —NH—R_{1X}.

In some embodiments, the radical of the TLR agonist has a structure represented by Formula XX:

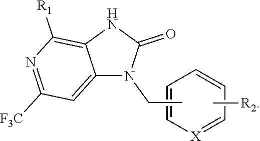

R₁ can be —NH₂ or —NH—R_{1X}, R₂ can be an H, an alkyl, an alkenyl, an alkynyl, an alicyclic, an aryl, a biaryl, a heteroaryl, —NH—R₂ₓ, —O—R₂ₓ, —S—R_{2X},

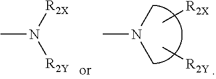

In some embodiments, each of R_{1X}, R_{2X}, and R_{2Y} is independently selected from the group consisting of an H, an alkyl, an alkenyl, an alkynyl, an alicyclic, an aryl, a biaryl, and a heteroaryl. In some embodiments,

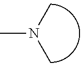

is a 3-10 membered N-containing non-aromatic mono- or bicyclic heterocycle. In some embodiments, X is CH, CR₂, or N. In some embodiments, R₁ is —NH₂ or —NH—R_{1X}; R₂ is an H, an alkyl, an alkenyl, an alkynyl, an alicyclic, an aryl, a biaryl, a heteroaryl, —NH—R_{2X}, —O—R_{2X}, —S—R_{2X},

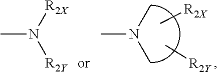

each of R_{1X}, R_{2X}, and R_{2Y} is independently selected from the group consisting of an H, an alkyl, an alkenyl, an alkynyl, an alicyclic, an aryl, a biaryl and a heteroaryl;

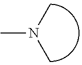

is a 3-10 membered N-containing non-aromatic mono- or bicyclic heterocycle; and X is CH, CR₂, or N.

In some embodiments, the radical of the TLR7 agonist has a structure represented by Formula XXX:

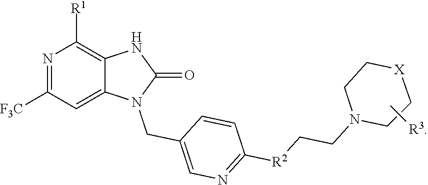

In some embodiments, the compound further comprises a linker Lₙ between the targeting moiety and the immune modulator or the pharmaceutically acceptable salt thereof, wherein the linker Lₙ is configured to avoid release of a free form of the TLR7 agonist, and n is an integer equal to or less than 50. In some embodiments, the linker Lₙ comprises PEG or a PEG derivative, n is an integer selected from the range 1-32, and the radical of folate receptor-binding ligand is a folate receptor β (FBβ) binding ligand.

In some embodiments, the compound has a structure represented by:

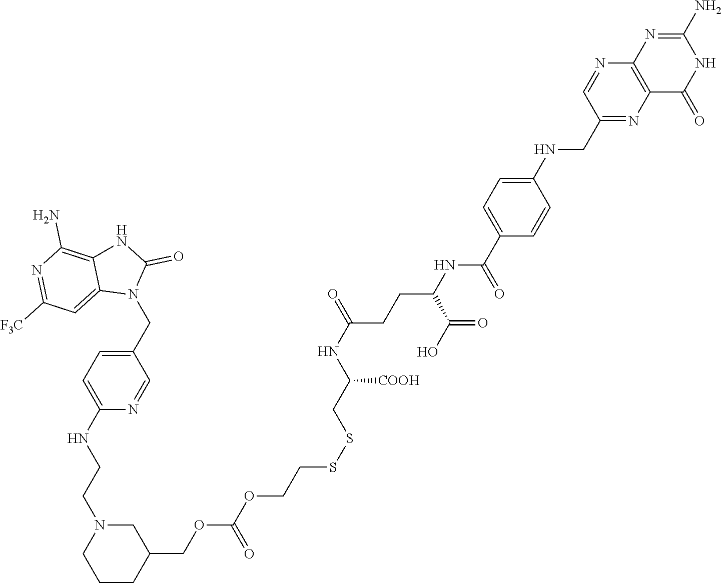

or is a pharmaceutically acceptable salt or hydrate thereof.

In some embodiments, the compound has a structure represented by:

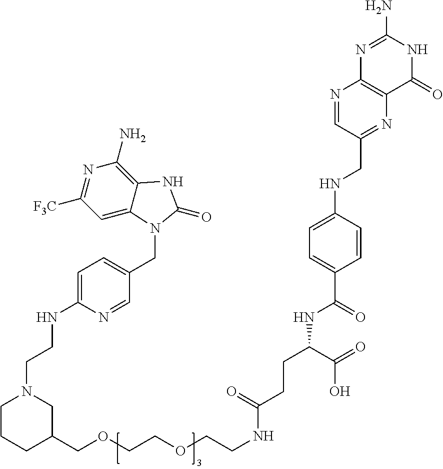

or is a pharmaceutically acceptable salt or hydrate thereof.

In some embodiments, the compound has a structure represented by:

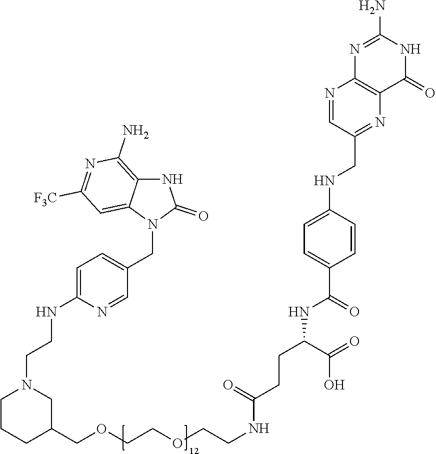

or is a pharmaceutically acceptable salt or hydrate thereof

In some embodiments, the compound has a structure represented by:

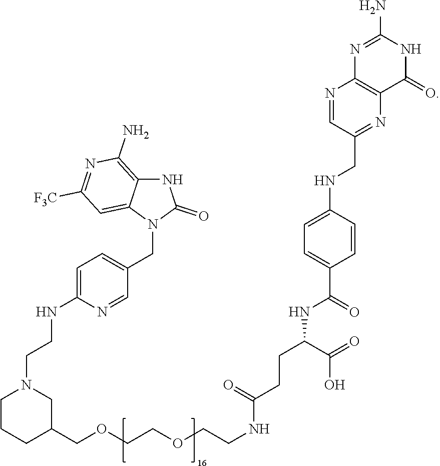

or is a pharmaceutically acceptable salt or hydrate thereof

In some embodiments provided herein is a pharmaceutical composition comprising one or more of the compounds described herein or a pharmaceutically acceptable salt or hydrate thereof, wherein the radical of the immune modulator is or comprises a TLR7 agonist having a structure represented by Formula XX.

In certain instances, provided herein is a method of treating a subject suffering from a fibrotic disease state or a cancer, the method comprising contacting a cell of the subject with at least one compound comprising a compound or pharmaceutically acceptable salt or hydrate described herein, wherein the radical of the immune modulator comprises an agonist of TLR 3, 7, 8, 9, or 7/8.

In some embodiments, provided herein is a compound comprising a folate ligand or a functional fragment or analog thereof attached to a TLR agonist via a linker, the TLR agonist having the following formula or a pharmaceutically acceptable salt thereof:

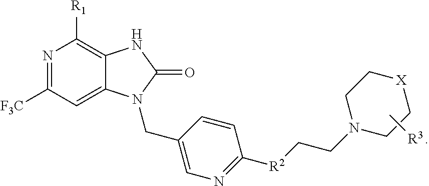

In some embodiments, R¹ is an amine group, R² is a single bond —NH—, and R³ is an H, an alkyl, a hydroxy group, or any other substituted group thereof, X is a CH₂, NH, oxygen (O), or sulfur (S), and the linker is attached at R¹, R² or R³.

Provided in some embodiments herein is a pharmaceutical composition comprising the compound of any one of the formulas provided herein, wherein the linker comprises a PEG linker or a PEG derivative linker and is either a non-releasable linker attached at R³ or is a releasable linker attached at R¹, R² or R³.

In some embodiments, the pharmaceutically acceptable salt is selected from hydrobromide, citrate, trifluoroacetate, ascorbate, hydrochloride, tartrate, triflate, maleate, mesylate, formate, acetate or fumarate.

Combinations for use employing any of the compounds/conjugates described herein are also provided. In certain embodiments, the combination is for use in the treatment of a cancer in a subject and the combination comprises: a first medicament comprising and a second medicament. The first medicament can comprise at least one compound or pharmaceutically acceptable salt or hydrate thereof comprising a radical of an immune modulator attached, via a linker, to a folate ligand or functional fragment or analog thereof, wherein the radical of the immune modulator targets a pattern recognition receptor or a DAMP, or a composition comprising the at least one compound or pharmaceutically acceptable salt or hydrate thereof, and one or more pharmaceutically acceptable carriers, adjuvants, diluents, excipients, and/or vehicles. Additionally, the second medicament can comprise one or more immune checkpoint inhibitors.

In the combination, the radical of the immune modulator of the first medicament can comprise a TLR 3 agonist, a TLR 7 agonist, a TLR 8 agonist, a TLR9 agonist, or a TLR 7/8 agonist (i.e., a TLR 3, 7, 8, 9, or 7/8 agonist). The at least one compound of the first medicament of the combination can have the following formula:

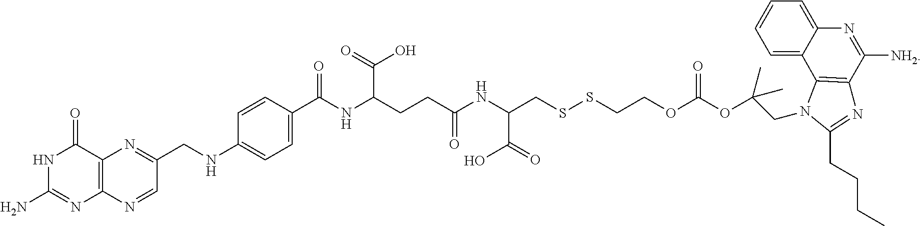

or can be a pharmaceutically acceptable salt or hydrate thereof. In certain embodiments, the radical of the immune modulator comprises a TLR agonist of Formula X or XX, or is a pharmaceutically acceptable salt or hydrate of Formula X or XX:

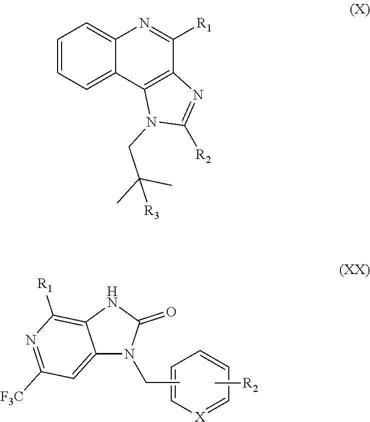

wherein, in Formulas X and XX:
R₁ is —NH₂ or —NH—R_{1X},
R₂ is an H, an alkyl, an alkenyl, an alkynyl, an alicyclic, an aryl, a biaryl, a heteroaryl, —NH—R_{2X}, —O—R_{2X}, —S—R_{2X},

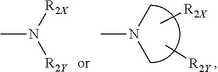

and

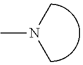

is a 3-10 membered N-containing non-aromatic mono- or bicyclic heterocycle, wherein:
in Formula X, R₃ is —OH, —SH, —NH₂ or —NH—R_{1X};
in Formula XX, X is a CH or an N; and
each of R_{1X}, R_{2X}, and R_{2Y} is independently selected from the group consisting of an H, an alkyl, an alkenyl, an alkynyl, an alicyclic, an aryl, a biaryl, and a heteroaryl.

In certain embodiments of the combination, the at least one compound of the first medicament comprises:

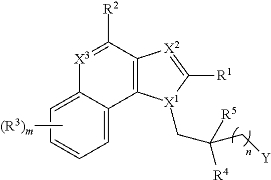

or is a pharmaceutically acceptable salt or hydrate thereof, wherein, in Formula 2-I:
R¹, R³, R⁴, and R⁵ are each independently H, an alkyl, an alkoxyl, an alkenyl, an alkynyl, an alicyclic, an aryl, a biaryl, a halo, a heteroaryl, —COR^{2x},

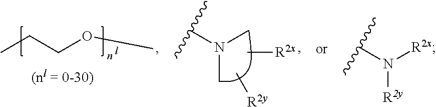

R² is a H, —OH, —NH₂, —NHR^{2x}, N₃, —NH—CH₂—NH₂, —CONH₂, —SO₂NH₂, —NH—CS—NH₂,

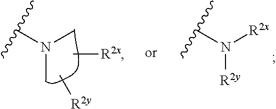

Y is a point of attachment to the linker and/or a targeting ligand of the at least one compound and comprises a H, —OH, —NH₂, —NHR^{2x}, —O—R^{2x}, —SO—R^{2x}, —SH, —SO₃H, —N₃, —CHO, —COOH, —CONH₂, —COSH, —COR^{2x}, —SO₂NH₂, alkenyl, alkynyl, alkoxyl, —NH—CH₂—NH₂, —CONH₂, —SO₂NH₂, —NH—CS—NH₂,

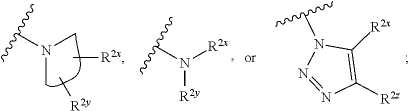

where:
   each of R^{2x} and R^{2y} is independently selected from the group consisting of H, —OH, —CH₂—OH, —NH₂, —CH₂—NH₂, —COOMe, —COOH, —CONH₂, —COCH₃, alkyl, alkenyl, alkynyl, alicyclic, aryl, biaryl, and heteroaryl, and each R^{2z} is independently selected from the group consisting of —NH₂, —NR^{2q}R^{2q′}, —O—R^{2q}, —SO—R^{2q}, and —COR^{2q}; wherein each of R^{2q} and R^{2q′} is independently alkyl or H; and

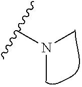 [SMILES: *N(C)C]

is a 3-10 membered N-containing heterocycle that is non-aromatic, mono- or bicyclic;
wherein, in Formula 2-I, each of X¹, X², and X³ is independently CR^{q} or N, and each R is independently H, halogen, or an optionally substituted alkyl; and
wherein, in Formula 2-I, n is 0-30, and m is 0-4.

In certain embodiments of the combination, the radical of the immune modulator of the first medicament comprises a TLR agonist having the following formula or a pharmaceutically acceptable salt thereof:

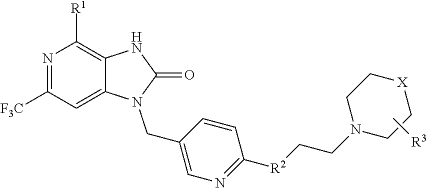

wherein:
R¹ is an amine group,
R² is a single bond —NH—.
R³ is an H, an alkyl, a hydroxy group, or any other substituted group thereof,
X is a CH₂, NH, O, or S, and
the linker is attached at R¹, R² or R³.

In certain embodiments of the combination, the at least one compound of the first medicament is

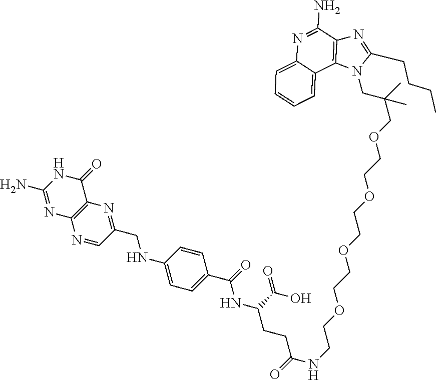

or is a pharmaceutically acceptable salt or hydrate thereof.

The linker of the first medicament of the combination can be a releasable linker. The linker of the first medicament of the combination can be a non-releasable linker. The linker of the first medicament of the combination can be or comprise a PEG linker or a PEG derivative linker and is a non-releasable linker.

In certain embodiments of the combination, the subject has a PD-L1, PD-1, or CTLA-4 positive tumor.

In certain embodiments, the one or more immune checkpoint inhibitors of the second medicament comprise a small molecule or other agent that disrupts an immune checkpoint of a cell of the subject. In certain embodiments of the combination, the one or more checkpoint inhibitors of the second medicament are each independently selected from the group consisting of pembrolizumab, nivolumab, ipilimumab, ceniplimab, atezolizumab, avelumab, durvalumab, pidilizumab, monoclonal antibody MEDI-0680, monoclonal antibody REGN2810, or fusion protein AMP-224 that targets PD-1, ociperlimab, islelizumab, a combination of ociperlimab and islelizumab, BMS-936559/MDX-1105, MPDL3280A/RG7446/atezolizumab, MSB0010718C/avelumab, or MEDI4736/durvalumab, tiragolumab, zimberelimab, tremelimumab, relatlimab, monoclonal antibody IMP321, nivolumab, etigilimab, domvanalimab, tiragolumab (RG6058), vibostolimab, avelumab, and durvalumab.

In certain embodiments of the combination, the one or more immune checkpoint inhibitors of the second medicament are one or more agents that bind to or inhibit an immune checkpoint of a cell selected from a group consisting of PD-1, PD-L1, CTLA-4, VISTA, LAG3, TIM3, TIGIT, PD-L2. IDO, AGR1, B7-H3. B7-H4, 2B4. BTLA. A2aR, and/or a member of the family of KIRs such as KIRs and C-type lectin receptors, and STAT3.

The cancer can be or comprise a cold tumor, a hot tumor, or an immune desert tumor.

In certain embodiments, any of the combinations described herein can be used for enhancing a potency of one or more immune checkpoint inhibitors administered to a subject with cancer and/or for treating a cancer.

Provided in some embodiments herein is a method of preventing or treating a cancerous disease state comprising contacting a cell with at least one compound (e.g., any compound provided by a formula provided herein) comprising an immune modulator or pharmaceutically acceptable salt thereof attached, via a linker, to a folate ligand or functional fragment or analog thereof, wherein the immune modulator or pharmaceutically acceptable salt thereof targets a pattern recognition receptor or a DAMP. In some embodiments, the cell comprises a cell of a subject experiencing, or at risk for experiencing, a fibrotic disease state and contacting the cell with at least one compound further comprises administering or applying to the subject a therapeutically effective amount of the at least one compound. In some embodiments, the subject is a patient experiencing IPF and the at least one compound is administered to the subject intravenously, orally, intramuscularly, intraperitoneally, topically or by inhalation. In some embodiments, the fibrotic disease state comprises IPF or a fibrotic disease of a liver, skin, bladder, heart, pancreas, prostate, or kidneys.

In some embodiments, the method further comprises obtaining, or having obtained, a sample from the subject; quantifying a level of expression of one or more biomarkers in the sample, each of the one or more biomarkers selected from the group consisting of CCL18, Arg1, MMP9, TIMP3, IL-1β, hydroxyproline, collagen, PDGF, TGFβ, FRβ, TNFα, IFN-γ, CD206, CD163, CD86, IL-6, CXCL10, and IFNα; comparing the level of expression of each of the one or more biomarkers in the sample to an expression level of such biomarker in a control; and administering or having administered to the subject a therapeutically effective amount of an unconjugated agonist or inhibitor if CCL18, Arg1, MMP9, TIMP 3, IL-1β, PDGF, TGFβ, FRβ, CD206, CD163, hydroxyproline, or collagen is upregulated relative to the expression level of the control or TNFα, IFN-γ, IL-6, CXCL10, IFNα or CD86 is downregulated or not expressed relative to the expression level of the control.

In some embodiments, the folate ligand or functional fragment or analog thereof is specific for FRβ and binds to a FRβ on the cell. Provided in some embodiments is a method of treating a subject experiencing a cancerous disease state (e.g., a cancer or a cancer recurrence/relapse) comprising enhancing a potency of one or more checkpoint inhibitors administered to the subject by administering one or more compounds (or pharmaceutically acceptable salts or hydrates thereof) comprising a targeting moiety (e.g., a folate ligand or functional fragment or analog thereof) attached, via a linker, to a radical of an immune modulator or a pharmaceutically acceptable salt thereof (e.g., any TLR agonist, including without limitation, a TLR 3, 7, 8, 9, or 7/8 agonist), wherein the targeting moiety targets a pattern recognition receptor or a DAMP of a cell. In certain embodiments, contacting a targeted cell of the subject with the one or more compounds or pharmaceutically acceptable salts or hydrates thereof reprograms M2-type macrophages of the subject to M1-type macrophages. In at least one exemplary embodiment, the radical of the immune modulator or pharmaceutically acceptable salt thereof is a TLR7 agonist and the linker is a releasable linker. In at least one additional embodiment, the linker is a non-releasable linker.

In certain embodiments, administering the at least one compound or pharmaceutically acceptable salt or hydrate thereof (e.g., of the first therapy or first medicament) activates anti-tumor cells or an anti-inflammatory signaling cascade in the subject. In at least one embodiment, such anti-tumor cells are T cells. Additionally or alternatively, such anti-tumor cells can be macrophages.

### DESCRIPTION OF THE DRAWINGS

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings, wherein.

FIG. 1A shows the chemical structure of an exemplary compound having a targeting moiety (e.g., a folate receptor ligand) attached to an immune modulator (e.g., a radical of a toll-like receptor (TLR) 7 (TLR7) agonist) via a non-releasable linker (e.g., comprising a polyethylene glycol (PEG) backbone portion).

FIG. 1B shows the chemical structure of an exemplary compound having a targeting moiety (e.g., folate receptor ligand) attached to an immune modulator (e.g., TLR7 agonist radical) via a releasable linker (e.g., comprising a disulfide portion in the backbone thereof), as well as an exemplary drug release mechanism.

FIG. 1C shows the chemical structure of exemplary compounds provided herein.

FIG. 1D shows how checkpoint proteins, such as programmed death-ligand 1 (PD-L1) on tumor cells and programmed cell death protein 1 (PD-1) on T cells, help keep immune responses in check. The binding of PD-L1 to PD-1 keeps T cells from killing tumor cells in the body (left panel). Blocking the binding of PD-L1 to PD-1 with an immune checkpoint inhibitor (anti-PD-L1 or anti-PD-1) allows the T cells to kill tumor cells (right panel).

FIG. 2 shows a flow chart representative of methods for treating a subject experiencing, or at risk for experiencing, a fibrotic disease or a cancer.

FIGS. 3A-3F are graphical data of various marker levels measured from human M2-type macrophages when contacted with an exemplary free (non-targeted) TLR7 agonist or an exemplary targeted (e.g., with a folate receptor-binding ligand) TLR7 agonist at various concentrations for each compound. Each value represents the mean S.D, for each group; #P<0.05, ##P<0.01, ###P<0.005, ####P<0.0001; treated groups versus M2-untreated group by Dunnett's multiple comparison test.

FIGS. 4A-4E and FIGS. 5A-5D show graphical data representative of various marker levels measured from M2 macrophages that were incubated with various concentrations of exemplary free- or targeted-TLR7 agonists for 2 hours (FIGS. 4A-4E), or 46 hours (FIGS. 5A-5D). Each value represents the mean±S.D, for each group; #P<0.05, ##P<0.01, ###P<0.005, ####P<0.0001; Compound 1A and Compound 1B treated groups in FIGS. 4A-5D versus M2-untreated group by Dunnett's multiple comparison test.

FIGS. 6A-6D show graphical data representative of various marker levels measured from M2 macrophages treated with various concentrations of exemplary free and targeted TLR7 agonists for: (i) 48 hours (FIGS. 6A and 6B); or (ii) 2 hours, then displaced with fresh medium and cultured for the remaining 46 hours (FIGS. 6C and 6D). Each value represents the mean±S.D, for each group; #P<0.05. ##P<0.01. ###P<0.005, ####P<0.0001; Compound 1A and Compound 1B treated groups versus M2-untreated group by Dunnett's multiple comparison test.

FIG. 6E shows flow cytometry data supporting that the THP-1 (a human monocytic cell line derived from an acute monocytic leukemia patient) induced macrophages were folate receptor beta (FRβ)-positive (FRβ⁺).

FIG. 6F show that exemplary targeted TLR7 agonists are stable.

FIG. 7A shows stained images of lungs taken from mice with bleomycin (BM)-induced experimental fibrosis and stained using anti-mouse FRβ antibody, with the hematoxylin-eosin (H&E) staining performed on days 7, 14, and 21 post-BM-induced lung injury.

FIG. 7B shows quantification of FRβ staining in the panels of FIG. 7A.

FIGS. 7C and 7D show FRβ immunohistochemistry (IHC) staining of human idiopathic pulmonary fibrosis (IPF) lung tissue (FIG. 7C) and healthy human lung tissue (FIG. 7D).

FIG. 7E shows images of mice tissues/organs taken from mice with BM or without (phosphate-buffered saline (PBS) control) BM-induced experimental fibrosis and imaged with a folate receptor-targeted fluorescent dye.

FIG. 7F shows a fluorescence-activated cell sorter (FACS) analysis of mice with BM-induced experimental fibrosis.

FIG. 8A illustrates the treatment plan of free and targeted TLR7 agonists in a BM model.

FIGS. 8B-8G show pro-fibrotic marker levels (FIGS. 8B-8D) and pro-inflammatory marker levels (FIGS. 8E-8G) measured from mice treated with the BM model of FIG. 8A.

FIG. 8H shows the number of cells in the bronchoalveolar lavage fluid (BALF) from mice treated with the BM model of FIG. 8A.

FIGS. 9A and 9B show survival curves (FIG. 9A) and body weight change (FIG. 9B) of mice with pulmonary fibrosis treated with non-targeted and targeted TLR7 drugs.

FIG. 10A shows the hydroxyproline content (pg/lung) of lung tissue as a measure of fibrosis.

FIGS. 10B and 10C show lung tissue in FIG. 9A with H&E staining (FIG. 10B) and Masson's trichrome (collagen) staining (FIG. 10C).

FIGS. 11A and 11B show survival curves (FIG. 11A) and body weight change (FIG. 11B) of mice with pulmonary fibrosis treated with exemplary targeted TLR7 agonists, with each value representing the mean±S.D, for each group.

FIG. 12 shows the dose-dependent effect of an exemplary targeted TLR7 agonist on the suppression of fibrosis in BM-induced mice. FIG. 12A shows graphical data related to the body weight of the BM-induced mice over time. FIG. 12B shows measurement of hydroxyproline content of the lung tissue treated with various doses of exemplary compound provided herein (e.g., Compound 1B). FIG. 12C shows images for histological analysis of lung tissue with various stains. Each value represents the mean±S.D, for each group: *P<0.05. **P<0.005, ***<0.0005; saline versus vehicle group, the treated groups versus vehicle group by Student's t test.

FIGS. 13A-13D show various marker levels measured from M2-type macrophages reprogrammed pursuant to methods hereof with various concentrations of an exemplary targeted TLR7 agonist for 48 hours and each value representing the mean I S.D, for each group.

FIGS. 14A-14C show various marker levels measured from M2-type macrophages reprogrammed pursuant to methods hereof with various concentrations of exemplary free and targeted TLR7 agonists. FIG. 14A shows CCL18, FIG. 14B shows CD206, and FIG. 14C shows IL-1β. Each value shown in FIGS. 14A-14C represents the mean±S.D, for each group; #P<0.05, ##P<0.005, ###P<0.0005; ###P<0.0001; Compound 3A, Compound 3B-treated, and Compound 3C-treated groups versus M2-untreated group by Dunnett's multiple comparison test.

FIG. 15 shows secreted chemokine (C-C motif) ligand 18 (CCL18) protein levels in each group of cells of FIGS. 14A-14C after treatment with exemplary free and targeted TLR7 agonists.

FIG. 16 illustrates a methodology for a BM murine model.

FIGS. 17A and 17B show the purity of an exemplary targeted TLR7 agonist.

FIGS. 18A-18F show data from the in vivo study of FIG. 16, including survival curves (FIG. 18A), body weight changes (FIGS. 18B and 18D), concentration of cells with BALF present (FIG. 18C), hydroxyproline concentration (pig HP/lobe) in live mice (FIG. 18E) and in all mice (i.e., inclusive of both live mice and those that died before day 21) (FIG. 18F).

FIGS. 19A-19F show that both targeted and nontargeted TLR7 agonists reprogram human monocyte-derived anti-inflammatory macrophages to an anti-fibrotic phenotype, with FIG. 19A showing Arg1, FIG. 19B showing CD206, FIG. 19C showing CD163, FIG. 19D showing CCL18, FIG. 19E showing CXCL10, and FIG. 19F showing IL-6. Mean±SD. Statistical significance between groups was determined using unpaired two-tailed t-test (*P<0.05, **P<0.01, ***P<0.001, ****P<0.001).

FIG. 20 shows comparison of plasma cytokine levels in healthy mice following treatment with Compound 1A versus Compound 1B (FIGS. 20A-20F), with FIGS. 20A and 20D showing IL-6, FIGS. 20B and 20E showing IFNα, and FIGS. 20C and 20F showing tumor necrosis factor a (TNF-α). FIG. 20G shows the change in body weight after treatment of mice with exemplary compounds, with change in body weight as a measure of systemic toxicity during alternate day dosing (n=2); mean±SD. Statistical significance between groups was compared using unpaired two-tailed t-test (*P<0.05, **P<0.01, ***P<0.001).

FIG. 21 shows healthy and fibrotic lungs described in FIG. 6 stained with 4′,6-diamidino-2-phenylindole (DAPI) (nuclei; blue), anti-F4/80 (macrophages; red), and anti-mannose receptor (CD206).

FIG. 22 shows the effect of various exemplary compounds on interleukin 6 (IL-6) expression in peripheral blood mononuclear cells.

FIGS. 23A and 23B show the in vitro effects of various exemplary compounds on IL-6 and C-X-C motif chemokine 10 (CXCL-10) induction in monocyte derived M2-macrophages for 48 hours.

FIGS. 23C and 23D show the in vivo effects of various exemplary compounds on IL-6 and tumor necrosis factor α (TNF-α) production.

FIGS. 24A-24F show data from in vivo combination studies of FA-TLR7-1A non-releasable conjugate (Compound 1000) (3 nmol/mouse) with anti-CTLA-4 (10 mg/kg, twice a week) in a 4T1 orthotopic solid tumor model, with FIG. 24A showing tumor volume measured every other day after the treatment; FIG. 24B showing a graph of tumor weight measured at the end of the study; FIG. 24C showing graphs of tumor volumes in the individual mice in the different treatment groups: FIG. 24D showing a graph of tumor volume following treatment with FA-TLR7-1A, PD-1, and combination FA-TLR7-TA+PD-L: FIG. 24E showing plots of single mice tumor volume following treatment with either PD-1 or a combination of FA-TLR7-1A+PD-1; and FIG. 24F showing a graph of tumor volume versus days post treatment initiation.

FIG. 25 shows graphs of immune cell analysis results from digested tumor tissues after in vivo combination treatment of FA-TLR7-IA slow-release conjugate (3 nmol/mouse) with anti-CTLA-4 (10 mg/kg, twice a week) in the 4T1 orthotopic solid tumor model.

FIG. 26 shows the IHC images of 4T1 tumor tissues after treatment with FA-TLR7-1A (Compound 1000), which were stained with DAPI (white, solid-line circle) and antibodies for Arg-1 (white arrow, upper row), iNOS (white, dashed-line circle), and CD8 (white arrow, lower row).

FIG. 27 is a summary of an in vivo combination therapy methodology comprising treatment using FA-TLR7-IA with anti-PD-1/anti-PD-L1 antibodies in a 4T1 tumor model.

FIG. 28 shows a graph of tumor volume from the in vivo combination study of FIG. 27, measured after treatment with a FA-TLR7-IA conjugate (Compound 1000) (3 nmol/mouse) in combination with anti-PD-1 (5 mg/kg, twice a week) in a MC38 solid tumor model.

FIG. 29A shows immune cell analysis data of digested MC38 tumor samples after treatment as described in FIG. 27.

FIG. 29B shows tumor volume measured after the cured mice from the combination study outlined in FIG. 28 and healthy control mice were rechallenged with 0.5 million MC38 cells.

FIG. 30 is a summary of an in vivo combination therapy methodology comprising treatment using a FA-TLR7-TA non-releasable conjugate (Compound 1000) with anti-CTLA-4 antibodies in a LL/2 Lewis lung carcinoma tumor model.

FIG. 31 shows a graph of tumor volume from the in vivo combination study of FIG. 30, measured every other day after treatment with a FA-TLR7-IA conjugate (Compound 1000) (3 nmol/mouse) in combination with anti-CTLA-4 (10 mg/kg, twice a week) in a LL/2 Lewis lung carcinoma tumor model.

FIG. 32A shows a graph of the change in mice body weight during the combination therapy treatment in a 4T1 tumor model of Example 16.

FIG. 32B shows a graph of the change in mice body weight during the combination therapy treatment of Example 17.

FIG. 32C shows a graph of the change in mice body weight as a measure of systemic toxicity after chronic administration of 60 nmoles/mice/day dose of TLR7-1A (Compound 1) or FA-TLR7-IA (Compound 1000) to healthy mice.

While the present disclosure is susceptible to various modifications and alternative forms, exemplary embodiments thereof are shown by way of example in the drawings and are herein described in detail.

### DETAILED DESCRIPTION

The compounds, combinations, compositions, and methods of the present disclosure generally target the innate immune system of a subject and reprogram the polarization of a macrophage from M2-type to M1-type in favor of the proinflammatory properties of the M1-type phenotype. For example, in at least one exemplary embodiment, such compounds and compositions comprise a targeting moiety to target folate receptor β (FRβ), such as a folate receptor-binding ligand, or an analog, functional fragment, derivative, or a radical thereof (e.g., a pteroyl amino acid), coupled with or linked to an immune modulator or a pharmaceutically acceptable salt thereof. The term “ligand” is a molecule, ion, or atom that is attached to the central atom or ion (e.g., a drug) of a compound.

As is described in detail below, such embodiments can utilize the limited expression of FRβ to localize systemically administered compound directly to FRβ-expressing cells (e.g., those of cancerous tissue) such that the immune modulator component can then convert—e.g., reprogram—activated myeloid cells (e.g., M2-like macrophages) into a proinflammatory M1 polarization. This targeting design can advantageously prevent the systemic activation of the immune system (i.e., reduces systemic exposure to such compound) and, thus, avoids toxicity.

Further exemplary embodiments can comprise a linker disposed between the targeting moiety and the immune modulator. Such linkers can be releasable or non-releasable. A compound/composition that comprises a releasable linker can, when administered, result in the targeting moiety and immune modulator being released from each other on or about the time the immune modulator becomes active. Additionally or alternatively, in embodiments where a compound comprises a non-releasable linker, when administered, the targeting moiety and immune modulator do not release quickly under physiological conditions. In this way, the components of the compound remain together following uptake by a targeted cell and/or activation of the immune modulator.

Various embodiments will now be described, as well as data relating to examples that support the same.

### Compounds

The compound can comprise a drug (e.g., a radical thereof) (e.g., an immune modulator) or pharmaceutically acceptable salt or hydrate thereof conjugated with a targeting moiety (e.g., a radical thereof). The immune modulator (e.g., a radical thereof) can be conjugated directly to the targeting moiety (e.g., a radical thereof) or through a linker (e.g., optionally comprising a spacer).

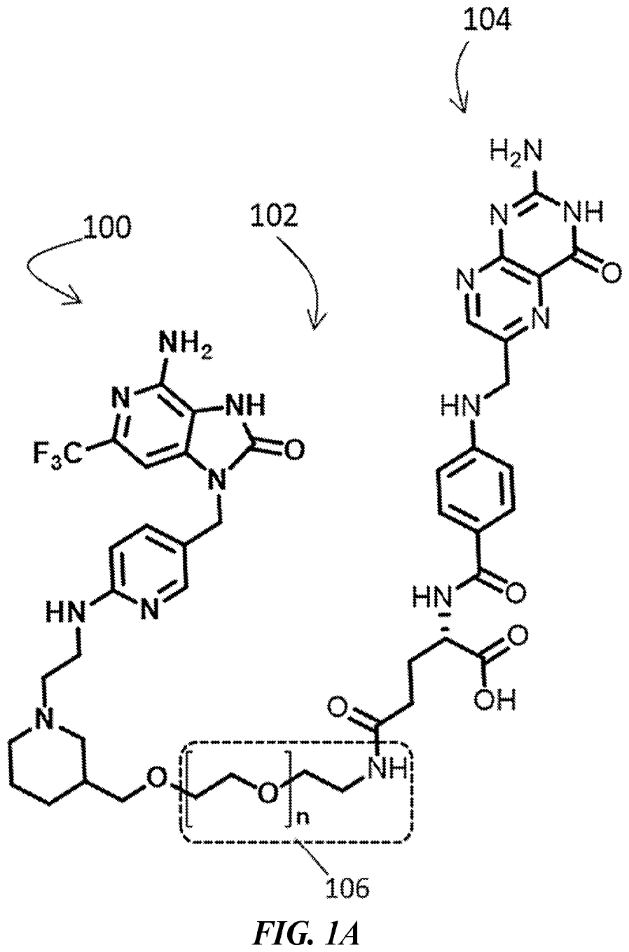

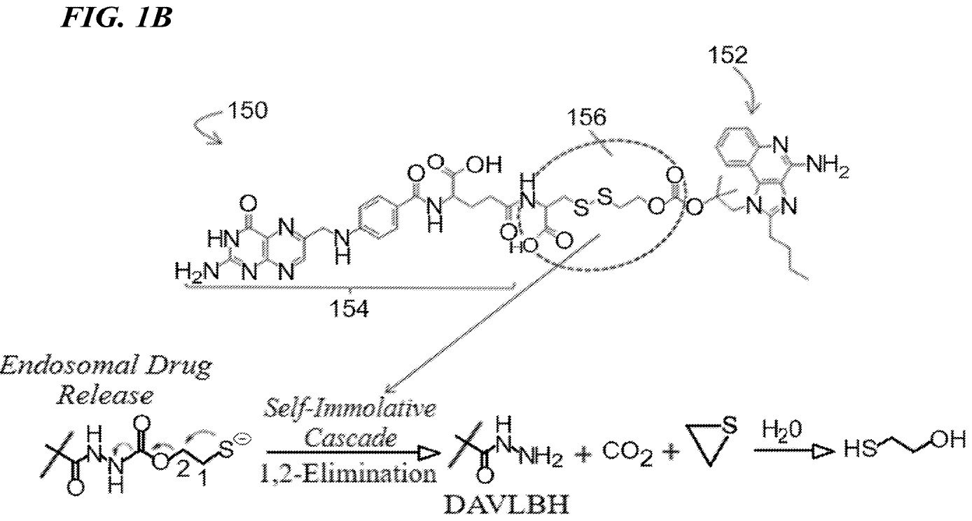

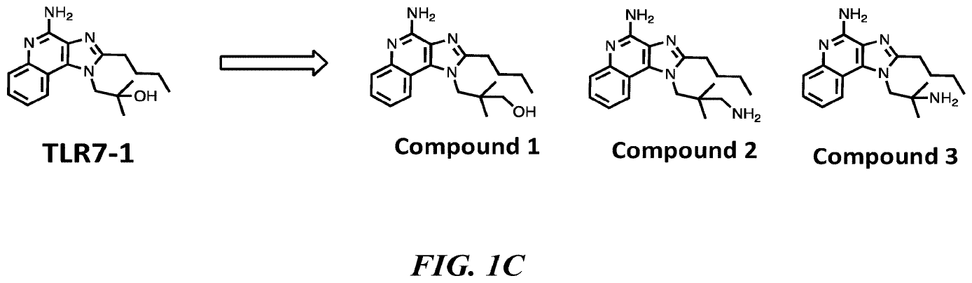

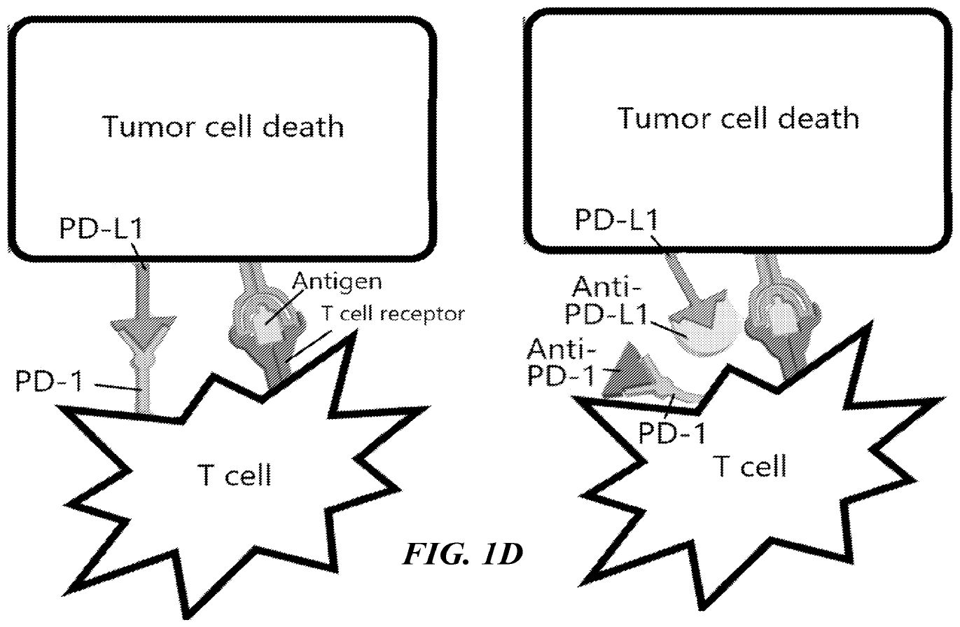

FIG. 1A shows at least one embodiment of compound 100. Here, compound 100 comprises an immune modulator 102 (or drug or radical thereof), for example, having Formula I, where R³ is a hydroxy group. The immune modulator 102 (e.g., a radical thereof) can be conjugated to a targeting moiety 104 (e.g., a radical thereof) through a linker 106. Here, the targeting moiety 104 (e.g., a radical thereof) is a folate and the (e.g., non-releasable) linker 106 is a polyethylene glycol (PEG) linker repeated n times, wherein n is an integer between 1 and 32.

In certain embodiments, compound 100 can be represented by the formula:

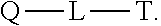

wherein Q is a radical of a folate receptor-binding ligand/targeting moiety 104, L is a linker 106, and T is a radical of a TLR agonist/immune modulator 102. The linker L can comprise any of the linker formulae presented herein.

Similarly, FIG. 1B shows at least one embodiment of compound 150. Compound 150 has an immune modulator/drug 152 (e.g., a radical thereof) that is a toll-like receptor 7 agonist (e.g., a radical thereof), e.g., having Formula III, conjugated to a targeting moiety 154 (e.g., a radical thereof) through a (e.g., releasable) linker 156.

### Immune Modulators

“Immune modulator” means any drug, warhead, or other composition or compound that stimulates or otherwise affects a subject's immune system by inducing activation or increasing activity of one or more of the components of the immune system. For example, and without limitation, immune modulators can include a compound or composition that targets one or more pattern recognition receptors or damage associated molecular patterns (DAMPs) in addition to, or in lieu of, targeting signaling pathways in immune cells. “Pattern recognition receptors” means and includes any immune receptors that are expressed on the membranes of leukocytes—e.g., at least macrophages—and can bind specific ligands that activate the receptor and ultimately lead to an innate immune response (and, in certain cases, eventually the development of antigen-specific acquired immunity). The immune modular (e.g., or radical of an immune modular) can comprise a pharmaceutically acceptable salt or hydrate thereof.

Exemplary examples of immune modulators can include, without limitation, agonists of TLRs, stimulator of interferon genes (STINGs), nucleotide-binding oligomerization domain (NOD)-like receptors (NLRs), retinoic acid-inducible gene-I (RIG-I)-like receptors (RLRs), absent in melanoma 2 (AIM2)-like receptors (ALRs), the receptor for advanced glycation end products (RAGE), or any other pattern recognition receptor that is located in the endosome or cytoplasm of a cell. The immune modulators can additionally or alternatively comprise a nuclear factor kappa-light-chain-enhancer of activated B cells (NFκβ) activator or an Iκβ kinase inhibitor, which work farther downstream in the pathway. Table 1 provides examples of such NFκβ activators or Iκβ kinase inhibitors that can be employed as the immune modulators.

TABLE 1
NFκβ Activators/Inducers

| Compound | Structure |
|---|---|
| AA | 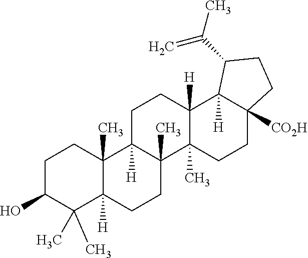 |
| BB | 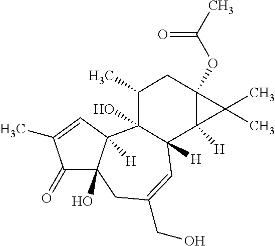 |
| CC | 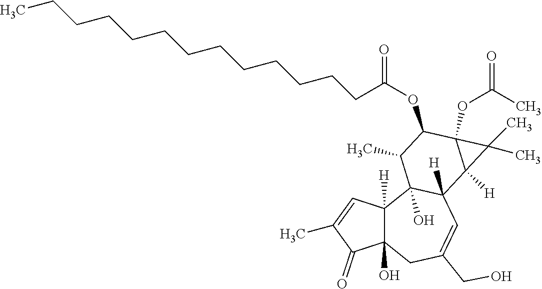 |
| DD | 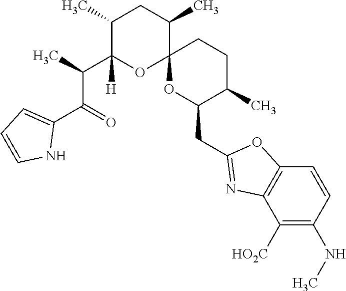 [SMILES: CNc1ccc2oc(C[C@H]3O[C@@]4(CC[C@H]3C)O[C@H]([C@H](C)C(=O)c3ccc[nH]3)[C@H](C)C[C@H]4C)nc2c1C(=O)O] |
| EE | 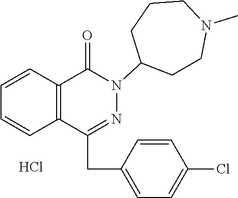 [SMILES: CN1CCCC(n2nc(Cc3ccc(Cl)cc3)c3ccccc3c2=O)CC1 ; Cl] |
| FF | 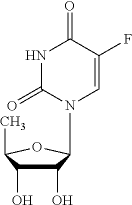 |
| GG | 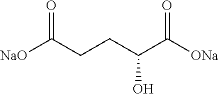 |

“TLRs” are a class of proteins that play a role in the innate immune system and are an example of pattern recognition receptors. TLRs can be single, membrane-spanning receptors that recognize structurally conserved molecules derived from microbes. TLRs can be expressed on the membranes of leukocytes including, for example, dendritic cells, macrophages, natural killer cells, cells of adaptive immunity (e.g., T and B lymphocytes) and non-immune cells (epithelial and endothelial cells and fibroblasts). Non-limiting examples of TLRs include TLR1, TLR2, TLR3, TLR4, TLR5, TLR6, TLR7, TLR8, TLR9, TLR10, TLR1T, TLR12, and TLR13. In some embodiments, a TLR agonist provided herein binds to one or more TLR. In some embodiments, a TLR agonist provided herein binds to TLR7, TLR8, or TLR9. In some embodiments, a TLR agonist provided herein binds to TLR7. In some embodiments, a TLR agonist provided herein binds to TLR7 and TLR8 (a TLR 7/8 agonist). In some embodiments, an agonist is a ligand that binds to and activates a receptor.

In some instances, such as wherein the compound is a (e.g., potent) TLR-7/8 agonist, the immune modulator (or radical thereof) could be highly toxic if delivered systemically in an unconjugated form. In some instances, it is desirable to reduce and/or eliminate systemic toxicity associated with such compounds. In some instances, a radical of a compound has reduced toxicity relative to the free form of such a compound (e.g., reduced by 10%-15%, 15%-20%, 20%-30%, 30%-40%, 40%-50%, 50%-60%, 60%-70%, 70%-75%, 75%, 75%-80%, 80%9%, 90%, or 90%-99% (all listed ranges inclusive of stated end points and all 1% increments encompassed therein)). Moreover, in some instances, conjugated forms of the compounds are efficacious at comparable or lower concentrations (e.g., having a median effective dose (ED₅₀) concentration of 120% of the free form or less, at 100% or less, at 80% or less, at 60%, or less, or at 40% or less) relative to a free form of the immune modulator.

The immune modulator, or pharmaceutically acceptable salt or hydrate thereof, can comprise any therapeutic agent (e.g., drug) suitable for reprogramming activated macrophages (M2-like phenotype) to an MI-like phenotype. In certain embodiments, the immune modulator operates in the endosome and/or cytoplasm of the cell (e.g., depending on its structure). In at least one embodiment, the immune modulator is one that positively controls a pattern recognition receptor and/or its downstream signaling pathways (in each case, part of the innate immune system), such as, for example. TLR, NLR, RLR, ALR, RAGE, and/or STING agonists and/or a kinase of the Pelle/interleukin-1 receptor-associated kinase (IRAK) family, such as an IRAK-M inhibitor). In other embodiments, the compound comprises a phosphoinositide 3-kinase (PI3K) inhibitor or other inhibitor that negatively controls the adaptive immune system (e.g., which can be employed alone or in conjunction with an immune modulator that targets a pattern recognition receptor or a DAMP). In some embodiments, especially when used in the treatment of cancer, the compound (or pharmaceutically acceptable salt or hydrate thereof) comprises a combination of (a) a radical of an immune modulator that targets a pattern recognition receptor or a DAMP and/or is an agonist of downstream signaling pathways of the subject's innate immune system, and (b) a mammalian target of rapamycin (mTOR) inhibitor (ATP-competitive or otherwise) such as, for example, rapamycin or CZ415.

In some embodiments, the immune modulator of the compound (or pharmaceutically acceptable salt or hydrate thereof) comprises a TLR agonist (e.g., a radical thereof), for example and without limitation, a TLR3 agonist, a TLR7 agonist, a TLR7/8 agonist, a TLR8 agonist, or a TLR9 agonist (e.g., all of which bind with a TLR present within the endosome of a cell). For example, and without limitation, in at least one exemplary embodiment, the immune modulator of the compound (or pharmaceutically acceptable salt or hydrate thereof) can be selected from the compounds listed in Table 2 below (and, for example, comprise a radical thereof).

TABLE 2
TLR agonists.

| Compound | Structure/Description | Type |
|---|---|---|
| A | 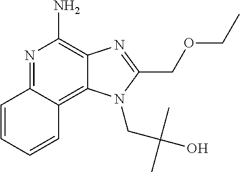 [SMILES: CCOCc1nc2c(N)nc3ccccc3c2n1CC(C)(C)O] | TLR7 agonist |
| B | 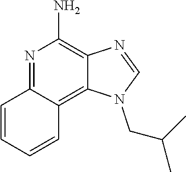 [SMILES: CC(C)Cn1cnc2c(N)nc3ccccc3c21] | TLR7 agonist |
| C | 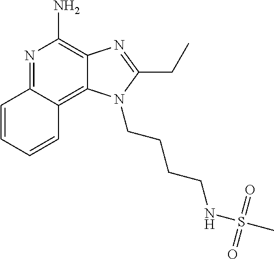 [SMILES: CCc1nc2c(N)nc3ccccc3c2n1CCCCNS(C)(=O)=O] | TLR7 agonist |
| D | 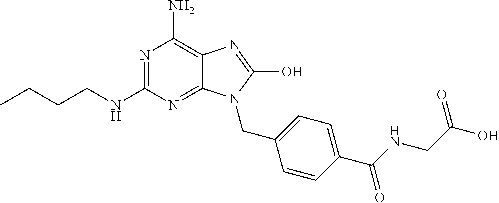 [SMILES: CCCCNc1nc(N)c2nc(O)n(Cc3ccc(C(=O)NCC(=O)O)cc3)c2n1] | TLR7 agonist |
| E | 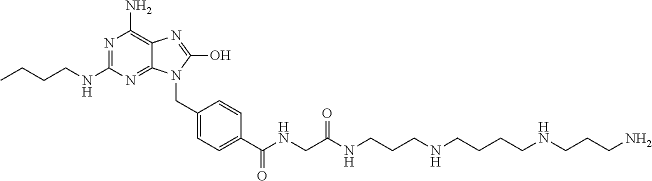 [SMILES: CCCCNc1nc(N)c2nc(O)n(Cc3ccc(C(=O)NCC(=O)NCCCNCCCCNCCCN)cc3)c2n1] | TLR7 agonist |
| F | 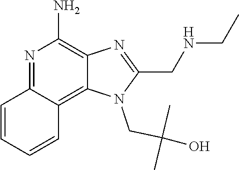 [SMILES: CCNCc1nc2c(N)nc3ccccc3c2n1CC(C)(C)O] | TLR7 agonist |
| G | 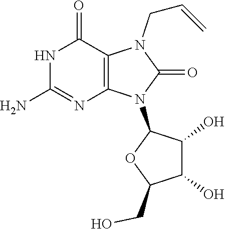 [SMILES: C=CCn1c(=O)n([C@@H]2O[C@H](CO)[C@@H](O)[C@H]2O)c2nc(N)[nH]c(=O)c21] | TLR7 agonist |
| H | 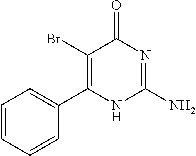 [SMILES: Nc1nc(=O)c(Br)c(-c2ccccc2)[nH]1] | TLR7 agonist |
| I | 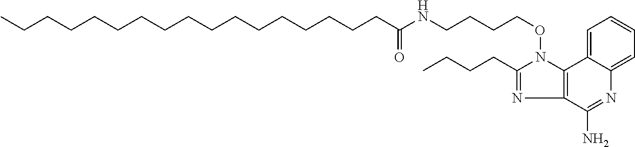 [SMILES: CCCCCCCCCCCCCCCCCC(=O)NCCCCOn1c(CCCC)nc2c(N)nc3ccccc3c21] | TLR7 agonist |
| J | 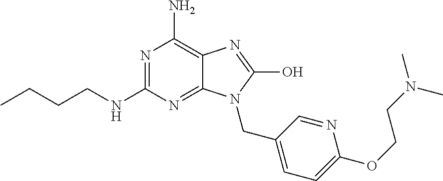 [SMILES: CCCCNc1nc(N)c2nc(O)n(Cc3ccc(OCCN(C)C)nc3)c2n1] | TLR7 agonist |
| K | 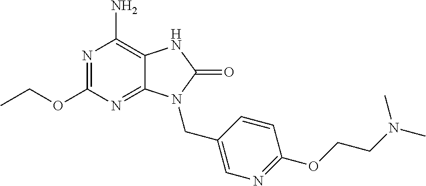 [SMILES: CCOc1nc(N)c2[nH]c(=O)n(Cc3ccc(OCCN(C)C)nc3)c2n1] | TLR7 agonist |
| L | 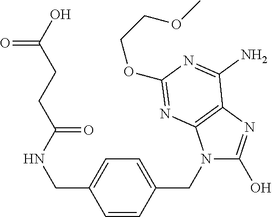 [SMILES: COCCOc1nc(N)c2nc(O)n(Cc3ccc(CNC(=O)CCC(=O)O)cc3)c2n1] | TLR7 agonist |
| M | 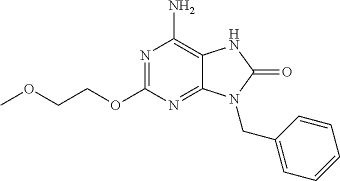 [SMILES: COCCOc1nc(N)c2[nH]c(=O)n(Cc3ccccc3)c2n1] | TLR7 agonist |
| N | 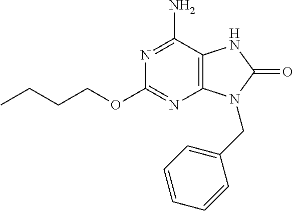 [SMILES: CCCCOc1nc(N)c2[nH]c(=O)n(Cc3ccccc3)c2n1] | TLR7 agonist |
| O | 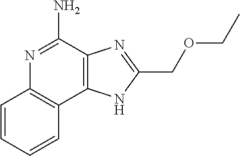 [SMILES: CCOCc1nc2c(N)nc3ccccc3c2[nH]1] | TLR7/8 agonist |
| P | 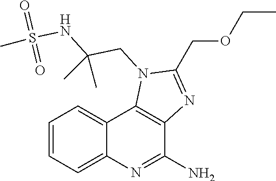 [SMILES: CCOCc1nc2c(N)nc3ccccc3c2n1CC(C)(C)NS(C)(=O)=O] | TLR7/8 agonist |
| Q | See, e.g., Lipanov et al., ″The structure of poly(dA): poly(dT) in a condensed state and in solution,″ | TLR7/8 |
| | Nucleic Acids Research 15 (14): 5833-5844 (1987). | agonist |
| R | 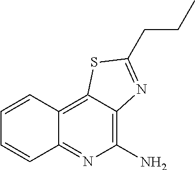 | TLR8 agonist |
| S | 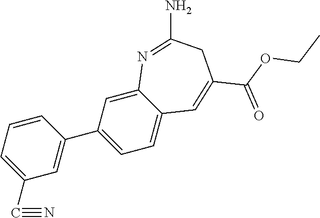 [SMILES: CCOC(=O)C1=Cc2ccc(-c3cccc(C#N)c3)cc2N=C(N)C1] | TLR8 agonist |
| T | 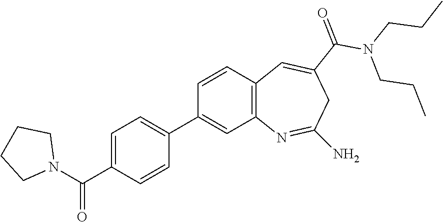 [SMILES: CCCN(CCC)C(=O)C1=Cc2ccc(-c3ccc(C(=O)N4CCCC4)cc3)cc2N=C(N)C1] | TLR8 agonist |
| U | Short synthetic single-stranded DNA molecules containing unmethylated CpG dinucleotides in particular | TLR9 |
| | sequence contexts (CpG motifs) (CpG ODN) | agonist |
| V | Synthetic oligonucleotide containing unmethylated CpG dinucleotides with potential immuno-potentiating | TLR9 |
| | activity (IMO 2005) | agonist |
| W | Short, synthetic, unmethylated CpG oligodeoxynucleotide (CpG ODN) with immunostimulatory activity | TLR9 |
| | (1018-ISS) | agonist |
| X | Comprises a strand of inosine poly(I) homopolymer annealed to a strand of cytidine (poly(I:C)) | TLR3 |
| | | agonist |
| Y | Poly(C)homopolymer | TLR3 |
| | | agonist |
| Z | 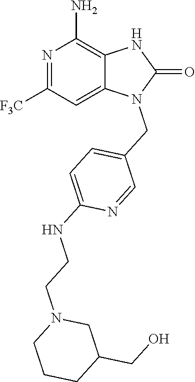 [SMILES: Nc1nc(C(F)(F)F)cc2c1[nH]c(=O)n2Cc1ccc(NCCN2CCCC(CO)C2)nc1] | TLR7 agonist |

In some embodiments, the immune modulator comprises a TLR7 agonist or radical thereof. In some embodiments, an immune modulator (or radical) (e.g., TLR7 agonist) has a structure of Formula I:

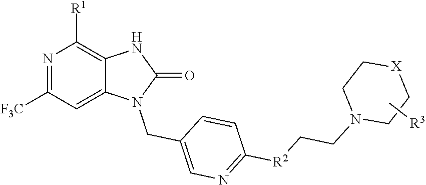

or is a pharmaceutically acceptable salt or hydrate thereof. In some embodiments, R¹ is an amine. In some embodiments, R² is a (e.g., single) bond, or an amine (e.g., —NH—). In certain embodiments, R³ is an H, an alkyl, a hydroxy group, or any other suitable substituent (e.g., as described herein). In some embodiments, X is CH₂, NH, O, or S. In some embodiments where a compound comprises a radical of Formula I, a targeting moiety is conjugated or connected thereto at any suitable location, such as at or through R¹, R², and/or R³ (e.g., through a linker and/or directly).

In at least one exemplary embodiment, a compound is or comprises an immune modulator (or radical thereof) (e.g., TLR7 agonist) of Formula Ia:

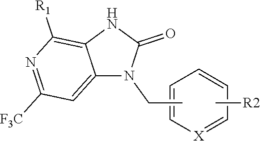

or is a pharmaceutically acceptable salt or hydrate thereof. In some embodiments. X is a CH or an N. In some embodiments. R₁ is —NH₂ or —NH—R_{1X}. In some embodiments, R₂ is H, alkyl, alkenyl, alkynyl, alicyclic, aryl, biaryl, heteroaryl, —NH—R_{2X}, —O—R_{2X}, —S—R_{2X},

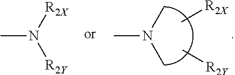

In specific embodiments, each of R_{1X}, R_{2X}, and R_{2Y} is independently selected from the group consisting of H, alkyl, alkenyl, alkynyl, alicyclic, aryl, biaryl, and heteroaryl. In some embodiments,

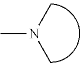

is a 3-10 membered N-containing non-aromatic mono- or bicyclic heterocycle. In some embodiments where a compound comprises a radical of formula Ia, a targeting moiety is conjugated or connected thereto at any suitable location, such as at or through R¹, R², and/or R³ (e.g., through a linker and/or directly).

In some embodiments, a compound comprises an immune modulator that has or comprises a structure of Formula II:

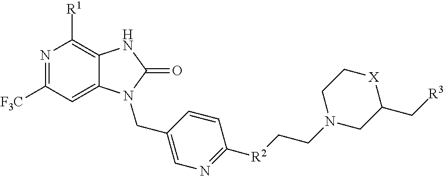

or is a pharmaceutically acceptable salt or hydrate thereof. In some embodiments, R¹ is an amine. In some embodiments, R² is a (e.g., single) bond or —NH—. In some embodiments, R³ is H, alkyl, hydroxy group, or any other substituent, such as described herein. In some embodiments, X is a CH₂, NH, O, or S. In some embodiments wherein a compound comprises a radical of formula II, a targeting moiety is conjugated or connected thereto at any suitable location, such as at or through R¹, R², and/or R³ (e.g., through a linker and/or directly).

In other embodiments, the immune modulator of a compound can include or comprise a drug comprising the TLR agonist (e.g., or a radical thereof) of Formula III or a pharmaceutically acceptable salt or hydrate thereof:

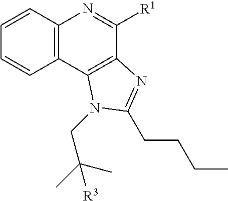

wherein, R¹ is an amine group and R³ is a hydroxy group. Further, if desired, a targeting moiety (e.g., or a radical thereof) or other ligand can be conjugated to the agonist of Formula III at R¹ or R³ (through a linker or directly). The TLR agonist (e.g., or radical thereof) of Formula III is a TLR7 agonist and at least ten times (10×) as potent as the TLR7 agonists conventionally available.

In certain embodiments, the immune modulator of the compound can be or can comprise a TLR7 agonist (e.g., or radical thereof) of Formula IV or a pharmaceutically acceptable salt or hydrate thereof:

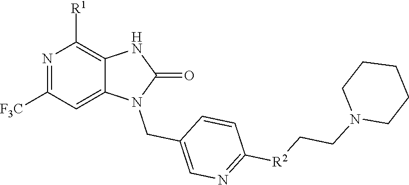

wherein R¹ is an amine group and R² is a single bond —NH—.

In certain embodiments, the immune modulator comprises a TLR agonist of Formula X or XX, or is a pharmaceutically acceptable salt of Formula X or XX:

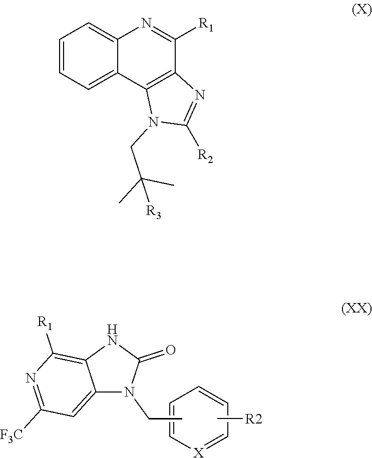

wherein, in Formulas X and XX, R₁ is —NH₂ or —NH—R_{1X}, R₂ is an H, an alkyl, an alkenyl, an alkynyl, an alicyclic, an aryl, a biaryl, a heteroaryl, —NH—R_{2X}, —O—R_{2X}, —S—R_{2X},

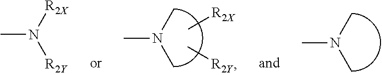

is a 3-10 membered N-containing non-aromatic mono- or bicyclic heterocycle, wherein, in Formula X, R₃ is —OH, —SH, —NH₂ or —NH—R_{1X}, wherein, in Formula XX, X is a CH or N, and each of R_{1X}, R_{2X}, and R_{2Y} is independently selected from the group consisting of an H, an alkyl, an alkenyl, an alkynyl, an alicyclic, an aryl, a biaryl, and a heteroaryl.

In certain embodiments, the immune modulator (e.g., TLR7 agonist) group of the compound is a radical having a structure of Formula XX, and more specifically of Formula XX′:

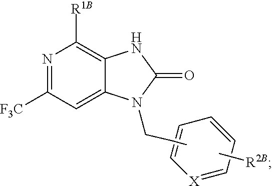

wherein:
R^{1B} is —NH₂ or —NH—R^{1X},
R^{2B} is a hydrogen (H), an alkyl, an alkenyl, an alkynyl, an alicyclic, an aryl, a biaryl, a heteroaryl, —NH—R^{2X}, —O—R^{2X}, —S—R^{2X},

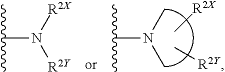

each of R^{1X}, R^{2X}, and R^{2Y} is independently selected from the group consisting of an H, an alkyl, an alkenyl, an alkynyl, an alicyclic, an aryl, a biaryl, and a heteroaryl, and

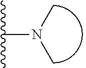 [SMILES: *N(C)C]

is a 3-10 membered, N-containing, non-aromatic, mono- or bicyclic heterocycle, and X is CH or nitrogen (N).

Alkyl, alkoxy, etc. each denote a straight (i.e., unbranched) or branched chain, or a combination thereof, which can be fully saturated, mono- or polyunsaturated and can include di- and multivalent radicals, having the number of carbon atoms designated (i.e., C₁-C₁₀ means one to ten carbons). Examples of saturated hydrocarbon radicals include, without limitation, groups such as methyl, ethyl, n-propyl, isopropyl, n-butyl, t-butyl, isobutyl, sec-butyl, (cyclohexyl)methyl, and homologs and isomers of, for example, n-pentyl, n-hexyl, n-heptyl, n-octyl, and the like. An unsaturated alkyl group is one having one or more double bonds or triple bonds. Examples of unsaturated alkyl groups include, without limitation, vinyl, 2-propenyl, crotyl-2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). In some embodiments, alkoxy refers to a radical bonded through an oxygen atom of the formula —O-alkyl.

In general, the term “acyl” or “acyl substituent” refers to a group derived by the removal of one or more hydroxyl groups from an oxoacid, including inorganic acids, and contains a double-bonded oxygen atom and an alkyl group. Further, reference to an individual radical such as “propyl” embraces only the straight chain radical, a branched chain isomer such as “isopropyl” being specifically referenced.

In certain embodiments, the TLR7 agonist has Formula X, and the radical of the TLR7 agonist is conjugated to the targeting moiety at any one of R^{1A}, R^{1B}, R^{3A}, or R^{3B} through a linker; and where the TLR7 agonist has Formula XX′, the TLR7 agonist is conjugated to the targeting moiety at one of R^{1A}, R^{1B}, R^{3A}, or R^{3B} through a linker.

In certain embodiments, provided is a compound comprising a targeting moiety comprising a folate ligand or a functional fragment or analog thereof attached to an immune modulator comprising a TLR agonist via a linker, the TLR agonist having the following structure represented by formula XXX:

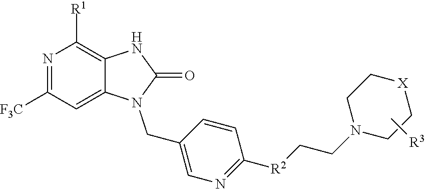

wherein R¹ is an amine group, R² is a single bond —NH—, and R³ is an H, an alkyl, a hydroxy group, or any other substituted group thereof, X is a CH₂, NH, O, or S, and the linker is attached at R¹, R² or R³. Additionally or alternatively, R₁ can be —NH₂ or —NH—R_{1X}; R₂ can be an H, an alkyl, an alkenyl, an alkynyl, an alicyclic, an aryl, a biaryl, a heteroaryl, —NH—R_{2X}, —O—R_{2X}, —S—R_{2X},

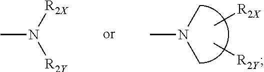

or each of R_{1X}, R_{2X}, and R_{2Y} can be independently selected from the group consisting of an H, an alkyl, an alkenyl, an alkynyl, an alicyclic, an aryl, a biaryl, and a heteroaryl;

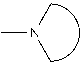

can be a 3-10 membered N-containing non-aromatic mono- or bicyclic heterocycle; and/or X can be CH, CR₂, or N.

In some embodiments, provided is a pharmaceutical composition comprising any formula or compound provided, wherein the linker comprises PEG or a PEG derivative and, in some instances, is either a non-releasable linker attached at R³ or is a releasable linker attached at R¹, R² or R³.

In some embodiments, the immune modulator (e.g., TLR7 or TLR 7/8 agonist) group of a compound is a radical having a structure of Formula XXX, and more specifically of Formula XXX′:

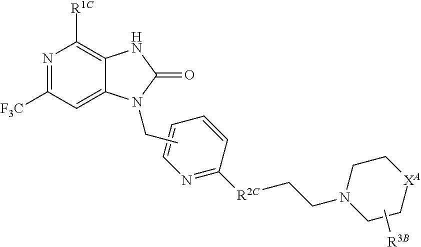

wherein,
R^{1C} is —NH₂ or —NH—R^{1X},
R^{2C} is a bond, NH, —NR^{1X}, or CH₂,
and
if applicable,

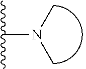

is a 3-10 membered. N-containing, non-aromatic, mono- or bicyclic heterocycle;
X^{A} is CH₂, NH₂, or —NH—R^{1X}; and
each R^{1X} is independently selected from the group consisting of an H, an alkyl, an alkenyl, an alkynyl, an alicyclic, an aryl, a biaryl, and a heteroaryl,
where the TLR7 agonist is conjugated to the targeting moiety at one of R^{1C}, R^{2C} or R^{3B} through a linker.

One embodiment provides a compound represented by the structure of Formula (2-II):

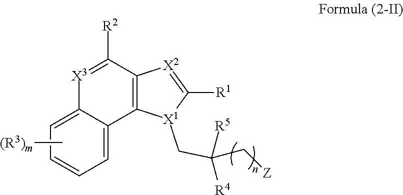

or a pharmaceutically acceptable salt or hydrate thereof wherein:
R¹, R³, R⁴, R⁵ are each independently a H, an alkyl, an alkoxyl, an alkenyl, an alkynyl, an alicyclic, an aryl, a biaryl, a halo, a heteroaryl, —COR^{2x},

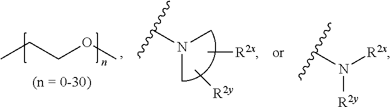

R² is a H, —OH, —NH₂, —NHR^{2x}, N₃, —NH—CH₂—NH₂, —CONH₂, —SO₂NH₂, —NH—CS—NH₂,

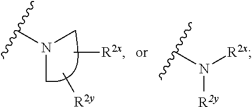

Z is a group of the formula G-L-, G-O-, G-L-O-, G-L-O-alkyl-, G-L-S-, G-SO₂—NH—, G-L-NR^{a}R^{b}—, G-L-S(O)ₓ-alkyl-, G-L-CO-, G-L-aryl-, G-L-NH—CO—NH—, G-L-NH—O—, G-L-NH—NH—, G-L-NH—CS—NH, G-L-C(O)-alkyl-, G-L-SO₂—,

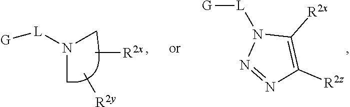

wherein:
L is a linker and G is a folate receptor-binding ligand,
R^{a} and R^{b} are each, independently, H, halo, hydroxy, alkoxy, aryl, amino, acyl or C(O)R^{c}, wherein R^{c} is alkyl, aryl, oxy or alkoxy; x is 0-3; each of R^{2x} and R^{2y} is independently selected from the group consisting of H, —OH, —CH₂—OH, —NH₂, —CH₂—NH₂, —COOMe, —COOH, —CONH₂, —COCH₃, alkyl, alkenyl, alkynyl, alicyclic, aryl, biaryl, and heteroaryl, and
each R^{2z} is independently selected from the group consisting of —NH₂, —NR^{2q}R^{2q′}, —O—R^{2q}, —SO—R^{2q}, and —COR^{2q};
wherein each R^{2q} and R^{2q′} is independently alkyl or H,

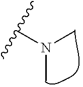 [SMILES: *N(C)C]

is a 3-10 membered N-containing non-aromatic, mono- or bicyclic heterocycle;
wherein, in Formula 2-II, X¹, X², and X³ are each independently CR^{q} or N; each R^{q} is independently hydrogen, halogen, or an optionally substituted alkyl; and
wherein, in Formula 2-II, n is 0-30, and m is 0-4.

At least one embodiment provides an immune modulator (or radical thereof) represented by the structure of Formula (2-IIA) or a pharmaceutically acceptable salt or hydrate thereof:

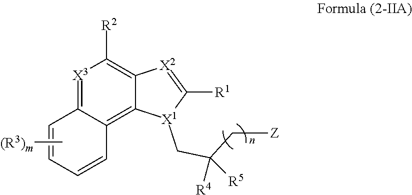

wherein:
R¹ is optionally substituted alkyl (e.g., acyclic or cyclic) (e.g., optionally substituted with one or more substituents, each substituent independently being halogen, alkyl, heteroalkyl, alkoxy, or cycloalkyl);
R² is H, —OR^{z}, —SO₂N(R^{z})₂, —NR^{2x}R^{2y}, or N₃ and:
   R^{2x} and R^{2y} are each independently hydrogen, —N(R^{z})₂, —CON(R^{z})₂, —C(R^{z})₂—N(R^{z})₂, —CS—N(R^{z})₂, or optionally substituted alkyl (e.g., optionally substituted with one or more substituents, each substituent independently being oxo, halogen, alkyl, heteroalkyl, alkoxy, or cycloalkyl); and each R^{z} is independently hydrogen, halogen, or optionally substituted alkyl; or
   R^{2x} and R^{2y} are taken together to form an optionally substituted heterocycloalkyl (e.g., wherein the optionally substituted heterocycloalkyl is a mono- or bicyclic heterocycloalkyl and/or wherein the optionally substituted heterocycloalkyl is a 3-10 membered heterocycloalkyl);
each R³ is independently halogen, —N₃, —CN, —NO₂, alkyl, heteroalkyl, cycloalkyl, heterocycloalkyl, alkoxy, aryl, heteroaryl, heterocycloalkyl, amino, hydroxy, carbonyl, or thiol, wherein the alkyl, alkoxy, heteroalkyl, cycloalkyl, or heterocycloalkyl is optionally substituted;
R⁴ and R⁵ are each independently alkyl, alkoxy, halogen, or cycloalkyl, wherein the alkyl, alkoxy, and cycloalkyl is optionally substituted;
each of X¹, X², and X³ is independently CR^{q} or N, and each R^{q} is independently hydrogen, halogen, or optionally substituted alkyl;
Z is L-G, wherein L is a linker and G is a folate receptor-binding ligand; and
n is 1-6, and m is 0-4.

At least one embodiment provides an immune modulator (or radical thereof), or a pharmaceutically acceptable salt or hydrate thereof, having the structure of Formula (2-II) or (2-IIA) wherein n is 1-30. In one embodiment, n is 1-6. In another embodiment, n is 1-3. In another embodiment, n is 1 or 2. In another embodiment, n is 0. In another embodiment, n is 1.

At least one embodiment provides an immune modulator (or radical thereof), or a pharmaceutically acceptable salt or hydrate thereof, having the structure of Formula (2-II) or (2-IIA) wherein R¹ is an optionally substituted alkyl. In one embodiment, R¹ is an optionally substituted C₃-C₆ alkyl. In another embodiment, R¹ is an optionally substituted acyclic C₃-C₆ alkyl. In at least one embodiment, R¹ is butyl.

At least one embodiment provides an immune modulator (or radical thereof), or a pharmaceutically acceptable salt or hydrate thereof, having the structure of Formula (2-II) or (2-IIA) wherein R² is —NR^{2x}R^{2y}. In at least one embodiment. R² is NH₂.

One embodiment provides an immune modulator (or radical thereof), or a pharmaceutically acceptable salt or hydrate thereof, having the structure of Formula (2-II) or (2-IIA) wherein R³ is H.

At least one embodiment provides an immune modulator (or radical thereof), or a pharmaceutically acceptable salt or hydrate thereof, having the structure of Formula (2-II) or (2-IIA) wherein R⁴ is alkyl. In at least one embodiment, R⁴ is methyl.

At least one embodiment provides an immune modulator (or radical thereof), or a pharmaceutically acceptable salt or hydrate thereof, having the structure of Formula (2-II) or (2-IIA) wherein R⁵ is alkyl. In at least one embodiment, R⁵ is methyl.

At least one embodiment provides an immune modulator (or radical thereof), or a pharmaceutically acceptable salt or hydrate thereof, having the structure of Formula (2-II) or (2-IIA) wherein R⁴ and R⁵ are each alkyl. In at least one embodiment, R⁴ and R⁵ are each methyl.

At least one embodiment provides an immune modulator (or radical thereof), or a pharmaceutically acceptable salt or hydrate thereof, having the structure of Formula (2-II) or (2-IIA) wherein m is 0. In another embodiment, m is 1. In another embodiment, m is 2. In another embodiment, m is 3. In another embodiment, m is 4.

At least one embodiment provides an immune modulator (or radical thereof), or a pharmaceutically acceptable salt or hydrate thereof, having the structure of Formula (2-II) or (2-IIA) wherein X′, X², and X³ are each N. In at least one embodiment, X¹ is N. In another embodiment, X² is N. In at least another embodiment, X³ is N.

At least one embodiment provides an immune modulator (or radical thereof) having the structure of Formula (2-II) or (2-IIA) wherein the compound is represented by the structure.

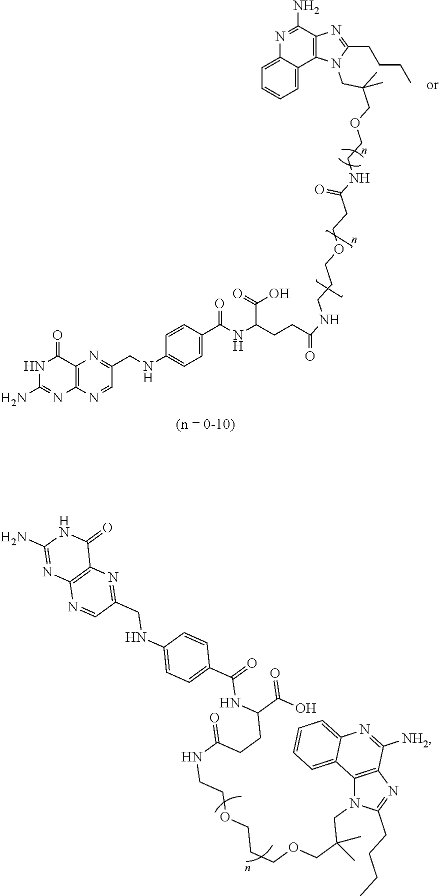

or is a pharmaceutically acceptable salt or hydrate of either of the foregoing structures.

At least one embodiment provides an immune modulator (or radical thereof) represented by the structure of Formula (2-III):

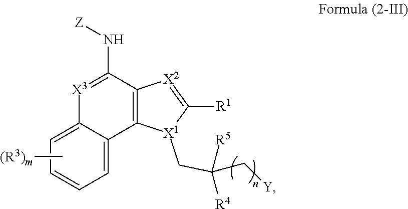

or a pharmaceutically acceptable salt or hydrate thereof, wherein:
R¹, R³, R⁴, and R⁵ are each independently a H, an alkyl, an alkoxyl, an alkenyl, an alkynyl, an alicyclic, an aryl, a biaryl, a halo, a heteroaryl, —COR^{2x},

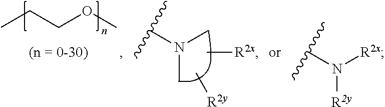

wherein each of R^{2x} and R^{2y} is independently selected from the group consisting of H, —OH, —CH₂—OH, —NH₂, —CH₂—NH₂, —COOMe, —COOH, —CONH₂, —COCH₃, alkyl, alkenyl, alkynyl, alicyclic, aryl, biaryl, and heteroaryl, and each R^{2z} is independently selected from the group consisting of —NH₂, —NR^{2x}R^{2y}, —O—R^{2x}, —SO—R^{2x}, and —COR^{2x};
Z is a group of the formula G-L-, G-L-CO—, G-L-C(O)-alkyl-, wherein L is a linker and G is a folate receptor-binding ligand; and
each of X¹, X², and X³ is independently CR or N, and
each R^{q} is independently hydrogen, halogen, or optionally substituted alkyl;
wherein, in Formula 2-111, n is 0-30, and m is 0-4.

At least one embodiment provides an immune modulator (or radical thereof) represented by the structure of Formula (2-IIIA), or pharmaceutically acceptable salt or hydrate thereof:

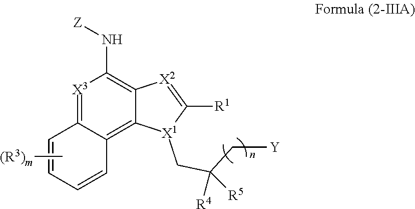

wherein:
R¹ is optionally a substituted alkyl (e.g., acyclic or cyclic) (e.g., optionally substituted with one or more substituents, each substituent independently being halogen, alkyl, heteroalkyl, alkoxy, or cycloalkyl);
Y is a point of attachment to the linker or targeting moiety of the compound and comprises H, —OR^{z}, —NR^{2x}R^{2y}, —SR^{z}, —SOR^{z}, —SO₃R^{z}, —N₃, —COR^{z}, —COOR^{z}, —CONR^{z}₂, —COSR^{z}, —SO₂N(R^{z})₂, or —CON(R^{z})₂; where:
   R^{2x} and R² are each independently H, —N(R^{z})₂, —CON(R^{z})₂, —C(R^{z})₂—N(R^{z})₂, —CS—N(R^{z})₂, or optionally substituted alkyl (e.g., optionally substituted with one or more substituents, each substituent independently being oxo, halogen, alkyl, heteroalkyl, alkoxy, or cycloalkyl);
   each R^{z} is independently H, a halogen, or an optionally substituted alkyl; or
   R^{2x} and R^{2y} are taken together to form an optionally substituted heterocycloalkyl (e.g., wherein the optionally substituted heterocycloalkyl is a mono- or bicyclic heterocycloalkyl and/or wherein the optionally substituted heterocycloalkyl is a 3-10 membered heterocycloalkyl);
each R³ is independently a halogen, —N₃, —CN, —NO₂, alkyl, heteroalkyl, cycloalkyl, heterocycloalkyl, alkoxy, aryl, heteroaryl, heterocycloalkyl, amino, hydroxy, carbonyl, or thiol, wherein the alkyl, alkoxy, heteroalkyl, cycloalkyl, or heterocycloalkyl is optionally substituted;
R⁴ and R⁵ are each independently alkyl, alkoxy, halogen, or cycloalkyl, wherein the alkyl, alkoxy, or cycloalkyl is optionally substituted;
each X¹, X², and X³ is independently CR⁴ or N, and each R^{q} is independently H, halogen, or an optionally substituted alkyl;
Z is L-G, wherein L is a linker and G is a folate receptor-binding ligand; and
n is 1-6, and m is 0-4.

At least one embodiment provides an immune modulator (or radical thereof), or a pharmaceutically acceptable salt or hydrate thereof, having the structure of Formula (2-III) or (2-IIIA) wherein n is 1-30. In one embodiment, n is 1-6. In another embodiment, n is 1-3. In another embodiment, n is 1 or 2. In another embodiment, n is 0. In another embodiment, n is 1. In another embodiment, n is 1 and Y is OH. In another embodiment, n is 1 and Y is NH₂.

At least one embodiment provides an immune modulator (or radical thereof), or a pharmaceutically acceptable salt or hydrate thereof, having the structure of Formula (2-III) or (2-IIIA) wherein Y is OH.

At least one embodiment provides an immune modulator (or radical thereof), or a pharmaceutically acceptable salt or hydrate thereof, having the structure of Formula (2-III) or (2-IIIA) wherein Y is NH₂.

At least one embodiment provides an immune modulator (or radical thereof), or a pharmaceutically acceptable salt or hydrate thereof, having the structure of Formula (2-III) or (2-IIIA) wherein n is 1 and Y is OH.

At least one embodiment provides an immune modulator (or radical thereof), or a pharmaceutically acceptable salt or hydrate thereof, having the structure of Formula (2-III) or (2-IIIA) wherein n is 1 and Y is NH₂.

At least one embodiment provides an immune modulator (or radical thereof), or a pharmaceutically acceptable salt or hydrate thereof, having the structure of Formula (2-III) or (2-IIIA) wherein n is 0 and Y is NH₂.

At least one embodiment provides an immune modulator (or radical thereof), or a pharmaceutically acceptable salt or hydrate thereof, having the structure of Formula (2-III) or (2-IIIA) wherein R¹ is an optionally substituted alkyl. In at least one embodiment, R¹ is an optionally substituted C₃-C₆ alkyl. In another embodiment, R¹ is an optionally substituted acyclic C₃-C₆ alkyl. In at least one other embodiment, R¹ is butyl.

At least one embodiment provides an immune modulator (or radical thereof), or a pharmaceutically acceptable salt or hydrate thereof, having the structure of Formula (2-III) or (2-IIIA) wherein R₃ is H.

At least one embodiment provides an immune modulator (or radical thereof), or a pharmaceutically acceptable salt or hydrate thereof, having the structure of Formula (2-III) or (2-IIIA) wherein R⁴ is alkyl. In at least one embodiment, R⁴ is methyl.

At least one embodiment provides an immune modulator (or radical thereof), or a pharmaceutically acceptable salt or hydrate thereof, having the structure of Formula (2-III) or (2-IIIA) wherein R⁵ is alkyl. In at least one embodiment. R⁵ is methyl.

At least one embodiment provides an immune modulator (or radical thereof), or a pharmaceutically acceptable salt or hydrate thereof, having the structure of Formula (2-III) or (2-IIIA) wherein R⁴ and R⁵ are each alkyl. In at least one embodiment, R⁴ and R⁵ are each methyl.

At least one embodiment provides an immune modulator (or radical thereof), or a pharmaceutically acceptable salt or hydrate thereof, having the structure of Formula (2-III) or (2-IIIA) wherein m is 0. In another embodiment, m is 1. In another embodiment, m is 2. In another embodiment, m is 3. In another embodiment, m is 4.

At least one embodiment provides an immune modulator (or radical thereof), or a pharmaceutically acceptable salt or hydrate thereof, having the structure of Formula (2-III) or (2-IIIA) wherein X¹, X², and X³ are each N. In at least one embodiment, X¹ is N. In another embodiment, X² is N. In another embodiment, X³ is N.

In some embodiments, the immune modulator (or radical thereof) is represented by any one or more of the structures:

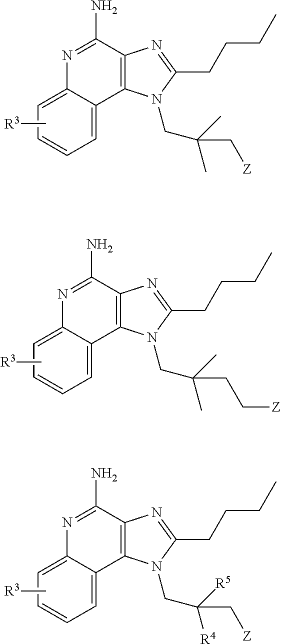

or is a pharmaceutically acceptable salt or hydrate thereof

In some embodiments, the immune modulator (or radical thereof) is represented by any one or more of the structures:

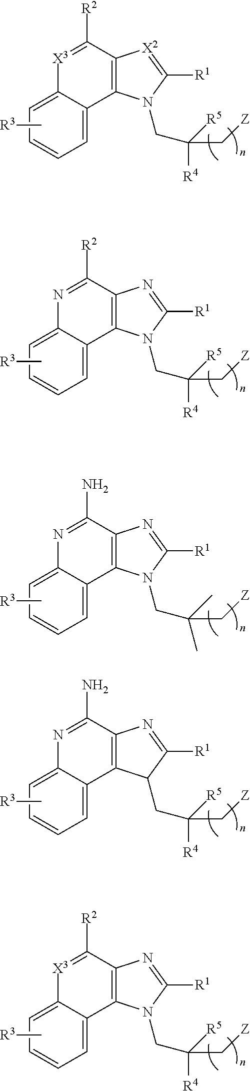

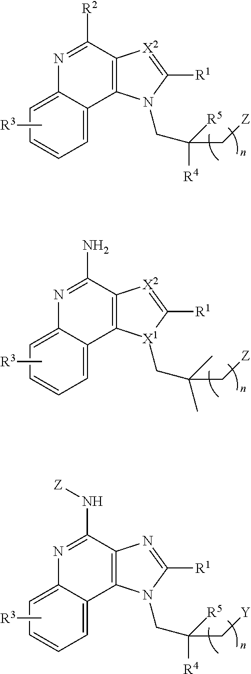

or is a pharmaceutically acceptable salt or hydrate of any of the foregoing structures.

### Targeting Moieties

As previously described, the immune modulators (or radicals thereof), including the TLR-7/8 compound, can be conjugated to a targeting moiety (e.g., via a linker).

The compound can further comprise a targeting moiety (or a radical thereof) attached to the immune modulator (or a radical thereof) that targets a pattern recognition receptor or a DAMP of a cell.

Toxicities associated with systemic administration of at least some of the conventional immune modulators identified herein have historically precluded their practical application with respect to treating cancers. For example, TLR agonists cannot be tolerated by an individual and, in some instances, can result in the death of a subject (e.g., if administered systemically via conventional modalities). In some embodiments, the compounds, such as, for example, those having Formulas I and/or II, are significantly more potent than the conventional drugs that can be used with the compounds, and, in some instances, a mechanism for circumventing systemic toxicity is preferable.

In some embodiments, the targeting moiety comprises a ligand that targets a particular area or tissue of an individual (e.g., with high specificity) and, in certain instances, can, for example, comprise hormones, antibodies, and/or vitamins.

In certain embodiments, the targeting moiety comprises a folate ligand or a functional fragment or analog thereof “Folate” means a folate receptor-binding molecule, including for example folic acid and analogs and derivatives of folic acid such as, without limitation, folinic acid, pteroylpolyglutamic acid, pteroyl-D-glutamic acid, and folate receptor-binding pterdines such as tetrahydropterins, dihydrofolates, tetrahydrofolates, and their deaza and dideaza analogs.

The terms “deaza” and “dideaza” analogs refer to the art-recognized analogs having a carbon atom substituted for one or two nitrogen atoms in the naturally occurring folic acid structure, or analog or derivative thereof. For example, the deaza analogs can include the 1-deaza, 3-deaza, 5-deaza, 8-deaza, and 10-deaza analogs of folate, folinic acid, pteropolyglutamic acid, and folate receptor-binding pteridines such as tetrahydropterins, dihydrofolates, and tetrahydrofolates. The dideaza analogs include, for example, 1,5-dideaza, 5,10-dideaza, 8,10-dideaza, and 5,8-dideaza analogs of folate. Other folates useful as complex-forming ligands are the folate receptor-binding analogs pemetrexed, proguanil, pyrimethamine, trimethoprim, pralatrexate, raltitrexed, aminopterin, amethopterin (also known as methotrexate), N¹⁰-methylfolate, 2-deamino-dydroxyfolate, deaza analogs such as 1-deazamethopterin or 3-deazamethopterin, and 3′,5′-dichloro-4-amino-4-deoxy-N¹⁰-methylpteroylglutamic acid (dichloromethotrexate).

Folic acid and the foregoing analogs and/or derivatives are also termed “a folate.” “the folate,” or “folates” reflecting their ability to bind to folate-receptors. Folic acid is a member of the B family of vitamins and can play an essential role in cell survival, for example, by participating in the biosynthesis of nucleic and amino acids. Folic acid can enhance the specificity of conjugated immune modulator drugs by targeting activated myeloid cells and conjugated anti-cancer drugs by targeting folate receptor-positive cancer cells. Such molecules, when conjugated with exogenous molecules, can be effective to enhance transmembrane transport, such as via folate-mediated endocytosis. The foregoing can be used in the folate receptor-binding ligands described herein.

In at least one embodiment, the targeting moiety comprises a molecule that has (e.g., a high) affinity for folate receptor beta (FRβ). In some instances, the targeting moiety has a specific affinity for any receptor that is particular to cells or tissues of a cancer.

FRβ can be significantly upregulated in activated myeloid cells (e.g., predominantly activated monocytes and M2-like macrophages), for example, with recorded data supporting that FRβ is only induced in cells of myelogenous origin following exposure to anti-inflammatory or proinflammatory stimuli. The folate receptor can be upregulated in (e.g., more than 90%) of non-mucinous ovarian carcinomas. In certain instances, the folate receptor is present in kidney, brain, lung, and breast carcinoma. For example, although there are a number of cancers that do not themselves express the folate receptor in sufficient numbers to provide the desired specificity, cancerous tumors do express myeloid-derived suppressor cells (MDSCs), for example, which do express FRβ and, for example, can be targeted by a targeting moiety provided herein. In some embodiments, folate receptors are not substantially present (e.g., present only at extremely low levels) in healthy (non-myeloid) tissues (e.g., whether lungs, liver, spleen, heart, brain, muscle, intestines, pancreas, bladder, etc.). In some instances, even quiescent tissue-resident macrophages that are abundant throughout the body are predominantly FRβ-negative. In some instances, uptake of folate-targeted imaging agents is in, for example, inflamed tissues, malignant lesions, and the kidneys. In certain instances, subjects devoid of cancer only retain folate-targeted drugs in the kidneys and sites of inflammation. In some instances, the discrepancy in folate receptor expression provides a mechanism for selectively targeting fibrotic cancer cells.

The compounds can leverage the limited expression of FRβ to target/localize systemically administered potent immune modulators (e.g., of the compounds) to fibrotic and/or cancerous tissue. The compounds can be delivered directly to FRβ-expressing cells, for example, which advantageously prevents the systemic activation of the immune system and, for example, can avoid (e.g., at least a portion of) the toxicity that has heretofore prevented systemic use of non-targeting compounds (e.g., drugs).

Provided in some instances are compounds comprising a folate ligand (or radical thereof), or a functional fragment or analog thereof, as a targeting moiety and an immune modulator (e.g., TLR7, TLR8, TLR 7/8, TLR9, or TLR3 agonist). In some instances, TLR7, TLR8, TLR 7/8, TLR9, and TLR3 are present in the endosome. In some embodiments, the compound, or radical thereof, binds to a TLR. In some embodiments, the TLR is TLR7.

A pyrido[2,3-d]pyrimidine analog ligand (e.g., or radical thereof), a functional fragment or analog thereof, or any other molecule, fragment or atom with an affinity (e.g., and without limitation, a high specificity) for FRβ can alternatively be used as the targeting moiety (or radical thereof). For example, folate analog molecules can have a relative affinity for binding FRβ of about 0.01 or greater as compared to folic acid at a temperature about 20° C./25° C./30° C./physiological. Similarly, a Galectin-3 ligand, a translocator protein (TSPO) ligand, and any other ligand or targeting moiety with a highly specific affinity for cancerous cells or tissue can be employed.

Specific examples of suitable targeting moieties (or radicals thereof) will now be provided; however, it will be understood that the targeting moiety (or radical thereof) can comprise any ligand (or radical thereof) useful to target FRβ and is not limited to the structures specified herein. The ligand (or radical thereof) can bind to FRβ.

In certain embodiments, a compound can include a targeting moiety (or radical thereof) that has a structure of Formula V or a functional fragment or analog thereof.

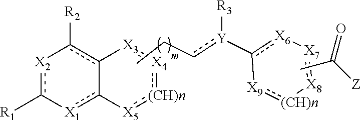

wherein
X₁, X₂, X₃, X₄, X₅, X₆, X₇, X₈, and X₉ are each independently N, NH, CH, CH₂, O, or S;
Y is C, CH, CH₂, N, NH, O, or S;
Z is glutamic acid, valine, or any other amino acid;
R₁ and R₂ are each independently NH₂, OH, SH, CH₃, or H;
R₃ is H or an alkyl;
m and n are each independently 0 or 1; and
[symbol] is representative of either a single or double bond C—C.

In a further aspect, by way of nonlimiting example, the targeting moiety (or radical thereof) of Formula V has a structure of VI (or a functional fragment or analog thereof):

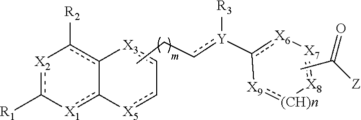

wherein
X₁, X₂, X₃, X₅, X₆, X₇, X₈, and X₉ are each independently N, NH, CH, CH₂, O, or S;
Y is C, CH, CH₂, N, NH, O, or S;
Z is glutamic acid, valine, or a any other amino acid;
R₁ and R₂ are each independently NH₂, OH, SH, CH₃, or H;
R₃ is H or an alkyl;
m and n are each independently 0 or 1; and
[symbol] is representative of either a single or double bond C—C.

Another specific targeting moiety (or radical thereof) of Formula V (or a functional fragment or analog thereof) has a structure of Formula VII:

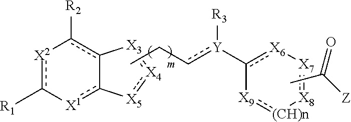

wherein
X₁, X₂, X₃, X₄, X₅, X₆, X₇, X₈, and X₉ are each independently N, NH, CH, CH₂, O, or S;
Y is C, CH, CH₂, N, NH, O, or S;
Z is glutamic acid, valine, or any other amino acid;
R₁ and R₂ are each independently NH₂, OH, SH, CH₃, or H;
R₃ is H or an alkyl:
m and n are each independently 0 or 1; and
[symbol] is representative of either a single or double bond C—C.

In some embodiments, the targeting moiety (or radical thereof) of Formula VI has the structure of Formula VIII:

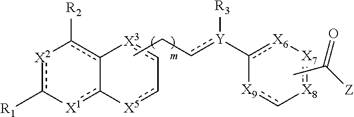

wherein
X₁, X₂, X₃, X₅, X₆, X₇, X₈, and X₉ are each independently N. NH, CH, CH₂, O, or S;
Y is C, CH, CH₂, N, NH, O, or S;
Z is glutamic acid, valine, or any other amino acid;
R₁ and R₂ are each independently NH₂, OH, SH, CH₃, or H;
R₃ is H or an alkyl;
m is 0 or 1; and
[symbol] is representative of either a single or double bond C—C.

In some embodiments, the targeting moiety (or radical thereof) of Formula VI has the structure of Formula IX:

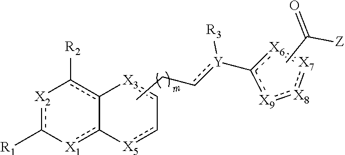

wherein
X₁, X₂, X₃, X₅, X₆, X₇, X₈, and X₉ are each independently N, NH, CH, CH₂, O, or S;
Y is C, CH, CH₂, N, NH, O, or S;
Z is glutamic acid, valine, or any other amino acid;
R₁ and R₂ are each independently NH₂, OH, SH, CH₃, or H;
R₃ is H or an alkyl;
m is 0 or 1; and
[symbol] is representative of either a single or double bond C—C.

In some embodiments, the targeting moiety (or radical thereof) of Formula VII has the structure of Formula X′″ or XI:

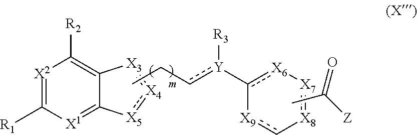

wherein
X₁, X₂, X₃, X₄, X₅, X₆, X₇, X₈, and X₉ are each independently N, NH, CH, CH₂, O, or S;
Y is C, CH, CH₂, N, NH, O, or S;
Z is glutamic acid, valine, or any other amino acid;
R₁ and R₂ are each independently NH₂, OH, SH, CH₃, or H;
R₃ is H or an alkyl;
m is 0 or 1; and
[symbol] is representative of either a single or double bond C—C; or

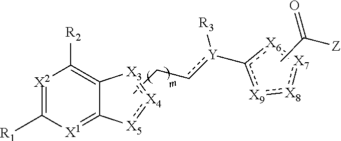

wherein
X₁, X₂, X₃, X₄, X₅, X₆, X₇, X₈, and X₉ are each independently N, NH, CH, CH₂, O, or S;
Y is C, CH, CH₂, N, NH, O, or S;
Z is glutamic acid, valine, or any other amino acid;
R₁ and R₂ are each independently NH₂, OH, SH, CH₃, or H;
R₃ is H or an alkyl;
m is 0 or 1; and
[symbol] is representative of either a single or double bond C—C.

The chemical structures and spectroscopic data of some additional embodiments of a targeting moiety (e.g., or radicals thereof) are provided in the following Table 3, Table 4, Table 5 and Table 6.

Table 3 provides non-limiting examples of additional embodiments of a targeting moiety (e.g., or radicals thereof) having the structure of Formula VIII.

TABLE 3
Formula VIII

| Ligand | Structure |
|---|---|
| a | 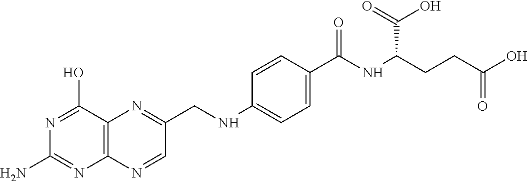 [SMILES: Nc1nc(O)c2nc(CNc3ccc(C(=O)N[C@@H](CCC(=O)O)C(=O)O)cc3)cnc2n1] |
| b | 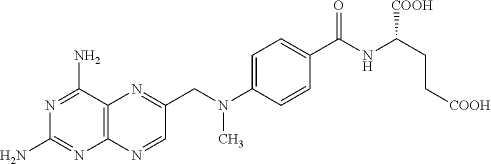 [SMILES: CN(Cc1cnc2nc(N)nc(N)c2n1)c1ccc(C(=O)N[C@@H](CCC(=O)O)C(=O)O)cc1] |
| c | 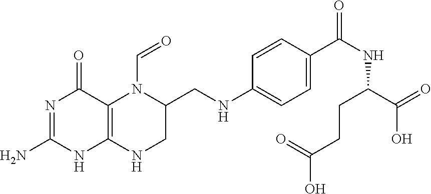 [SMILES: Nc1nc(=O)c2c([nH]1)NCC(CNc1ccc(C(=O)N[C@@H](CCC(=O)O)C(=O)O)cc1)N2C=O] |
| d | 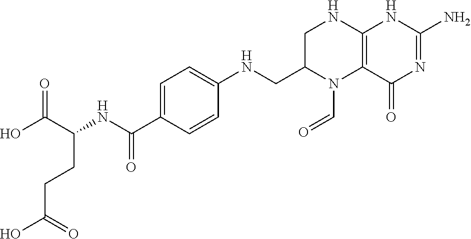 [SMILES: Nc1nc(=O)c2c([nH]1)NCC(CNc1ccc(C(=O)N[C@H](CCC(=O)O)C(=O)O)cc1)N2C=O] |
| e | 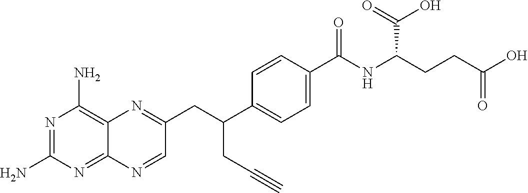 [SMILES: C#CCC(Cc1cnc2nc(N)nc(N)c2n1)c1ccc(C(=O)N[C@@H](CCC(=O)O)C(=O)O)cc1] |
| f | 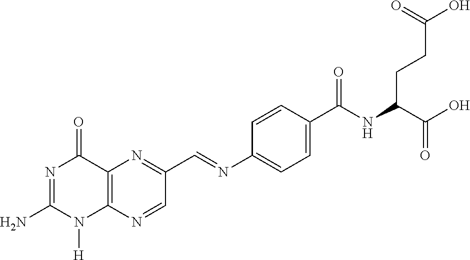 |
| g | 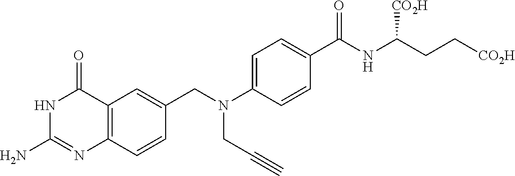 [SMILES: C#CCN(Cc1ccc2nc(N)[nH]c(=O)c2c1)c1ccc(C(=O)N[C@@H](CCC(=O)O)C(=O)O)cc1] |
| h | 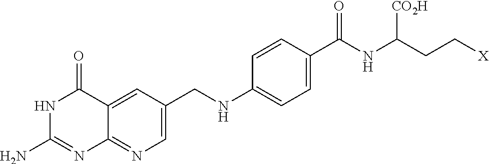 |
| | wherein X is PO(OH)₂, CH₂NH₂, or SO₂OH |
| i | 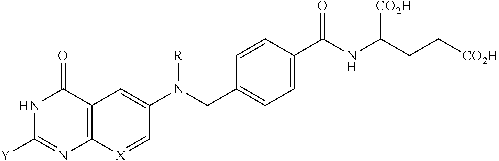 |
| | wherein |
| | X is N or CH; |
| | Y is NH₂, H, or CH₃; and |
| | R is H, CH₃, or CHO |
| j | 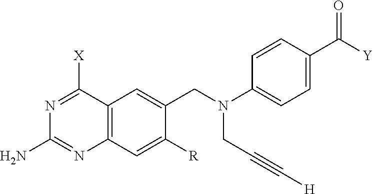 |
| | wherein |
| | X is OH or OCH₃; |
| | R is H or CH₃; and |
| | Y is glutamic acid, valine, or any other amino acid. |

Table 4 provides non-limiting examples of additional embodiments of a targeting moiety (e.g., or radicals thereof) having the structure of Formula IX.

TABLE 4
Formula IX

| Ligand | Structure |
|---|---|
| aa | 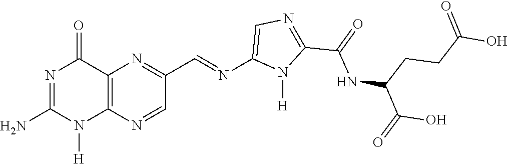 |
| bb | 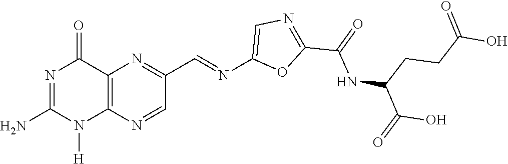 |
| cc | 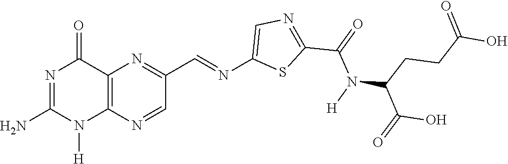 |
| dd | 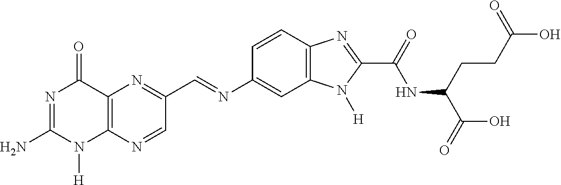 |
| ee | 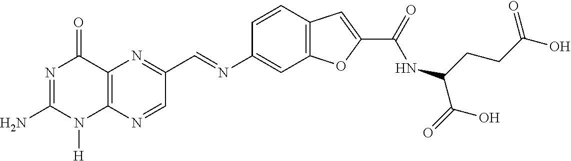 |
| ff | 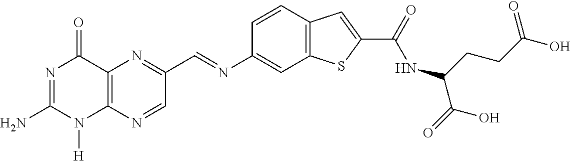 |

Table 5 provides non-limiting examples of additional embodiments of a targeting moiety having the structure of Formula X′″.

TABLE 5
Formula X′′′

| Ligand | Structure |
|---|---|
| aaa | 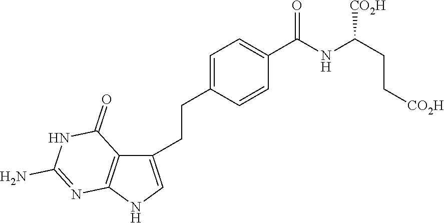 [SMILES: Nc1nc2[nH]cc(CCc3ccc(C(=O)N[C@@H](CCC(=O)O)C(=O)O)cc3)c2c(=O)[nH]1] |
| bbb | 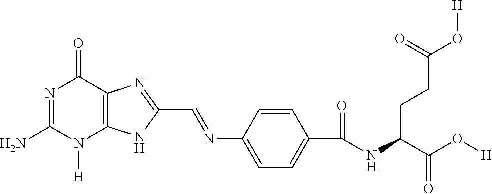 [SMILES: Nc1nc(=O)c2nc(/C=N/c3ccc(C(=O)N[C@@H](CCC(=O)O)C(=O)O)cc3)[nH]c2[nH]1] |
| ccc | 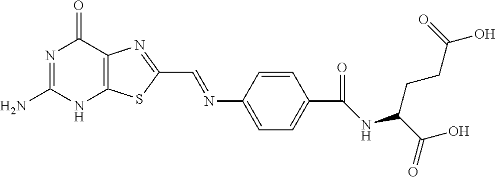 |
| ddd | 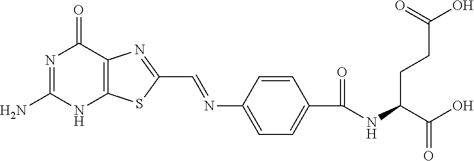 [SMILES: Nc1nc(=O)c2nc(/C=N/c3ccc(C(=O)N[C@@H](CCC(=O)O)C(=O)O)cc3)sc2[nH]1] |

As previously noted, instead of a folate, the targeting moiety (e.g., a radical thereof) can be one or more nonclassical antifolate analogs such as, for example, pyrido[2,3-d]pyrimidine or similar analogs (or radicals thereof) having the formulas (e.g., radicals of the formulas) set forth in Table 6 below (or an analog or functional fragment thereof):

TABLE 6
Nonclassical antifolate analogs

| Ligand | Formula |
|---|---|
| aaaa | 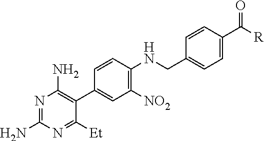 |
| | wherein |
| | R is NH₂ or NHMe or NHCH(CO₂Et)(CH₂)₂CO₂Et or |
| | NHCH(CO₂Et)(CH₂)₂CO₂H or |
| | 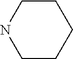 |
| bbbb | 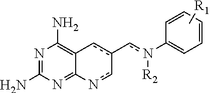 |
| | wherein |
| | Ri is 3,4,5-(OCH₃)₃ or 3,4-(OCH₃)₂ or 4-OCH₃; |
| | R₂ is an H or alkyl chain or CHO; and |
| | is representative of either a single or double bond C—C |
| cccc | 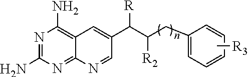 |
| | wherein |
| | n is 0 or 1; |
| | R₁ and R₂ are each independently an H or an alkyl; and |
| | R3 is an H or 3′,4′,5′-OMe or 2′,3′,4′-OMe or 2′,4′,5′-OMe |
| | or 2′,4′,6′-OMe or 3′,4′-OMe or 3′,5′-OMe or 2′,5′-OMe or |
| | 2′,3′-C₄H₄ or 4′-OMe,2′,3′-C₄H₄ or 6′-OMe,2′,3′-C₄H₄ or |
| | 4′-O-C₆H₅ or 4′-CONH-L-glutamic acid. |
| dddd | 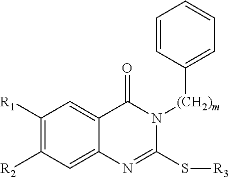 |
| | wherein |
| | n is 0 or 1; |
| | R₁ is CH₃, Cl or OCH₃; |
| | R₂ is H or OCH₃; and |
| | R₃ is one of the following: |
| | 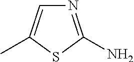 |
| | 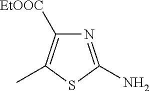 |
| | 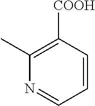 |
| | 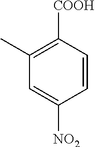 |
| eeee | 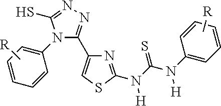 |
| | wherein |
| | Ris H, 4-Cl, 2-CH₃O, 4-CCH₃O, 2,4-(CH₃O)₂, 4-CH₃, or 4- |
| | C₆H₅O. |
| ffff | 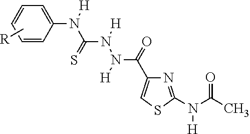 |
| | wherein |
| | Ris H, 4-Cl, 2-CH₃O, 4-CCH₃O, 2,4-(CH₃O)₂, 4-CH₃, or 4- |
| | C₆H₅O. |
| gggg | 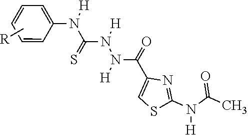 |
| | wherein |
| | R is H, 4-Cl, 2-CH₃O, 4-CH₃O, 2,4-(CH₃O)₂, 4-CH₃, or 4- |
| | C₆H₅O. |
| hhhh | 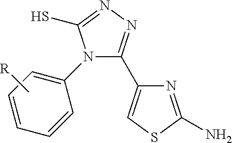 |
| | wherein |
| | R is H, 4-Cl, 2-CH₃O, 4-CH₃O, 2,4-(CH₃O)₂, 4-CH₃, |
| | or 4-C₆H₅O. |
| iiii | 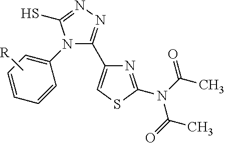 |
| | wherein |
| | Ris H, 4-Cl, 2-CH₃O, 4-CH₃O, 2,4-(CH₃O)₂, 4-CH₃, |
| | or 4-C₆H₅O. |
| jjjj | 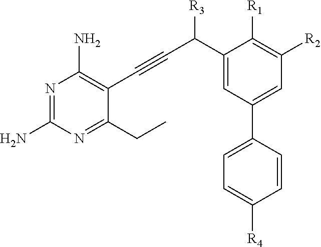 |
| | wherein |
| | R₁ and R₂ are each independently H or OMe; |
| | R₃ is H or an alkyl; and |
| | R₄ is o-COOH or m-COOH or p-COOH. |
| kkkk | 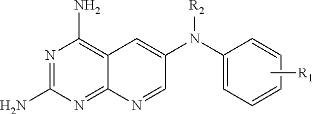 |
| | wherein |
| | R₁ is H or 2′-OMe or 4′-OMe or 2′,5′-diOMe or 3′,4′,5′- |
| | triOMe or 4′-Me or 4′-i-Pr or 3′,4′-(C₄H₄) or 2′,3′-(C₄H₄) |
| | or 4′-NO₂ or 2′,5′-diF or 3′,4′,5′--triF; and |
| | R₂ is H or an alkyl. |

One provides a compound, or a pharmaceutically acceptable salt thereof, having the structure Formula (2-II), (2-IIA), (2-III) or (2-IIIA) (described above) wherein G is a folate receptor-binding ligand. In one embodiment, G is or is derived from folate, folic acid, or a functional fragment or derivative thereof. In one embodiment, G is a folate or folate derivative. In another embodiment, G is a pteroic acid or pteroyl derivative.

One embodiment provides a compound, or a pharmaceutically acceptable salt or hydrate thereof, having the structure embodiment Formula (2-II), (2-IIA), (2-III) or (2-IIIA) wherein G is a group or comprise a group of Formula (2-IV):

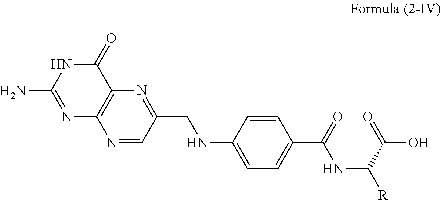

wherein, each R is independently,

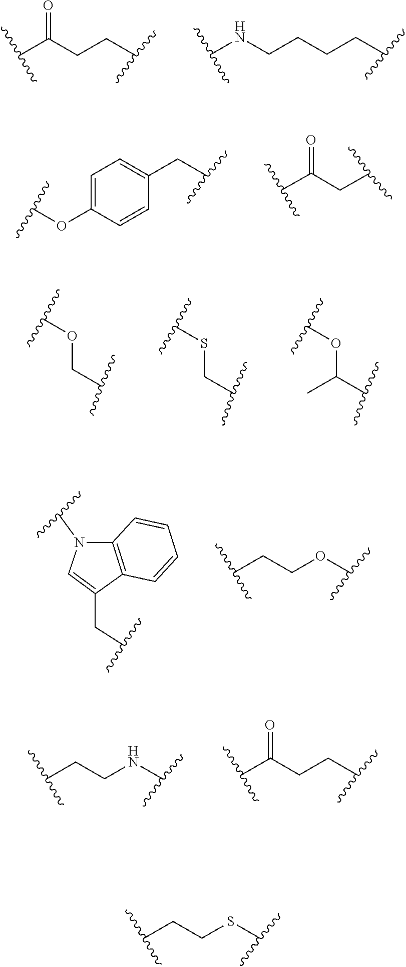

wherein R is a naturally occurring or unnatural amino acid or its derivative or fragments.

One embodiment provides a compound, or a pharmaceutically acceptable salt or hydrate thereof, having the structure of Formula (2-II), (2-IIA), (2-III) or (2-IIIA)) wherein G is a group or comprises a group of Formula (2-V):

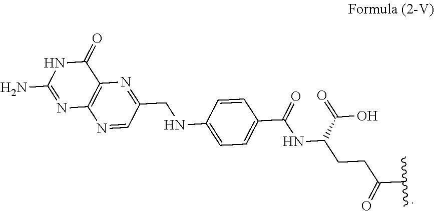

One embodiment provides a compound, or a pharmaceutically acceptable salt or hydrate thereof, having the structure Formula (2-II), (2-IIA), (2-III) or (2-IIIA) wherein G is a group or comprises a group of Formula (2-VI):

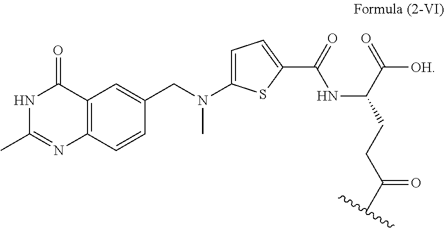 [SMILES: *C(=O)CC[C@H](NC(=O)c1ccc(N(C)Cc2ccc3nc(C)[nH]c(=O)c3c2)s1)C(=O)O]

### Linkers

A compound can comprise one or more linkers, wherein a radical of the targeting moiety is conjugated to a radical of the immune modulator through the one or more linkers. The term “linker” includes a chain of atoms that is bio-functionally adapted to form a chemical bond with an A, B, or S and connects two or more functional parts of a molecule to form the compound. Illustratively, the chain of atoms can be selected from carbon (C), N, oxygen (O), sulfur (S), silicon (Si), and phosphorus (P), or C, N, O, S, and P, C, N, O, and S. The chain of atoms can covalently connect different functional capabilities of the compound, such as a folate and the drug (i.e., immune modulator).

The linker can comprise a wide variety of links, such as in the range from about 2 to about 100 atoms in the contiguous backbone and can comprise a releasable or non-releasable linker.

In some embodiments, a linker comprises PEG, a PEG derivative, or any other linker known in the art or hereinafter developed that can achieve the purpose set forth herein. In some embodiments, the compound further comprises a linker (“L” or “Lₙ”) between or otherwise connecting the targeting moiety and the immune modulator. In some embodiments, the linker Lₙ is configured to avoid release of the immune modulator, and n is an integer equal to or less than 50. In some embodiments, the linker Lₙ comprises PEG or a PEG derivative, n is an integer selected from the range 1 to 32, and the targeting moiety (e.g., a radical thereof) comprises a radical of folate receptor-binding ligand comprising FRβ binding ligand. In some embodiments, n is 1-50, 1-10.2-8, or 2-4.

In some embodiments, the linker is repeated n times (“Lₙ”), where n is a positive integer. For example, and without limitation, n can be any integer selected from a range of 1-16, 1-32, 1-64, or 1-96. The number of repeats in the linker can be selected to achieve the desired functionality, size, and/or potency of the compound and/or in view of the desired application.

The linker is releasable (i.e., “quick-release”) or non-releasable (i.e., “slow-release”). In some instances, the target for a compound comprising a non-releasable linker is the endosome (e.g., of the cell of interest), for example, whereas the target for a releasable linker, in some instances, the endosome, the cytoplasm, or both (e.g., of the cell of interest). In some embodiments, L is a quick-release, hydrolyzable linker. In some embodiments, L is a slow-release, non-hydrolyzable linker. In some embodiments, L is an optionally substituted heteroalkyl.

The terms “releasable” and “quick-release” (which are used interchangeably herein) in the context of a linker means a linker that includes at least one bond that can be cleaved (e.g., chemically or enzymatically hydrolyzed) to varying degrees under certain conditions and, in particular, can be fragmented or cleaved in less than about 1 week when under, or otherwise exposed to, certain metabolic, physiological, or cellular conditions that can initiate a cascade of fragmentation or bond cleavage (which may, for example, result in the release of one or more of the moieties connected through one or more portions of the linker (e.g., targeting moiety and immune modulator)). A quick-release linker can comprise, for example, by reducing agent-labile, pH-labile, acid-labile, base-labile, oxidatively labile, metabolically labile, biochemically labile, enzyme-labile or p-aminobenzylic based multivalent releasable bond.

Bond cleavage can occur by standard chemical hydrolysis reactions that occur, for example, at physiological pH, or as a result of compartmentalization into a cellular organelle such as an endosome having a lower pH than cytosolic pH. Bond cleavage can also occur by acid-catalyzed elimination. Alternatively, fragmentation can be initiated by a nucleophilic attack on a disulfide group of the quick-release linker, causing cleavage to form a thiolate, for example. In any of these cases, the quick-release nature of such linkers can be realized by whatever mechanism can be relevant to the chemical, metabolic, physiological, or biological conditions present. In certain embodiments, a quick-release linker comprises one or more sulfide bridges. In some instances, the releasable linker is broken into two or more fragments. In some instances, the releasable linker is separated from the targeting moiety. In some embodiments, the targeting moiety and the immune modulator are released from each other, and the immune modulator becomes active.

In contrast, the terms “non-releasable” and “slow-release” (which are used interchangeably herein) in the context of a linker means a linker that includes at least one bond that is not easily or quickly broken (i.e. the bond does not cleave) and, while potentially cleavable or fragmentable to varying degrees under certain conditions, does not cleave, fragment, or otherwise release one or more of the moieties connected through one or more portions of the linker (e.g., targeting moiety and immune modulator) when subjected to certain metabolic, physiological, or cellular conditions that can initiate a cascade of fragmentation (e.g., after administration to a subject) for more than about 1 week (e.g., 1 week), more than about 1 month (e.g., 1 month), more than about 4 months (e.g., 4 months), more than about 6 months (e.g., 6 months), or more than about 1 year (e.g., 1 year). In certain embodiments, a slow-release linker comprises one or more amide bonds.

In some embodiments, a compound comprises a non-releasable linker that does not release any component of the compound (e.g., a targeting ligand (e.g., a fully amorphous (FA)-ligand or pteroyl amino acid) or an immune modulator (e.g., a TLR7 agonist)). In some embodiments, the non-releasable linker lacks a disulfide bond (e.g., S-S) or an ester in the backbone. In some embodiments, the compound comprises a targeting moiety and an immune modulator connected by a backbone that is substantially stable for the entire duration of the compound's circulation in a subject (e.g., during endocytosis into the target cell endosome). In some embodiments, a compound comprising the non-releasable linker is particularly beneficial when the immune modulator targets TLRs, NOD-like receptors, and/or other pattern recognition receptors present within the endosome of a cell. The non-releasable linker can comprise: an amide, ester, ether, amine, and/or thioether (e.g., thio-maleimide). While specific examples are provided herein, it will be understood that any molecule(s) can be used in the non-releasable linker provided that at least one bond that is not easily or quickly broken under physiological conditions is formed.

Perhaps more specifically, in some embodiments, a non-releasable linker comprises a linker that, at a neutral pH, for example, less than ten percent (10%) (e.g., less than 5%, less than 4%, less than 3%, less than 2%, less than 1%, less than 0.1%, less than 0.01%, or less than 0.001%) will hydrolyze in an aqueous (e.g., buffered (e.g., phosphate buffer) solution) within a period of time (e.g., 24 hours). In some embodiments, where a non-releasable linker is employed, less than about ten percent (10%), and preferably less than five percent (5%) or none, of the compound administered releases the free drug (e.g., in systemic circulation prior to uptake by the targeted cells/tissue). In some embodiments where the compound comprises a non-releasable linker, within one (1) hour of administration, less than five percent (5%) of the free drug is released from the compound while the compound is in systemic circulation.

In some embodiments, the targeting moiety does not cleave from the drug/immune modulator for the compound to be therapeutically effective in vivo. In some embodiments, this is advantageous as it allows for the use of targeting compositions comprising potent drugs (e.g., TLR7 agonists), for example, because only a negligible amount (if any) of the drug (e.g., immune modulator, e.g., TLR7 agonist) is released (e.g., systemically) prior to the targeted delivery of the compound.

Tuning the releasing properties of a compound's active components can be a difficult aspect of the preparation of effective pharmaceutical compositions. The compounds comprising the non-releasable linkers can avoid the difficulties of the preparation of effective pharmaceutical compositions (e.g., by removing the necessity of timing the release). In some embodiments, the immune modulator or warhead of the compound is active when bound (e.g., conjugated to the targeting compound). In some embodiments, while the warhead/immune modulator is active, the non-releasable linker and the targeting moiety prevent the release of toxic cytokines (e.g., by the subject's body) that activate the immune system (such as, for example, interleukin 6 (IL-6)) (e.g., because the compound is specifically targeted (using, for example, folate or an analog thereof)). In certain instances, the immune modulator cannot access the appropriate (e.g., targeted) receptor within the endosome of the cell until the compound binds to the targeted receptor (for example, a folate receptor), for example, even though the warhead/immune modulator of the compound is active when connected to the non-releasable linker.

By way of nonlimiting examples, the linker 106 of FIG. 1A is a non-releasable PEG linker, whereas the linker 156 of FIG. 1B is a self-immolative, releasable linker (e.g., comprising a disulfide bond (e.g., S-S)). For example, the scheme shown in FIG. 1B illustrates the self-immolative cascade of compound 150 upon cleavage from the targeting moiety 154.

In some embodiments, the linker 156 is formed such that the drug (e.g., immune modulator) is cleaved from the targeting moiety 154 only after sufficient time has passed for the compound to circulate within a subject's systemic circulation following administration (e.g., clear from non-targeted tissues, and be captured and internalized by the targeted cell and/or receptor). In some embodiments, the time period for the release will vary (e.g., from subject to subject (e.g., based on a variety of factors)). In some embodiments, a releasable linker is engineered such that it will not cleave/release until at least 24 hours post administration or even over a period of a week. In some embodiments, the compound can safely pass through the subject's system and any amount not captured by the targeted cells (e.g., those expressing FRβ, for example) can be excreted prior to release/activation thus preventing toxicity (e.g., because the immune modulator is not active when bound to a releasable linker).

Both releasable and non-releasable linkers can be engineered to optimize biodistribution, bioavailability, and PK/PD (e.g., of the compound) and/or to increase uptake (e.g., of the compound) into the targeted tissue pursuant to methodologies commonly known in the art or hereinafter developed such as through PEGlaytion and the like. In some embodiments, the linker is configured to avoid significant release of a pharmaceutically active amount of the drug in circulation prior to capture by a cell (e.g., a cell of interest (e.g., a macrophage in cancer tissue to be treated)).

In some embodiments, the compounds comprising releasable linkers are designed to diffuse across the membrane of the endosome and, for example, into the cytoplasm of the targeted cell. In some embodiments, releasable linkers are designed such that the immune modulator is not released until the compound reaches the cytoplasm.

In some embodiments, a compound comprises a releasable linker (e.g., to facilitate the release of the immune modulator in the cytoplasm, e.g., where the immune modulator comprises a PI3K kinase, IRAK, or an activator of 1-kappa-D (Iκβ) kinase (e.g., using Prostratin or the like) or nuclear factor kappa-light-chain-enhancer of activated B cells (NF-κβ) (see, e.g., Table 1), or an Myeloid differentiation primary response 88 (MyD88) agonist). In some embodiments, the releasable linker prevents the release of the immune modulator, for example, until after the targeting moiety binds the appropriate target (e.g., a macrophage folate receptor) is internalized into the endosome of the targeted cell and/or diffuses into the cytoplasm (e.g., which is where the desired pattern recognition receptor is located). In some embodiments, the releasable linker releases the immune modulator within the endosome.

In some embodiments, the linker can comprise one or more spacers (e.g., which can also be used to specifically design characteristics of the compound such as, for example, to facilitate a particular release time, facilitate an increase in uptake into a targeted tissue, and/or optimize biodistribution, bioavailability, and/or PK/PD of a compound). A spacer can comprise one or more alkyl chains. PEGs, peptides, sugars, peptidoglycans, clickable linkers (e.g., triazoles), rigid linkers such as poly prolines and poly piperidines, and the like.

In some embodiments, a linker comprising PEG₁₂ significantly reduces—if not altogether avoids—nonspecific uptake of the compounds provided herein (e.g., into a non-targeted organ (e.g., into the liver and/or kidneys of a subject following administration)). In some embodiments, the compounds avoid delivery to the liver and kidneys. In some embodiments, the targeting moieties (in their free form, a radical thereof, or a compound thereof) do not bind with uptake receptors on non-targeted cells (e.g., provided the organs are not the targeted sites, and, as such, stimulation of the immune complex in those organs can be avoided, which is highly beneficial in a clinical context).

A compound comprising a non-releasable linker can reduce or eliminate toxicity of an immune modulator released from the compound in its free form (e.g., a free form of a compound and/or ligand provided herein). In certain embodiments, a compound, or a pharmaceutically acceptable salt or hydrate thereof, has or comprises the structure of Formulae (2-II). (2-IIA), (2-III), or (2-IIIA) described below, wherein L is a quick-release linker (e.g., amide, ester, ether, or sulfonamide).

In another embodiment, L is an optionally substituted heteroalkyl. The term “heteroalkyl,” by itself or in combination with another term means, unless otherwise stated, a stable straight or branched chain, or combination(s) thereof, consisting of at least one carbon atom and at least one heteroatom selected from the group consisting of O, N, P, Si, and S, and wherein the nitrogen and sulfur atoms can optionally be oxidized, and the nitrogen heteroatom can optionally be quarternized. The heteroatom(s) O, N, P, S, and Si can be placed at any interior position of the heteroalkyl group or at the position at which the alkyl group is attached to the remainder of the molecule. Examples include, without limitation, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH₂=CH—O—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms can be consecutive, such as, for example, —CH₂—NH—OCH₃.

In some embodiments, the heteroalkyl is unsubstituted. In other embodiments, the heteroaryl is substituted with at least one substituent selected from the group consisting of alkyl, hydroxyl, acyl, oxo, PEG, carboxylate, and halo. “Halo” or “halogen” by itself or as part of another substituent means, unless otherwise stated, a fluorine, chlorine, bromine, or iodine atom.

In another embodiment. L is a substituted heteroalkyl with at least one disulfide bond in the backbone thereof

In another embodiment, L is a peptide or a peptidoglycan with at least one disulfide bond in the backbone thereof. The terms “polypeptide,” “peptide,” and “protein” are used interchangeably herein to refer to a polymer of amino acid residues, a polypeptide, or a fragment of a polypeptide, peptide, or fusion polypeptide. The terms apply to amino acid polymers in which one or more amino acid residue is an artificial chemical mimetic of a corresponding naturally occurring amino acid, as well as to naturally occurring amino acid polymers and non-naturally occurring amino acid polymers.

In some embodiments, L comprises —CONH—CH(COOH)—CH₂—S—S—CH₂—CRₐR_{b}—O—CO—, —CONH—CH(COOH)CRₐR_{b}—O—CO—, —C(O)NHCH(COOH)(CH₂)₂—CONH—CH(COOH)CRₐR_{b}—O—CO— or —C(O)NHCH(COOH)(CH₂)₂—CONH—CH(COOH)—CH₂—S—S—CH₂—CRₐR_{b}—O—CO—, wherein Rₐ and R_{b} are independently H, alkyl, or heteroalkyl (e.g., PEG).

In another embodiment, L is a cleavable linker that can be cleaved by enzymatic reaction, reaction oxygen species (ROS) or reductive conditions.

In some embodiments, L has the formula: —NH—CH₂—CR⁶R⁷—S—S—CH₂—CH₂—O—CO—, wherein R⁶ and R⁷ are each, independently, H, alkyl, or heteroalkyl.

The linker can further comprise a spacer. In certain embodiments, the spacer is a hydrophilic spacer. In some embodiments, the compound has the structure of Formula XII (e.g., a sub-structure of the TLR7 agonist of Formula III conjugated with folate via a releasable linker containing a first hydrophilic spacer):

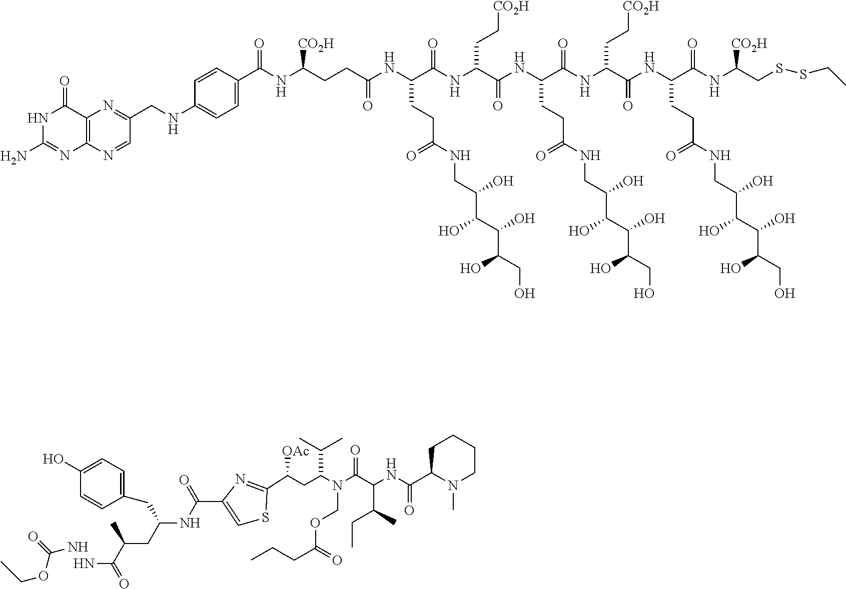

or is a pharmaceutically acceptable salt or hydrate thereof.

In some embodiments, the compound has a structure of Formula XIII (e.g., a substructure of the TLR7 agonist of Formula III conjugated with folate via a non-releasable linker (covalent bond) comprising a second hydrophilic spacer):

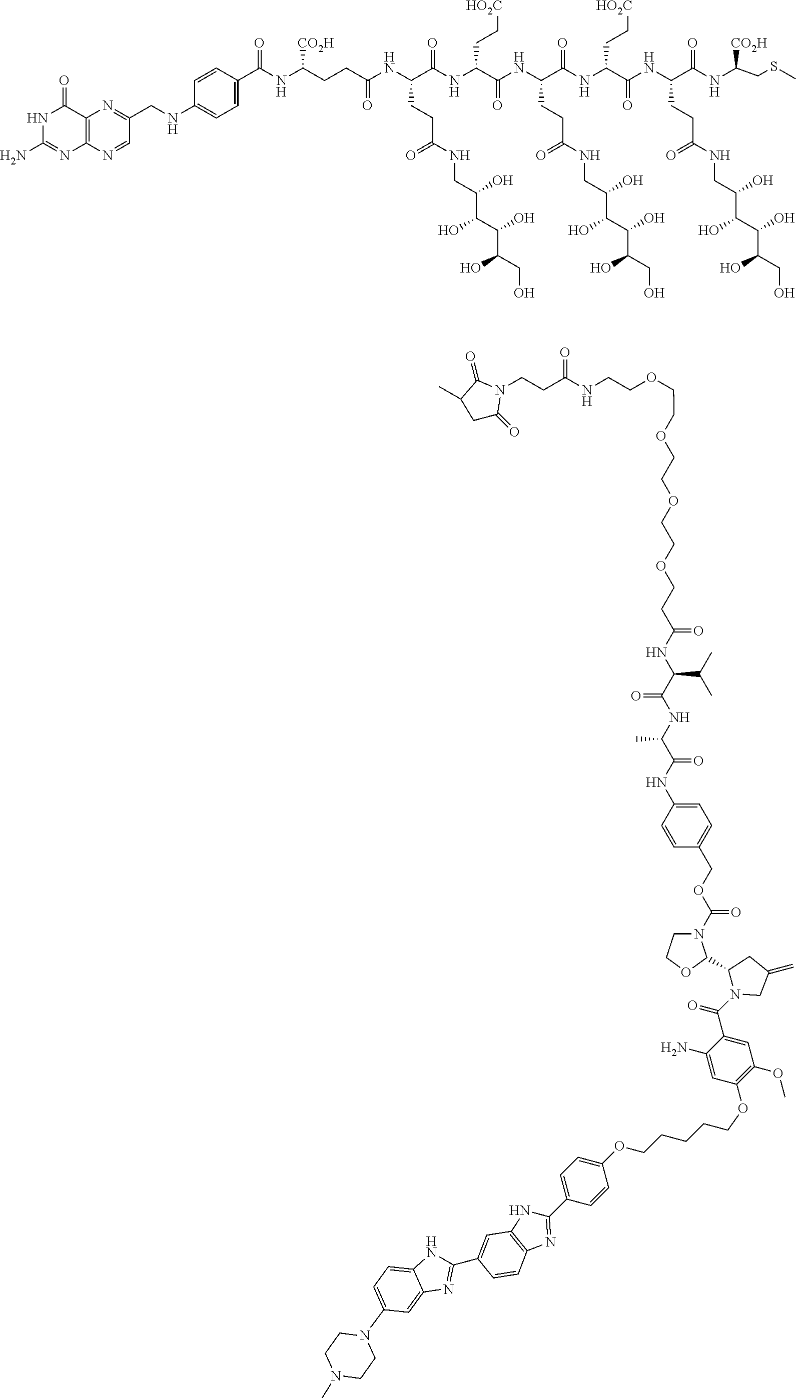

or is a pharmaceutically acceptable salt or hydrate thereof.

In some embodiments. L is a group or comprises a group of the formula:

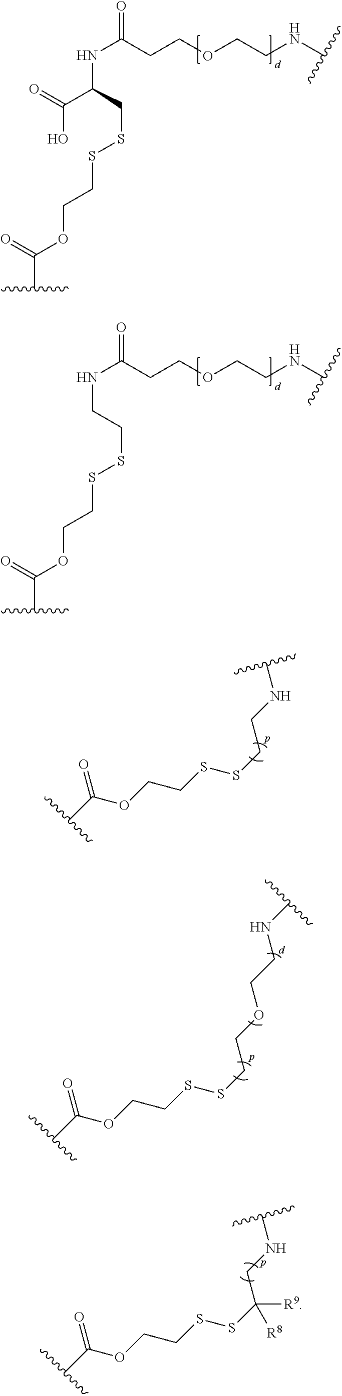

wherein each

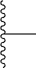

is a point of attachment; p is 0to 30; and d is Ito 40; and
wherein R⁸ and R⁹ are each, independently, H, alkyl, cyclic, aryl, or heteroalkyl.

One embodiment provides a compound, or a pharmaceutically acceptable salt thereof, having the structure of Formula (2-II), (2-IIA), (2-III) or (2-IIIA) wherein L is a non-cleavable linker.

One embodiment provides a compound, or a pharmaceutically acceptable salt or hydrate thereof, having the structure of Formula (2-II), (2-IIA), (2-III) or (2-1IIA) wherein L is a non-hydrolyzable linker.

In some embodiments, L is selected from the group consisting of alkylene, heteroalkylene, —O— alkynylene, alkenylene, acyl, aryl, heteroaryl, amide, oxime, ether, ester, triazole, PEG, and carboxylate.

In one embodiment, L is an alkyl ether. In another embodiment. L is an amide. In another embodiment, L is a peptide or a peptidoglycan. In another embodiment, L is an amino acid. In another embodiment, L is a PEG (e.g., —OCH₂—CH₂—O—). In another embodiment, L is poly saccharide.

In some embodiments. L comprises a structure of:

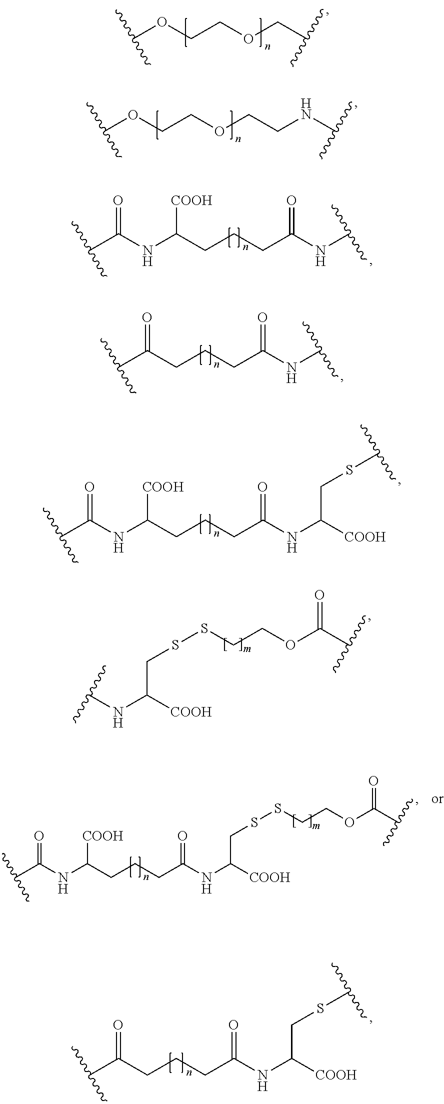

wherein each

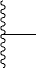

is a point of attachment and n and m are each independently 0 to 10.

In some embodiments, the linker comprises a structure of:

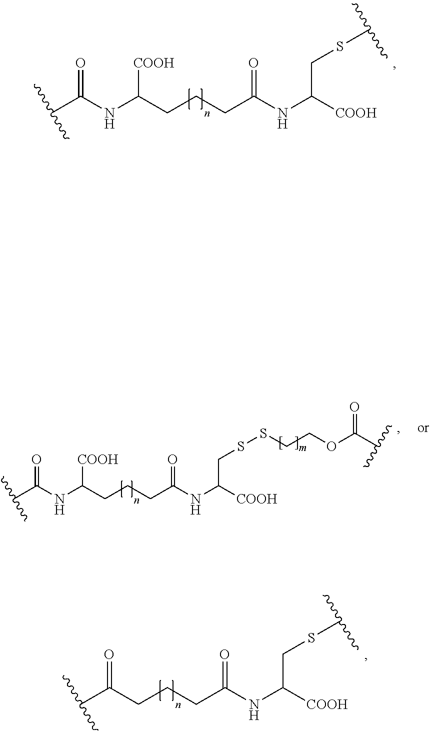

wherein each

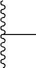

is a point of attachment, and n and m are each independently 0 to 10.

In some embodiments, the L comprises a structure of:

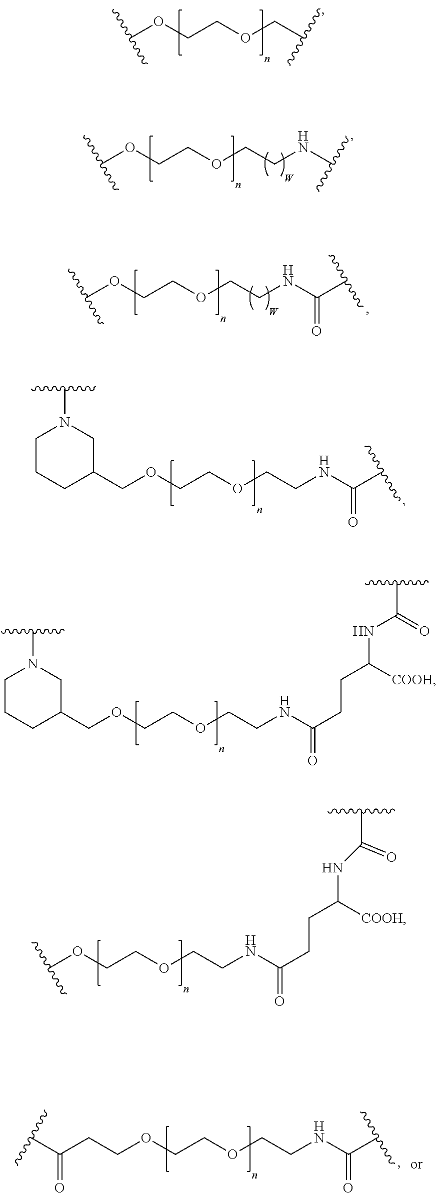

wherein each

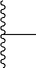

is a point of attachment and n is 1 to 32. In at least one exemplary embodiment, n is 1 to 30 and w is 0 to 5.

In some embodiments, the linker comprises the structure of:

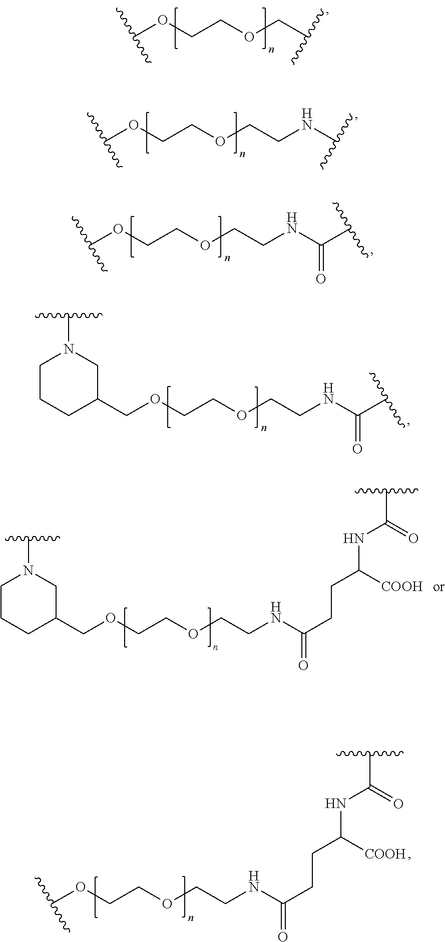

wherein n is 1 to 16 and each

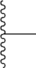

is a point of attachment.

In some embodiments, the L comprises the structure of:

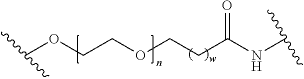

wherein each

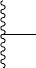

is a point of attachment, n is 1 to 30, and w is 0 to 5.

In one embodiment, L is selected from the following list:

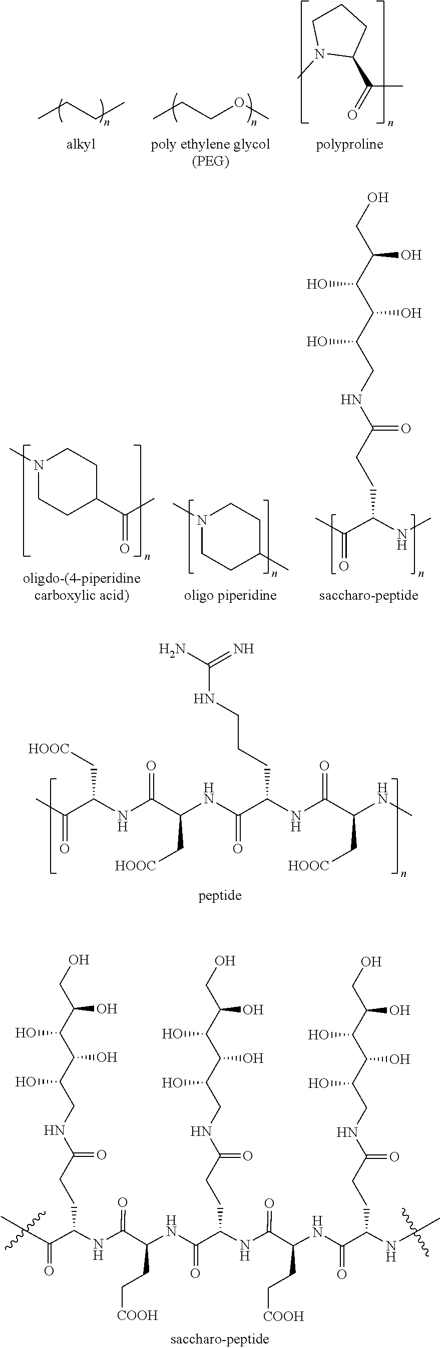

wherein n is 0-30.

Specific examples of exemplary conjugated compounds are provided herein. It will be appreciated by those of skill in the art that compound can exhibit polymorphism; the compound can comprise any racemic, optically-active, polymorphic, or stereoisomeric form, or mixtures thereof, of a compound that exhibits the useful properties described, it being well-known in the art how to prepare optically active forms (e.g., by resolution of the racemic form by recrystallization techniques, by synthesis from optically-active starting materials, by chiral synthesis, or by chromatographic separation using a chiral stationary phase) and how to determine antitumor activity using the standard tests described herein, or using other similar tests which are well-known in the art. Further, unless otherwise expressly stated, structures depicted herein are also meant to include all stereochemical forms of the structure, i.e., the right hand (R) and left hand (S) configurations of each asymmetric center. Therefore, single stereochemical isomers as well as enantiomeric and diasteromeric mixtures of the present compound are within the scope of the present disclosure.

Specific values listed herein for radicals, substituents, and ranges are for illustration purposes only unless otherwise specified; such examples do not exclude other defined values or other values within defined ranges for the radicals and substituents. For example, (C₁-C₆)alkyl can be methyl, ethyl, propyl, isopropyl, butyl, iso-butyl, sec-butyl, pentyl, 3-pentyl, or hexyl; (C₁-C₃)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; (C₁-C₃)alkoxy can be methoxy, ethoxy, or propoxy; and (C₂-C₆)alkanoyloxy can be acetoxy, propanoyloxy, butanoyloxy, isobutanoyloxy, pentanoyloxy, or hexanoyloxy.

Further, where a moiety is substituted with an R substituent or a substituted group, the group may be referred to as “R-substituted.” Where a moiety is R-substituted or is otherwise described as generally comprising a substituted group, the moiety is substituted with at least one R substituent, and each substituent is optionally different. It will be appreciated that the substituted group (or R substituent) may comprise any molecule or combination molecules provided the inclusion thereof does not substantially affect the overall structure and shape of the compound, nor alters any hydrogen bonds that are essential to the underlying compound achieving its intended purpose (e.g., binding to a targeted pattern recognition receptor).

Where substituent groups are specified by the conventional chemical formulae, written from left to right, they equally encompass the chemically identical substituents that would results from writing the structure from right to left, e.g., —CH₂O— is equivalent to —OCH₂—.

In some embodiments, a compound comprises a radical of a targeting moiety conjugated with a radical of an immune modulator or a pharmaceutically acceptable salt or hydrate thereof such that the immune modulator (or radical thereof) or pharmaceutically acceptable salt or hydrate thereof remains pharmaceutically active when conjugated. The targeting moiety can comprise any targeting moiety described herein and, in at least one embodiment, comprises a folate ligand, any other folate receptor-binding molecule (e.g., or a functional fragment or analog of either of the foregoing) or a pyrido[2,3-d]pyrimidine analog. In some embodiments, the targeting moiety (or compound or radical thereof) is specific for FRβ.

In some embodiments, a compound comprises one or more linkers, wherein a radical of the targeting moiety is conjugated to a radical of the immune modulator through the one or more linkers. For example, where the immune modulator or pharmaceutically acceptable salt or hydrate thereof has Formula I or II, a radical of the immune modulator can be conjugated to a radical of the targeting moiety at one of R¹, R², or R³, through a linker or directly. Similarly, where the immune modulator or pharmaceutically acceptable salt or hydrate thereof has Formula III, a radical of the immune modulator can be conjugated to a radical of the targeting moiety at one of R¹ or R³, through a linker or directly. Alternatively, where the immune modulator or pharmaceutically acceptable salt or hydrate thereof has Formula IV, a radical of the immune modulator can be conjugated to a radical of the targeting moiety at one of R¹ or R² through a linker or directly. As described herein, a linker can be releasable or non-releasable.

It is understood that any combination of a radical of a compound (e.g., a radical of a compound in any one of Tables 1 or 2), a linker (e.g., as provided herein), and a radical of a ligand (e.g., a radical of a ligand in any one of Tables 3-6) can be combined to form a compound provided herein. In some embodiments, the radical of the compound or the radical of the ligand is a carbon atom or a heteroatom (e.g., O, S, N, etc.). In some embodiments, the radical of the compound is C or O. In some embodiments, the radical of the ligand is C or O. In some embodiments, the point of attachment of the compound and the ligand (e.g., through a linker) is determined by the placement of the radical. In some embodiments, the linkers comprise a spacer (e.g., as described elsewhere herein). It is also understood that any compound provided herein can be synthesized in a similar process as provided in the methods provided in the Examples.

Non-limiting examples of compound provided herein are provided in Table 7.

TABLE 7
Examples of Compound.

| Compound | Linker | Ligand |
|---|---|---|
| A• | 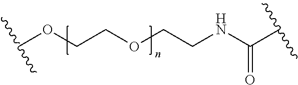 | a• |
| | wherein n is 1 to 16 | |
| A• | 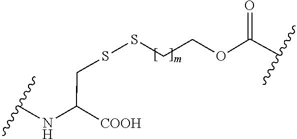 | a• |
| | m is 1 to 5 | |
| Z• | 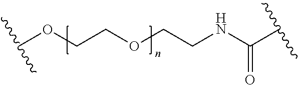 | a• |
| | wherein n is 1 to 16 | |
| Z• | 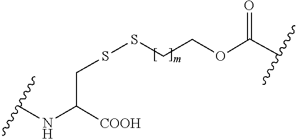 | a• |
| | m is 1 to 5 | |
| F• | 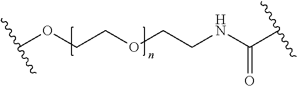 | a• |
| | wherein n is 1 to 16 | |
| F• | 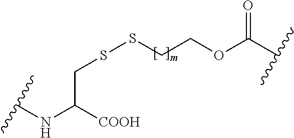 | a• |
| | m is 1 to 5 | |
| L• | 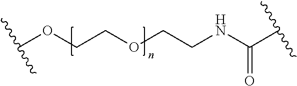 | a• |
| | wherein n is 1 to 16 | |
| L• | 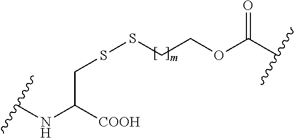 | a• |
| | m is 1 to 5 | |
| D• | 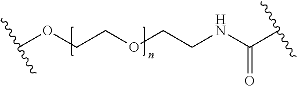 | ac• |
| | wherein n is 1 to 16 | |
| D• | 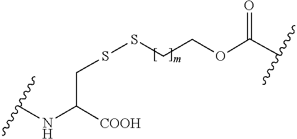 | a• |
| | m is 1 to 5 | |
| A• | 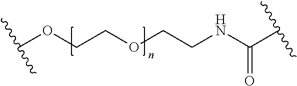 | be• |
| | wherein n is 1 to 16 | |
| A• | 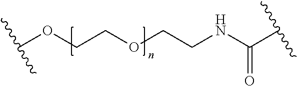 | f• |
| | wherein n is 1 to 16 | |
| A• | 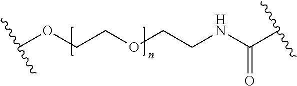 | h• |
| | wherein n is 1 to 16 | |
| A• | 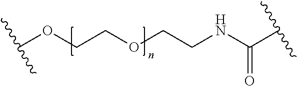 | aa• |
| | wherein n is 1 to 16 | |
| A• | 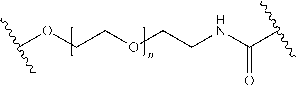 | bb• |
| | wherein n is 1 to 16 | |
| A• | 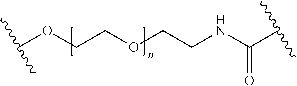 | cc• |
| | wherein n is 1 to 16 | |
| A• | 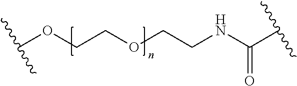 | aaa• |
| | wherein n is 1 to 16 | |
| Z• | 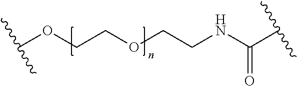 | b• |
| | wherein n is 1 to 16 | |
| Z• | 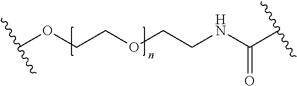 | f• |
| | wherein n is 1 to 16 | |
| Z• | 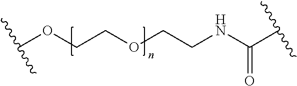 | h• |
| | wherein n is 1 to 16 | |
| Z• | 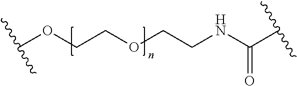 | aa• |
| | wherein n is 1 to 16 | |
| Z• | 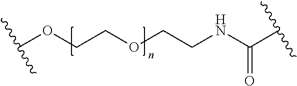 | bb• |
| | wherein n is 1 to 16 | |
| Z• | 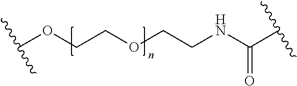 | cc• |
| | wherein n is 1 to 16 | |
| Z• | 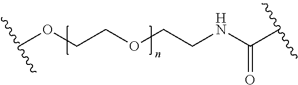 | aaa• |
| | wherein n is 1 to 16 | |

Non-limiting examples of compound provided herein are provided in Table 8.

TABLE 8
Additional Examples of Compound

| Compound | Structure |
|---|---|
| 1A | 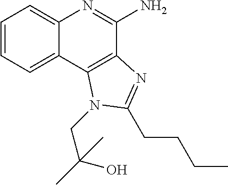 [SMILES: CCCCc1nc2c(N)nc3ccccc3c2n1CC(C)(C)O] |
| 1B | 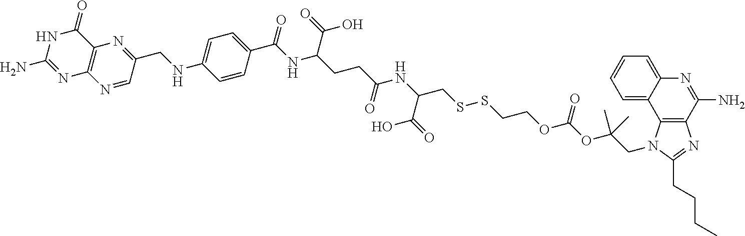 [SMILES: CCCCc1nc2c(N)nc3ccccc3c2n1CC(C)(C)OC(=O)OCCSSCC(NC(=O)CCC(NC(=O)c1ccc(NCc2cnc3nc(N)[nH]c(=O)c3n2)cc1)C(=O)O)C(=O)O] |
| 2A | 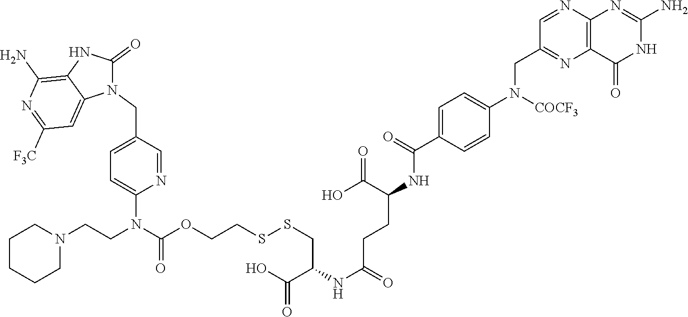 |
| 3A | 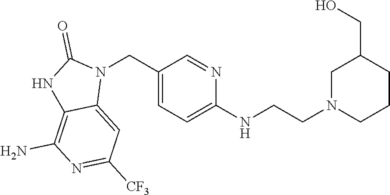 [SMILES: Nc1nc(C(F)(F)F)cc2c1[nH]c(=O)n2Cc1ccc(NCCN2CCCC(CO)C2)nc1] |
| 3 | 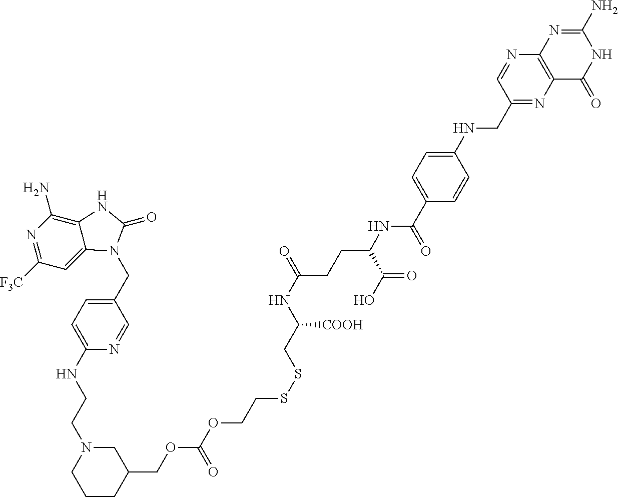 |
| 3C | 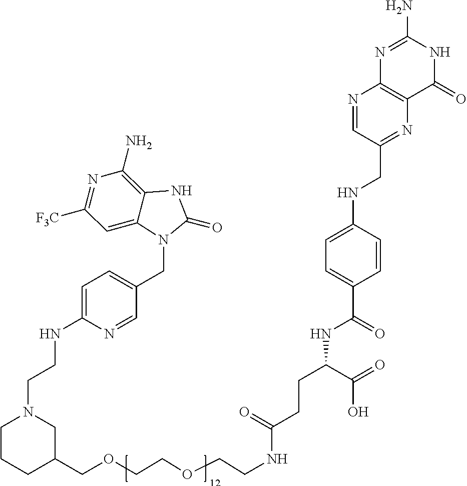 |
| 3D | 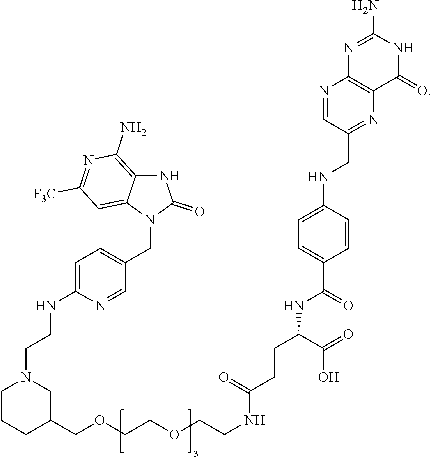 |
| 4A | 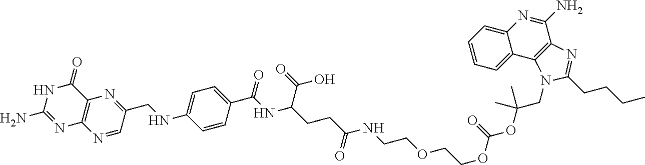 [SMILES: CCCCc1nc2c(N)nc3ccccc3c2n1CC(C)(C)OC(=O)OCCOCCNC(=O)CCC(NC(=O)c1ccc(NCc2cnc3nc(N)[nH]c(=O)c3n2)cc1)C(=O)O] |
| 5A | 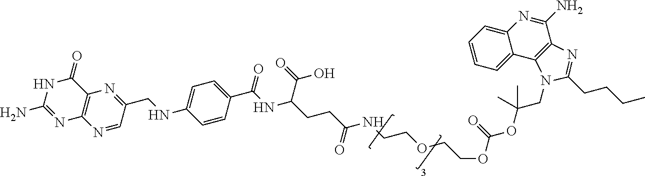 |
| 6A | 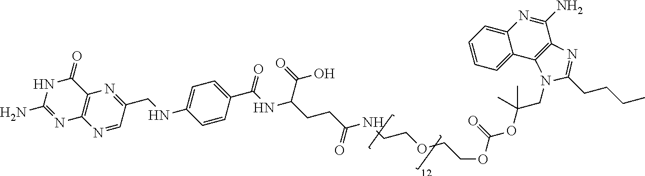 |
| 7A | 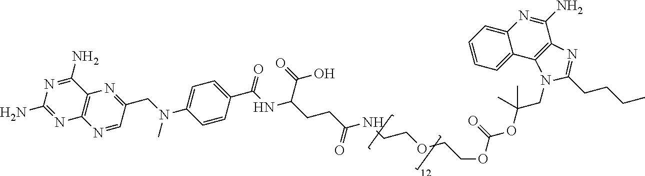 |
| 8A | 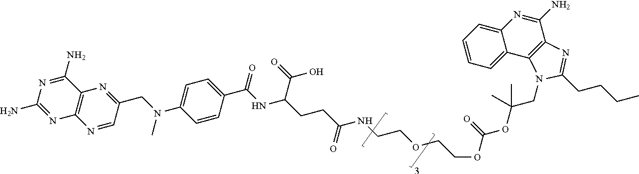 |
| 9A | 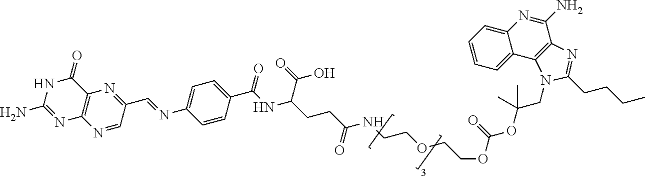 |
| 10A | 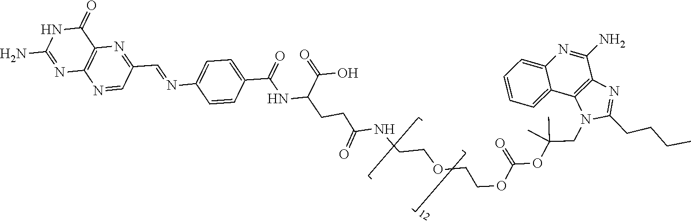 |
| 11A | 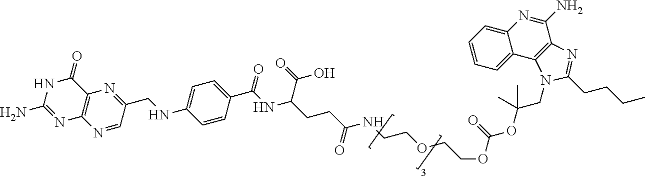 |
| 12A | 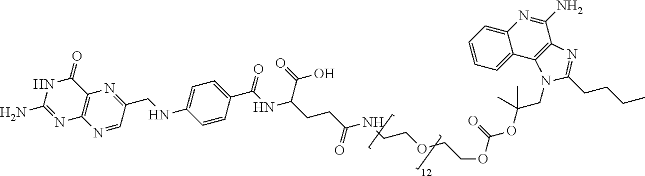 |

In some instances, a conjugated compound provided herein has the structure of Formula XIV (e.g., or a functional fragment or analog thereof, which includes the TLR7 agonist of Formula III conjugated with a folate via a releasable linker):

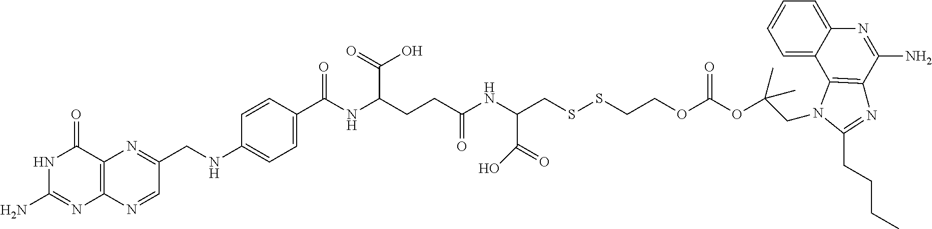 [SMILES: CCCCc1nc2c(N)nc3ccccc3c2n1CC(C)(C)OC(=O)OCCSSCC(NC(=O)CCC(NC(=O)c1ccc(NCc2cnc3nc(N)[nH]c(=O)c3n2)cc1)C(=O)O)C(=O)O]

or is a pharmaceutically acceptable salt or hydrate thereof.

In another embodiment, a conjugated compound provided herein has the structure of Formula XV (e.g., or a functional fragment or analog thereof, which includes the TLR7 agonist of Formula II conjugated with a folate via a releasable linker (e.g., Compound 3B)):

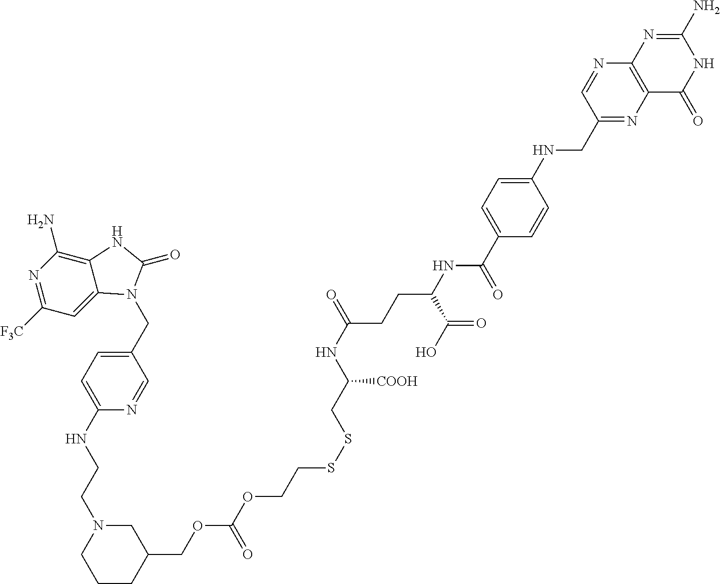

or is a pharmaceutically acceptable salt or hydrate thereof.

In yet another embodiment, a conjugated compound provided herein has the structure of Formula XVI (e.g., or a functional fragment or analog thereof, which includes the TLR7 agonist of Formula II conjugated with a folate via a non-releasable linker comprising three PEGs (e.g., Compound 3D)):

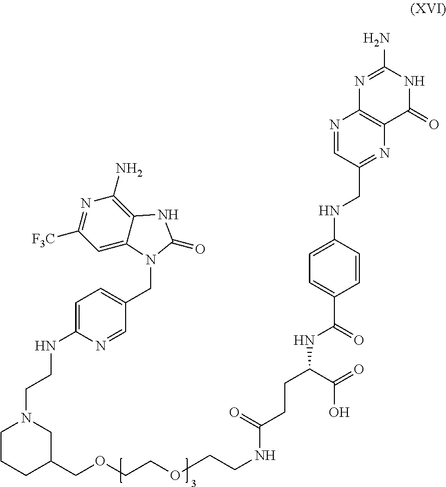

or is a pharmaceutically acceptable salt or hydrate thereof

In still another embodiment, a conjugated compound provided herein has the structure of Formula XVII (e.g., or a functional fragment or analog thereof, which includes the TLR7 agonist of Formula II conjugated with a folate via a non-releasable linker comprising twelve PEGs (e.g., Compound 3C)):

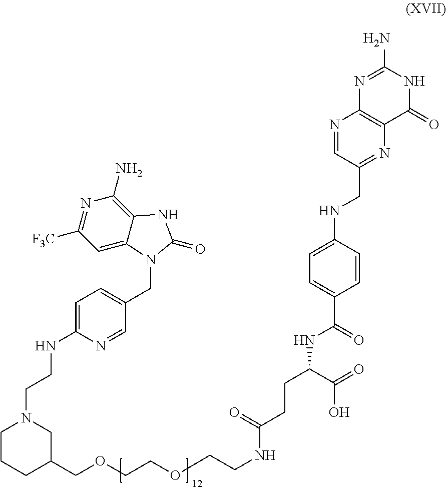

or is a pharmaceutically acceptable salt or hydrate thereof.

Further embodiments of a conjugated compound provided herein has the structure of Formula XVIII (e.g., or a functional fragment or analog thereof, which includes the TLR7 agonist of Formula II conjugated with a folate via a non-releasable linker comprising sixteen PEGs (e.g., Compound 3D′)):

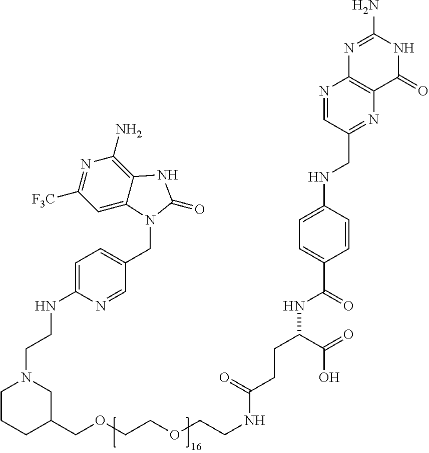

or is a pharmaceutically acceptable salt or hydrate thereof.

Further embodiments of a conjugated compound provided herein has the structure of Formula XIX (e.g., or a functional fragment or analog thereof, which includes the TLR7 agonist of Formula III conjugated with a folate (Compound 1B)):

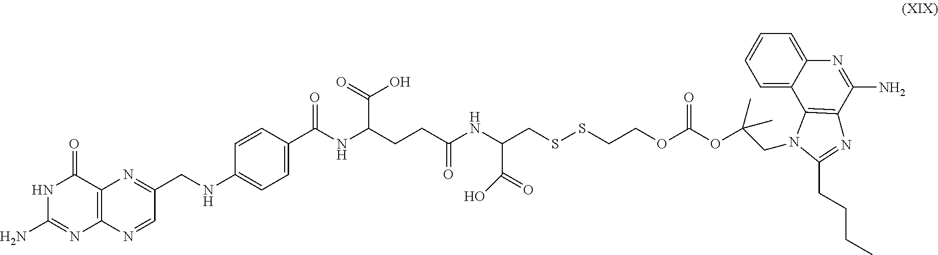 [SMILES: CCCCc1nc2c(N)nc3ccccc3c2n1CC(C)(C)OC(=O)OCCSSCC(NC(=O)CCC(NC(=O)c1ccc(NCc2cnc3nc(N)[nH]c(=O)c3n2)cc1)C(=O)O)C(=O)O]

or is a pharmaceutically acceptable salt or hydrate thereof.

In some embodiments, TLR-7/8 agonists conjugated with folate provide specificity for a diseased cell type. In one embodiment, folate-TLR7/8 agonist compound can be delivered (e.g., specifically) into the endosome of FRβ+ macrophages, e.g., while limiting system exposure to the TLR-7/8 agonists.

One embodiment provides a compound represented by the structure of Formula (2-I):

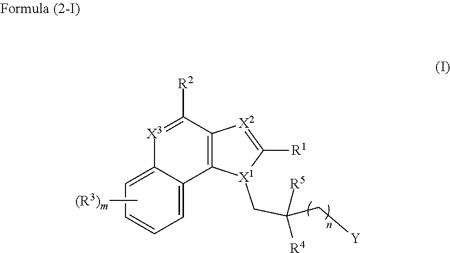

or a pharmaceutically acceptable salt or hydrate thereof,
wherein, in Formula (2-I):
R¹, R³, R⁴, and R⁵ are each independently a H, an alkyl, an alkoxyl, an alkenyl, an alkynyl, an alicyclic, an aryl, a biaryl, a halo, a heteroaryl, —COR^{2x},

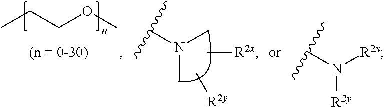

R² is a H, —OH, —NH₂, —NHR^{2x}, N₃, —NH—CH₂—NH₂, —CONH₂, —SO₂NH₂, —NH—CS—NH₂,

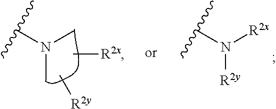

Y is a H, —OH, —NH₂, —NHR^{2x}, —O—R^{2x}, —SO—R^{2x}, —SH, —SO₃H, —N₃, —CHO, —COOH, —CONH₂, —COSH, —COR^{2x}, —SO₂NH₂, alkenyl, alkynyl, alkoxyl, —NH—CH₂—NH₂, —CONH₂, —SO₂NH₂, —NH—CS—NH₂,

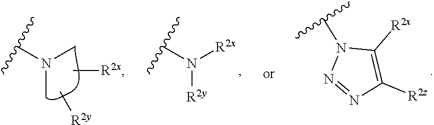

where:
   each of R^{2x}, and R^{2y} is independently selected from the group consisting of H, —OH, —CH₂—OH, —NH₂, —CH₂—NH₂, —COOMe, —COOH, —CONH₂, —COCH₃, alkyl, alkenyl, alkynyl, alicyclic, aryl, biaryl, and heteroaryl, and each R^{2z} is independently selected from the group consisting of —NH₂, —NR²R₂′, —O—R², —SO—R^{2q} and —COR^{2q}
   wherein each of R^{2q} and R^{2q′} is independently alkyl or H; and

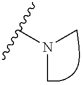 [SMILES: *N(C)C]

is a 3-10 membered N-containing heterocycle that is non-aromatic, and mono- or bicyclic;
wherein, in Formula (2-I), each of X¹, X², and X³ is independently CR^{q} or N, and each R^{q} is independently H, halogen, or an optionally substituted alkyl; and
wherein, in Formula (2-I), n is 0-30, and m is 0-4.

Another embodiment provides a compound, having the structure of Formula (2-IA):

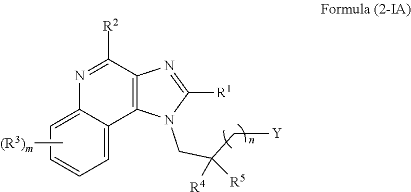

wherein:
R¹ is an optionally substituted C₃-C₈ alkyl (e.g., acyclic or cyclic) (e.g., optionally substituted with one or more substituents, each substituent independently being halogen, alkyl, heteroalkyl, alkoxy, or cycloalkyl);
R² is H, —OR^{z}, —SO₂N(R^{z})₂, —NR^{2x}R^{2y}, or N₃;
Y is H, —OR^{z}, —NR^{2x}R^{2y}, —SR, —SOR, —SO₃R^{z}, —N₃, —COR, —COOR^{z}, —CON(R^{z})₂, —COSR^{z}, —SO₂N(R^{z})₂, or —CON(R^{z})₂;
R^{2x} and R^{2y} are each independently hydrogen, —N(R^{z})₂, —CON(R^{z})₂, —C(R^{z})₂—N(R^{z})₂, —CS—N(R^{z})₂, or optionally substituted alkyl (e.g., optionally substituted with one or more substituents, each substituent independently being oxo, halogen, alkyl, heteroalkyl, alkoxy, or cycloalkyl), each R^{Z} is independently hydrogen, halogen, or optionally substituted alkyl; or R^{2x} and R^{2y} are taken together to form an optionally substituted heterocycloalkyl (e.g., wherein the optionally substituted heterocycloalkyl is a mono- or bicyclic heterocycloalkyl and/or wherein the optionally substituted heterocycloalkyl is a 3-10 membered heterocycloalkyl); each R³ is independently a halogen, —N₃, —CN, —NO₂, —COR, —COOR^{z}, —CON(R^{z})₂, —COSR^{z}, —SO₂N(R^{z})₂, —CON(R^{z})₂, alkyl, heteroalkyl, cycloalkyl, heterocycloalkyl, alkoxy, amino, hydroxy or thiol, wherein the alkyl, alkoxy, heteroalkyl, cycloalkyl, or heterocycloalkyl and is optionally substituted;
R⁴ and R⁵ are each independently alkyl, alkoxy, halogen, or cycloalkyl, wherein the alkyl, alkoxy, and cycloalkyl are optionally substituted; and
n is 1-6, and m is 0-4,
   or a pharmaceutically acceptable salt or hydrate thereof

At least one embodiment provides a compound, or a pharmaceutically acceptable salt or hydrate thereof, having the structure of Formula (2-I) or (2-IA) wherein n is 1-30. In one embodiment, n is 1-6. In another embodiment, n is 1-3. In another embodiment, n is 1 or 2. In another embodiment, n is 0. In another embodiment, n is 1. In another embodiment, n is 1 and Y is —OH. In another embodiment, n is 1 and Y is —NH₂. In one embodiment, the compound is represented by the structure of toll like receptor (TLR) 7 (TLR7)-1 (Compound 1). In one embodiment, the compound is represented by the structure of TLR7-1 (Compound 2). In one embodiment, the compound is represented by the structure of TLR7-1 (Compound 3). The structures of such compounds are depicted in FIG. 1C.

At least one embodiment provides a compound, or a pharmaceutically acceptable salt or hydrate thereof, having the structure of Formula (2-I) or (2-IA) wherein Y is —OH, OCH₃, —NH₂, —NHNH₂, —NHCONH₂, —SH, —SO₂NH₂, —N₃, —COOH, —COCH₃, —COOCH₃, or —CONH.

At least one embodiment provides a compound, or a pharmaceutically acceptable salt thereof, having the structure of Formula (2-I) or (2-IA) wherein Y is an H, —NH₂, —NHR^{2x}, —O—R^{2X}, —SO—R^{2x}, —SH, —SO₃H, —N₃, —CHO, —COOH, —CONH₂, —COSH, —COR^{2x}, —SO₂NH₂, alkenyl, alkynyl, alkoxyl, —NH—CH₂—NH₂, —CONH₂, —SO₂NH₂, —NH—CS—NH₂,

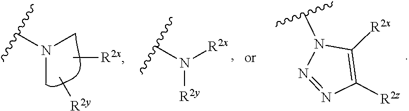

One embodiment provides a compound, or a pharmaceutically acceptable salt or hydrate thereof, having the structure of Formula (2-I) or (2-IA) wherein Y is OH.

At least one embodiment provides a compound, or a pharmaceutically acceptable salt or hydrate thereof, having the structure of Formula (2-I) or (2-IA) wherein Y is NH₂.

At least one embodiment provides a compound, or a pharmaceutically acceptable salt or hydrate thereof, having the structure of Formula (2-I) or (2-IA) wherein n is 1 and Y is OH.

At least one embodiment provides a compound, or a pharmaceutically acceptable salt or hydrate thereof, having the structure of Formula (2-I) or (2-IA) wherein n is 1 and Y is NH₂.

At least one embodiment provides a compound, or a pharmaceutically acceptable salt or hydrate thereof, having the structure of Formula (2-I) or (2-IA) wherein n is 0 and Y is NH₂.

At least one embodiment provides a compound, or a pharmaceutically acceptable salt or hydrate thereof, having the structure of Formula (2-I) or (2-IA) wherein R¹ is an optionally substituted alkyl. In one embodiment, R¹ is an optionally substituted C₃-C₆ alkyl. In another embodiment, R¹ is an optionally substituted acyclic C₃-C₆ alkyl. In another embodiment, R¹ is butyl.

At least one embodiment provides a compound, or a pharmaceutically acceptable salt or hydrate thereof, having the structure of Formula (2-I) or (2-IA) wherein R² is —NR^{2x}R^{2y}. In one embodiment, R² is NH₂.

At least one embodiment provides a compound, or a pharmaceutically acceptable salt or hydrate thereof, having the structure of Formula (2-I) or (2-IA) wherein R³ is H.

One embodiment provides a compound, or a pharmaceutically acceptable salt or hydrate thereof, having the structure of Formula (2-I) or (2-IA) wherein R⁴ is alkyl. In one embodiment, R⁴ is methyl.

At least one embodiment provides a compound, or a pharmaceutically acceptable salt or hydrate thereof, having the structure of Formula (2-I) or (2-IA) wherein R⁵ is alkyl. In one embodiment. R⁵ is methyl.

At least one embodiment provides a compound, or a pharmaceutically acceptable salt or hydrate thereof, having the structure of Formula (2-I) or (2-IA) wherein R⁴ and R⁵ are each alkyl. In one embodiment, R⁴ and R⁵ are each methyl.

At least one embodiment provides a compound, or a pharmaceutically acceptable salt or hydrate thereof, having the structure of Formula (2-I) or (2-IA) wherein m is 0. In another embodiment, m is 1. In another embodiment, m is 2. In another embodiment, m is 3. In another embodiment, m is 4.

At least one embodiment provides a compound, or a pharmaceutically acceptable salt or hydrate thereof, having the structure of Formula (2-I) wherein X¹, X², and X³ are each N. In one embodiment, X¹ is N. In another embodiment, X² is N. In another embodiment, X³ is N.

At least one embodiment provides a compound, or a pharmaceutically acceptable salt or hydrate thereof, having the structure of Formula (2-I) with the proviso that compounds where n is 0 are excluded.

At least one embodiment provides a compound, or a pharmaceutically acceptable salt or hydrate thereof, having the structure of Formula (2-I) with the proviso that compounds where n is 0 and Y is OH are excluded.

At least one embodiment provides a compound, or a pharmaceutically acceptable salt or hydrate thereof, having the structure of Formula (2-I) with the proviso that compounds where n is 0, Y is OH, R¹ is butyl, R² is NH₂, R³ is H and R⁴ and R¹ are each methyl are excluded.

At least one embodiment provides a compound, or a pharmaceutically acceptable salt or hydrate thereof, having the structure of Formula (2-I) with the proviso that the compound TLR7-1 is excluded.

In some embodiments, the compound is represented by any one or more of the formulae:

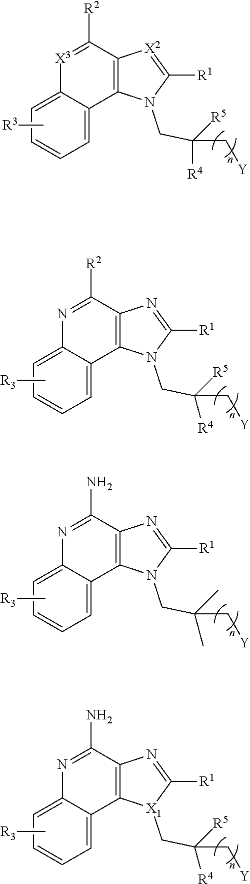

or is a pharmaceutically acceptable salt or hydrate of any of the foregoing structures, wherein:
R¹ is an optionally substituted C₃-C₈ alkyl (e.g., acyclic or cyclic) (e.g., optionally substituted with one or more substituents, each substituent independently being halogen, alkyl, heteroalkyl, alkoxy, or cycloalkyl);
R² is H, —OR^{z}, —SO₂N(R^{z})₂, —NR^{2x}R^{2y}, or N₃;
Y is a point of attachment to the linker or targeting ligand of the compound (e.g., of the first therapy or first medicament hereof) and comprises H, —OR^{z}, —NR^{2x}R^{2y}, —SR^{z}, —SOR^{z}, —SO₃R^{z}, —N₃, —COR^{z}, —COOR^{z}, —CON(R^{z})₂, —COSR^{z}, —SO₂N(R^{z})₂, or —CON(R^{z})₂;
R^{2x} and R^{2y} are each independently hydrogen, —N(R^{z})₂, —CON(R^{z})₂, —C(R^{z})₂—N(R^{z})₂, —CS—N(R^{z})₂, or optionally substituted alkyl (e.g., optionally substituted with one or more substituents, each substituent independently being oxo, halogen, alkyl, heteroalkyl, alkoxy, or cycloalkyl), each R^{z} is independently hydrogen, halogen, or optionally substituted alkyl; or R^{2x} and R^{2y} are taken together to form an optionally substituted heterocycloalkyl (e.g., wherein the optionally substituted heterocycloalkyl is a mono- or bicyclic heterocycloalkyl and/or wherein the optionally substituted heterocycloalkyl is a 3-10 membered heterocycloalkyl);
each R³ is independently a halogen, —N₃, —CN, —NO₂, —COR^{z}, —COOR^{z}, —CON(R^{z})₂, —COSR^{z}, —SO₂N(R^{z})₂, —CON(R^{z})₂, alkyl, heteroalkyl, cycloalkyl, heterocycloalkyl, alkoxy, amino, hydroxy or thiol, wherein the alkyl, alkoxy, heteroalkyl, cycloalkyl, or heterocycloalkyl and is optionally substituted;
R⁴ and R⁵ are each independently alkyl, alkoxy, halogen, or cycloalkyl, wherein the alkyl, alkoxy, and cycloalkyl are optionally substituted; and
n is 1-6, and m is 0-4.

In some embodiments, the compound is represented by any one or more of the formulae:

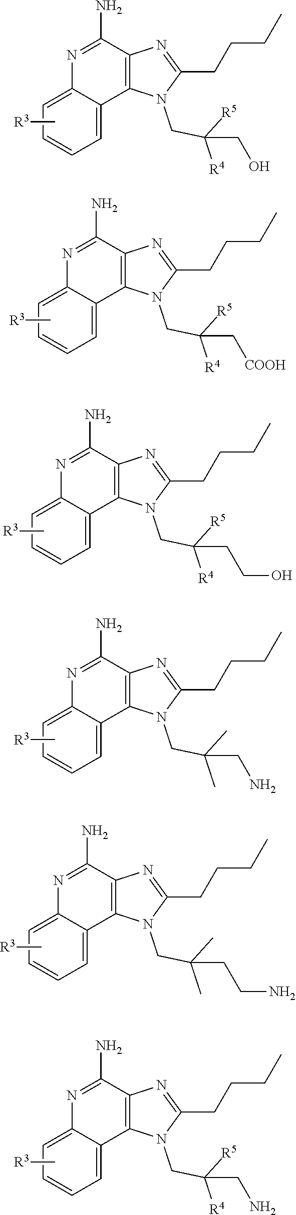

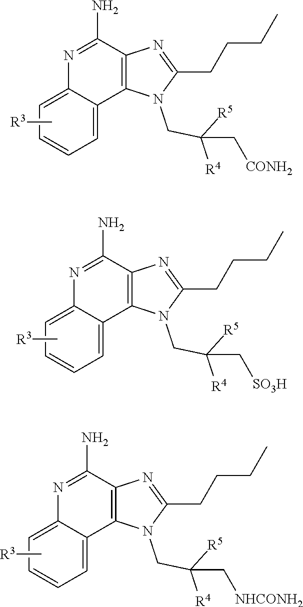

or a pharmaceutically acceptable salt or hydrate thereof

In some embodiments, the compound is represented by any one or more of the formulae:

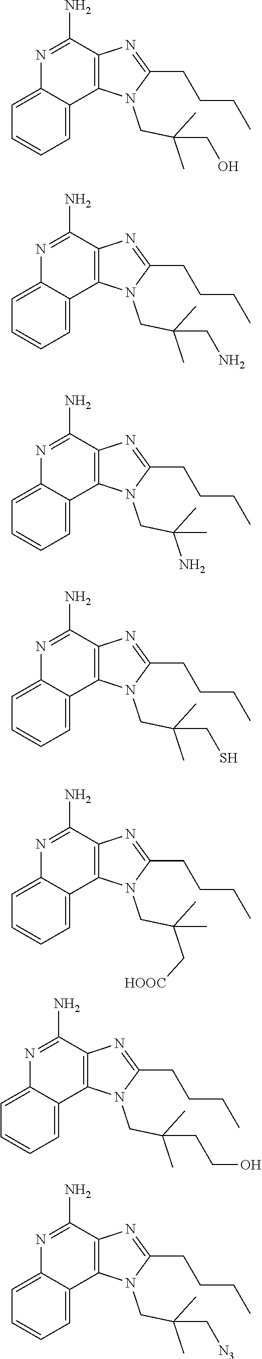

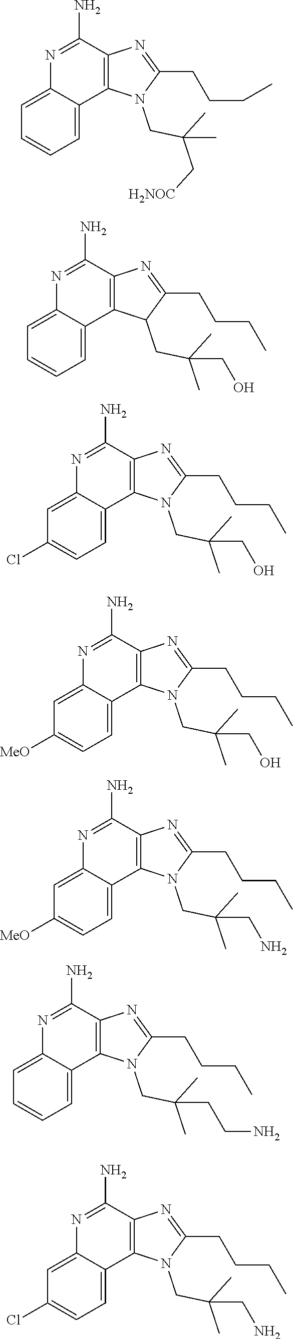

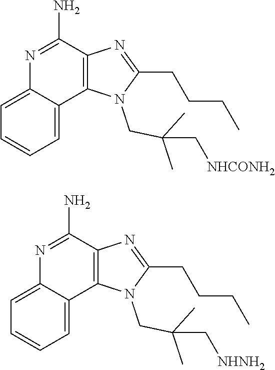

or a pharmaceutically acceptable salt or hydrate thereof.

As noted above, the present disclosure further relates to compounds (e.g., radicals thereof) (e.g., TLR 7 and/or 8 (TLR7/8) agonists) that are conjugated, directly or via a linker, to a targeting moiety (e.g., radicals thereof) that targets a pattern recognition receptor or DAMP of a cell. In some embodiments, the targeting ligand comprises a folate ligand or functional fragment or analog thereof, e.g., pteroyl amino acids. In some embodiments, the linkers are non-releasable. In some embodiments, the compound provides targeting molecules (e.g., radicals thereof) having non-releasable linkers thereby reducing systemic exposure of TLR7/8 agonists. In some embodiments, the compound provides targeting molecules (e.g., radicals thereof) having non-releasable linkers, thereby reducing systemic adverse effects of TLR7/8 agonists.

One embodiment provides a compound, or a pharmaceutically acceptable salt or hydrate thereof, having the structure:

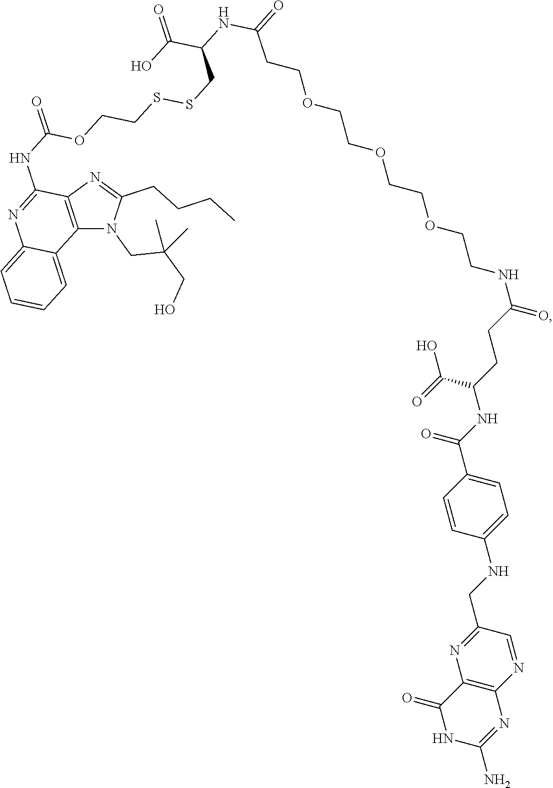

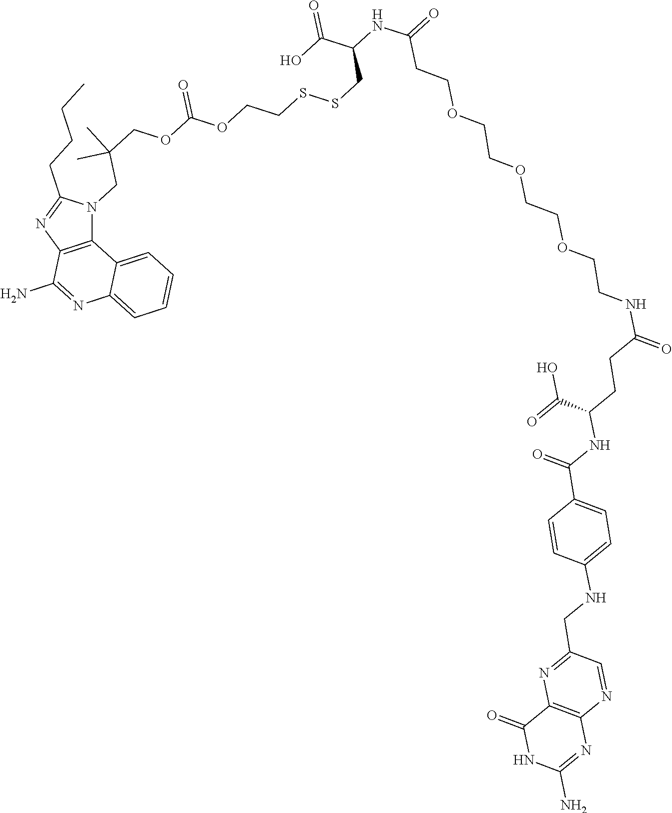 [SMILES: CCCCc1nc2c(N)nc3ccccc3c2n1CC(C)(C)COC(=O)OCCSSC[C@H](NC(=O)CCOCCOCCOCCNC(=O)CC[C@H](NC(=O)c1ccc(NCc2cnc3nc(N)[nH]c(=O)c3n2)cc1)C(=O)O)C(=O)O]

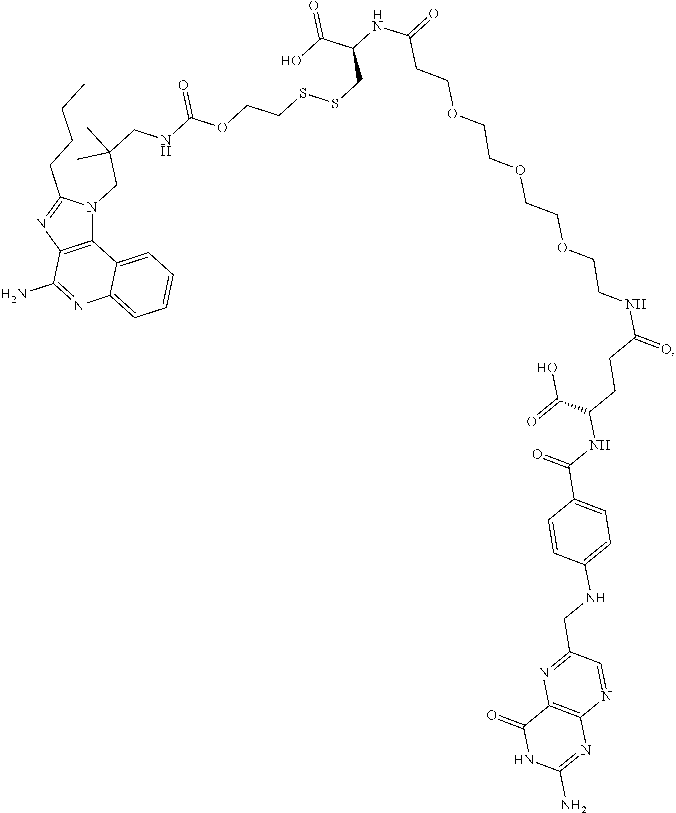

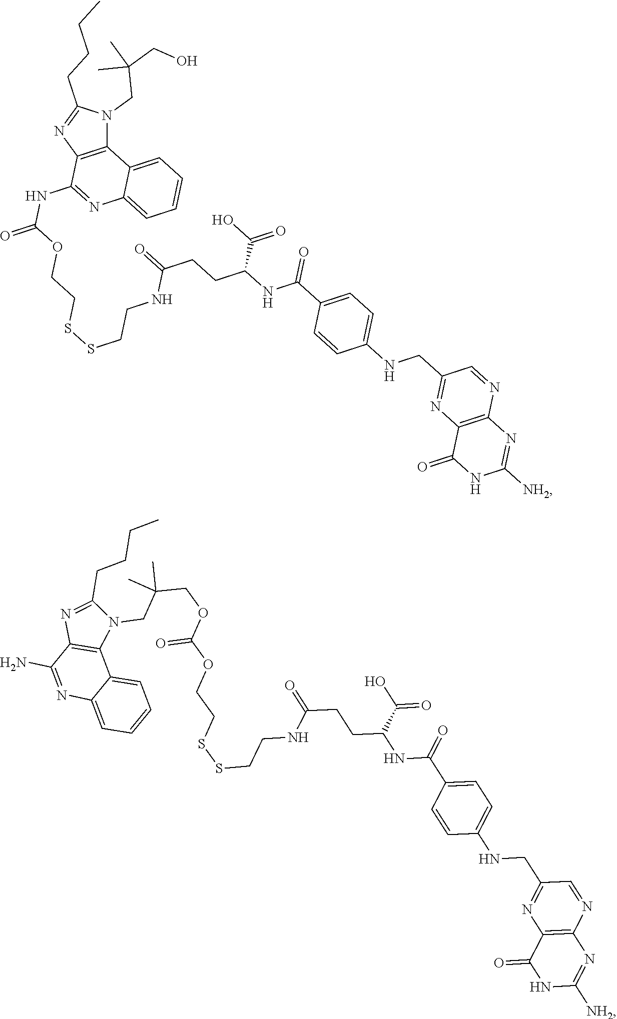

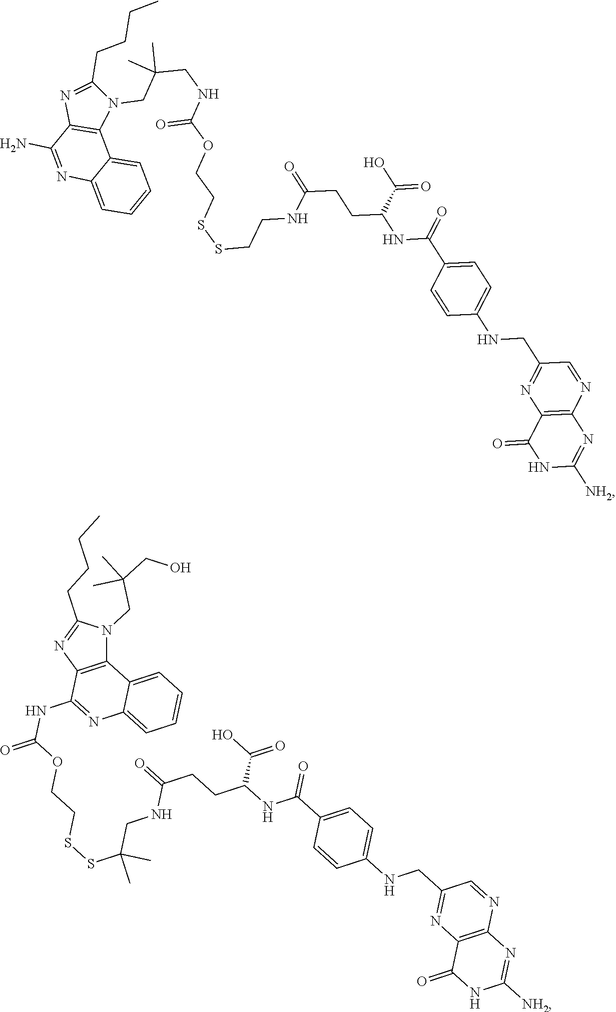

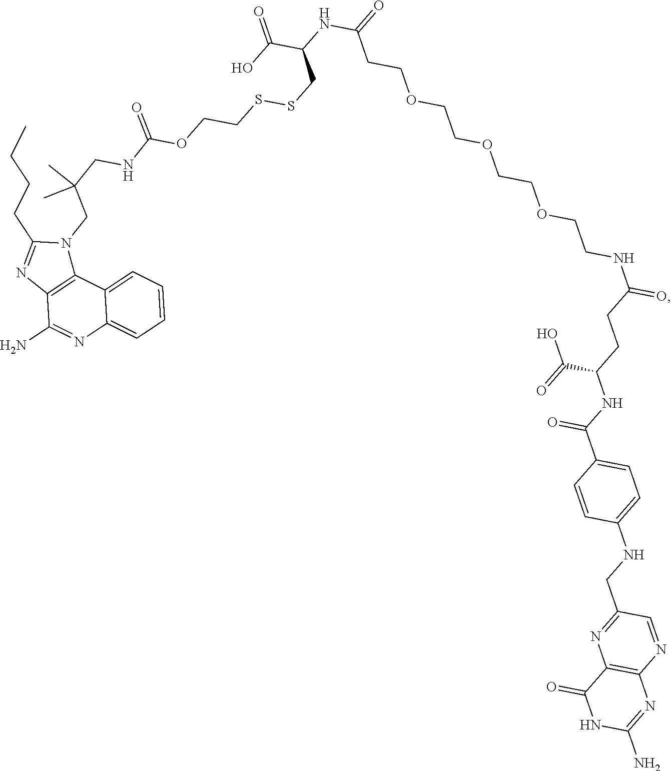 [SMILES: CCCCc1nc2c(N)nc3ccccc3c2n1CC(C)(C)CNC(=O)OCCSSC[C@H](NC(=O)CCOCCOCCOCCNC(=O)CC[C@H](NC(=O)c1ccc(NCc2cnc3nc(N)[nH]c(=O)c3n2)cc1)C(=O)O)C(=O)O]

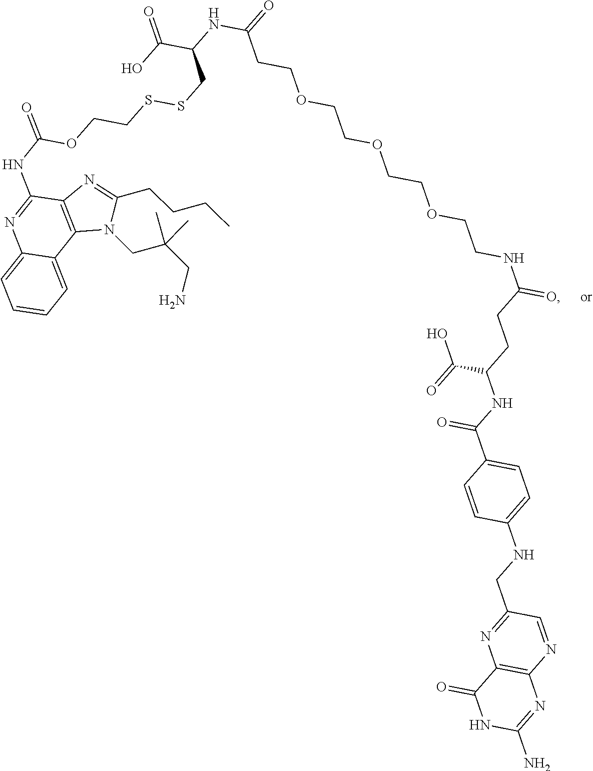

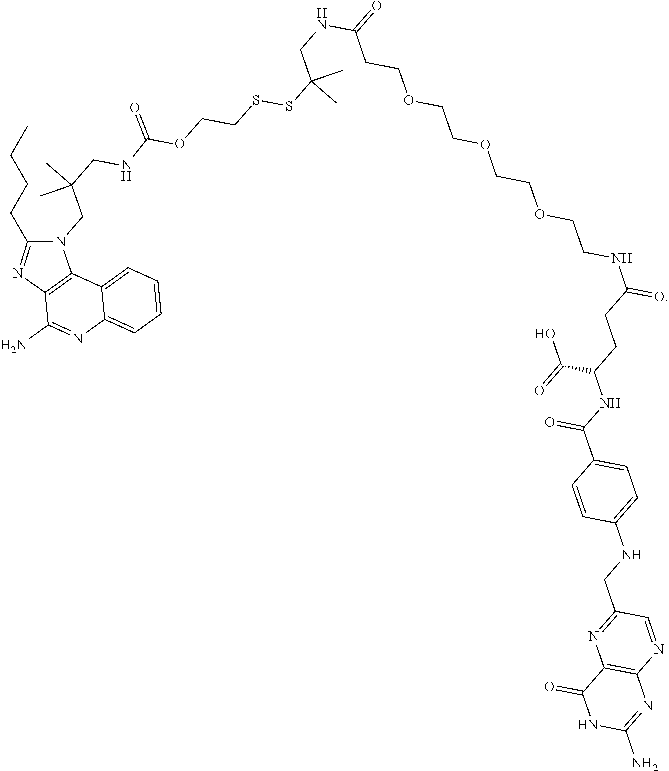

One embodiment provides a compound, or a pharmaceutically acceptable salt or hydrate thereof, having the structure:

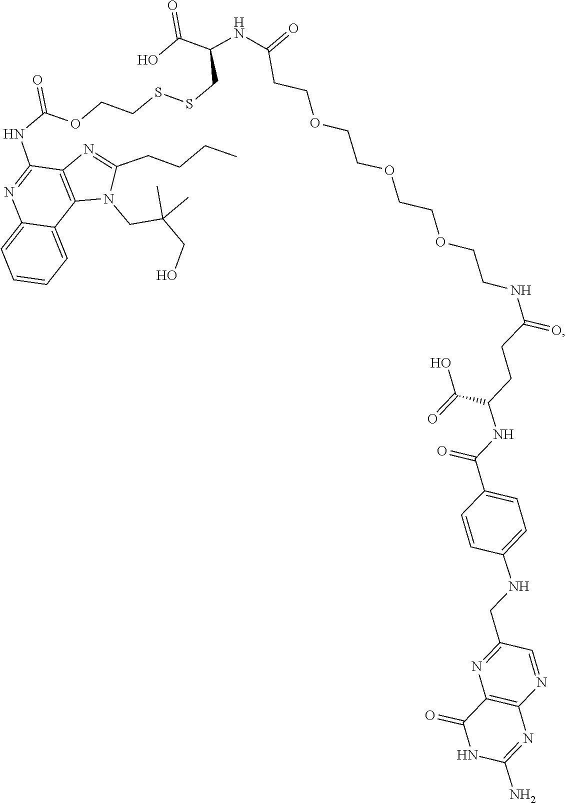

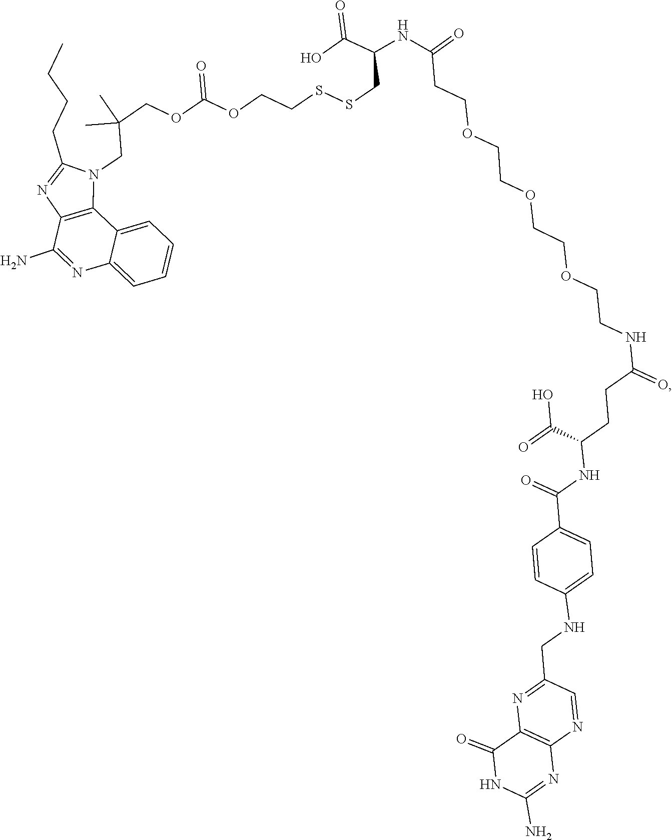 [SMILES: CCCCc1nc2c(N)nc3ccccc3c2n1CC(C)(C)COC(=O)OCCSSC[C@H](NC(=O)CCOCCOCCOCCNC(=O)CC[C@H](NC(=O)c1ccc(NCc2cnc3nc(N)[nH]c(=O)c3n2)cc1)C(=O)O)C(=O)O]

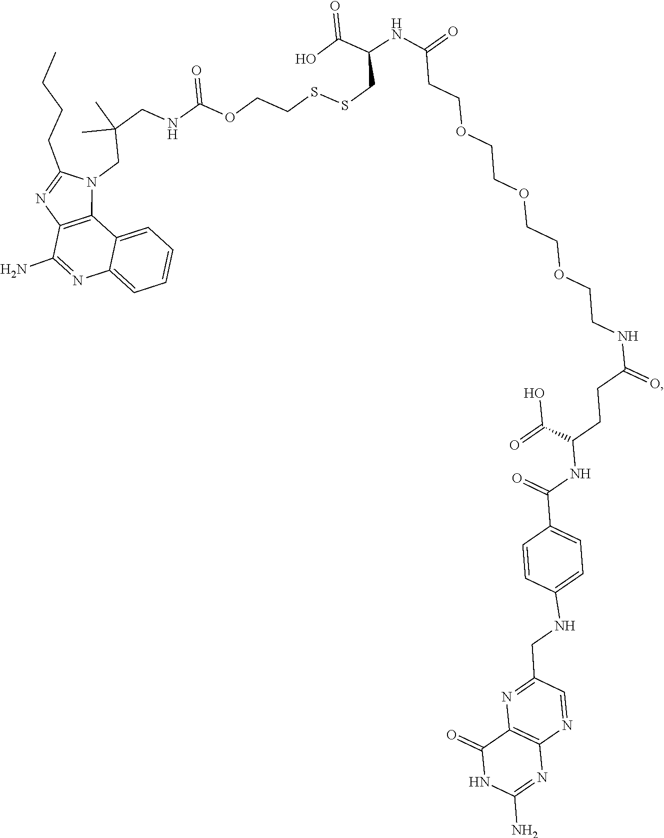

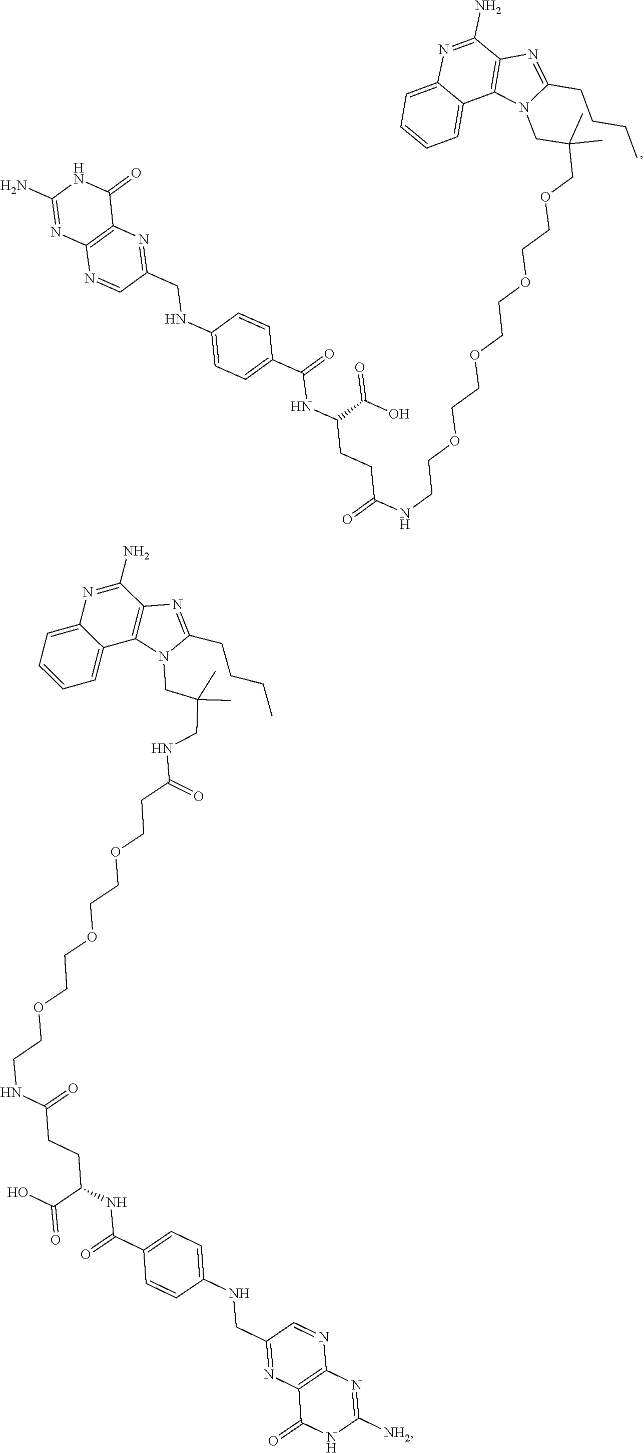

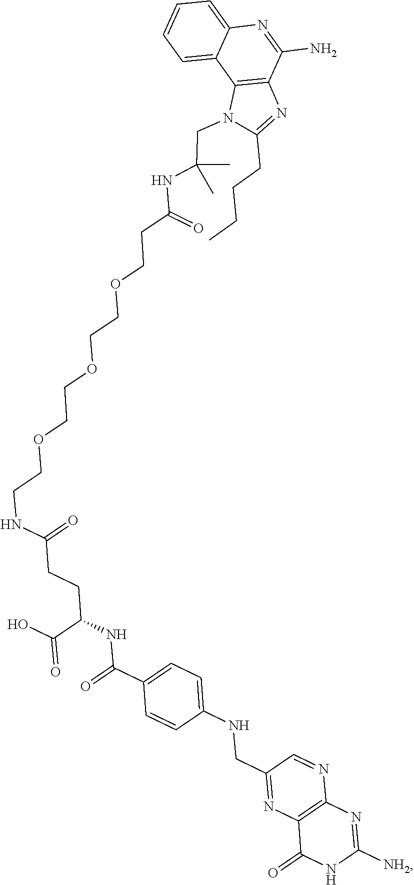 [SMILES: CCCCc1nc2c(N)nc3ccccc3c2n1CC(C)(C)NC(=O)CCOCCOCCOCCNC(=O)CC[C@H](NC(=O)c1ccc(NCc2cnc3nc(N)[nH]c(=O)c3n2)cc1)C(=O)O]

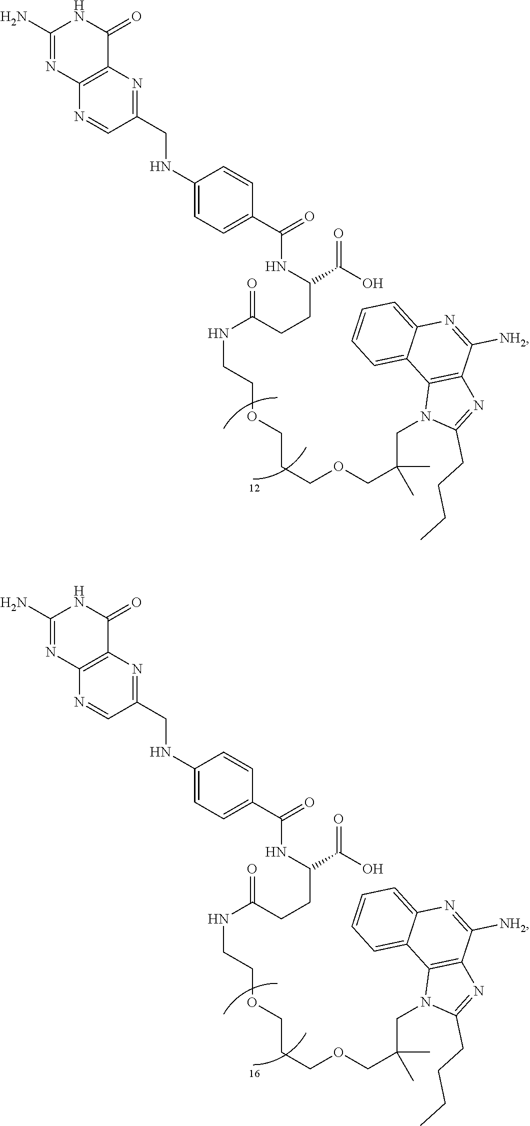

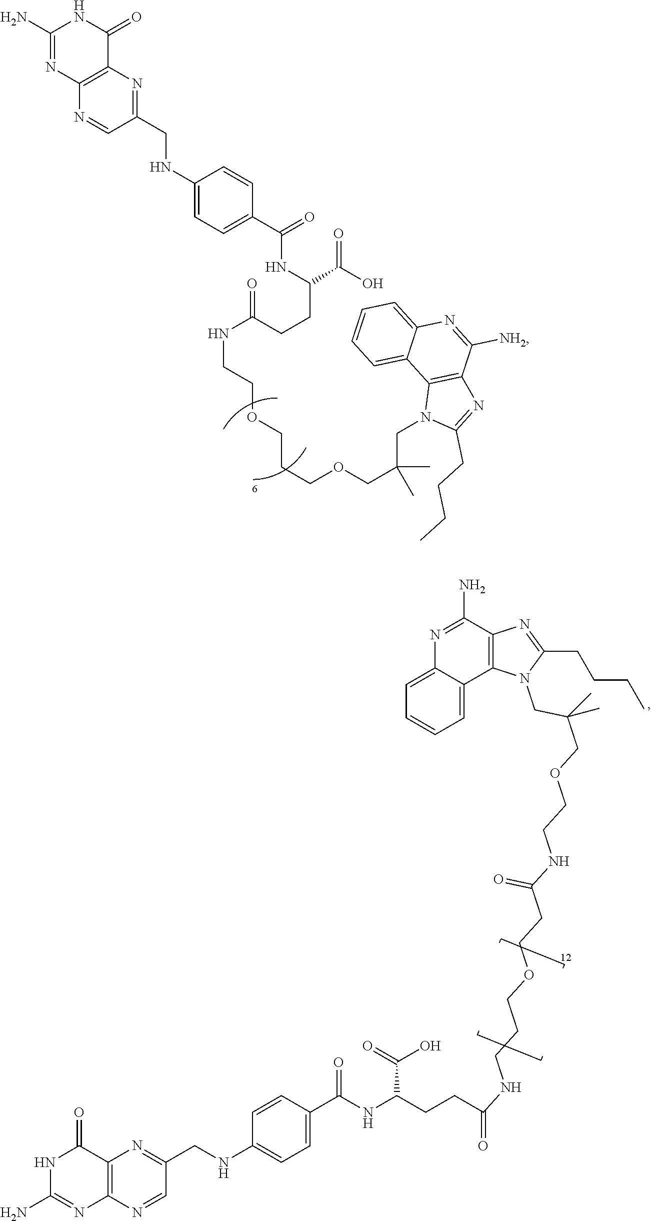

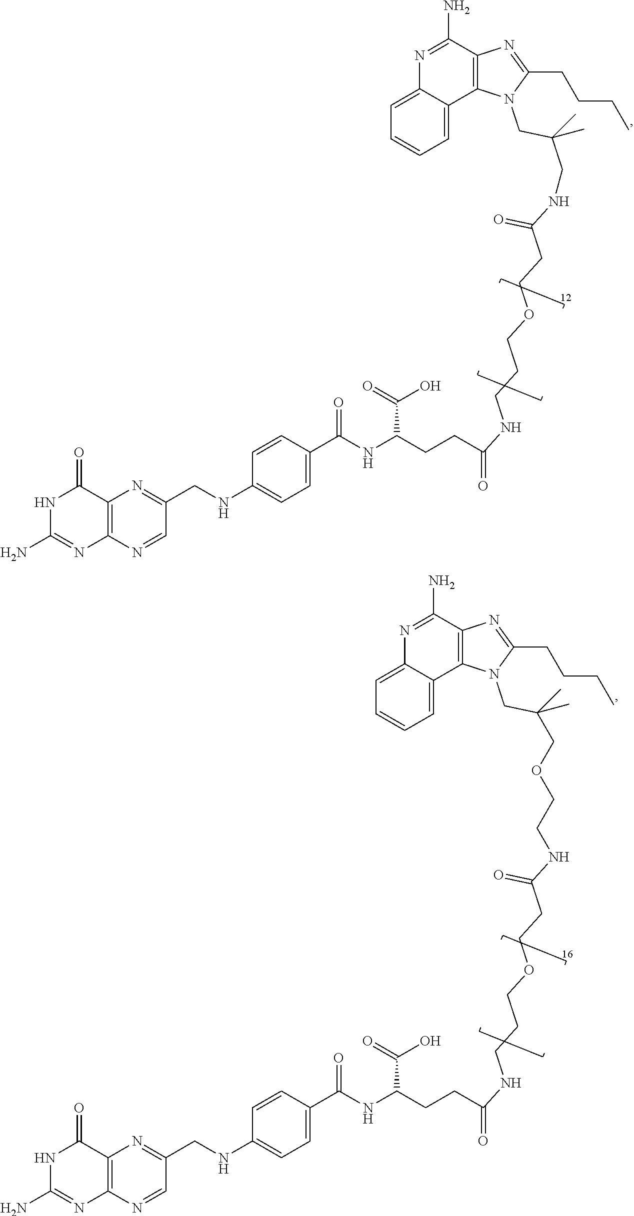

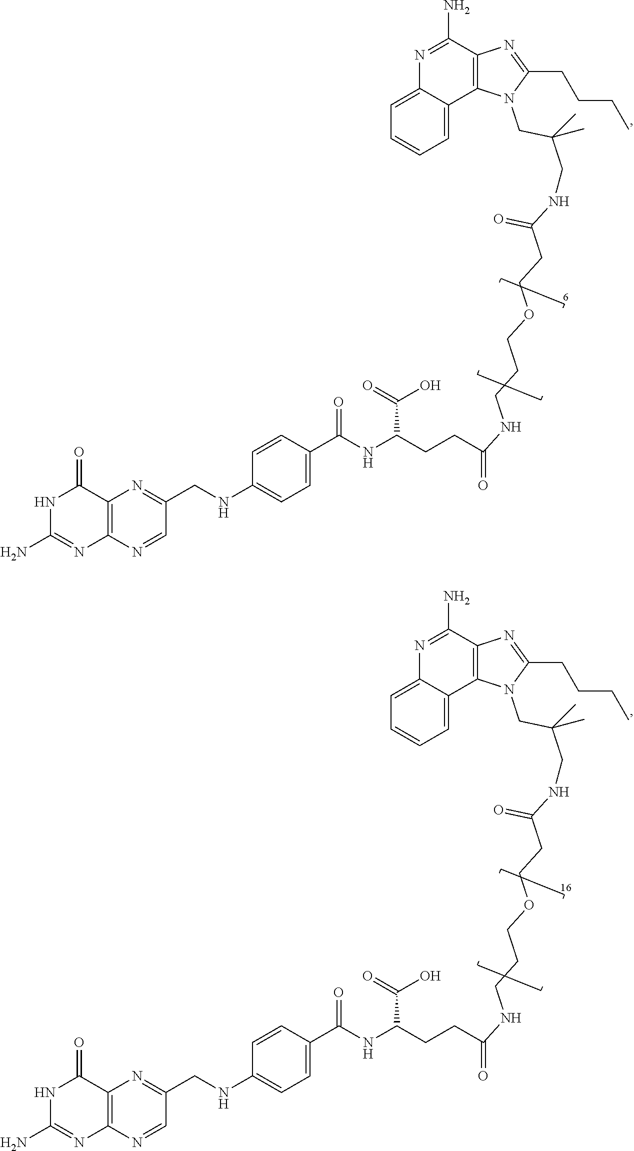

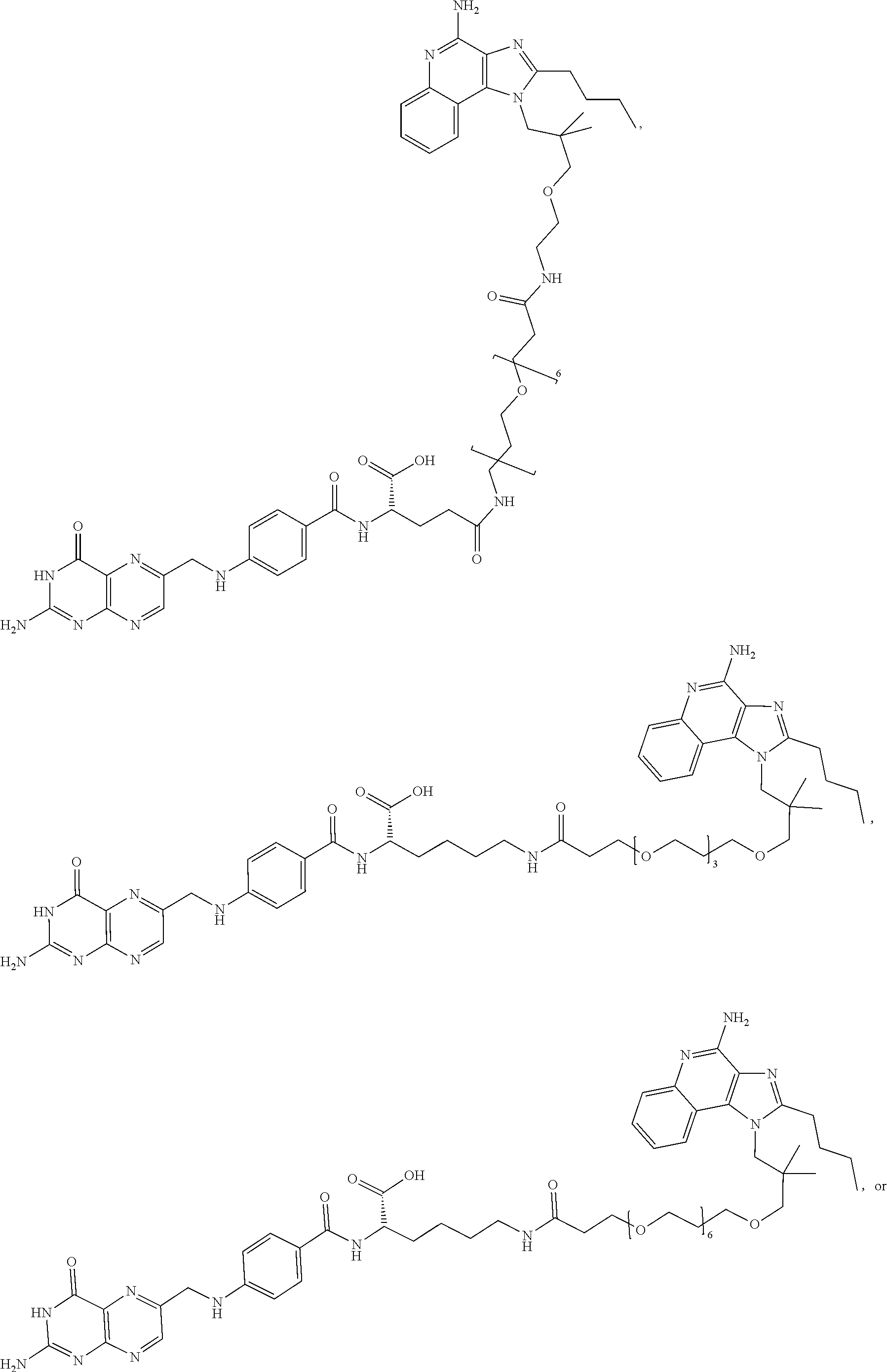

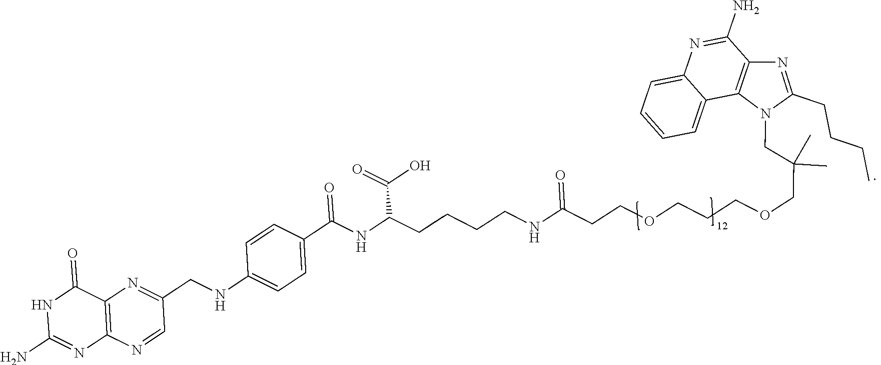

The compounds can be prepared by conventional methods of organic synthesis practiced by those skilled in the art. The general reaction sequences outlined below represent a general method useful for preparing the compounds and are not meant to be limiting in scope or utility.

Descriptions of compounds are limited by principles of chemical bonding known to those skilled in the art. Accordingly, where a group can be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The terms “identical” or percent “identity.” in the context of two or more polypeptide sequences, refer to two or more sequences or subsequences that are the same or have a specified percentage of peptides that are the same (i.e. about 60% identity, preferably 65%, 70%, 75%, 80%, 85%, 90%, 91%, 92%, 93%, 94% 95%, 96%, 97%, 98% 99%, or higher identity over a specified region when compared and aligned for maximum correspondence over a comparison window or designated region such as a targeting end, folate end, linker, or warhead) as measured using sequence comparison algorithms known in the art, or by manual alignment and visual inspection. Such sequences are then said to be “substantially identical.” In other words, identity exists over one or more regions of the overall sequence as long as the general shape and structure of the molecule, and hydrogen bond(s) where appropriate, are maintained such that it substantially fits into the targeted binding site and functions as an agonist thereto.

The compounds can be administered in unit dosage forms and/or compositions containing one or more pharmaceutically acceptable carriers, adjuvants, diluents, excipients, and/or vehicles, and combinations thereof. The term “administering” and its formatives generally refer to any and all means of introducing compounds described herein to the host subject including, but not limited to, by oral, intravenous, intramuscular, subcutaneous, transdermal, inhalation, buccal, ocular, sublingual, vaginal, rectal, and like routes of administration.

### Salts and Hydrates

The compounds (e.g., the conjugates or portions thereof) can be presented as a pharmaceutically acceptable salt. A “pharmaceutically acceptable salt” of a compound refers to those salts whose counter ions can be used in pharmaceuticals. Such salts include (i) acid addition salts, which can be obtained by reaction of the free base of the parent compound with inorganic acids, such as hydrochloric acid, hydrobromic acid, nitric acid, phosphoric acid, sulfuric acid, perchloric acid, and the like, or with organic acids, such as acetic acid, oxalic acid, (D) or (L) malic acid, maleic acid, methane sulfonic acid, ethane sulfonic acid, p-toluene sulfonic acid, salicylic acid, tartaric acid, citric acid, succinic acid, malonic acid, and the like, and (ii) salts formed when an acidic proton present in the parent compound either is replaced by a metal ion, e.g., an alkali metal ion, an alkaline earth ion, or an aluminum ion, or coordinates with an organic base, such as ethanolamine, diethanolamine, triethanolamine, trimethamine, N-methyl glucamine, and the like. Pharmaceutically acceptable salts are well-known to those skilled in the art, and any such pharmaceutically acceptable salt is contemplated herein.

In various embodiments, suitable basic salts are formed from bases which form non-toxic salts. Illustrative examples include arginine, benzathine, calcium, choline, diethylamine, diolamine, glycine, lysine, magnesium, meglumine, olamine, potassium, sodium, tromethamine, and zinc salts. Hemisalts of acids and bases also may be formed, e.g., hemisulphate and hemicalcium salts.

In some embodiments, the pharmaceutically acceptable salt is selected from hydrobromide, citrate, trifluoroacetate, ascorbate, hydrochloride, tartrate, triflate, maleate, mesylate, formate, acetate or fumarate.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

The compound, or pharmaceutically acceptable salt thereof, may exist in unsolved forms as well as solvated forms, including hydrated forms. Solvated forms can be equivalent to unsolvated forms. In each embodiment hereof, it will be understood that the formulae include and represent not only all pharmaceutically acceptable salts of the compounds, but also include any and all hydrates and/or solvates of the compound formulae or salts thereof. The term “solvate” means a compound, or a salt thereof, that further includes a stoichiometric or non-stoichiometric amount of solvent bound by non-covalent intermolecular forces. Where the solvent is water, the solvate is a hydrate.

Certain functional groups, such as the hydroxy, amino, and like, can form complexes and/or coordination conjugates with water and/or various solvents. Accordingly, the formulae are to be understood to include and represent those various hydrates and/or solvates. Non-hydrates and/or non-solvates of the bispecific adapters are also included.

### Pharmaceutical Compositions

In view of the above, also provided is a composition (e.g., a pharmaceutical composition) for the treatment of cancer comprising at least one compound hereof and a pharmaceutically acceptable carrier or excipient. The term “composition” generally refers to any product comprising more than one ingredient, including the compounds described herein. “Pharmaceutically acceptable carrier” includes any of the standard pharmaceutical carriers, such as, but not limited to, a buffering agent, a preserving agent, an anesthetic agent, a solubilizing agent, an isotonic agent, a wetting agent, and a stabilizer. The term also encompasses any of the agents approved by a regulatory agency, such as the U.S. Food and Drug Administration, or listed in the U.S. Pharmacopeia for use in animals (e.g., mammals, such as humans). The carrier can be a phosphate-buffered saline solution, water, or an emulsion such as an oil/water or water/oil emulsion.

It is to be understood that the compositions can be prepared from isolated compounds described herein or from salts, solutions, hydrates, solvates, and other forms of the compounds. It is appreciated that certain functional groups, such as the hydroxy, amino, and like groups can form complexes with water and/or various solvents, in the various physical forms of the compounds. It is also to be understood that the compositions can be prepared from various amorphous, non-amorphous, partially crystalline, crystalline, and/or other morphological forms of the compounds described herein, and the compositions can be prepared from various hydrates and/or solvates of the compounds described herein. Accordingly, pharmaceutical compositions that recite the compounds include each of, or any combination of, or individual forms of, the various morphological forms and/or solvate or hydrate forms of the compounds described herein.

The compounds can be formulated as pharmaceutical compositions and administered to a mammalian host, such as a human patient, in a variety of forms adapted to the chosen route of administration. For example, the pharmaceutical composition can be formulated for and administered via oral or parenteral, intravenous, intraarterial, intraperitoneal, intrathecal, epidural, intracerebroventricular, intraurethral, intrasternal, intracranial, intratumoral, intramuscular, topical, inhalation and/or subcutaneous routes. Indeed, in at least one embodiment, a compound and/or composition as described herein can be administered directly into the blood stream, into muscle, or into an internal organ.

For example, in at least one embodiment, the compounds can be systemically administered (orally, for example) in combination with a pharmaceutically acceptable vehicle such as an inert diluent or an assimilable edible carrier. For oral therapeutic administration, the active compound can be combined with one or more excipients and used in the form of ingestible tablets, buccal tablets, troches, capsules, elixirs, suspensions, syrups, wafers, and the like. The percentage of the compositions and preparations can vary and can be between about I to about 99% weight of the active ingredient(s) and a binder, excipients, a disintegrating agent, a lubricant, and/or a sweetening agent (as are known in the art). The amount of active compound in such therapeutically useful compositions is such that an effective dosage level will be obtained.

The preparation of parenteral compounds/compositions under sterile conditions, for example, by lyophilization, can readily be accomplished using standard pharmaceutical techniques well known to those skilled in the art. In at least one embodiment, the solubility of a compound used in the preparation of a parenteral composition can be increased by the use of appropriate formulation techniques, such as the incorporation of solubility-enhancing agents.

As previously noted, the compounds/compositions can also be administered via infusion or injection (e.g., using needle (including microneedle) injectors and/or needle-free injectors). Solutions of the active composition can be aqueous, optionally mixed with a nontoxic surfactant and/or can contain carriers or excipients such as salts, carbohydrates and buffering agents (preferably at a pH of from 3 to 9), but, for some applications, they can be more suitably formulated as a sterile non-aqueous solution or as a dried form to be used in conjunction with a suitable vehicle such as sterile, pyrogen-free water or phosphate-buffered saline (PBS). For example, dispersions can be prepared in glycerol, liquid PEGs, triacetin, and mixtures thereof and in oils. Under ordinary conditions of storage and use, these preparations can further contain a preservative to prevent the growth of microorganisms.

The pharmaceutical dosage forms suitable for injection or infusion can include sterile aqueous solutions or dispersions or sterile powders comprising the active ingredients that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example and without limitation, water, ethanol, a polyol (e.g., glycerol, propylene glycol, liquid PEG(s), and the like), vegetable oils, nontoxic glyceryl esters, and/or suitable mixtures thereof. In at least one embodiment, the proper fluidity can be maintained by the formation of liposomes, by the maintenance of the required particle size in the case of dispersions or by the use of surfactants. The action of microorganisms can be prevented by the addition of various antibacterial and antifungal agents such as parabens, chlorobutanol, phenol, sorbic acid, thimerosal, and the like. In certain cases, it will be desirable to include one or more isotonic agents such as sugars, buffers, or sodium chloride. Prolonged absorption of the injectable compositions can be brought about by the incorporation of agents formulated to delay absorption, for example, aluminum monostearate and gelatin.

Sterile injectable solutions can be prepared by incorporating the active compound and/or composition in the required amount of the appropriate solvent with one or more of the other ingredients set forth above, as required, followed by filter sterilization. In the case of sterile powders for the preparation of sterile injectable solutions, the preferred methods of preparations are vacuum drying and the freeze-drying techniques, which yield a powder of the active ingredient plus any additional desired ingredient present in the previously sterile-filtered solutions.

For topical administration, it can be desirable to administer the present compounds to the skin as compositions or formulations in combination with a dermatologically acceptable carrier, which can be a solid or a liquid. For example, in certain embodiments, solid carriers can include finely divided solids such as talc, clay, microcrystalline cellulose, silica, alumina and the like. Similarly, useful liquid carriers can comprise water, alcohols or glycols or water-alcohol/glycol blends, in which the present compounds can be dissolved or dispersed at effective levels, optionally with the aid of non-toxic surfactants. Additionally or alternatively, adjuvants such as fragrances and antimicrobial agents can be added to optimize the properties for a given use. The resultant liquid compositions can be applied from absorbent pads, used to impregnate bandages and/or other dressings, sprayed onto the targeted area using pump-type or aerosol sprayers, or simply applied directly to a desired area of the subject.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like for application directly to the skin of the subject.

The terms “therapeutically effective,” “therapeutically effective dose.” “therapeutically effective amount,” “prophylactically effective amount.” or “prophylactically effective dose” mean (unless specifically stated otherwise) a quantity of a compound which, when administered either one time or over the course of a treatment cycle affects the health, wellbeing or mortality of a subject (e.g., and without limitation, delays the onset of and/or reduces the severity of one or more of the symptoms associated with a cancer). Useful dosages of the compounds can be determined by comparing their in vitro activity, and the in vivo activity in animal models. Methods of the extrapolation of effective dosages in mice and other animals to human subjects are known in the art. Indeed, the dosage of the compound can vary significantly depending on the condition of the host subject, the cancer type being treated, how advanced the pathology is, the route of administration of the compound and tissue distribution, and the possibility of co-usage of other therapeutic treatments (such as radiation therapy or additional drugs in combination therapies). The amount of the composition required for use in treatment (e.g., the therapeutically or prophylactically effective amount or dose) will vary not only with the particular application, but also with the salt selected (if applicable) and the characteristics of the subject (such as, for example, age, condition, sex, the subject's body surface area and/or mass, tolerance to drugs) and will ultimately be at the discretion of the attendant physician, clinician, or otherwise. Therapeutically effective or prophylactically effective amounts or doses can range, for example, from about 0.05 mg/kg of patient body weight to about 30.0 mg/kg of patient body weight, or from about 0.01 mg/kg of patient body weight to about 5.0 mg/kg of patient body weight, including but not limited to 0.01 mg/kg, 0.02 mg/kg, 0.03 mg/kg, 0.04 mg/kg, 0.05 mg/kg, 0.1 mg/kg, 0.2 mg/kg, 0.3 mg/kg, 0.4 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, and 5.0 mg/kg, all of which are kg of patient body weight. All ranges specified in this paragraph are inclusive of the stated end points and include all 0.05 g/m² increments encompassed in each specified range. The total therapeutically or prophylactically effective amount of the compound can be administered in single or divided doses and can, at the practitioner's discretion, fall outside of the typical range given herein.

In another embodiment, the compound can be administered in a therapeutically or prophylactically effective amount of from about 0.5 g/m to about 500 mg/m², from about 0.5 g/m² to about 300 mg/m², or from about 100 g/m² to about 200 mg/m². In other embodiments, the amounts can be from about 0.5 mg/m² to about 500 mg/m², from about 0.5 mg/m² to about 300 mg/m², from about 0.5 mg/m² to about 200 mg/m², from about 0.5 mg/m² to about 100 mg/m², from about 0.5 mg/m² to about 50 mg/m², from about 0.5 mg/m² to about 600 mg/m², from about 0.5 mg/m² to about 6.0 mg/m², from about 0.5 mg/m² to about 4.0 mg/m², or from about 0.5 mg/m² to about 2.0 mg/m². All ranges specified in this paragraph are inclusive of the stated end points and include all 0.5 g/m² increments encompassed in each specified range. The total amount can be administered in single or divided doses and can, at the physician's discretion, fall outside of the typical range given herein. These amounts are based on m of body surface area.

In other embodiments, the amount of the compound (or pharmaceutically acceptable salt or hydrate thereof) to be administered to a subject can range, for example, from about 50 nmol/kg to about 3,000 nmol/kg of subject body weight, about 50 nmol/kg to about 2,000 nmol/kg, about 50 nmol/kg to about 1,000 nmol/kg, about 50 nmol/kg to about 900 nmol/kg, about 50 nmol/kg to about 800 nmol/kg, about 50 nmol/kg to about 700 nmol/kg, about 50 nmol/kg to about 600 nmol/kg, about 50 nmol/kg to about 500 nmol/kg, about 50 nmol/kg to about 400 nmol/kg, about 50 nmol/kg to about 300 nmol/kg, about 50 nmol/kg to about 200 nmol/kg, about 50 nmol/kg to about 100 nmol/kg, about 100 nmol/kg to about 300 nmol/kg, about 100 nmol/kg to about 500 nmol/kg, about 100 nmol/kg to about 1.000 nmol/kg, or about 100 nnmol/kg to about 2,000 nmol/kg of subject body weight. In other embodiments, the dose can be about 100 nmol/kg, about 150 nmol/kg, about 200 nmol/kg, about 250 nmol/kg, about 300 nmol/kg, about 350 nmol/kg, about 400 nmol/kg, about 450 nmol/kg, about 500 nmol/kg, about 600 nmol/kg, about 700 nmol/kg, about 800 nmol/kg, about 900 nmol/kg, about 1,000 nmol/kg, about 2,000 nmol/kg, or about 3,000 nmol/kg of subject body weight. In other embodiments, between about 20 g/kg to about 3 mg/kg of subject body weight can be administered. The amount can be between about 0.2 mg/kg to about 0.4 mg/kg of subject body weight or about 50 μg/kg subject body weight. All ranges specified in this paragraph are inclusive of the stated end points and include all 1 nmol/kg or 10 g/kg increments, as applicable, encompassed in each specified range.

In some embodiments, in connection with measuring expression of certain biomarkers and/or analysis of cytokine levels in a sample from a subject, of significance of the present disclosure is not the particular methods used to detect the marker or set of markers, but what the markers are used to detect. There are many methods that can be used to detect the expression, quantification, or profile of one or more biomarkers. Once the marker or set of markers to be detected or quantified is identified, any of several techniques (that are now known or hereinafter developed) can be used, with the provision of appropriate reagents. One of skill in the art, when provided with the one or more biomarkers to be identified, will be capable of selecting the appropriate assay (e.g., a PCR-based or a microassay-based assay for nucleic acid markers, an enzyme-linked immunosorbent assay (ELISA), protein or antibody microarray or similar immunologic assay, etc.) for performing the methods disclosed herein.

### Combinations

Further provided is a combination of a first therapy comprising any of the compounds or pharmaceutical compositions hereof and a second therapy comprising one or more checkpoint inhibitors.

The second therapy can comprise one or more immune checkpoint inhibitors that can bind to and/or antagonize an immune checkpoint molecule. An “immune checkpoint inhibitor” or “antagonist” (as used in this context) is a molecule that inhibits, reduces, or blocks activity of an immune checkpoint molecule to inhibit a suppressive effect that the immune checkpoint molecule has on the immune system. The inhibitor or antagonist can directly bind the immune checkpoint molecule, or a ligand of the immune checkpoint molecule that mediates the activity of the immune checkpoint molecule.

An agent that selectively binds to an immune checkpoint molecule without limitation can be, for example, an antibody or antigen-binding fragment thereof (including a humanized antibody or antigen-binding fragment thereof), a protein or a peptide, a small molecule, or a nucleic acid. An immune checkpoint molecule that is a nucleic acid can be, e.g., an antisense molecule, a single- or double-stranded DNA oligonucleotide, a single- or double-stranded RNA oligonucleotide, a peptide nucleic acid (PNA), a single- or double-stranded RNAi molecule, an shRNA, or an siRNA. A small molecule is an organic compound drug. An agent that selectively binds to an immune checkpoint molecule can bind to nucleic acids or amino acids of the immune checkpoint molecule sequence. An agent that selectively binds an immune checkpoint molecule can bind to any region of the immune checkpoint molecule.

The checkpoint inhibitor can comprise a small molecule or other agent that disrupts an immune checkpoint of a cell of the subject such as, for example, programmed death 1 (PD-1), programmed death ligand 1 (PD-L1), cytotoxic T-lymphocyte-associated antigen 4 (CTLA-4), V-domain Ig suppressor of T cell activation (VISTA), lymphocyte activating 3 (LAG3), T cell immunoglobulin and mucin domain-containing protein 3 (TIM3), T cell immunoreceptor with Ig and ITIM domains (TIGIT), programmed death ligand 2 (PD-L2), indoleamine 2,3-deioxygenase (IDO), arginase-1 (AGR1), B7 family inhibitory ligand B7-H3 (B7-H3), B7 family inhibitory ligand B7-H4 (B7-H4), 2B4 (cluster of differentiation 244), B- and T-lymphocyte attenuator (BTLA), adenosine A2A receptor (A2aR), and/or a member of the family of killer cell immunoglobulin-like receptors (KIRs) such as KIRs and C-type lectin receptors, and signal transducer and activator of transcription (STAT3). The disclosure is not limited to targeting the aforementioned receptors for immune checkpoint blockade and other inhibitory checkpoint molecules can be targeted by immune checkpoint inhibitors in a combination therapy with the compound(s) and/or composition(s) hereof Activity of the immune checkpoint molecule is referred to as its suppressive effect on an immune checkpoint. An immune checkpoint inhibitor can reduce or block the activity of an immune checkpoint molecule.

Immune checkpoints refer to inhibitory pathways hardwired into the immune system that are crucial for maintaining self-tolerance and modulating the duration and amplitude of physiological immune responses in peripheral tissues to minimize collateral tissue damage. Immune checkpoint molecules can be stimulatory or inhibitory to an immune checkpoint. The present disclosure and claims refer to inhibitory molecules of immune checkpoints as “immune checkpoint molecules.” Preliminary clinical findings with agents that block immune checkpoint molecules (e.g., PD-1, PD-L1, or CTLA-4), suggest opportunities to enhance antitumor immunity with the potential to produce effective clinical responses. Combining immune checkpoint blockade using immune checkpoint inhibitor(s) with administration of the compounds and/or compositions hereof can enhance treatment efficacy in a subject having a cancer or a relapse of cancer.

An immune checkpoint inhibitor is a type of drug that blocks the signaling of immune checkpoint molecule(s) made by some types of immune system cells, such as T cells, and some cancer cells. Immune checkpoint inhibitors therefore can cause immune checkpoint blockade.

Immune checkpoint molecules (e.g., PD1) can help keep immune responses in check and can keep T cells from killing cancer cells. When these molecules are blocked, the “brakes” on the immune system are released (inhibition of the immune system is reduced or blocked) and T cells can more effectively kill cancer cells. Examples of checkpoint proteins found on T cells or cancer cells include PD-1/PD-L1 and CTLA-4. In some embodiments, immune checkpoint molecules are proteins. In certain embodiments, immune checkpoint molecules are nucleic acids that encode the proteins. In some embodiments, immune checkpoint inhibitors bind to and/or antagonize immune checkpoint molecules. In some embodiments, immune checkpoint inhibitors are used in combination with the compounds and/or compositions hereof to treat a subject having cancer.

In certain embodiments, the checkpoint inhibitor(s) can be selected from the group consisting of pembrolizumab, nivolumab, ipilimumab, cemiplimab, atezolizumab, avelumab, durvalumab, pidilizumab, monoclonal antibody MEDI-0680, monoclonal antibody REGN2810, or fusion protein AMP-224 that targets PD-1, ociperlimab, islelizumab, a combination of ociperlimab and islelizumab, BMS-936559/MDX-1105, MPDL3280A/RG7446/atezolizumab, MSB0010718C/avelumab, or MEDI4736/durvalumab, tiragolumab, zimberelimab, tremelimumab, relatlimab, monoclonal antibody IMP321, nivolumab, etigilimab, domvanalimab, tiragolumab (RG6058), vibostolimab, avelumab, and durvalumab.

In certain embodiments, the checkpoint inhibitor is a PD-1 antagonist or a PD-L1 antagonist. In certain embodiments, the checkpoint inhibitor is a CTLA-4 antagonist. In certain embodiments, the checkpoint inhibitor is a VISTA antagonist. In certain embodiments, the checkpoint inhibitor is a LAG3 antagonist. In certain embodiments, the checkpoint inhibitor is a TIM3 antagonist. In certain embodiments, the checkpoint inhibitor is a TIGIT antagonist.

The PD-1 antagonist can be, for example, an agent that binds to and antagonizes PD-1. In some embodiments, the agent that binds to and antagonizes PD-1 is a peptide that binds PD-1. In some embodiments, the agent that binds to and antagonizes PD-1 is a humanized antibody that selectively binds PD-1. In some embodiments, the humanized antibody that selectively binds PD-1 is nivolumab, pembrolizumab, pidilizumab, monoclonal antibody MEDI-0680, monoclonal antibody REGN2810, or fusion protein AMP-224 that targets PD-1. In some embodiments, the humanized antibody that selectively binds PD-1 is nivolumab, pembrolizumab, or pidilizumab. The PD-1 antagonist can be ociperlimab, islelizumab, or a combination of ociperlimab and islelizumab.

The PD-L1 antagonist can be, for example, an agent that binds to and antagonizes PD-L1. The agent that binds to and antagonizes PD-L1 can be, for example, a peptide that binds PD-L1. In certain embodiments, the agent that binds to and antagonizes PD-L1 is a humanized antibody that selectively binds PD-L1. The humanized antibody that selectively binds PD-L1 can be BMS-936559/MDX-1105, MPDL3280A/RG7446/atezolizumab, MSB0010718C/avelumab, or MED14736/durvalumab. The PD-L1 antagonist can be tiragolumab, atezolizumab, avelumab, durvalumab, or zimberelimab.

Examples of drugs that target PD-L1 include, without limitation, Atezolizumab, Avelumab, and Durvalumab. In certain embodiments, the checkpoint inhibitor is an antibody that targets CTLA-4, such as Ipilimumab. Furthermore, the checkpoint inhibitor can comprise a checkpoint inhibitor that targets T cell immunoglobulin and mucin domain containing protein-3 (CD366 or TIM3), which is a transmembrane protein. In alternative embodiments, the checkpoint inhibitors comprise small molecules or other agents that disrupt the immune checkpoint that is exploited by cancer cells to evade cell-mediated or other immune-mediated targeting.

The CTLA-4 antagonist can be, for example, an agent that binds to and antagonizes CTLA-4. In some embodiments, the agent that binds to and antagonizes CTLA-4 is a peptide that binds CTLA-4. In some embodiments, the agent that binds to and antagonizes CTLA-4 is a humanized antibody that selectively binds CTLA-4. In some embodiments, the humanized antibody that selectively binds CTLA-4 inhibitor is ipilimumab or tremelimumab. In some embodiments, the CTLA-4 antagonist is (i) an antisense molecule directed against CD80, CD86, and/or CTLA-4, (ii) an adnectin directed against CD80, CD86, and/or CTLA-4, (iii) a single stranded or double stranded RNAi inhibitor of CD80, CD86, and/or CTLA-4, or (iv) a small molecule inhibitor of CD80, CD86, or CTLA-4.

The VISTA antagonist can be, for example, an agent that binds to and antagonizes VISTA. In some embodiments, the agent that binds to and antagonizes VISTA is a peptide. In certain embodiments, the agent that binds to and antagonizes VISTA is an inhibitory antibody directed to VISTA. In some embodiments, the agent that binds to and antagonizes VISTA is a humanized antibody. In some embodiments, the agent that binds to and antagonizes VISTA is (i) an antisense molecule directed against VISTA, (ii) an adnectin directed against VISTA. (iii) a single stranded or double stranded RNAi inhibitor of VISTA, or (iv) a small molecule inhibitor of VISTA.

The LAG3 antagonist can be, for example, an agent that binds to and antagonizes LAG3. In some embodiments, the agent that binds to and antagonizes LAG3 is a peptide that binds LAG3. In some embodiments, the agent that binds to and antagonizes LAG3 is a humanized antibody that selectively binds LAG3. In some embodiments, the humanized antibody that selectively binds LAG3 is relatlimab, or monoclonal antibody IMP321.

The TIM3 antagonist can be, for example, an agent that binds to and antagonizes TIM3. In some embodiments, the agent that binds to and antagonizes TIM3 is a peptide that binds TIM3. In some embodiments, the agent that binds to and antagonizes TIM3 is a humanized antibody that selectively binds TIM3. In some embodiments, the humanized antibody that selectively binds TIM3 is INCAGN02390.

The TIGIT antagonist can be, for example, an agent that binds to and antagonizes TIM3. In some embodiments, the agent that binds to and antagonizes TIM3 is a peptide that binds TIM3. In some embodiments, the agent that binds to and antagonizes TIM3 is a humanized antibody that selectively binds TIM3. In some embodiments, the humanized antibody that selectively binds TIM3 is etigilimab, domvanalimab, tiragolumab (RG6058), or vibostolimab

As mentioned, immune checkpoint blockade can be used as a combination therapy with the compounds and compositions hereof. In certain approaches, administering both the compounds/compositions and the immune checkpoint inhibitor therapy results in a greater than additive inhibition of growth of the cancer.

Where multiple therapeutics and/or therapies are co-administered, dosages can be adjusted accordingly, as is recognized in the pertinent art. “Co-administration” and combination therapy are not limited to simultaneous administration, but also include treatment regimens in which a compound or composition is administered at least once during a course of treatment that involves administering immune checkpoint inhibitor therapy to a subject. In some embodiments, the one or more immune checkpoint inhibitors of the second therapy is/are administered on different day than the first therapy comprising the compound/composition. In some embodiments, the second therapy is administered on the same day as the first therapy. In some embodiments, the second therapy (i.e., the one or more immune checkpoint inhibitors) is administered on a different day than the first therapy (i.e., the compound/composition hereof), but within one day of, within five days of, within one week of, within eight days of, within two weeks of, within three weeks of, within one month of, within two months of, or within three months of administration of the first therapy to the subject.

In some embodiments, the immune checkpoint inhibitor(s) of the second therapy is/are administered intravenously or subcutaneously.

In certain embodiments, the combination can be used to treat a relapse of a cancer in a subject or resistance in a subject to immune checkpoint blockade therapy. In certain embodiments, the combination can be used to treat cancer in a subject.

### Uses and Methods

In addition to the compounds described herein, methods for providing treatment for and/or preventing relapse of a cancer are also provided. In some embodiments, provided herein is a method of treating an oncological disease or disorder in an individual in need thereof, comprising administering to the individual any compound provided herein, or a pharmaceutically acceptable salt or hydrate thereof, or a (e.g., pharmaceutical) composition comprising any compound provided herein, or a pharmaceutically acceptable salt thereof such as a compound having the structure of any one of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, Formula XVII, Formula XVIII, Formula XX, Formula XXX, Formula 2-I, Formula 2-II, Formula 2-III, Formula 2-IV, Formula 2-V, or Formula 2-VI.

In some embodiments, provided herein is a method of treating an oncological disease or disorder in an individual in need thereof, comprising administering to the individual any compound provided herein, or a pharmaceutically acceptable salt or hydrate thereof, or a (e.g., pharmaceutical) composition comprising any compound provided herein, or a pharmaceutically acceptable salt or hydrate thereof, such as a compound having the structure of any one of Formula I, Formula II, Formula III, Formula IV, Formula V, Formula VI, Formula VII, Formula VIII, Formula IX, Formula X, Formula XI. Formula XII, Formula XIII, Formula XIV, Formula XV, Formula XVI, Formula XVII, Formula XVIII, Formula XX, Formula XXX, Formula 2-I, Formula 2-II, Formula 2-III, Formula 2-IV, Formula 2-V, or Formula 2-VI. In some embodiments, the oncological disease or disorder is cancer. In some embodiments, the cancer is selected from bladder cancer, brain cancer, liver cancer, renal cancer, skin cancer, thymus carcinoma, gastrointestinal stromal tumor (GIST), esophageal cancer, pancreatic cancer, and breast cancer.

In some embodiments, provided herein is a method of treating a cancer-related disease or disorder in an individual in need thereof, comprising administering to the individual any compound provided herein, or a pharmaceutically acceptable salt or hydrate thereof, or a (e.g., pharmaceutical) composition comprising any compound provided herein, or a pharmaceutically acceptable salt or hydrate thereof, such as a compound having the structure of any one of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, Formula XVII, Formula XVIII, Formula XX, Formula XXX, Formula 2-I, Formula 2-II, Formula 2-III, Formula 2-IV, Formula 2-V, or Formula 2-VI.

In at least one embodiment, a method is provided for treating a subject suffering from, or at risk for experiencing, a disease state, wherein the disease state comprises a cancer (or recurrence thereof) and the method comprises contacting a cell of the subject with at least one compound. The at least one compound can comprise any of the compounds hereof and, in at least one exemplary embodiment, comprises a targeting moiety specific for FRβ. In some instances, contacting a cell can be achieved through administering the at least one compound to the subject intravenously, orally, intramuscularly, intraperitoneally, topically, orally, or through inhalation or any of the other administration modalities described herein. Additionally or alternatively, the at least one compound can comprise a composition containing one or more pharmaceutically acceptable carriers, adjuvants, diluents, excipients, and/or vehicles, or combinations thereof. The dosage of the at least one compound administered can be modified as appropriate by the clinician: however, the at least one compound is preferably dosed in an amount that is therapeutically effective or prophylactically effective and, in at least one embodiment, the dosage is in a range of between 1 nmol/kg body weight of the subject and 50 nmol/kg body weight of the subject.

In certain embodiments, provided herein is a method of treating or preventing recurrence or relapse of a cancerous disease state comprising contacting a cell with at least one compound comprising an immune modulator or pharmaceutically acceptable salt or hydrate thereof attached, via a linker, to a folate ligand or functional fragment or analog thereof, wherein the immune modulator or pharmaceutically acceptable salt thereof targets a pattern recognition receptor or a DAMP.

In certain embodiments, the method of treating a subject with cancer can comprise administering a first therapy to the subject, the first therapy comprising at least one compound or composition hereof, and administering a second therapy to the subject, the second therapy comprising one or more checkpoint inhibitors (i.e., one or more agents that bind to and/or antagonize PD-1, PD-L1, CTLA-4, VISTA, LAG3, TIM3, TIGIT, PD-L2, IDO, AGR1, B7-H3, B7-H4, 2B4, BTLA, A2aR, and/or a member of the family of KIRs such as KIRs and C-type lectin receptors, and STAT3). The disclosure is not limited to targeting the aforementioned receptors for immune checkpoint blockade and other inhibitory checkpoint molecules can be targeted by immune checkpoint inhibitors in a combination therapy with the compound(s) and/or composition(s) hereof

The at least one compound of the first therapy of the method can comprise any compound, or a pharmaceutically acceptable salt or hydrate thereof, described or a (e.g., pharmaceutical) composition comprising any compound provided herein, or a pharmaceutically acceptable salt or hydrate thereof, such as a compound having the structure of any one of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, Formula XVII, Formula XVIII, Formula XX, Formula XXX, Formula 2-I, Formula 2-II, Formula 2-III, Formula 2-IV, Formula 2-V, or Formula 2-VI. In some embodiments, the immune modulator comprises an agonist of TLR 3, 7, 8, 9 or 7/8.

In certain embodiments, the at least one compound comprises a radical of an immune modulator or pharmaceutically acceptable salt or hydrate thereof attached, via a linker, to a targeting ligand such as, for example, a folate ligand or functional fragment or analog thereof. The immune modulator or pharmaceutically acceptable salt or hydrate thereof can target a pattern recognition receptor. Similarly, the composition comprising the at least one compound can comprise any of the pharmaceutical compositions described. In certain embodiments, the composition comprises at least one compound hereof and one or more pharmaceutically acceptable carriers, adjuvants, diluents, excipients, and/or vehicles, or combinations thereof.

In certain embodiments of the methods, the radical of the immune modulator of the compound comprises a TLR 3, 7, 8, 9, or 7/8 agonist. In certain embodiments, the at least one compound (e.g., of the first therapy) has the following formula:

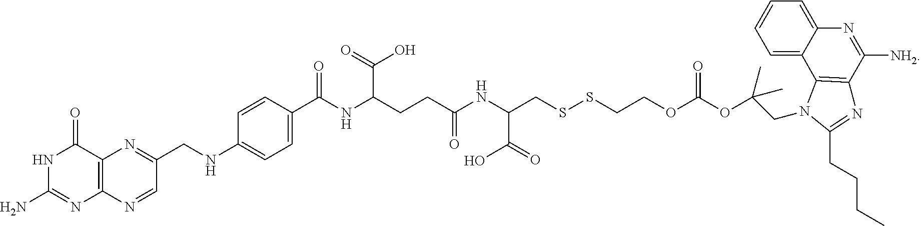

or is a pharmaceutically acceptable salt or hydrate thereof. In certain embodiments, the radical of the immune modulator of the first therapy comprises a TLR agonist of Formula X or XX, or is a pharmaceutically acceptable salt or hydrate of Formula X or XX:

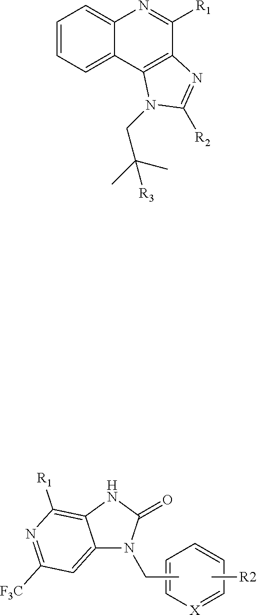

wherein, in Formulas X and XX:
R₁ is —NH₂ or —NH—R_{1X},
R₂ is an H, an alkyl, an alkenyl, an alkynyl, an alicyclic, an aryl, a biaryl, a heteroaryl, —NH—R_{2X}, —O—R_{2X}, —S—R_{2X},

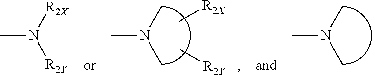

is a 3-10 membered N-containing non-aromatic mono- or bicyclic heterocycle, wherein:
in Formula X, R₃ is —OH, —SH, —NH₂ or —NH—R_{1X};
in Formula XX, X is a CH or an N; and
each of R_{1X}, R_{2X}, and R_{2Y} is independently selected from the group consisting of an H, an alkyl, an alkenyl, an alkynyl, an alicyclic, an aryl, a biaryl, and a heteroaryl.

The at least one compound of the first therapy can comprise:

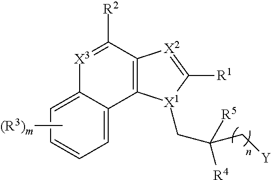

or be a pharmaceutically acceptable salt or hydrate thereof, wherein, in Formula 2-I:
   R¹, R³, R⁴, and R⁵ are each independently a hydrogen (H), an alkyl, an alkoxyl, an alkenyl, an alkynyl, an alicyclic, an aryl, a biaryl, a halo, a heteroaryl, —COR^{2x},

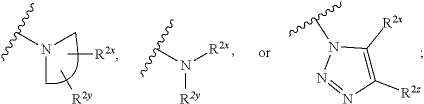

R² is a H, —OH, —NH₂, —NHR^{2x}, N₃, —NH—CH₂—NH₂, —CONH₂, —SO₂NH₂, —NH—CS—NH₂,

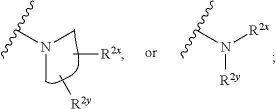

Y is the point of attachment to the linker and/or targeting ligand of the conjugate and comprises a H, —OH, —NH₂, —NHR^{2x}, —O—R^{2X}, —SO—R^{2x}, —SH, —SO₃H, —N₃, —CHO, —COOH, —CONH₂, —COSH, —COR^{2x}, —SO₂NH₂, alkenyl, alkynyl, alkoxyl, —NH—CH₂—NH₂, —CONH₂, —SO₂NH₂, —NH—CS—NH₂,

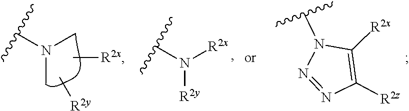

where:
each of R^{2x} and R^{2y} is independently selected from the group consisting of H, —OH, —CH₂—OH, —NH₂, —CH₂—NH₂, —COOMe, —COOH, —CONH₂, —COCH₃, alkyl, alkenyl, alkynyl, alicyclic, aryl, biaryl, and heteroaryl, and each R^{2z} is independently selected from the group consisting of —NH₂, —NR^{2q}R^{2q′}, —O—R^{2q}, —SO—R^{2q}, and —COR^{2q′} wherein each of R^{2q} and R^{2q′} is independently alkyl or H; and

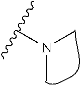 [SMILES: *N(C)C]

is a 3-10 membered N-containing heterocycle that is non-aromatic, mono- or bicyclic;
wherein, in Formula 2-I, each of X¹. X², and X³ is independently CR^{q} or N, and each R^{q} is independently H, halogen, or an optionally substituted alkyl; and
wherein, in Formula 2-I, n is 0-30, and m is 0-4.

In certain embodiments, the radical of the immune modulator of the first therapy comprises a TLR agonist having the following formula:

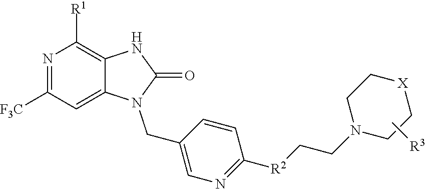

wherein: R¹ is an amine group, R² is a single bond —NH—, R³ is an H, an alkyl, a hydroxy group, or any other substituted group thereof, X is a CH₂, NH, O, or S, and the linker is attached at R¹, R₂ or R³.

In certain embodiments of the methods hereof, the at least one compound (e.g., of the first therapy) is or comprises a structure of the following formula:

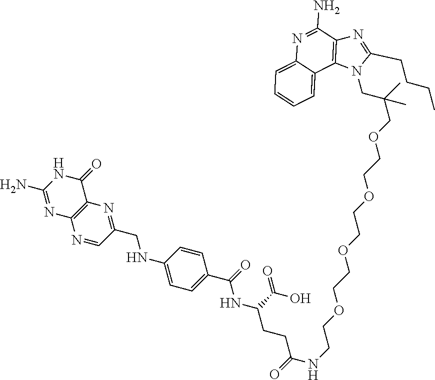

or is a pharmaceutically acceptable salt or hydrate thereof.

The linker of the at least one compound (or pharmaceutically acceptable salt or hydrate thereof) of the first therapy can be any linker described herein. In certain embodiments, the linker is a releasable linker. In certain embodiments, the linker is a non-releasable linker. In certain embodiments, the linker of the at least one compound (or pharmaceutically acceptable salt or hydrate thereof) of the first therapy comprises a PEG linker or a PEG derivative linker and is a non-releasable linker.

The subject can be experiencing, or at risk for experiencing, a cancer or a relapse of a cancer. In certain embodiments, the step of administering the first therapy further comprises administering or applying to the subject a therapeutically effective amount of the at least one compound or pharmaceutically acceptable salt or hydrate thereof.

In certain embodiments, administration of the second therapy (i.e., the one or more checkpoint inhibitors) disrupts an immune checkpoint of a cell of the subject. The one or more checkpoint inhibitors of the second therapy can be or comprise a small molecule or other agent that disrupts an immune checkpoint of a cell of the subject. Any of the checkpoint inhibitors described herein can be used. In certain embodiments, one or more of the checkpoint inhibitors of the second therapy are independently selected from the group consisting of pembrolizumab, nivolumab, ipilimumab, cemiplimab, atezolizumab, avelumab, durvalumab, pidilizumab, monoclonal antibody MEDI-0680, monoclonal antibody REGN2810, or fusion protein AMP-224 that targets PD-1, ociperlimab, islelizumab, a combination of ociperlimab and islelizumab, BMS-936559/MDX-1105. MPDL3280A/RG7446/atezolizumab. MSB0010718C/avelumab, or MEDI4736/durvalumab, tiragolumab, zimberelimab, tremelimumab, relatlimab, monoclonal antibody IMP321, nivolumab, etigilimab, domvanalimab, tiragolumab (RG6058), and vibostolimab. The one or more immune checkpoint inhibitors of the second therapy can inhibit an immune checkpoint of a cell selected from a group consisting of PD-1, PD-L1, CTLA-4, VISTA, LAG3, TIM3, TIGIT, PD-L2, IDO, AGR1, B7-H3, B7-H4, 2B4, BTLA, A2aR, and/or a member of the family of the KIRs such as KIRs and C-type lectin receptors, and STAT3.

The compound (or pharmaceutically acceptable salt or hydrate thereof) or composition comprising same, or a combination thereof and the one or more immune checkpoint inhibitors can be administered to the subject using any suitable method known in the art. Examples of suitable routes of administration include, but are not limited to, oral, intravenous, intramuscular, subcutaneous, and transdermal. The compounds, compositions, and/or a combination thereof and the one or more immune checkpoint inhibitors can be administered directly into the blood stream, into muscle, or into an internal organ. Suitable routes for parenteral administration include, but are not limited to, intravenous, intra-arterial, intraperitoneal, intrathecal, epidural, intracerebroventricular, intraurethral, intrasternal, intracranial, intratumoral, intramuscular, and subcutaneous. Use can be made of needle injectors, including microneedles, needle-free injectors, and infusions. The compounds, compositions, and/or a combination thereof and the one or more immune checkpoint inhibitors can be administered in unit dosage forms and/or formulations containing conventional non-toxic pharmaceutically acceptable carriers or excipients (or vehicles or adjuvants) either together in the same composition or in separate compositions.

In the method, the compound(s) or pharmaceutically acceptable salt or hydrate thereof (or pharmaceutical composition comprising the compound(s) or pharmaceutically acceptable salt(s) or hydrate(s) thereof and a pharmaceutically acceptable carrier or excipient) and the one or more immune checkpoint inhibitors can be administered simultaneously or sequentially, in either order, by the same or different routes. When administered simultaneously by the same route, the formulations can be the same or different. In various embodiments, the compound (or pharmaceutically acceptable salt or hydrate thereof) can be administered to the subject after the immune checkpoint inhibitor(s). The timing between the administration of immune checkpoint inhibitor(s) and the administration of the compound(s) can vary widely depending on factors that include the type of immune checkpoint inhibitor(s) being used, the binding specificity of the compound (or pharmaceutically acceptable salt or hydrate thereof), the identity of the targeting moiety of the compound, the type of cancer, the location in the subject of the cancer, the means used to administered to the subject the immune checkpoint inhibitor(s) and the compound, as well as the health, age, and weight of the subject.

The compound(s) can be administered before or after the immune checkpoint inhibitor(s), such as within about 3, 6, 9, 12, 15, 18, 21 or 24 hours, or within about 0.5, 1, 1.5, 2, 2.5, 3, 4, 5, 6, 7, 8, 9, 10 or more days. The rate of administration of the compound and/or immune checkpoint inhibitor(s) can be adjusted, for example (e.g., as a function of dosing schedule, such as continuous, once daily, twice daily, thrice daily, once weekly, twice weekly, or thrice weekly). By “continuous” is meant for at least one hour, at least four hours, at least six hours, at least eight hours, at least 10 hours, at least 12 hours, or at least 24 hours, or a regimen of daily or weekly administration, such as once/day twice/day, thrice/day, every other day, once/week, twice/week, thrice/week or any other suitable regimen. In the method, the immune checkpoint inhibitor(s) (or pharmaceutical composition comprising the immune checkpoint inhibitor(s) and a pharmaceutically acceptable carrier or excipient) and the compound can be administered intravenously.

In certain approaches, administering both the first and second therapies to the subject results in a greater than additive inhibition of growth of the cancer.

The first therapy can be administered to a subject intravenously, orally, intramuscularly, intraperitoneally, topically or by inhalation. The second therapy can be administered to a subject intravenously, orally, intramuscularly, intraperitoneally, topically or by inhalation.

The term “subject” means an animal, such as a mammal, and in particular a human. In veterinary applications, the subject can be a laboratory, an agricultural, a domestic, or a wild animal. Examples of such animals include, but are not limited to, a rodent, a rabbit, a monkey, a chimpanzee, a dog, a cat, a cow, a horse, a pig, a sheep, a goat, a bear, a panda, a lion, a tiger, a leopard, an elephant, a zebra, a giraffe, a gorilla, a dolphin, or a whale.

In some embodiments, the methods are used to treat cancers, for example, regardless of if the cancer expresses the folate receptor. In some embodiments, folic acid and other folate receptor-binding ligands (or radicals thereof), such as, for example folate, are used as targeting moieties, since for example, they have affinity for FRβ.

The methods hereof can further comprise imaging the cancer in the subject. Imaging the cancer can comprise imaging by optical imaging, positron emission tomography (PET), or single photon emission computed tomography (SPECT), for example.

In the methods described herein, the cancer can additionally be imaged prior to administration to the subject of the first therapy or the second therapy. The cancer additionally, or alternatively, can be imaged during or after administration of the first and/or second therapies to assess metastasis, for example, and the efficacy of treatment. For example, imaging can occur by PET imaging, magnetic resonance imaging (MRI), or SPECT/computed tomography (CT) imaging. The imaging method can be any suitable imaging method known in the art.

The cancer can be any cancer. “Cancer” has its plain and ordinary meaning when read in light of the specification and can include, but is not limited to, a group of diseases involving abnormal cell growth with the potential to invade or spread (i.e., metastasize) to other parts of the body. Examples include, but are not limited to, a cancer of the brain, thyroid, lung, pancreas, kidney, stomach, gastrointestinal stroma, endometrium, breast, cervix, ovary, colon, or prostate, leukemias, lymphomas, other blood-related cancers, and head and neck cancer. In certain embodiments, the cancer being treated is a tumor. In certain embodiments, the cancer is malignant. In certain embodiments, the cancer comprises a PD-L1U tumor.

In certain embodiments, the cancer comprises a hot tumor. In certain embodiments, the cancer comprises a cold tumor. In certain embodiments, the cancer comprises an immune desert tumor. A cold tumor is typically characterized by the absence of T cell infiltration (i, e., an immune desert) or one where T cells accumulate only at the edges of the tumor site. These types of tumors display immune ignorance, low immunogenicity, and poor resistance to immune checkpoint blockade therapy. Conversely, hot tumors are typically characterized by a high degree of T cell infiltration or a tumor where there is preexisting immunity, high immunogenicity, and generally exhibit a better response to immune checkpoint blockade therapy.

In some embodiments, the cancer is imaged prior to administration of the first and second therapies to the subject. Imaging can be done by PET, MRI or SPECT/CT for example.

Administration of the first and second therapies can be performed to treat a relapse of a cancer in a subject or resistance to checkpoint blockade therapy in a subject.

In certain embodiments, a use of a (i) compound, a pharmaceutically acceptable salt, hydrate, or solvate of the compound, or (ii) a composition comprising a compound, a pharmaceutically acceptable salt, hydrate, or solvate thereof in the manufacture of a medicament for the treatment of cancer in a subject is provided. The compound can be any compound described herein. The composition can be any composition described herein. The medicament can be for use in combination with administration of an immune checkpoint inhibitor therapy to the subject such as, for example, one or more immune checkpoint inhibitors described herein.

Still further, a method for enhancing a potency of one or more immune checkpoint inhibitors is provided. The method can comprise administering, to the subject, one or more compounds hereof (or pharmaceutically acceptable salts or hydrates thereof) comprising a radical of an immune modulator or pharmaceutically acceptable salt or hydrate thereof attached, via a linker, to a folate ligand or functional fragment or analog thereof; and contacting a targeted cell of the subject with the one or more compounds to reprogram M2-type macrophages of the subject to M1-type macrophages. The one or more compounds of such method can comprise any of the compounds described herein. The immune modulator or pharmaceutically acceptable salt or hydrate thereof can target a pattern recognition receptor. The one or more compounds can be formulated into a composition hereof by further comprising one or more pharmaceutically acceptable carriers, adjuvants, diluents, excipients, and/or vehicles, or combinations thereof. The immune modulator or pharmaceutically acceptable salt or hydrate thereof can comprise a TLR 3, 7, 8, 9, or 7/8 agonist. In certain embodiments, administering the at least one compound activates anti-tumor cells or an anti-inflammatory signaling cascade in the subject. The anti-tumor cells can be, for example, T cells, macrophages, or both T cells and macrophages.

In some embodiments, the method further comprises obtaining, or having obtained, a sample from the subject; quantifying a level of expression of one or more biomarkers in the sample, each of the one or more biomarkers selected from the group consisting of CCL 18, arginase 1 (Arg1), matrix metallopeptidase 9 (MMP9), metalloproteinase 3 (TIMP 3), IL-1β, hydroxyproline, collagen, PDGF, TGFβ, FRβ, TNFα, IFN-γ, anti-mannose receptor (CD206), cluster of differentiation 86 (CD86), cluster of differentiation 163 (CD163), IL-6, chemokine 10 (CXCL10), immune interferon (IFNα); comparing the level of expression of each of the one or more biomarkers in the sample to an expression level of such biomarker in a control; and administering or having administered to the subject a therapeutically effective amount of an unconjugated agonist or inhibitor if CCL18, Arg1, MMP9, TIMP 3, IL-1β, PDGF, TGFβ, FRβ, CD206, CD163, hydroxyproline, or collagen are upregulated relative to the expression level of the control or TNFα, IFN-γ, IL-6, CXCL10, IFNα or CD86 are downregulated or not expressed relative to the expression level of the control. In some embodiments, the folate ligand or functional fragment or analog thereof is specific for FRβ and binds to a FRβ on the cell.

### Reprogramming M2-Like Macrophages to M1-Like Macrophages

In at least one embodiment, a method is provided for treating and/or preventing a relapse of a cancer. The method comprises administering to the subject a therapeutically effective amount of one or more compounds comprising a targeting moiety (such as a folate receptor-binding ligand) attached to a drug (via a linker or otherwise) for reprogramming the M2-like macrophages in the tissue or organ to a MI-like phenotype. For example, the drug can be a TLR agonist (for example, having formula I, III, or IV) or any other molecule or compound that is effective to reprogram a macrophage from the M2 phenotype to the M1 phenotype conjugated to folate. In at least one embodiment, the drug can be selected from a TLR 3 agonist, a TLR7 agonist, a TLR 7/8 agonist, a TLR8 agonist, and a TLR9 agonist. In some embodiments, the drug can reprogram M2-like macrophages to a M1 phenotype, thereby reducing anti-inflammatory cytokine and growth factor production. For example, in at least one embodiment, reprogramming of the M2-like macrophages to a M1 phenotype results in the activation of anti-tumor cells and/or an anti-inflammatory signaling cascade within the tumor microenvironment (TME).

There are two main immunity strategies found in vertebrates: the innate immune system and the adaptive immune system. The innate, or non-specific, immune response, is the first line of defense against non-self pathogens and consists of physical, chemical and cellular defenses. The adaptive immune system, on the other hand, is called into action against pathogens that evade or overcome the primary innate immune defenses.

Inflammatory response plays a critical role in immunity. When tissues are damaged or a pathogen is detected, for example, an inflammatory response is initiated, and the immune system is mobilized. The immune cells of the innate immune system (i.e., neutrophils and eosinophils) are the first recruited to the site of tissue injury or damage or pathogen location via blood vessels and the lymphatic system, followed by macrophages.

The cells of the innate immune system can express special pattern recognition receptors that sense and bind with specific protein sequences present in microbial pathogens or other non-self molecules.

Examples of two classes of molecules that can bind to pattern recognition receptors include pathogen-associated molecular patterns associated with microbial pathogens and damage-associated molecular patterns associated with components of the host's cells that are released during cell damage or death. Recognition of these protein sequences by the pattern recognition receptors can initiate signal transduction pathways that trigger the expression of certain genes whose products control innate immune responses (e.g., in some cases, instructing the development of antigen-specific acquired immunity). Accordingly, the pattern recognition receptors mediate these signaling pathways and, in certain cases, can be used to control positively or negatively innate—and even adaptive—immune response.

Macrophages are a diverse group of white blood cells known for eliminating pathogens through phagocytosis and are broadly classified as either having an M1 or M2 phenotype depending on which specific differentiation they undergo in response to the local tissue environment. In some instances, macrophages are polarized towards the M1 phenotype by exposure to interferon gamma (IFN-γ), lipopolysaccharide (LPS), and/or granulocyte-macrophage colony stimulating factor (GM-CSF). In certain instances, the M1 phenotype is characterized by the production of high levels of pro-inflammatory cytokine(s) (such as interleukin 1 β (IL-1β), tumor necrosis factor (TNF), interleukin 12 (IL-12), interleukin 18 (IL-18), and/or interleukin 23 (IL-23)), an ability to mediate resistance to pathogens, strong microbicidal properties, high production of reactive nitrogen and oxygen intermediates, and/or promotion of T helper type 1 (Th1) cell responses.

In other words, M1 polarization can activate anti-tumor cells, such as T cells (including, without limitation, Th1 cells) and macrophages (including, without limitation, immune protective M1 tumor-associated macrophages (TAMs) and “classically activated”/M1 myeloid-derived suppressor cells (MDSCs)) in the TME). In some instances, M1 polarization is associated with the “attack and kill” phase of the innate immune response, which can induce lysis in various types of cancer cells. Additionally, the proinflammatory, tumoricidal cytokine release (as well as the release of similar chemokines, leukotrienes, prostaglandins, and complement from the M1 macrophages) can result in an overall proinflammatory signaling cascade to reactive T cell function and affect the regulation of multiple genes and their transcription factors, which may ensue in the production of other cytokines, an increase in the number of surface receptors for other molecules which ultimately induces the recruitment of inflammatory cells. Aside from local effects, these mediators may also produce systemic effects such as the production of acute inflammatory response proteins. In certain instances, M1 polarization operates to inhibit or prevent initial establishment of infection and/or remove damaged/cancerous tissue; tumor growth and metastasis formation can be decreased by M2 TAM depletion, inhibition of M2 macrophage recruitment and pro-tumoral function, and by reprogramming M2 macrophages (e.g., TAMs and/or MDSCs) into the proinflammatory M1 phenotype.

In certain instances, after the innate immune system performs this “attack and kill” phase, a macrophage may reprogram itself to become a healing system (i.e. M2-type) and, for example, release growth factors to promote healing. Such growth factors may include (without limitation) certain cytokines such as interleukin 4 (IL-4), interleukin 10 (IL-10), platelet-derived growth factor (PDGF), transforming growth factor-β1 (TGFβ), chemokine (C-C motif) ligand 18 (CCL18), and/or interleukin 13 (IL-1β). In certain instances, exposure to such cytokines/growth factors alternatively activates the M2 macrophage phenotype.

In contrast to M1, M2 macrophages can be associated with wound healing and tissue repair. In some instances, M2 macrophages are characterized by their involvement in tissue remodeling, immune regulation/suppression, and/or tumor promotion. In specific instances, M2 macrophages produce polyamines to induce cell proliferation and/or proline to induce collagen production. While this healing response is beneficial in a healthy subject, the presence of M2 macrophages can have significantly detrimental effects through immune suppression and/or the promotion of tumor growth and fibrosis for those subjects suffering from cancer. Chemokines and other factors can be released to promote the infiltration of immune cells to the damaged tissue (e.g., an innate immune response), which, for example, include monocytes and macrophages that assume an M2-like phenotypes and, for example, release anti-inflammatory cytokines. The chronic secretion of these cytokines can then activate tissue-resident and infiltrating fibroblasts/fibrocytes to become myofibroblasts that, in turn, secret collagen and other extracellular matrix proteins that can stiffen the surrounding tissue. In some instances, these M2 macrophages exacerbate the disease by promoting fibrosis. In some instances, the growth factors and other cytokines produced by the M2 phenotype drive cancerous tumor growth through similar pathways.

In certain cancers, macrophages can be disproportionately biased towards the anti-inflammatory (M2-like) phenotype. In certain instances, immune modulators can convert—e.g., reprogram—activated myeloid cells (e.g., M2-like macrophages) into a proinflammatory M1 polarization (e.g., where they produce little or no growth factors and/or related cytokines and, for example, slow or even eliminate the progression of the disease state (i.e. cancer)). In certain instances, the combinations, compositions and methods reverse the proinflammatory to anti-inflammatory shift observed during the course of the development of certain cancers. In some embodiments, the combinations, compositions and methods decrease the amount/expression of fibrotic biomarkers (e.g., those associated with anti-inflammatory activity (e.g., CCL18, hydroxyproline, and collagen)) in an individual or a sample taken from a subject, which is indicative of macrophage conversion to the M1 phenotype and, thus, anti-tumor cell activation (e.g., T cells and/or macrophages) and the initiation of an anti-inflammatory signaling cascade. An “individual,” “subject” or “patient” can be a mammal, preferably a human, but can also be an animal.

A “marker” or “biomarker” may be described as being differentially expressed when the level of expression in a subject who is experiencing an active disease state is significantly different from that of a subject or sample taken from a healthy subject or one not experiencing the disease state. A differentially expressed marker may be overexpressed or underexpressed as compared to the expression level of a normal or control sample, or subjects' baseline (in the embodiment mentioned in the immediately preceding paragraph, the biomarker is decreased or underexpressed). The increase or decrease, or quantification of the markers in a biological sample, can be determined by any of the several methods known in the art for measuring the presence and/or relative abundance of a gene product or transcript. The level of markers may be determined as an absolute value, or relative to a baseline value, and the level of the subject's markers compared to a cutoff index. Alternatively, the relative abundance of the marker or markers can be determined relative to a control, which can be a clinically normal subject. Further, the terms “gene overexpression” and “overexpression” (when used in connection with a gene) and their formatives have the meaning ascribed thereto by one of ordinary skill in the relevant arts, which includes (without limitation) the overexpression or misexpression of a wild-type gene product that may cause mutant phenotypes and/or lead to abundant target protein expression.

In some embodiments, the combinations, compositions, and methods increase anti-tumor biomarkers (e.g., TNFα, and IFN-γ). In some embodiments, combinations and compositions are provided that reverse the M2-like phenotypic shift (e.g., providing provide an effective treatment to cancers or conditions thereof.

Administering the at least one compound (or pharmaceutically acceptable salt or hydrate thereof) or composition of the first therapy can reprogram M2-type macrophages of the subject to M1-type macrophages and enhance a potency of the one or more checkpoint inhibitors of the second therapy relative to a baseline potency of the one or more checkpoint inhibitors (e.g., baseline potency being when the checkpoint inhibitors are administered not in combination with the first therapy).

In some embodiments, administration of the compounds or compositions (e.g., to a subject) can convert a macrophage in fibrotic tissue in the subject from an M2-like phenotype to an M1-like phenotype. The M2-type macrophages of the subject can be MDSCs, TAMs, or both MDSCs and TAMs.

In some embodiments, a decrease in cytokines that stimulate collagen synthesis (i.e., CCL18, PDGF, and IL-1Q) occurs after administration of a compound or composition hereof, as well as the concurrent increase in cytokines that inhibit collagen production (e.g., IFN-γ). Notably, in at least one embodiment, after administration of the compounds or compositions, the cytokine profiles are consistent with the reprogramming of the M2-like phenotype to the M1-like phenotype. A “profile” or “assay” is a set of one or more markers and their presence, absence, and/or relative level or abundance (relative to one or more controls). For example, a cytokine profile is a dataset of the presence, absence, relative level or abundance of cytokines present within a sample. A genomic or nucleic acid profile is a dataset of the presence, absence, relative level or abundance of expressed nucleic acids (e.g., transcripts, mRNA, or the like). A profile may alternatively be referred to as an expression profile.

In some embodiments, the net consequence of the reprogramming is an increase in alveolar air sacs, a decrease in extracellular matrix deposition, and a reduction in hydroxyproline/collagen biosynthesis; an effective reversal of the disease (e.g., see Example 4).

It is to be understood that, while particular drugs and formulae are described herein, any compound (e.g., drug) useful for reprogramming activated myeloid cells into an antifibrotic M1-like phenotype can be used in the compounds, compositions, combinations and methods hereof (for example, any compound (e.g., drug) capable of binding with a pattern recognition receptor and inhibiting at least a portion of the innate immune system response downstream thereof). In some embodiments, analogs and/or derivatives a compound or pharmaceutically acceptable salt or hydrate thereof can be used in the targeting compounds, compositions, and combinations.

Further, more than one compound can be administered and, in some instances, the compounds can comprise different drugs. For example, the different drugs can be selected from a TLR7 agonist and a TLR9 agonist. In yet another embodiment, one or more compounds can be administered in a composition along with one or more conjugated and/or unconjugated drugs. In some embodiments, any of the compounds and drugs can be used in accordance with the methods described herein and, in some instances, depending on the desired application, can be combined with other drugs that deplete or inhibit myeloid-derived suppressor cells (e.g., in connection with treatment for cancer), downregulate the production of growth factors (e.g., pirfenidone), directly modifies the fibroblasts via inhibiting mammalian target of rapamycin complex 1 (mTORC1) signaling, and/or any other proinflammatory and/or anticancer drugs and therapies. “Downregulation” and its formatives (such as “down-regulation” or “down-regulated,” for example) may be used interchangeably and refer to a decrease in the level of a marker, such as a gene, nucleic acid, metabolite, transcript, protein, or polypeptide. Similarly. “upregulation” and its formatives (“p-regulation” or “up-regulated,” for example) may also be used interchangeably and refer to an increase in the level of a marker, such as a gene, nucleic acid, metabolite, transcript, protein, or polypeptide. Also, a pathway, such as a signal transduction or metabolic pathway may be up- or down-regulated.

In certain embodiments, the method can comprise providing a compound hereof, a pharmaceutical composition comprising the same, or a combination hereof (e.g., a therapeutically effective amount of a compound or composition hereof and one or more immune checkpoint inhibitors); and exposing M2-type macrophages of the subject with thereto; wherein the M2-type macrophages convert to an M1-phenotype which facilitates activation of the subject's own immune response concurrently with administration of the compounds, compositions, and/or combinations hereof.

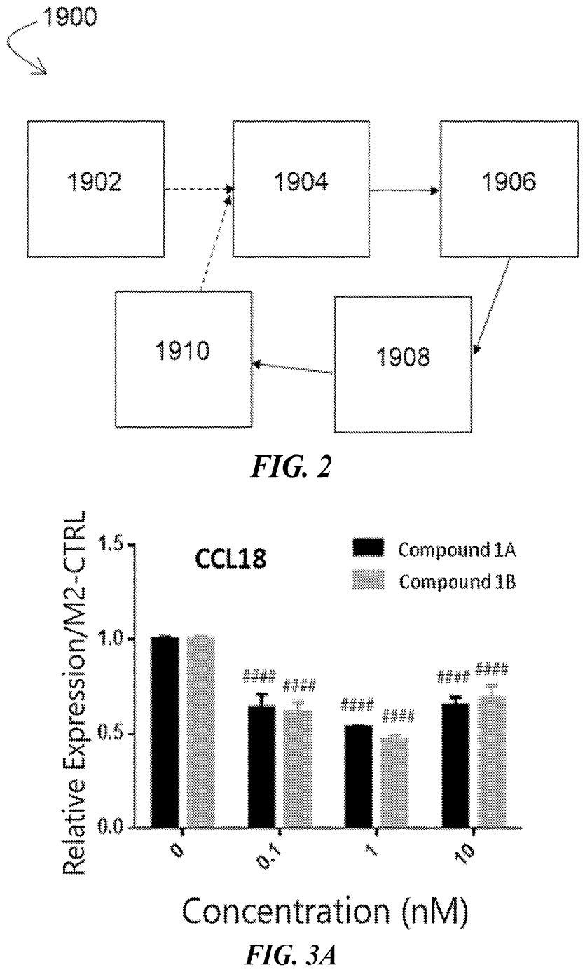

Now referring to FIG. 2, a flow chart representative of a method 1900 for using one or more of the compounds (or pharmaceutically acceptable salts or hydrates thereof). In at least one instance, method 1900 comprises the steps of contacting a cell of a subject with (administering) at least one compound comprising an immune modulator (or pharmaceutically acceptable salt or hydrate thereof), for example and without limitation a TLR7 agonist, attached, via a linker, to a folate ligand or a functional fragment or analog thereof (step 1902). In at least one exemplary embodiment, the immune modulator or pharmaceutically acceptable salt thereof targets a pattern recognition receptor or a DAMP (i.e. by way of the targeting ligand thereof). The cell can comprise, for example, a cell of a subject experiencing, or at risk for experiencing, a cancer relapse or resistance and the at least one compound can comprise any of the compounds provided herein.

In at least one embodiment, the step 1902 of contacting a cell with at least one compound (or pharmaceutically acceptable salts or hydrates thereof) further comprises administering or applying to the subject a therapeutically effective amount of the at least one compound (or pharmaceutically acceptable salts or hydrates thereof). Additionally or alternatively, the at least one compound (or pharmaceutically acceptable salts or hydrates thereof) can be formulated into a composition further containing one or more pharmaceutically acceptable carriers, adjuvants, diluents, excipients, and/or vehicles, or combinations thereof.

In at least one embodiment, the disease state comprises a cancer that is resistant to, or relapse after, immune checkpoint therapy. Since checkpoint inhibitors target adaptive immune cells, their combination with a drug that reprograms the innate immune system can be beneficial. Moreover, because it has been established that MDSCs and TAMs can be reprogrammed to disrupt the pro-growth cycle with FA-TLR7 and TLR7/8 agonists, systemic administration of these FA-TLR7 and/or FA-TLR7/8 agonists in combination with immune checkpoint inhibitors can target cancers that show relapse or resistance or treatment inefficacy with the administration of checkpoint blockade alone.

In addition to step 1902, method 1900 can optionally comprise steps 1904-1910. At step 1904, a biological sample is obtained from the subject and, at step 1906, the level of expression of one or more biomarkers in the sample is quantified. For example, the sample can be obtained from an amount of peripheral blood drawn from the subject.

The quantification step 1906 can be performed using any appropriate method known in the art and can include, for example, qPCR, mass spectrometry, ELISA, and/or any other modality that is capable to measure/quantify biomarker expression. In at least one exemplary embodiment, the one or more biomarkers are selected from the group consisting of CCL 18, Arg1, MMP9, TIMP 3, IL-1β, PDGF, TGFβ, FRβ, hydroxyproline, collagen, TNFα, IFN-γ, CD206, CD163, IL-6, CXCL10, IFNα and CD86.

At step 1908, the level of expression of each of the one or more biomarkers in the sample is compared to an expression level of such biomarker in a control. The control can be a healthy individual or simply an individual that is not experiencing the disease state at issue. In at least one embodiment, a clinical difference between the expression level(s) of the one or more biomarkers in the sample and the expression level of the related biomarker(s) in the control can be indicative that the subject suffers from the disease state at issue. For example, and without limitation, if the comparison step 1908 indicates that expression of one or more of the biomarkers CCL18, Arg1, CD163, MMP9, TIMP3, IL-1β, PDGF, TGFβ, FRβ, hydroxyproline, collagen, and/or CD206 (i.e. the “anti-inflammatory biomarkers”) are upregulated as compared to the control, it is indicative of the subject experiencing an anti-inflammatory immune response, which is linked to the M2-like macrophage phenotype. Accordingly, in at least one embodiment, such result is indicative of the need to administer one or more compounds of the present disclosure to reprogram such M2-like macrophages to the M1 phenotype.

In contrast, if the comparison step 1908 indicates that expression of the aforementioned biomarkers are downregulated as compared to the control, or if the expression of one or more of TNFα, IFN-γ, and/or CD86 (the “proinflammatory biomarkers”) are upregulated as compared to the control, this, in certain embodiments, is indicative of the subject either showing a positive response to a previously administered compound (if applicable) and/or that the subject is experiencing a proinflammatory immune response, which is linked to the M1 phenotype and activation of one or more anti-tumor cells and/or an anti-inflammatory signaling cascade.

Optionally, at step 1910, if expression of one or more of the anti-inflammatory biomarkers in the sample are upregulated as compared to the respective expression level(s) in the control, or if the expression of one or more proinflammatory biomarkers are downregulated in the sample as compared to the respective expression level(s) in the control, an alternative therapy can be administered. In at least one embodiment, the alternative therapy can comprise administering a therapeutically effective amount of a derivative of the at least one compound previously administered at step 1902, where the derivative comprises the previously administered at least one compound modified with respect to either employing a different targeting moiety, a different linker size, and/or a different immune modulator in an attempt to better optimize the efficacy of the at least one compound for the subject. Additionally or alternatively, other treatments can be employed, including those conventionally known for treatment of the cancer disease at issue (e.g., imaging, surgery, chemotherapy, radiation, etc.). Steps 1904-1910 can be included and/or repeated as necessary or desired to satisfy the established standard and/or confirm the active ingredient(s) is/are effective to ameliorate the cancer disease state manifestations.

As stated above, the methods can be used to treat and/or prevent a cancer or relapse thereof (whether folate receptor-positive or folate receptor-negative). For example, in certain instances, such a method comprises administering to the subject a therapeutically effective amount and/or a prophylactically effective amount of one or more compounds (or pharmaceutically acceptable salts or hydrates thereof) comprising a targeting moiety attached to a drug (via a linker or otherwise) to reprogram the M2-like macrophages in the cancerous and/or tumor cells to a M1-like phenotype, such as, for example, a targeted TLR-7 agonist. Where the cancer is folate receptor-negative, such administration can additionally act to deplete or inhibit the MDSCs present in such tissue/tumor. Additional drugs can also be administered in connection with such methods, including, for example, a PI3k inhibitor, a signal transducer and activator of transcription 6 (STAT6) inhibitor, a mitogen-activated protein kinase (MAPK) inhibitor, an inducible nitric oxide synthase (iNOS) inhibitor, and an anti-inflammatory drug (e.g., methotrexate). In at least one embodiment, the drug can inactivate MDSCs.

The compounds and compositions can be used alone or in combination with checkpoint inhibitor immunotherapy such that the growth of cancer in the subject is inhibited. For example, in addition to step 1902, method 1900 can further comprise a step of administering checkpoint inhibitor immunotherapy (as described above).

Such combination therapies are particularly beneficial in treating cancers that are prone to relapse or single therapy resistance, resulting in the immune recognition of that cancer with the production of systemic immune response and, thus, effectively attacking or even eliminating (e.g., causing remission) of any remaining living cancer cells that possess the recognized antigen(s). As an important result, any remaining cancer could be eliminated or ameliorated. Accordingly, in certain approaches, methods of this disclosure comprise treating a subject who has a cancer that exhibits resistance to treatment with a checkpoint inhibitor.

Such combination therapy methods can be performed using any suitable checkpoint inhibitor(s) (as described above) and can include using more than one of these types of agents. The immune checkpoint inhibitor(s) used in combination with the inventive compound compounds or compositions can be any immune checkpoint inhibitor. As is known in the art, examples of an immune checkpoint inhibitor include (without limitation) the transmembrane programmed cell death 1 protein (PDCD1, PD-1; also known as CD279) and its ligand PD-Li (also known as CD274). In normal, nonmalignant physiology, PD-L1 on the surface of a cell binds to PD1 on the surface of an immune cell, which inhibits the activity of the immune cell. PD-Li upregulation on cancer cell surfaces is thought to facilitate evasion of the host immune system, at least in part by inhibiting T cells that would otherwise target the tumor cell. In certain embodiments, the one or more checkpoint inhibitors are each independently selected from the group consisting of pembrolizumab, nivolumab, ipilimumab, cemiplimab, atezolizumab, avelumab, durvalumab, pidilizumab, monoclonal antibody MEDI-0680, monoclonal antibody REGN2810, or fusion protein AMP-224 that targets PD-1, ociperlimab, islelizumab, a combination of ociperlimab and islelizumab, BMS-936559/MDX-1105, MPDL3280A/RG7446/atezolizumab, MSB0010718C/avelumab, or MED14736/durvalumab, tiragolumab, zimberelimab, tremelimumab, relatlimab, monoclonal antibody IMP321, nivolumab, etigilimab, domvanalimab, tiragolumab (RG6058), vibostolimab, avelumab, and durvalumab.

In certain approaches, administering both the compounds hereof (or pharmaceutically acceptable salts or hydrates thereof) and the checkpoint inhibitor therapy results in a greater than additive inhibition of growth of the cancer.

Where multiple therapeutics are co-administered, dosages can be adjusted accordingly, as is recognized in the pertinent art.

Where a method of combination therapy comprises administering more than one treatment to a subject, it is to be understood that the order, timing, number, concentration, and volume of the administration is limited only by the medical requirements and limitations of the treatment (i.e. two treatments can be administered to the subject, e.g., simultaneously or sequentially (in either order), or according to any other regimen).

In describing representative embodiments, the disclosure can have presented a method and/or process as a particular sequence of steps. To the extent that the method or process does not rely on the particular order of steps set forth herein, the method or process should not be limited to the particular sequence of steps described. As one of ordinary skill in the art would appreciate, other sequences of steps can be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences can be varied and still remain within the spirit and scope of the present disclosure.

While various embodiments of compounds, compositions, and methods have been described in considerable detail herein, the embodiments are merely offered by way of non-limiting examples. Many variations and modifications of the embodiments described herein will be apparent to one of ordinary skill in the art in light of the disclosure. It will therefore be understood by those skilled in the art that various changes and modifications can be made, and equivalents can be substituted for elements thereof, without departing from the scope of the disclosure. Indeed, this disclosure is not intended to be exhaustive or too limiting. The scope of the disclosure is to he defined by the appended claims, and by their equivalents.

Additionally, while many of the examples provided herein use mouse models, it will be appreciated by one of ordinary skill in the art that gene expression patterns in mouse models show extraordinarily significant correlations with those of the human conditions and many pathways are commonly regulated by multiple conditions in humans and mice. Accordingly, gene expression patterns and disease progression in mouse models closely recapitulate those in human conditions—particularly with respect to inflammatory diseases and cancers—and, as such, support that the working examples set forth herein correlate with the human data, specified conditions, and applications.

It is therefore intended that this description and the appended claims will encompass, all modifications and changes apparent to those of ordinary skill in the art based on this disclosure.

### General

For the purposes of promoting an understanding of the principles of the present disclosure, reference will now be made to the embodiments illustrated in the drawings and specific language will be used to describe the same. It will nevertheless be understood that no limitation of scope is intended by the description of these embodiments. On the contrary, this disclosure is intended to cover alternatives, modifications, and equivalents as can be included within the spirit and scope of this application as defined by the appended claims. As previously noted, while this technology can be illustrated and described in one or more preferred embodiments, the compositions, compounds and methods hereof can comprise many different configurations, forms, materials, and accessories.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

In the following description, numerous specific details are set forth to provide a thorough understanding of the present disclosure. Particular examples can be implemented without some or all of these specific details and it is to be understood that this disclosure is not limited to particular biological systems, particular cancers, or particular organs or tissues, which can, of course, vary, but remain applicable in view of the data provided herein.

Various techniques and mechanisms of the present disclosure will sometimes describe a connection or link between two components. Words such as attached, linked, coupled, connected, and similar terms with their inflectional morphemes are used interchangeably, unless the difference is noted or made otherwise clear from the context. These words and expressions do not necessarily signify direct connections but include connections through mediate components. It should be noted that a connection between two components does not necessarily mean a direct, unimpeded connection, as a variety of other components can reside between the two components of note. Consequently, a connection does not necessarily mean a direct, unimpeded connection unless otherwise noted.

Further, wherever feasible and convenient, like reference numerals are used in the figures and the description to refer to the same or like parts or steps. The drawings are in a simplified form and not to precise scale. It is understood that the disclosure is presented in this manner merely for explanatory purposes and the principles and embodiments described herein can be applied to compounds and/or composition components that have configurations other than as specifically described herein. Indeed, it is expressly contemplated that the components of the composition and compounds of the present disclosure can be tailored in furtherance of the desired application thereof.

In certain embodiments, the compounds, compositions and methods hereof are useful for the prevention and/or treatment of cancer. In certain embodiments, the compounds and/or compositions provided are also useful for the treatment of cancer. In some embodiments, the compounds, compositions and methods provided herein leverage strategies to (e.g., selectively) target the innate immune system and reprogram the polarization of a macrophage from M2 to M1 and, for example, leverage the checkpoint blockade properties thereof. In some embodiments, the compounds comprise toll-like receptor TLR7 and/or 8 agonists. In certain embodiments, the compounds provided herein are provided or used alone, in conjunction with a targeting agent, and/or in a combination therapy with other interventions.

### Certain Definitions

The following terms and phrases shall have the meanings set forth below. Unless defined otherwise, all technical and scientific terms used herein have the same meaning as commonly understood to one of ordinary skill in the art.

The term “about” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to l % of a given value. By way of further example, “about” or “approximately” can mean within 90%, within 95%, 99%, 99.5%, 99.9%, 99.99%, or at least about 99.999% or more of a stated value or of a stated limit of a range. Alternatively, particularly with respect to biological systems or processes, the term can mean within an order of magnitude, preferably within 5-fold, and more preferably within 2-fold, of a value. Unless otherwise stated, the term “about” means within an acceptable error range for the particular value, such as +1-20%, preferably +1-10% and more preferably ±1-5%.

Where a range of values is provided, it is understood that each intervening value, between the upper and lower limit of that range and any other stated or intervening value in that stated range, is encompassed. The upper and lower limits of these smaller ranges may independently be included in the smaller ranges, and are also encompassed, subject to any specifically excluded limit in the stated range. Where the stated range includes one or both limits, ranges excluding either or both of those limits are also included.

A phrase referring to “at least one of” a list of items refers to any combination of those items, including single members. As an example, “at least one of a, b, or c” is intended to cover: a, b, c, a-b, a-c, b-c, and a-b-c.

The terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation.

The term “or” is used to refer to a nonexclusive “or” unless otherwise indicated. In addition, it is to be understood that the phraseology or terminology employed herein, and not otherwise defined, is for the purpose of description only and not of limitation.

The terms and expressions employed are used as terms of description and not of limitation. Where certain terms are defined and are otherwise described or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings may be used in the “Detailed Description,” such use is solely for ease of reference and is not intended to limit any disclosure made in one section to that section only; rather, any disclosure made under one subheading is intended to constitute a disclosure under each and every other subheading.

It is recognized that various modifications are possible within the scope of the claimed invention. Thus, although the present invention has been specifically disclosed in the context of preferred embodiments and optional features, those skilled in the art may resort to modifications and variations of the concepts disclosed herein. Such modifications and variations are considered within the scope of the invention as claimed herein.

### EXAMPLES

The following examples serve to illustrate the present disclosure. The examples are not intended to limit the scope of the claimed invention in any way.

### Chemistry Examples

### Example A: Synthesis of Compound 1A

Compound 1A was synthesized according to scheme 1 below and as reported by Nikunj M. Shukla, Cole A. Mutz, Subbalakshmi S. Malladi. Hemamli J. Warshakoon, Rajalakshmi Balakrishna, and Sunil A. David. “Regioisomerism-dependent TLR7 agonism and antagonism in an imidazoquinoline; Structure-Activity Relationships in Human Toll-Like Receptor 7-Active Imidazoquinoline Analogues,” J Med Chem. 2012 Feb. 9; 55(3): 1106-1116.

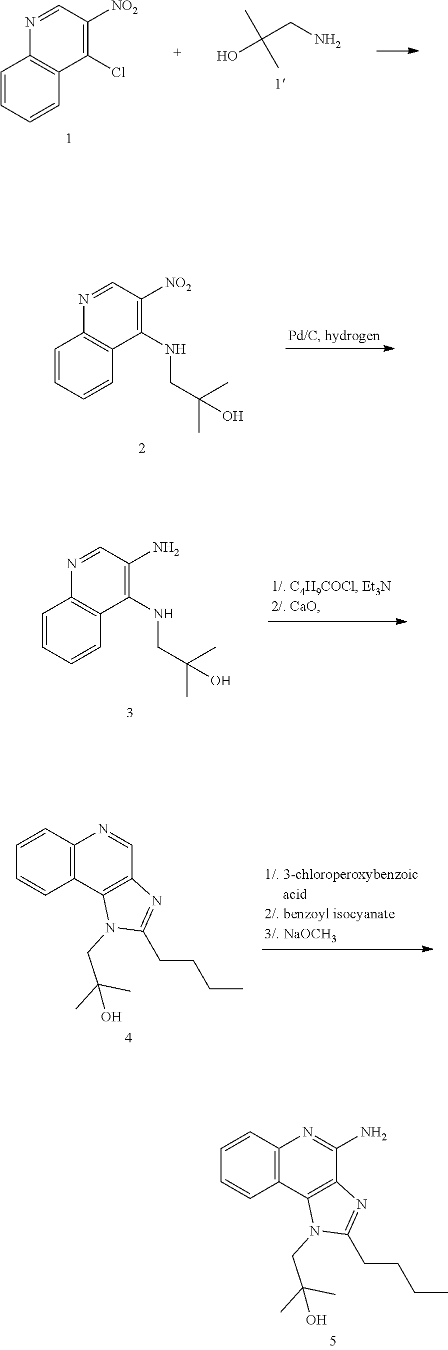

### Step 1: Synthesis of 1-amino-2-methylpropan-2-ol (compound)

2,2-dimethyloxirane (0.1 g, 1.388 mmol) was added dropwise to 20 mL ice cooled solution of ammonium hydroxide. The reaction mixture was stirred for 12 hours at room temperature. The solvent was removed under vacuum and the residue was dissolved in methanol. Di-tert-butyl dicarbonate (0.75 g, 3.47 mmol) was added to the reaction mixture and stirred for 4 hours. The mixture was purified using column chromatography (24% ethyl acetate (EtOAc)/hexane) to obtain tert-butyl 2-hydroxy-2-methylpropylcarbamate. The pure tert-butyl 2-hydroxy-2-methylpropylcarbamate was dissolved in 5 mL of trifluoroacetic acid and stirred for 35 minutes. The solvent was removed under reduced pressure to afford 1-amino-2-methylpropan-2-ol as the trifluoroacetate salt 1′. 1H NMR 500 MHz (500 MHz, CDCl3, δ in ppm): δ 8.62 (s, 2H), 3.02 (d, 2H), 2.06-2.04 (m, 2H), 1.37-1.34 (s, 6H).

### Step 2: Synthesis of 2-methyl-l-(3-nitroquinolin-4-ylamino)propan-2-ol (compound 2)

The trifluoroacetate salt of l-amino-2-methylpropan-2-ol (compound) (450 mg, 2.4 mmol) was added to the solution of 4-chloro-3-nitroquinoline (compound 1) (250 mg, 1.2 mmol) and Et₃N (0.5 ml, 3 mmol) in 4:1 mixture of toluene and 2-propanol. The mixture was heated to 70° C. for half an hour until a solid started precipitating. The reaction mixture was then cooled, filtered, washed with toluene/2-propanol (7:3), ether and cold water. The residue was dried at 80° C., to obtain 2-methyl-1-(3-nitroquinolin-4-ylamino)propan-2-ol (compound 2). Liquid chromatography-mass spectrometry (LCMS) analysis: [M+H]⁺ m/z=261.

### Step 3: Synthesis of 1-(3-aminoquinolin-4-ylamino)-2-methylpropan-2-ol (compound 3)

2-Methyl-1-(3-nitroquinolin-4-ylamino)propan-2-ol (compound 2) (450 mg, 1.72 mmol) was dissolved in methanol and hydrogenated over Pd/C as catalyst with hydrogen balloon for 4 hours. The solution was then filtered using celite, followed by evaporation of solvent under reduced pressure to afford 1-(3-aminoquinolin-4-ylamino)-2-ethylpropan-2-ol (compound 3). LCMS: [M+H]⁺ m/z=231. H NMR 500 MHz (CDCl3, δ in ppm): δ 8.12 (s, 1H), 7.61-7.58 (m, 1H), 7.48-7.40 (m, 2H), 4.90 (s, 2H), 3.47 (2H), 1.35-1.21 (s, 6H).

### Step 4: Synthesis of 1-(4-Amino-2-butyl-1H-imidazo[4,5-c]quinoline]-1-yl)-2-methylpropan-2-ol (compound 5, TLR7A)

To a solution of compound 3 (100 mg, 0.43 mmol) in anhydrous THF were added triethylamine (66 mg, 0.65 mmol) and valeryl chloride (62 mg, 0.52 mmol). The reaction mixture was then stirred for 6-8 hours, followed by removal of the solvent under vacuum. The residue was dissolved in EtOAc, washed with water and brine, and then dried over Na₂SO₄ to obtain the intermediate amide compound. This was dissolved in methanol (MeOH), followed by the addition of calcium oxide, and was heated in microwave at 110° C., for 1 hour. The solvent was then removed and the residue was purified using column chromatography (9% MeOH/dichloromethane) to obtain the compound 4 (58 mg). To a solution of compound 4 in a solvent mixture of MeOH:dichloromethane:chloroform (0.1:1:1) was added 3-chloroperoxybenzoic acid (84 mg, 0.49 mmol), and the solution was refluxed at 45-50° C., for 40 min. The solvent was then removed and the residue was purified using column chromatography (20% MeOH/dichloromethane) to obtain the oxide derivative (55 mg). This was then dissolved in anhydrous dichloromethane, followed by the addition of benzoyl isocyanate (39 mg, 0.26 mmol) and heated at 45° C., for 15 min. The solvent was then removed under vacuum, and the residue was dissolved in anhydrous MeOH, followed by the addition of excess sodium methoxide. The reaction mixture was then heated at 80° C., for an hour. The solvent was removed under vacuum, and the residue was purified using column chromatography (11% MeOH/dichloromethane) to obtain the compound 5. LCMS: [M+H]⁺ m/z=312. H NMR 500 MHz (CDCl3, 6 in ppm): δ 8.16-8.15 (d, 1H), 7.77-7.46 (d, 1H), 7.46-7.43 (m, 1H), 7.33-7.26 (m, 1H), 3.00-2.97 (m, 2H), 1.84-1.78 (m, 2H), 1.47-1.41 (m, 2H), 1.36 (s, 6H), 0.98-0.95 (m 3H).

### Example B: Synthesis of Compound 1B

Compound 1A can thereafter be used to synthesize Compound 1B according to scheme 2 below.

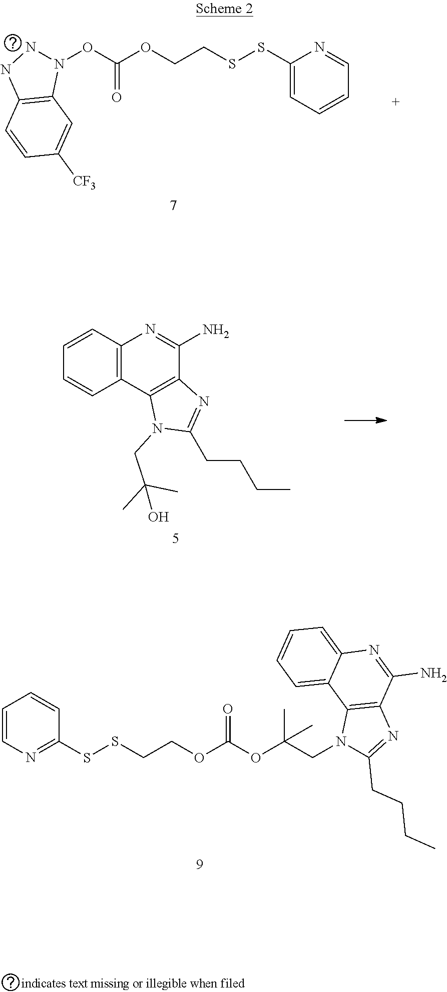 [SMILES: O=C(OCCSSc1ccccn1)ON1NNc2ccc(C(F)(F)F)cc21 ; CCCCc1nc2c(N)nc3ccccc3c2n1CC(C)(C)O ; CCCCc1nc2c(N)nc3ccccc3c2n1CC(C)(C)OC(=O)OCCSSc1ccccn1]

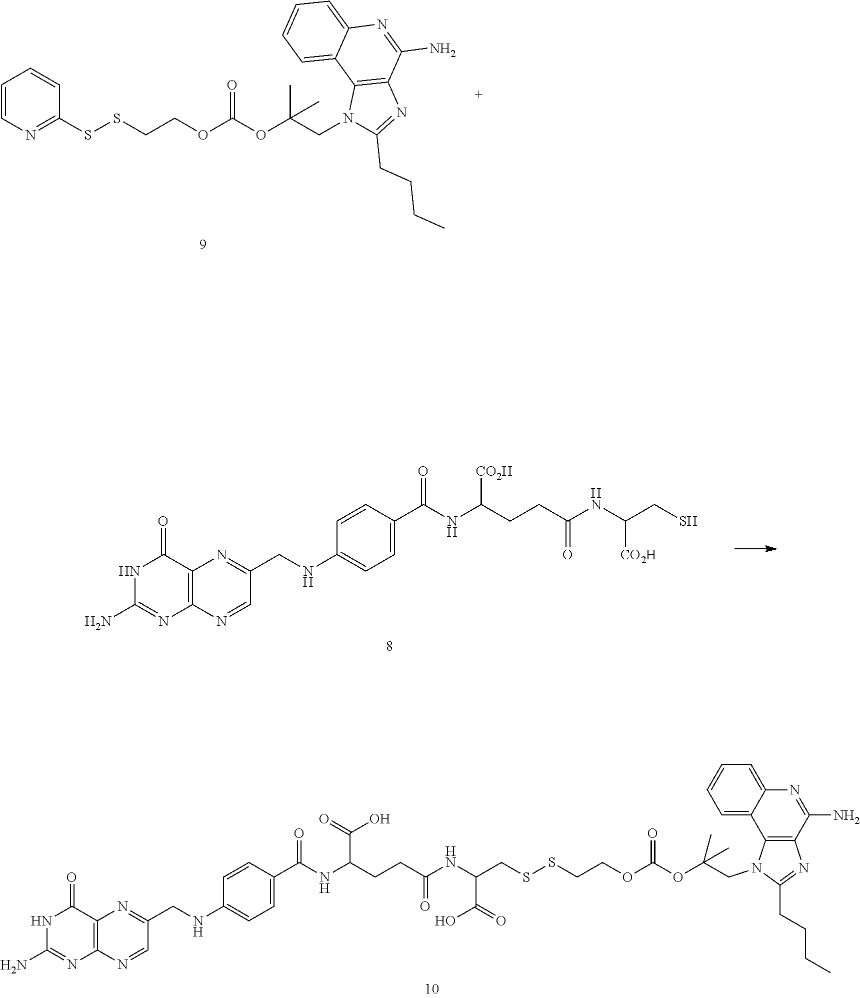 [SMILES: CCCCc1nc2c(N)nc3ccccc3c2n1CC(C)(C)OC(=O)OCCSSc1ccccn1 ; Nc1nc2ncc(CNc3ccc(C(=O)NC(CCC(=O)NC(CS)C(=O)O)C(=O)O)cc3)nc2c(=O)[nH]1 ; CCCCc1nc2c(N)nc3ccccc3c2n1CC(C)(C)OC(=O)OCCSSCC(NC(=O)CCC(NC(=O)c1ccc(NCc2cnc3nc(N)[nH]c(=O)c3n2)cc1)C(=O)O)C(=O)O]

Compound 1A, folate, and linker are commercially available or can be prepared according to methods known to the person skilled in the art.

Heterobifunctional linker 7 (88 mg, 0.213 mmol) was added to a solution of compound 5 (33 mg, 0.106 mmol) and dimethylaminopyridine (39 mg, 0.319 mmol) in 4 mL of methylene chloride at room temperature under nitrogen atmosphere and the mixture was stirred at reflux temperature for 7 hours at which time thin layer chromatography (TLC) analysis of the mixture indicated >80% conversion. The mixture was concentrated and purified by column chromatography using 10% acetonitrile in methylene chloride as eluant. The pure product compound 9 was obtained as a light yellow solid. A solution of compound 8 (1 eq.) in dimethyl sulfoxide (DMSO) was added in 3 portions at 20 min intervals to a solution of drug-linker intermediates compound 9 (1.0 eq.-1.5 eq.) in DMSO with dimethylaminopyridine (1 eq.). After 1-2 hours of stirring at room temperature under argon, LCMS analysis of the mixture indicated formation of the desired folate-drug compound (compound 10) as the major product. The mixture was purified by preparative high-performance liquid chromatography (HPLC). LCMS: [M+H]⁺ m/z=959. ¹H NMR (500 MHz, DMSO-d6) δ 8.58 (s, 1H), 8.49 (d, J=8.8 Hz, 1H), 7.90 (d, J=8.3 Hz, 1H), 7.83-7.74 (m, 1H), 7.54 (d, J=8.0 Hz, 2H), 7.48 (t, J=7.6 Hz, 1H), 7.41 (s, 1H), 7.06 (s, 1H), 6.81 (d. J=6.2 Hz, 1H), 6.61 (d, J=8.3 Hz, 2H), 6.27 (s, 1H), 4.43 (d. J=5.9 Hz, 2H), 4.28 (t, J=6.6 Hz, 2H), 4.00 (d, J=25.7 Hz, 3H), 3.03 (t, J=7.5 Hz, 2H), 2.97 (dd, J=13.0, 6.5 Hz, 1H), 2.09 (s, 2H), 1.81 (s, 7H), 1.40 (q, J=7.4 Hz, 2H), 1.22 (s, 2H), 1.13 (s, 2H), 0.91 (t, J=7.4 Hz, 3H).

### Example C: Synthesis of Compound 2A

Compound 2A can be synthesized according to Scheme 3 and Scheme 4.

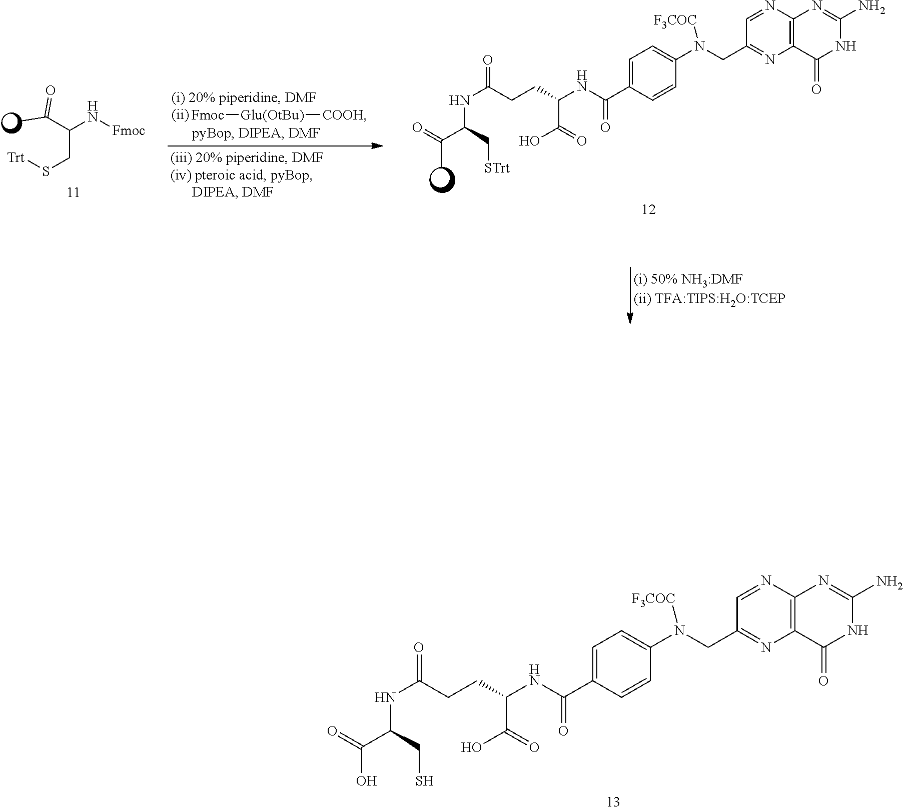

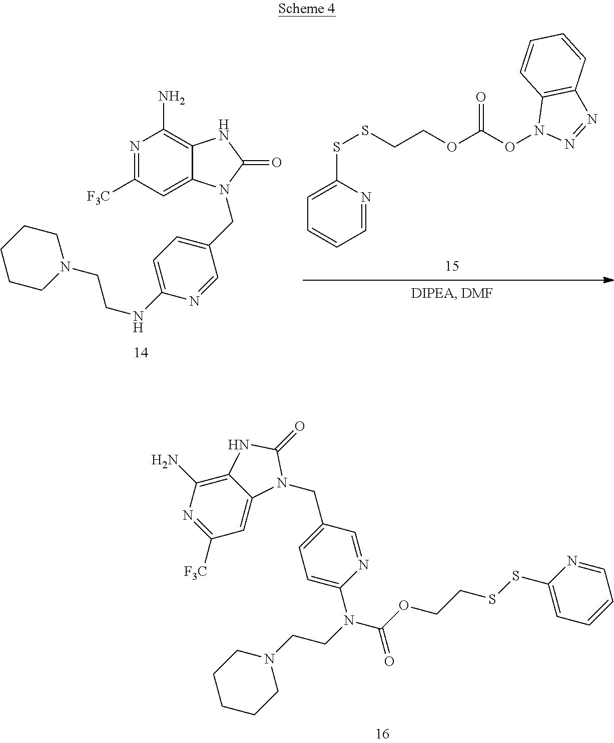

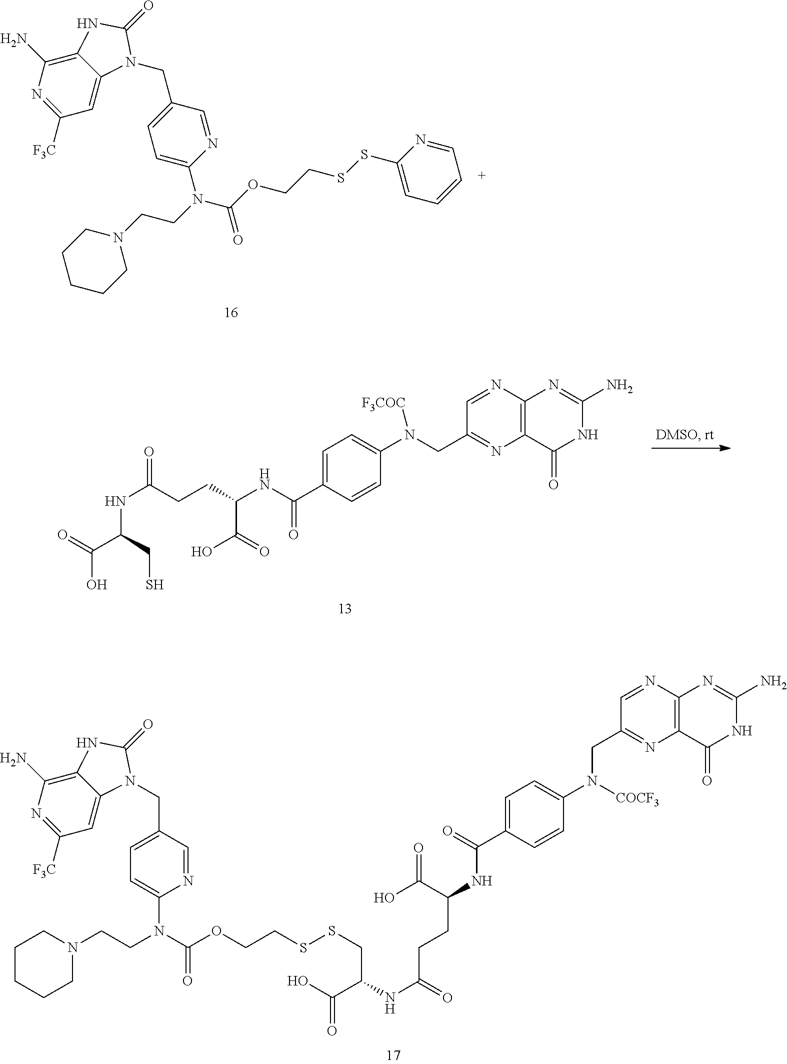

Cysteine loaded Wang resin (11) was initially deprotected using 20% piperidine in dimethylformamide (DMF). The free amine was treated with Fmoc-Glu(OtBu)—COOH in presence of benzotriazole-1-yl-oxytripyrrolidinophosphonium hexafluorophosphate (PyBop), N,N-Diisopropylethylamine (DIPEA) and DMF. The coupled product was deprotected using 20% piperidine in DMF and treated with pteroic acid in presence of PyBop, DIPEA and DMF, producing compound 12. The trifluoroacetyl group was deprotected with 50% ammonia-DMF solution. Finally, the resin was cleaved using a trifluoracetic acid:triisopropyl silane:water:tris(2-carboxyethyl)phosphine cocktail solution and purified using HPLC to get the folate-cysteine (13) as a yellow color solid.

Compound 14 was initially treated with a heterobifunctional linker reagent (15) to get the folate-cystine disulfide intermediate (16). This was then reacted with folate-cysteine (13) in DMSO and was purified using HPLC to produce compound 17 (e.g., Compound 2A). Characterization of all compounds was with LCMS using ammonium bicarbonate and acetonitrile as the buffer system. Observed mass from LCMS for Compound 2A was [M+H]+=1082.2.

### Example D: Synthesis of TLR7 Agonist TLR7-1A

Solvents, reagents and starting materials were purchased from commercial vendors and used as received unless otherwise described. All reactions were performed at room temperature unless otherwise stated. Starting materials were purchased from commercial sources or synthesized according to the methods described herein or using literature procedures.

Synthesis of TLR7 agonist TLR7-1A is described in Scheme 5:

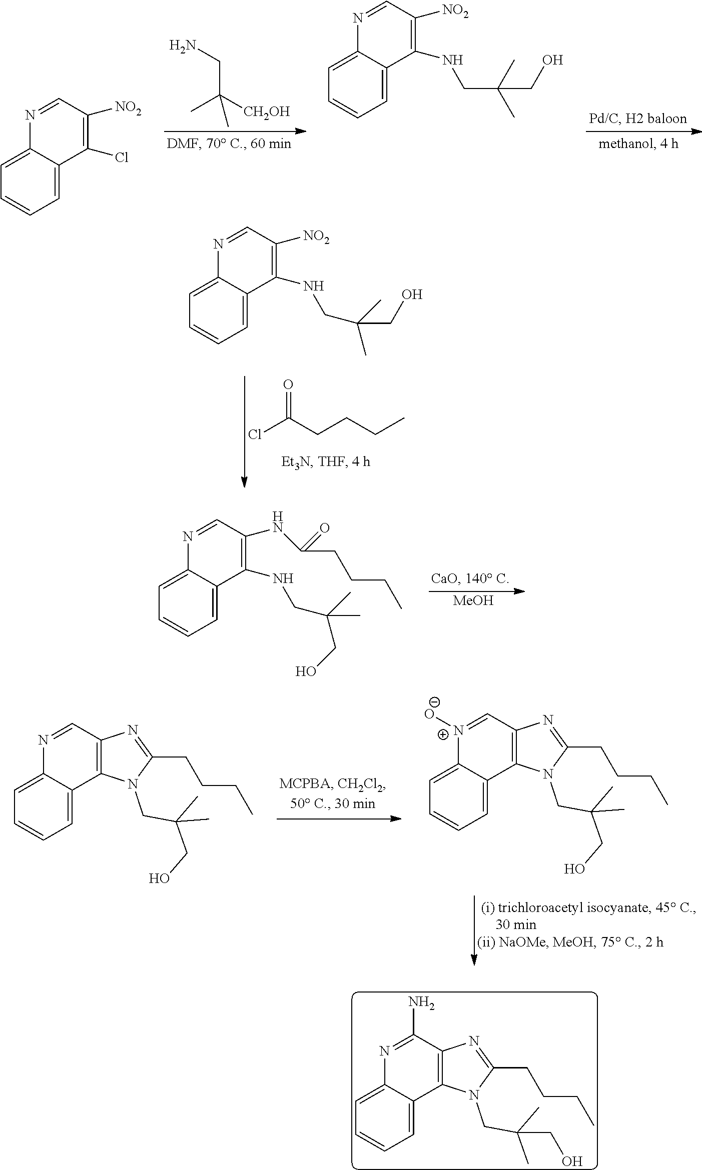

### Example E: Synthesis of TLR7 Agonist TLR7-1B

Synthesis of TLR7 agonist TLR7-1B is described in Scheme 6:

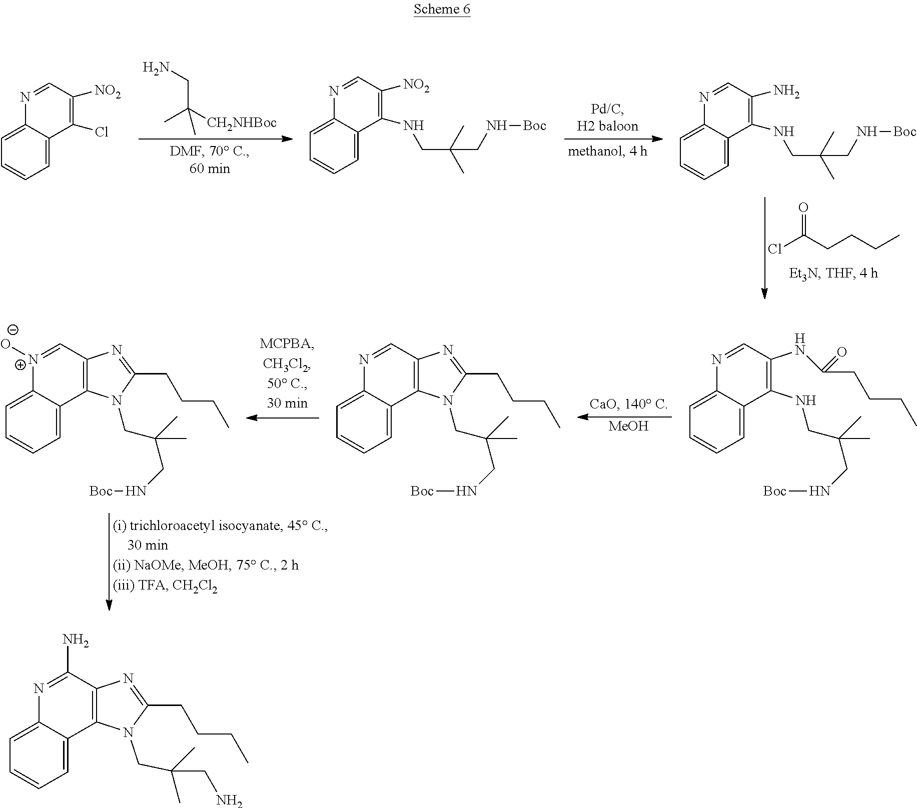

### Example F: Synthesis of TLR7 Agonist TLR7-1C

Synthesis of TLR7 agonist TLR7-1C is described in Scheme 7:

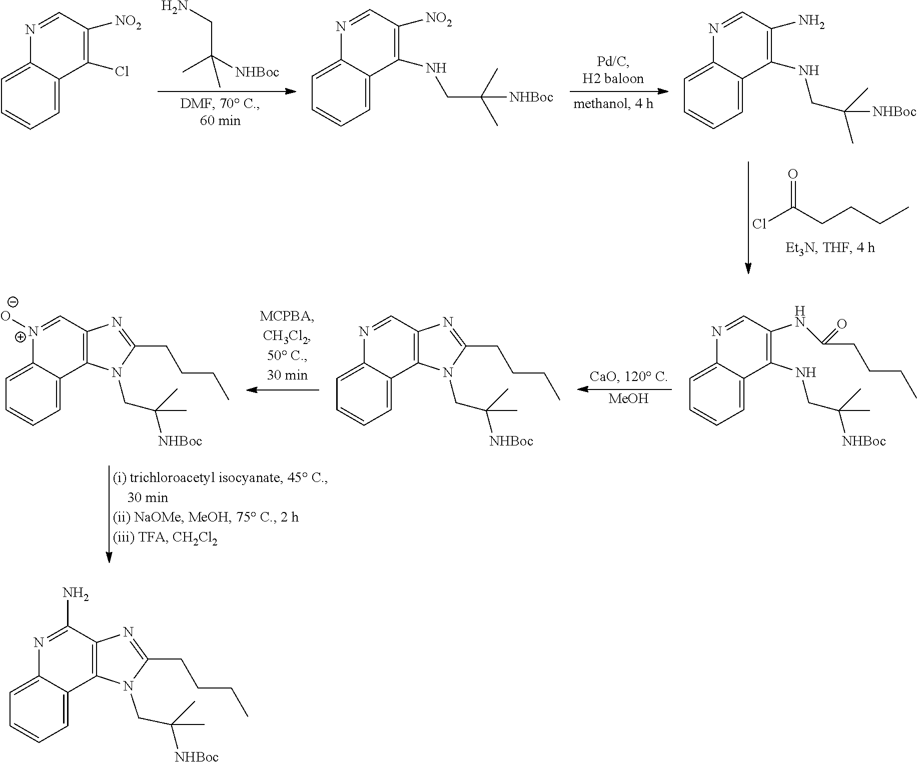

### Example G: Synthesis of Releasable TLR7-Folate Compound

Synthesis of releasable TLR7-folate compound is described in Scheme 8:

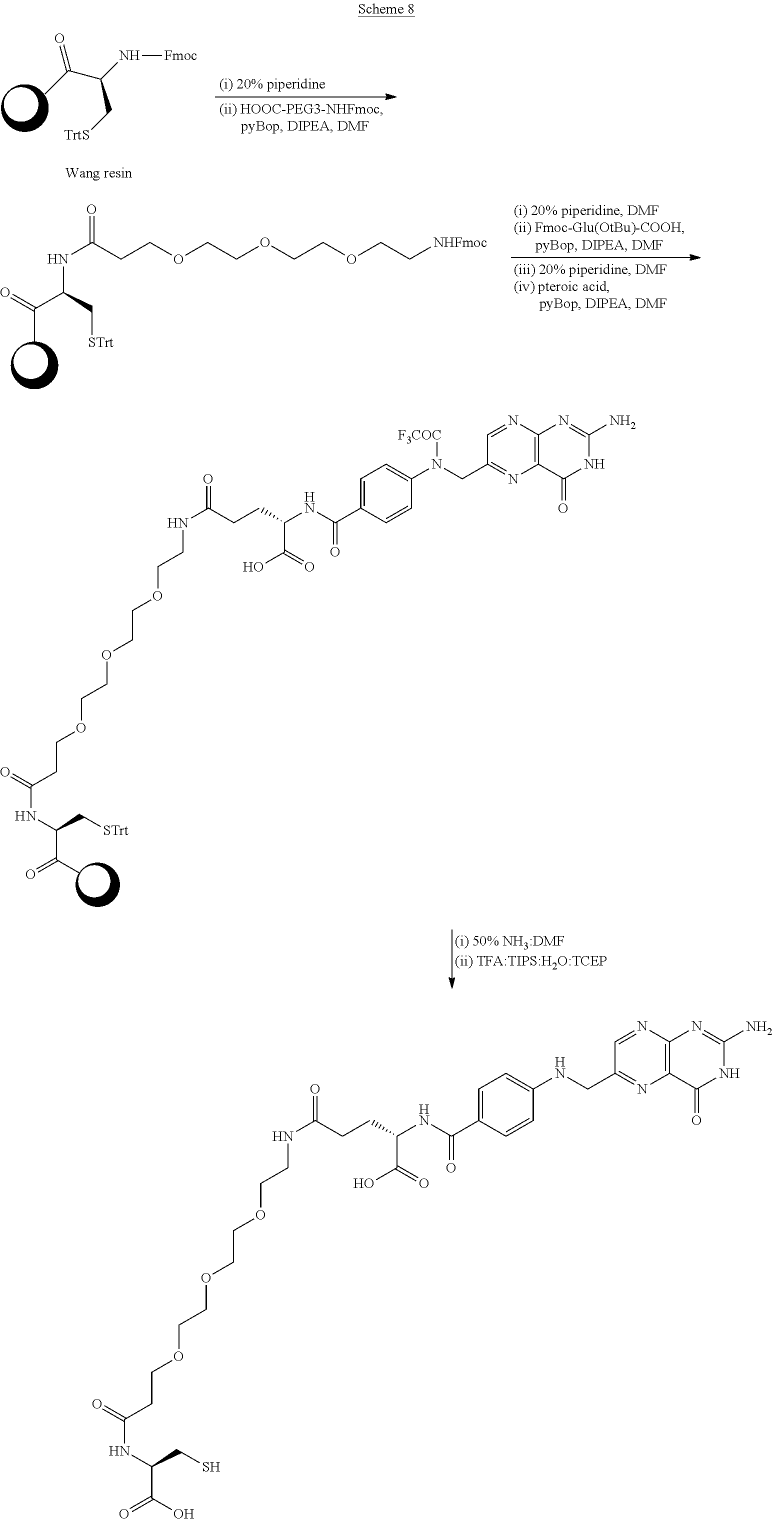

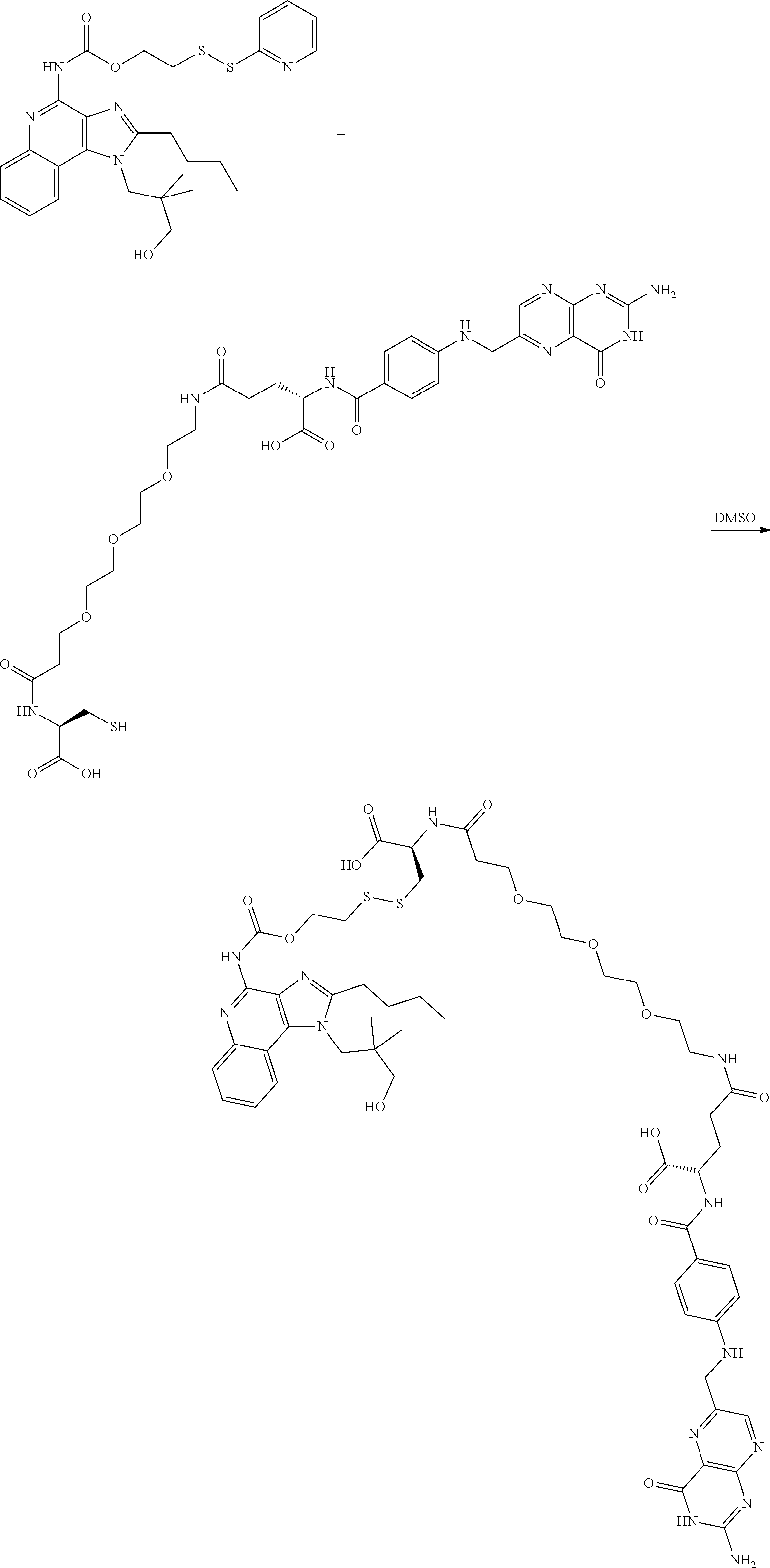

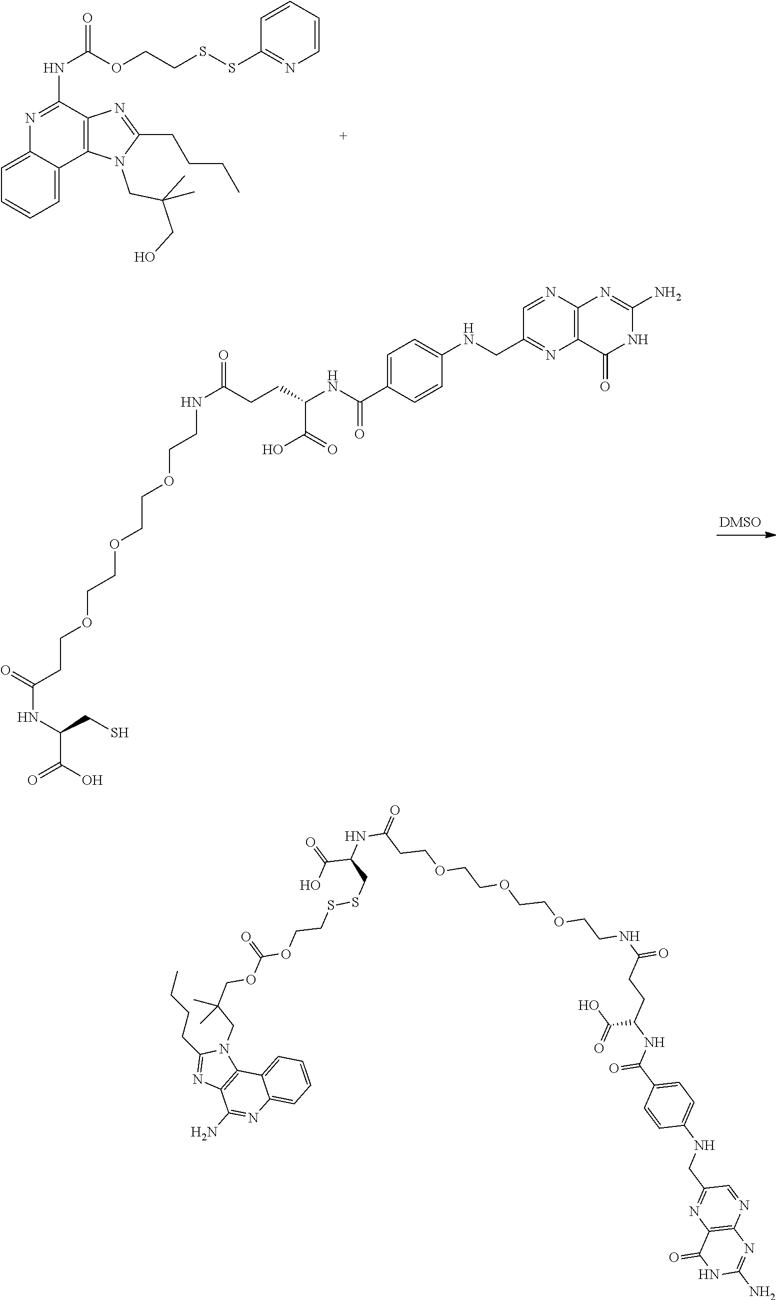

### Example H: Synthesis of Non-Releasable TLR7-Folate Compound

Synthesis of non-releasable TLR7-folate compound is described in Scheme 9:

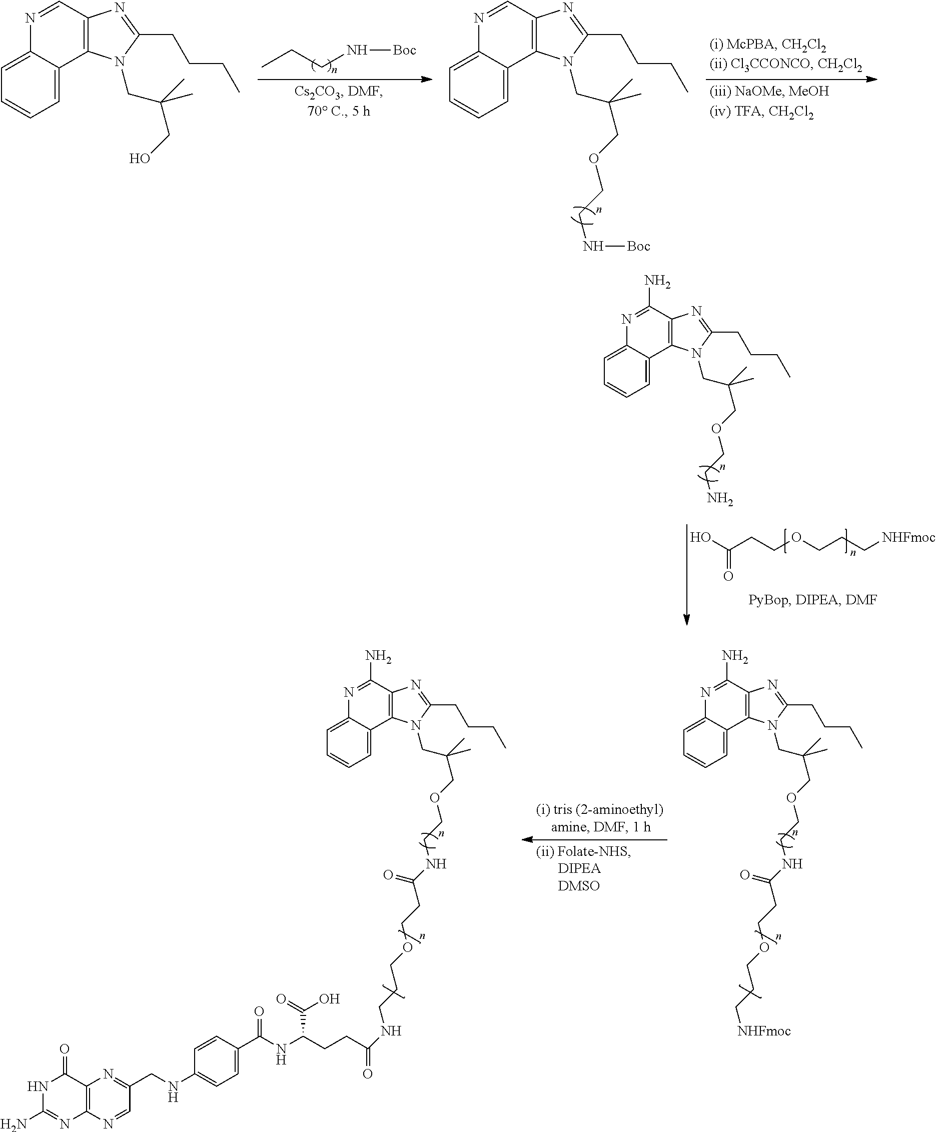

### Example I: Synthesis of Non-Releasable TLR7-Folate Compound

Synthesis of non-releasable TLR7-folate compound (Compound 1000) is described in Scheme 10:

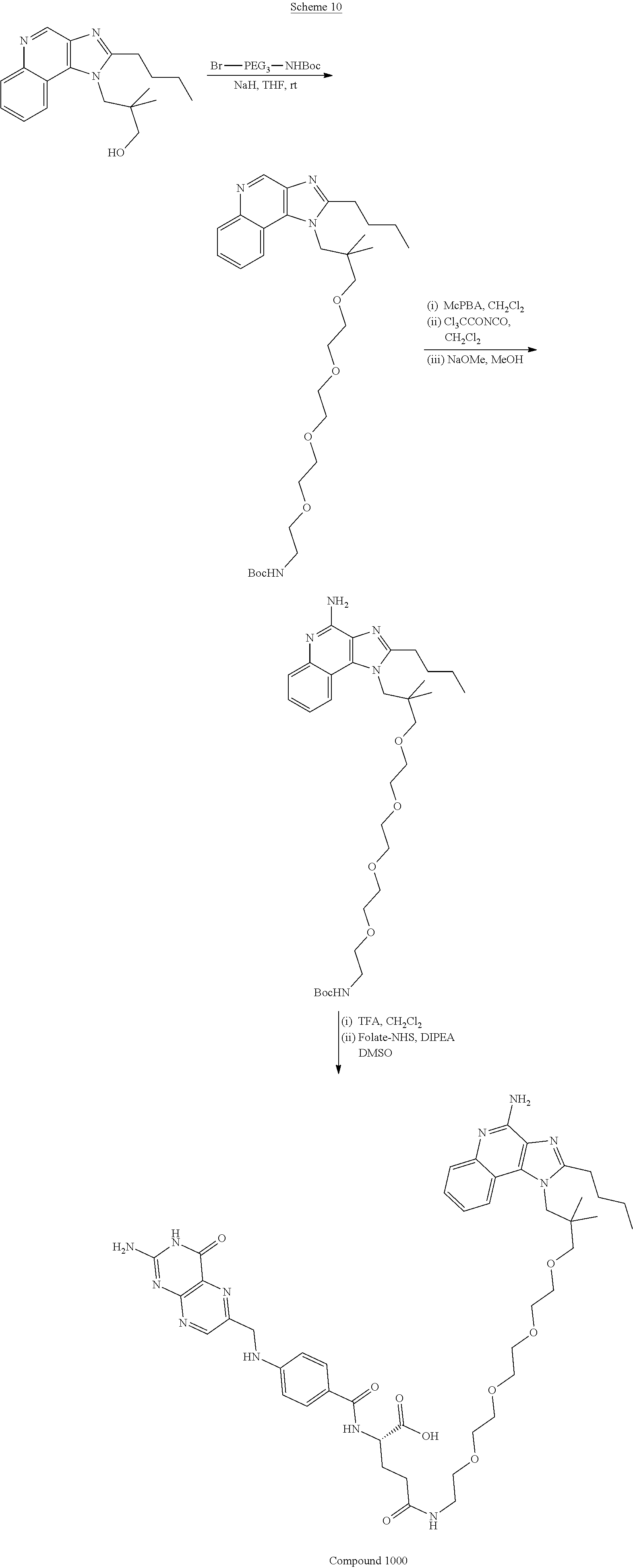

### Example J: Synthesis of Non-Releasable TLR7-Folate Compound

Synthesis of non-releasable TLR7-folate compound is described in Schemes 11 and 12:

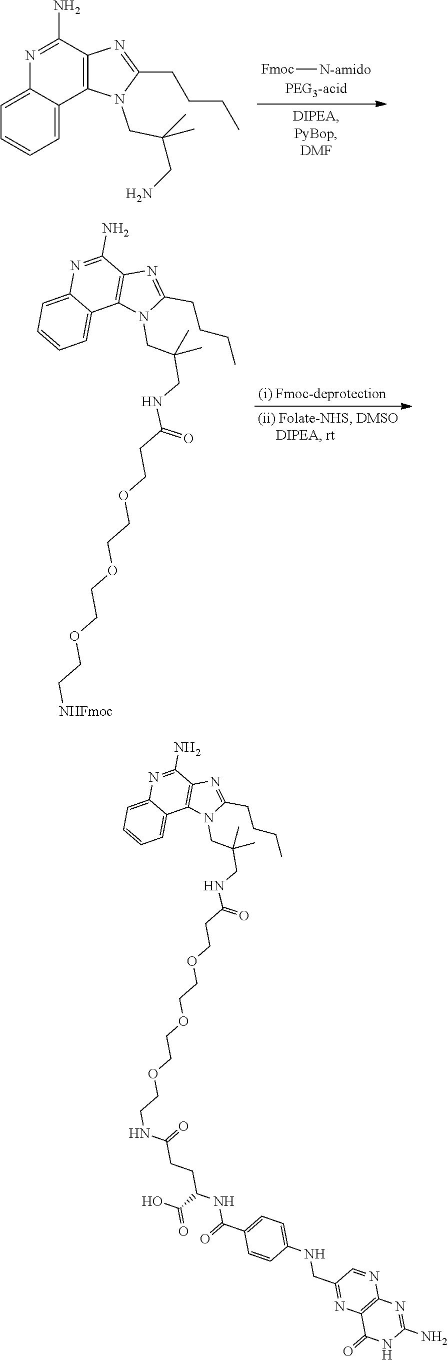

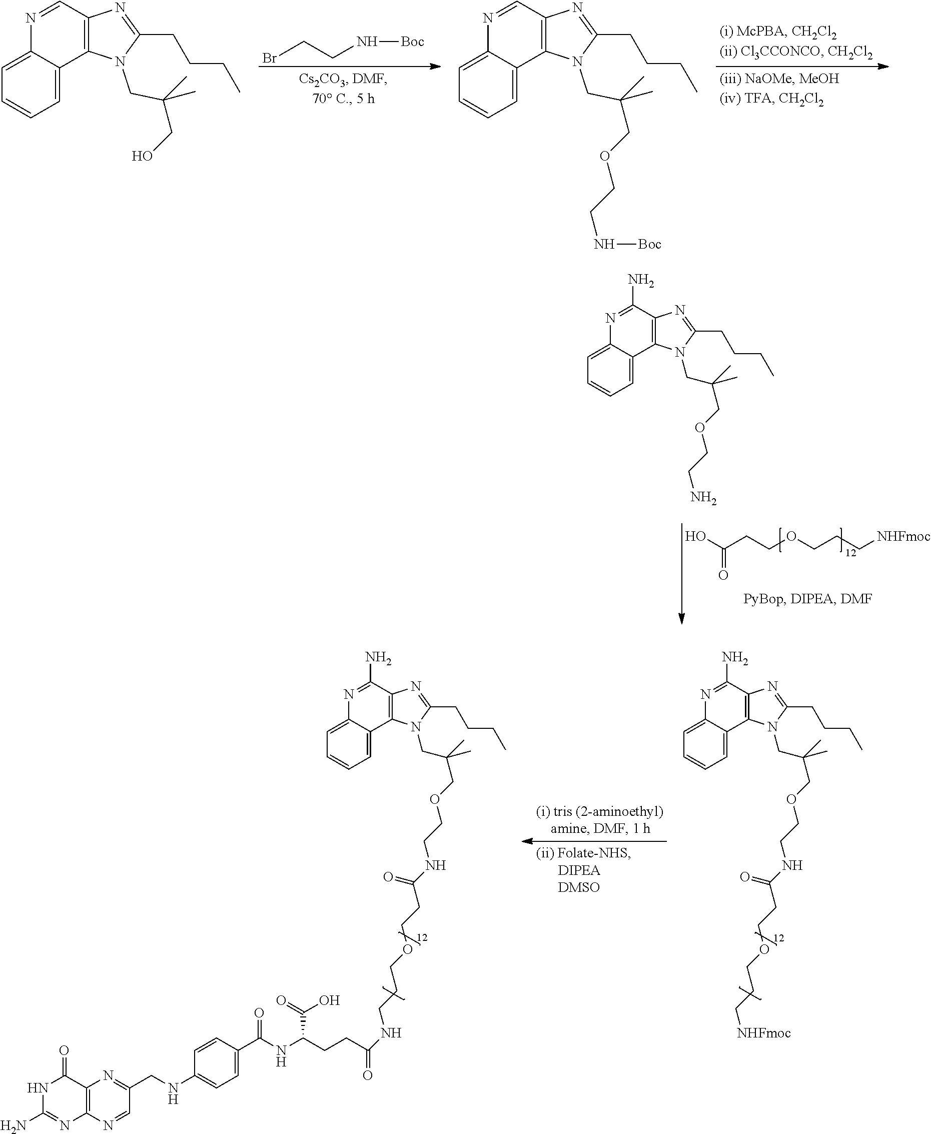

In conjunction with the current state of the relevant arts, especially in view of the Schemes set forth above, the present disclosure provides sufficient detail such that one of ordinary skill in the art can leverage the concepts set forth herein to synthesize all other compounds of the present disclosure.

### EXAMPLES

### Methods & Materials

Human monocytic THP-1 cells were obtained from American Type Culture Collection and cultured in folate-deficient RPMI 1640 medium (Invitrogen, Carlsbad, CA) containing 10% of heat inactivated fetal bovine serum and 1% Penicillin/streptomycin (Invitrogen. Carlsbad, CA). THP-1 cells were initially selected as a model system because this human monocytic cell line is known to acquire an M2-like phenotype and produce significant quantities of anti-inflammatory cytokines upon stimulation with IL-4, IL-6 plus IL-1β.

All reagents were purchased from commercial sources and were used without further purification. All other cell culture reagents, syringes, and disposable items were purchased from VWR (Chicago, IL).

Anti-CTLA-4 (Cat #BE0131, clone: 9H10), anti-PD-1 (Cat #BE0273, clone: 29F.1A12) and anti-PDL1 (Cat #BE0101, clone: 10F.9G2) antibodies were purchased from Bio X Cell (Lebanon, NH).

Flowcytometry antibodies, Zombie Violet™ Fixable Viability Kit, fixation/permeabilization buffer and RBC lysis buffer were obtained from BioLegend (San Diego, CA).

Mouse tumor dissociation kit (Cat #130-096-730) was purchased from Miltenyi Biotech. All the flow cytometry sample analyses were carried out on Attune NXT analyzer.

IFN-γ, IL-4, interleukin-6 (IL-6), and interleukin-13 (IL-13) were obtained from Biolegend. Phorbol 12-myristate 13-acetate (PMA), lipopolysaccharide (LPS), all other reagents and solvents were purchased from Sigma.

### Example 1

### Differentiation and Polarization of THP-1 Cells into M2-Like Macrophages In Vitro

THP-1 cells were seeded into 96-well plates at a density of 60,000 cells/well. Cells were differentiated into unpolarized macrophages by 48 hours incubation with 200 nM PMA followed by 24 hours incubation in fresh RPMI medium. The resulting macrophages were polarized to an M2-like phenotype by incubation with 20 ng/ml IL-4, 20 ng/ml IL-1β, and 5ng/nL IL-6 for 3 days and then reprogrammed with different concentrations of Compound 1A and Compound 1B for 48 hours and harvested for gene analysis by quantitative polymerase chain reaction (qPCR). Cultures were maintained at 37° C., in a humidified 5% CO₂ incubator.

To evaluate whether a potent TLR7 agonist (e.g., Compound 1A; e.g., of formula III) could reprogram the anti-inflammatory macrophages into a proinflammatory phenotype, IL-4, IL-6 plus IL-13 stimulated THP-1 cells were incubated with different concentrations of nontargeted Compound 1A and the mRNA levels of several anti-inflammatory markers were examined—namely, CCL18, CD206, IL-1β, and PDGFα and β.

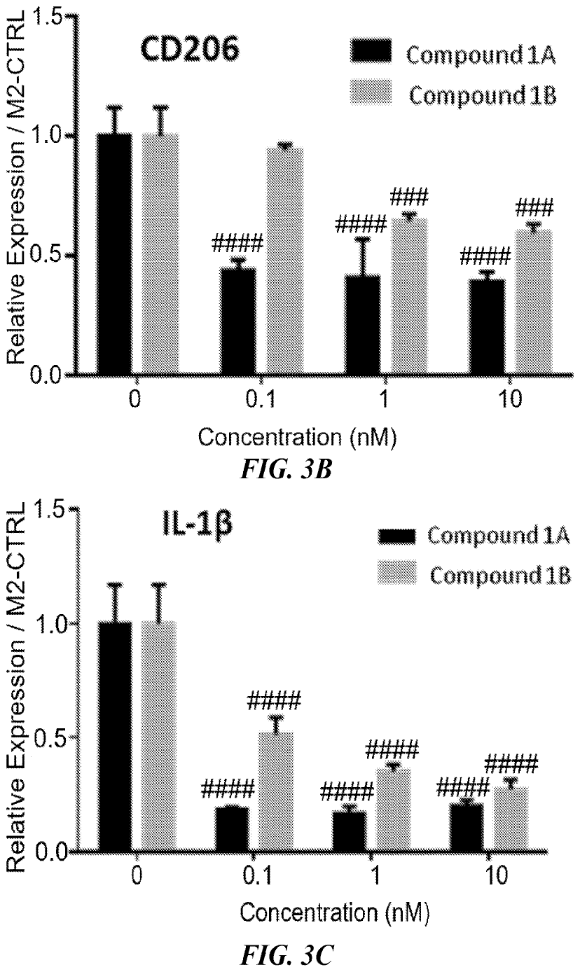

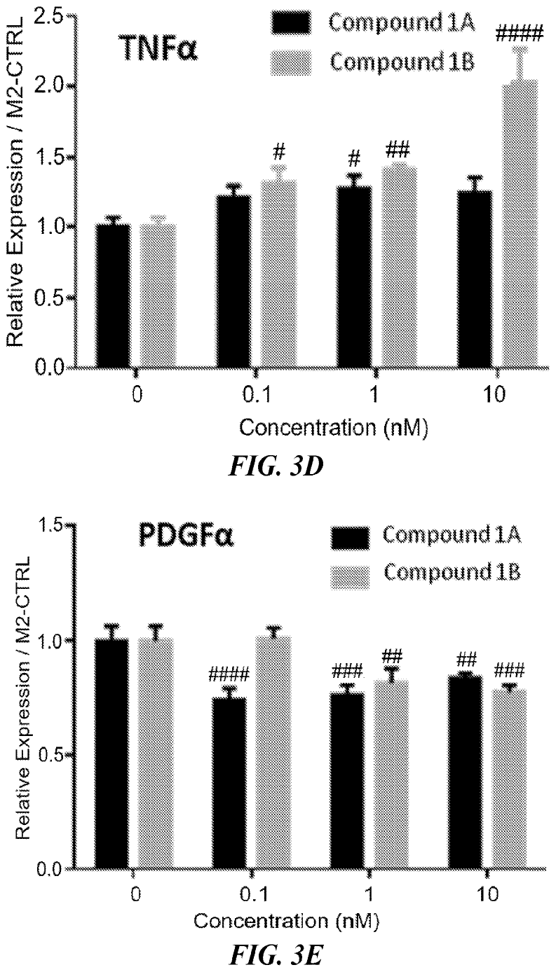

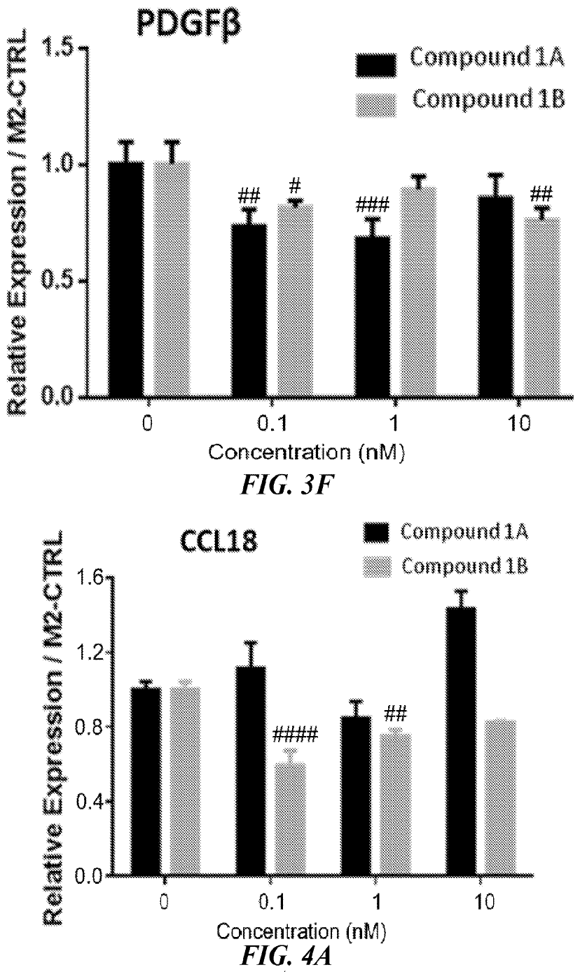

As shown in FIG. 3A-3C, incubation with the Compound 1A for 48 hours induced a decrease in CCL18, CD206 and IL-1β expression, suggesting that the TLR7 agonist can indeed promote a shift in these anti-inflammatorily polarized THP-1 cells towards a less fibrotic/more inflammatory phenotype. Moreover, when expression of TNFα, a marker of a proinflammatory phenotype was examined, an increase in its expression was observed (FIG. 3D), confirming that the THP-1 shift from anti- to pro-inflammatory properties had occurred.

### Example 2

### Evaluation of the Macrophage Reprogramming

To confirm that folate-conjugated TLR7 agonist can cause the same THP-1 reprogramming seen in Example 1, Compound 1B was prepared in which a releasable linker connecting folate to Compound 1A was constructed with a disulfide, self-immolative bond to allow for release of Compound 1A following internalization of Compound 1B into the reducing environment of intracellular endosomes.

Different concentrations of either Compound 1A or Compound 1B were incubated with the above polarized THP-1 macrophages for the indicated times, after which the culture medium was harvested for analysis of secreted cytokines and the collection of cells for qPCR analysis.

Total RNA was isolated from 1×10⁵-2×10⁵ macrophages using a Quick-RNA™ MicroPrep kit (Zymo Research, Irvine, CA) according to the manufacturer-recommended protocol. The RNA samples were then reverse-transcribed into cDNA using high-capacity cDNA reverse transcription kits (Applied Biosystems, Foster City, CA; #4368814), qPCR analyses were performed using the iTaq™ Universal SYBR Green SuperMix (Bio-Rad Laboratories GmbH, Hercules, CA; #1725121), iCycler thermocycler, and iCycler iQ 3.0 software (Bio-Rad Laboratories GmbH, Hercules, CA) to track the expression of markers characteristic of macrophage polarization states, IL-6, CXCL10, IFNα, IFN-γ and CD86 were used as markers for an M1 phenotype, while CCL18. CD206, CD163 and Arg1 were employed as markers for the M2 phenotype, IL-1β, PDGFβ, MMP9 and TIMP 3 were measured as indicators of an anti-inflammatory phenotype. IRAK-4 was used as an indicator of TLR7 stimulation. To control for specificity of the amplification products, a melting curve analysis was performed. No amplification of nonspecific products was observed in any of the reactions. Each sample was analyzed independently in triplicate for each marker.

Upon repeating the studies described above (see grey bars, FIGS. 3A-3F), the same qualitative changes were observed, only the magnitude of the impact of Compound 1B was somewhat reduced. This reduction in potency was expected because the nontargeted TLR7 agonist enters the cultured cells immediately, whereas its folate-targeted counterpart is designed to enter cells only after folate receptor-binding and receptor-mediated endocytosis.

Data shown in FIGS. 3A-3C support that administration of either the non-targeted TLR7 agonist or the targeted TLR7 agonist successfully reprogrammed M2-type macrophages to M1-type macrophages (i.e., downregulated the M2-type anti-inflammatory macrophages) and the data shown in FIGS. 3D-3F support that administration of the tested compounds upregulated the M1-type macrophages.

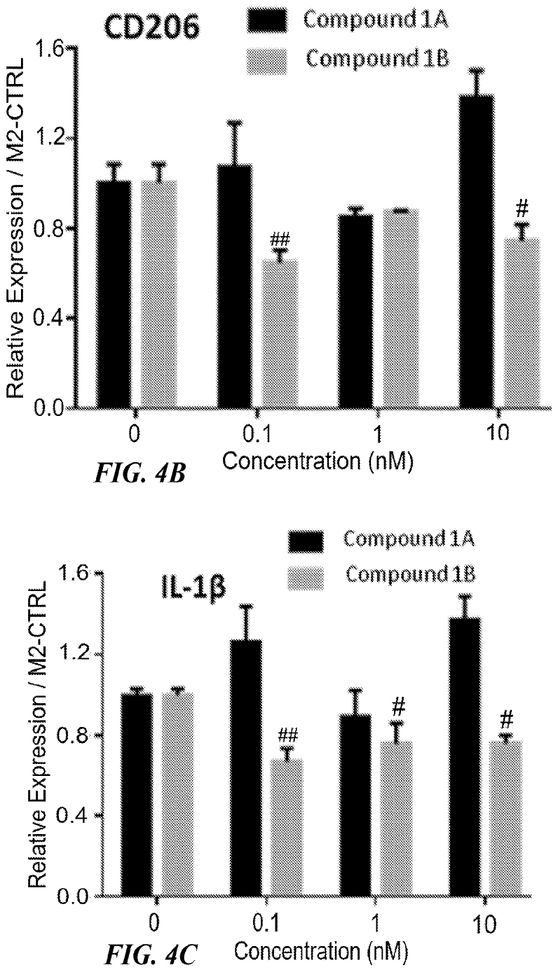

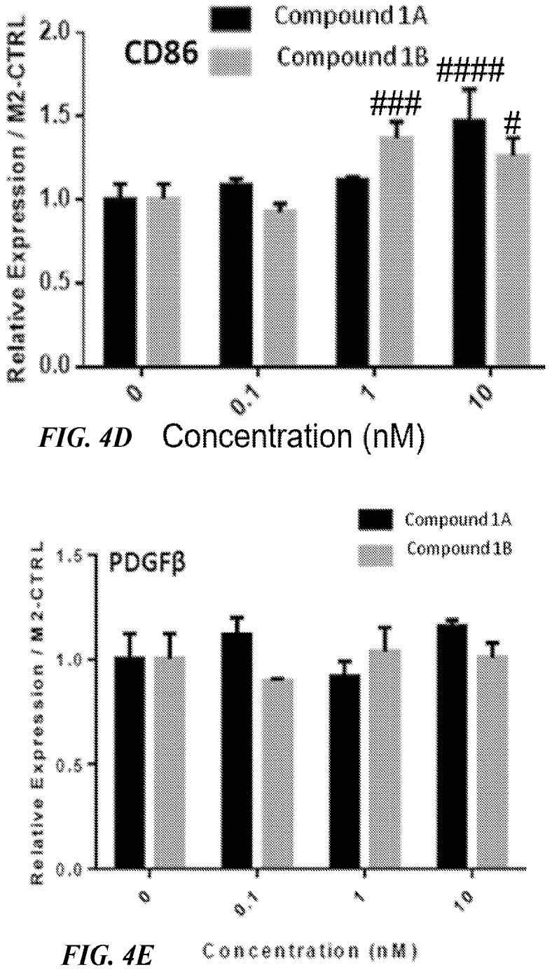

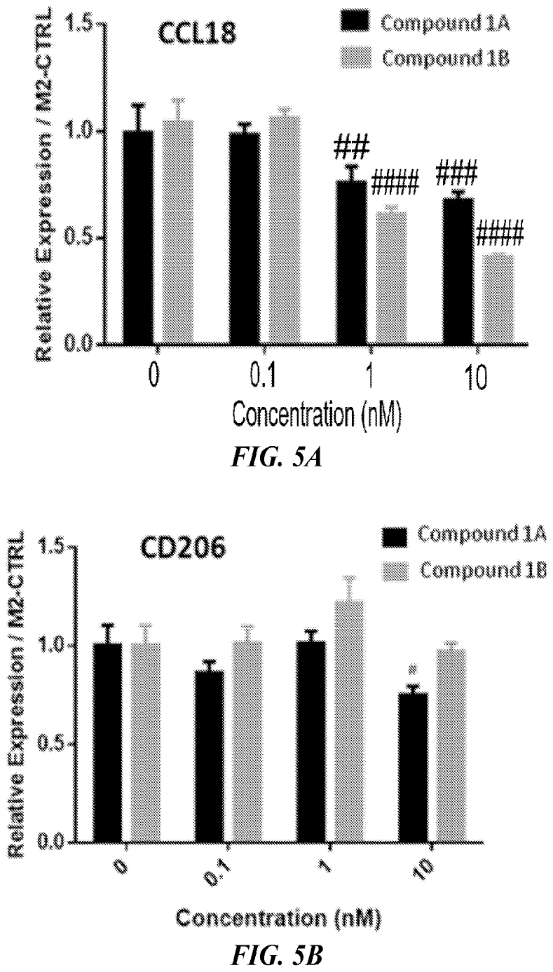

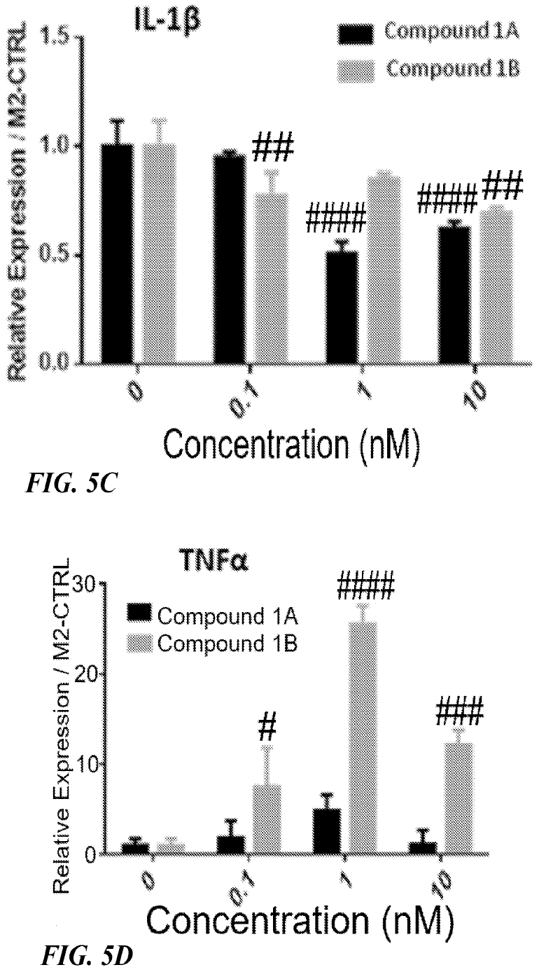

FIGS. 4A-4E and FIGS. 5A-5D show graphical data representative of various marker levels measured from THP-1 cells induced to M2 macrophages that were subsequently incubated with different concentrations of Compound 1B or Compound 1A for 2 hours, washed with PBS, for the data shown in FIG. 5A-5D, again incubated for 46 hours (for the data shown in FIG. 4A-4E, the cells were harvested immediately after the initial 2 hours of incubation). In both data sets, the cells were harvested for gene analysis by qPCR. FIG. 4A-4C shows CCL18 mRNA levels (FIG. 4A and FIG. 5A), CD206 mRNA levels (FIG. 4B and FIG. 5B), IL-1β mRNA levels (FIG. 4C and FIG. 5C), and PDGFβ mRNA levels (FIG. 4E). The data supports that the M2-type anti-inflammatory phenotype was downregulated following administration of the tested compounds. In particular, Compound 1B downregulated anti-inflammatory/M2-type markers of macrophages more than Compound 1A. Furthermore, FIG. 4D shows CD86 mRNA levels and FIG. 5D shows TNFα levels, which data supports that the Mi-like phenotype was upregulated following administration of the tested compounds. FIGS. 4A-4E and FIGS. 5A-5D support that the M2-type anti-inflammatory phenotype was downregulated following administration of the free and targeted TLR7 agonist. While collected, data is not shown for PDGFα as no significant difference following treatment was observed.

Because low molecular weight water-soluble drugs like Compound 1A and Compound 1B are often excreted from the body within 2 hours of injection, a more physiologically relevant in vitro model of drug exposure in vivo is to limit incubation of a cell with drug for only two hours and then examine drug efficacy after an additional 46 hours of incubation in the absence of the drug. As shown in FIGS. 4A-4E, when THP-1 cells were incubated with the TLR7 agonists for 2 hours prior to replacement of the drug-containing medium with drug-free medium, Compound 1B was observed to have superior potency relative to Compound 1A, especially in the case of TNFα induction where the folate-targeted compound was dramatically improved. This is most likely because the folate-targeted TLR7 agonist was captured by the folate receptor positive cells, whereas Compound 1A was not retained by the same cells.

These data support that Compound 1B should be more effective in reprogramming anti-inflammatory macrophages in vivo, with the added advantage that the folate-conjugated drug (e.g., Compound 1B) should also cause less systemic toxicity because it is concentrated in the FRβ-expressing macrophages and unable to enter folate receptor negative cells that predominate throughout the body (e.g., Compound 1B is designed to be impermeable to folate receptor negative cells).

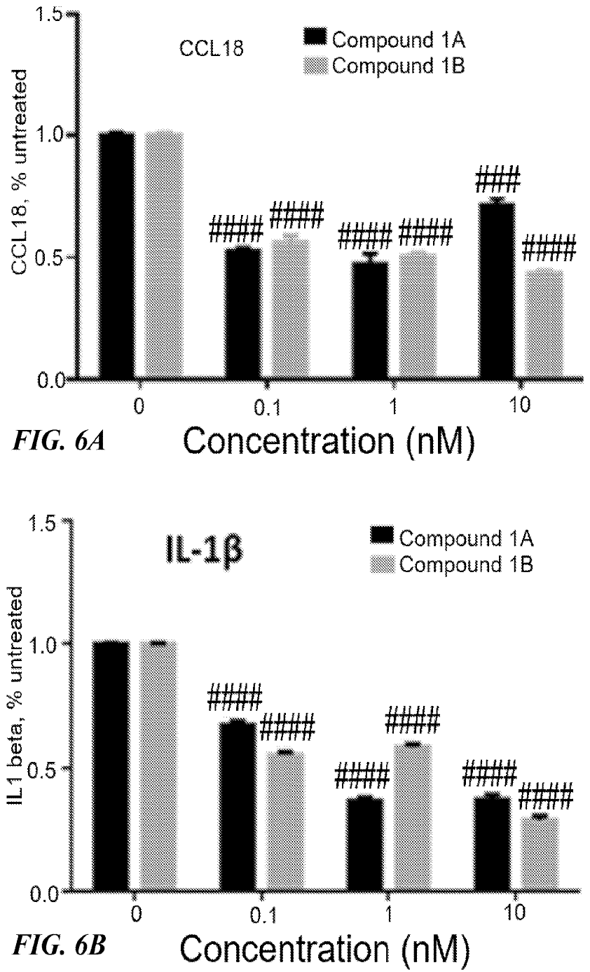

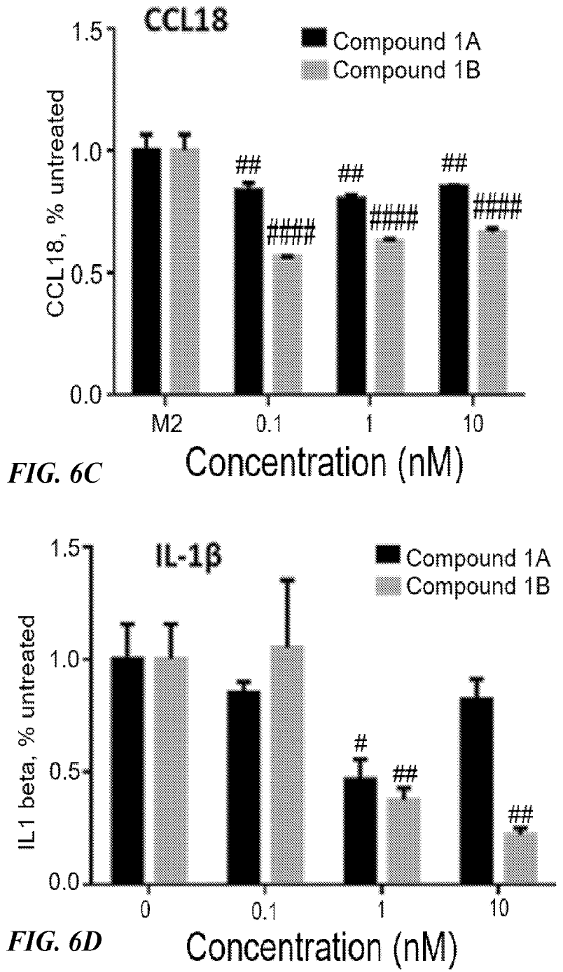

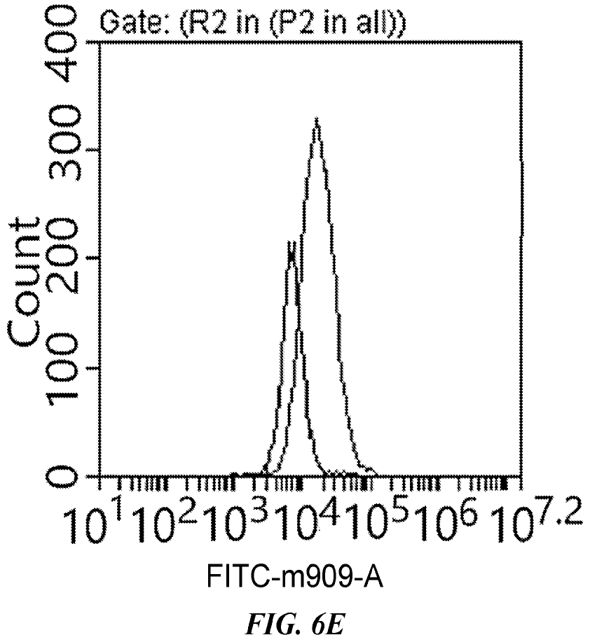

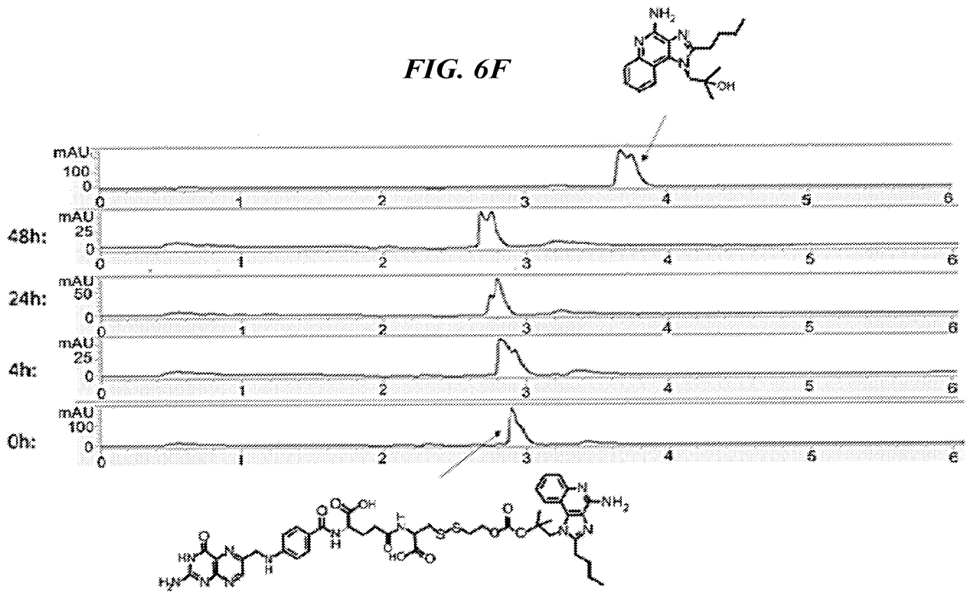

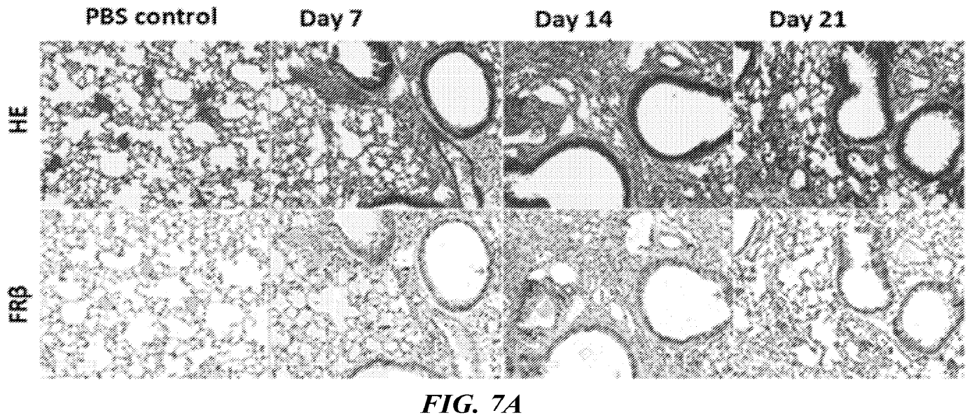

FIGS. 6A-6D show graphical data representative of various marker levels measured from M2-induced THP-1 macrophages treated with different concentrations of drugs for 48 hours (FIGS. 6A and 6B) or for 2 hours, then displaced with fresh medium and cultured for the remaining 46 hours (FIGS. 6C and 6D). In both cases, cell supernatants were collected and secreted CCL18 protein and IL-1β was detected by ELISA. The data supports that administration of the TLR7 compound or the folate-targeted TLR7 compound downregulates the secretion of CCL18 and IL-1β at low concentration ranges (0.1-10 nM).

Further, to ensure that the above mRNA analyses accurately reflected the levels of anti-inflammatory cytokines produced by IL-4, IL-6 plus IL-13 stimulated THP-1 cells, the concentrations of CCL18 and IL-1β polypeptides in the THP-1 supernatants were quantitated by ELISA assay. As shown in FIGS. 6A and 6B, both Compound 1A and Compound 1B induced reductions in CCL18 and IL-1β when incubated continuously with agonist for 48 hours, however, Compound 1B again was found to be superior when drug exposure was limited to only 2 hours (see FIGS. 6C and 6D).

### Example 3

### Characterization of FRβ Expression by Flow Cytometry

To measure the expression of FRβ on THP-1 derived macrophages, fluorescence-activated cell sorter (FACS) analysis was performed. Cells were detached using Accutase® Cell Detachment Solution (Biolegend, San Diego, CA; #423201) and gently lifted with a cell scraper. Cells were washed with PBS and nonspecific binding was blocked by incubation with Fc receptor blocking solution (Biolegend, San Diego, CA; #422301) at room temperature for 10 min. Biotinylated anti-human FRβ monoclonal antibody (m909) was then added and the cells were incubated for an additional 30 min on ice prior to washing in staining buffer (PBS supplemented with 2% FBS). Cells were then incubated on ice for 20 min in fluorescein-labeled streptavidin (BD Biosciences, Franklin Lakes, NJ; #554060), washed twice in PBS, stained with 7AAD (viability stain) for 15 min and analyzed by flow cytometry using BD Accuri C₆ Software (BD Biosciences, Franklin Lakes, NJ). FIG. 6E shows the flow cytometry data, supporting that the THP-1 macrophages and were FRβ+ and, thus, suitable for the in vitro study of Compound 1B and other studies described herein.

FIG. 6F confirms that Compound 1B remained stable during the incubation period, which was 37° C., in the culture media. Indeed, Compound 1B retained the original structure after 48 hours incubation.

### Example 4

### Bleomycin Induced Pulmonary Fibrosis and Anti-Inflammatory Macrophage Reprogramming In Vivo

Studies were also performed to determine if macrophages in pulmonary fibrotic lungs might be specifically targeted with folate-linked drugs in vivo. After testing multiple protocols for induction of pulmonary fibrosis in mice, a protocol was selected where 0.75 mg/kg bleomycin (BM) is instilled into the lungs of C57BL/6 mice via an incision in the trachea and the mice are allowed to progress through both inflammatory and fibrotic stages of fibrosis prior to initiation of therapy. (The BM model is widely regarded to be helpful in terms of enabling mechanistic investigations relevant to fibrogenesis in an in vivo context.)

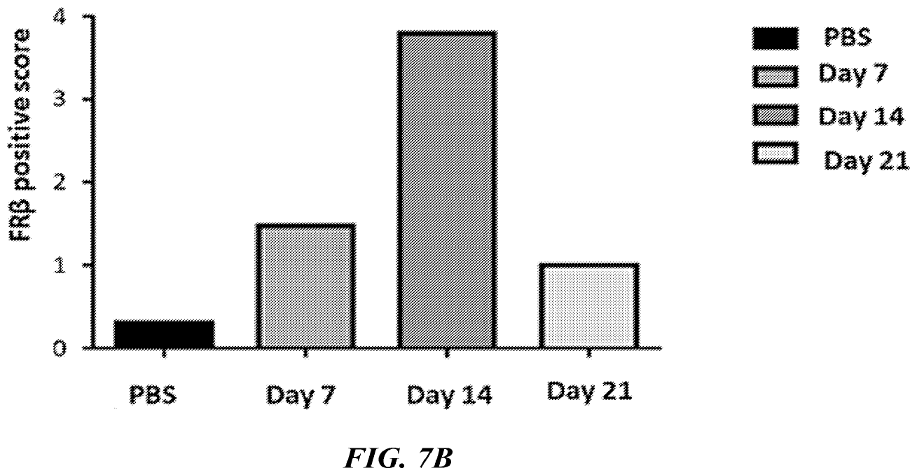

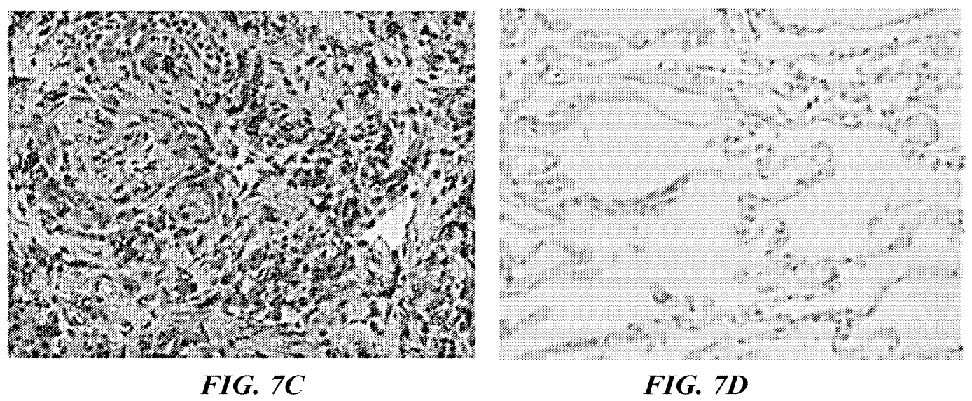

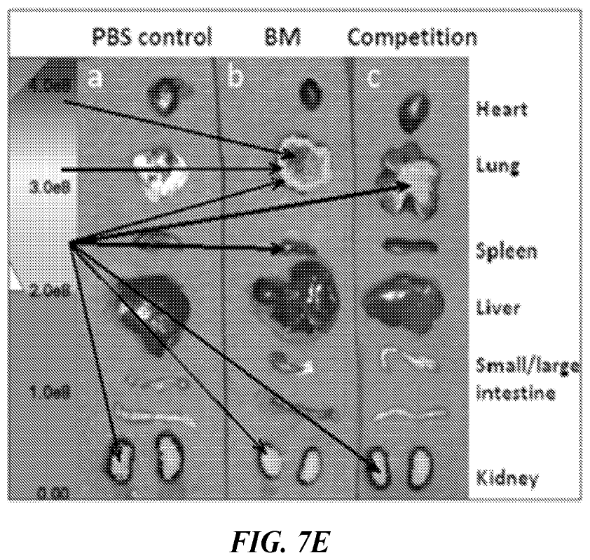

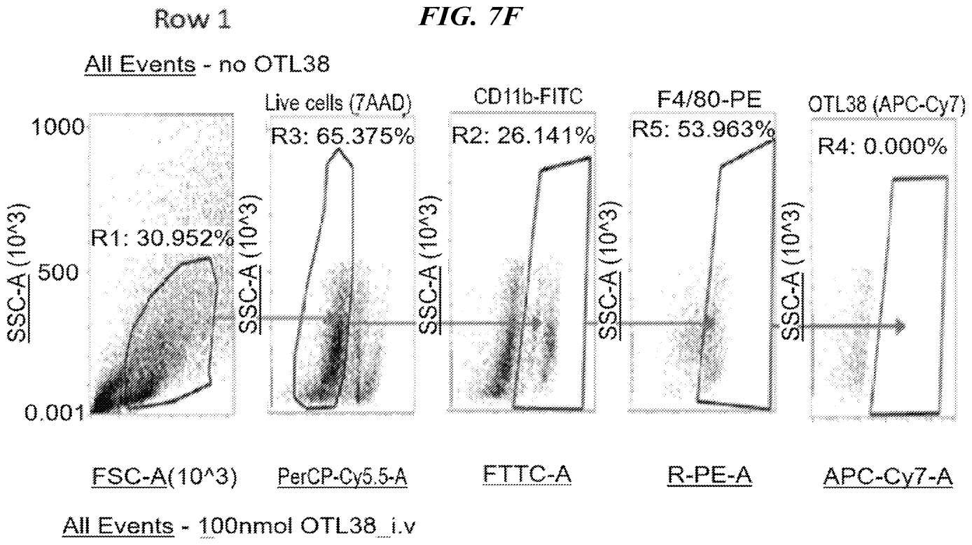

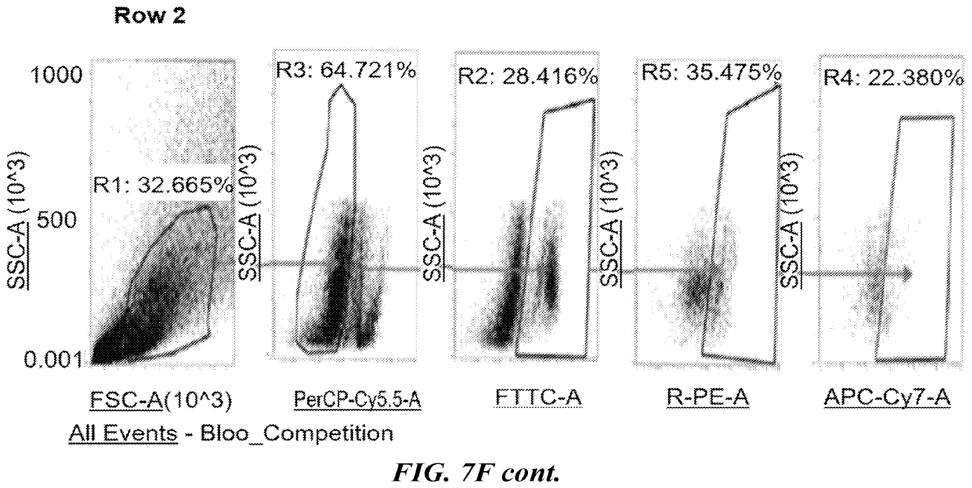

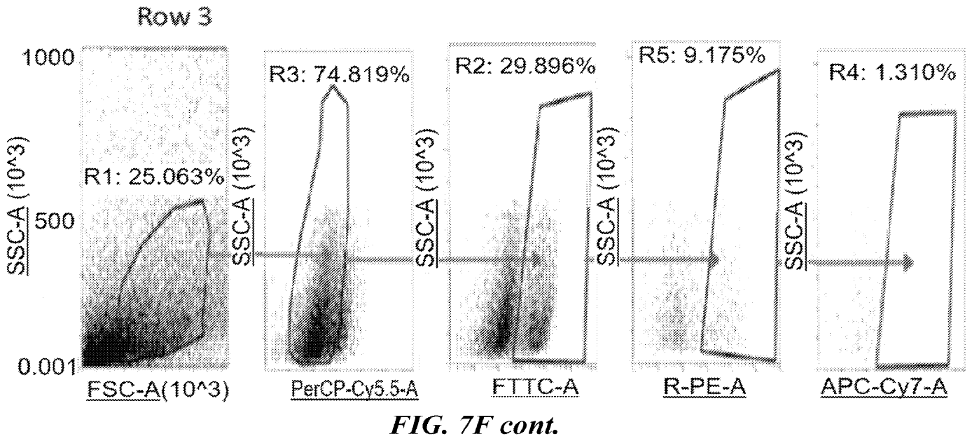

As shown in FIGS. 7A-7D, mice treated using this protocol typically display fibrosis by day 7 post-BM treatment and this nascent fibrosis develops into severe fibrosis by day 14. Progress of the pathology then continues for 2-5 additional days before it begins to spontaneously resolve by day 21.

More specifically, eight-week-old C57BL6 male mice from Charles River (average weight 22 g to 25 g) were housed under pathogen-free conditions at room temperature (22° C.) under a 12 hours light-dark cycle. Mice were placed on a folate deficient chow (Envigo Teklad Global Rat Food Pellets) for 1 week prior to the BM or PBS instillation. Fresh water and folate-deficient diet were freely available. All animal procedures were approved by the Purdue Animal Care and Use Committee in accordance with National Institute of Health guidelines.

Thereafter, the mice were anesthetized with ketamine/xylazine and the necks of the mice were shaved using hair remover lotion and then sterilized with 70% alcohol. A small incision was made on the neck to visualize the trachea. Mice were positioned at a 75-degree angle and injected intratracheally with 100 μL sterile PBS or BM (Cayman Chemicals, Ann Arbor, MI; #13877) dissolved in PBS (0.75 mg/kg) using a 1 cc syringe with 26 G needle. Body weights were monitored every other day throughout the experiment.

To evaluate if anti-inflammatory lung macrophages in these mice can be specifically targeted with folate-linked drugs, 10 days after the instillation, 10 nmol (for in vivo imaging) or 100 nmol (for in vivo labeling) of a folate-linked, near infrared fluorescent dye (OTL38) with or without 200-fold excess of FA-glucosamine (a competitor of OTL38) was injected into the tail veins of BM-treated mice and the dye uptake in the major organs was evaluated.

After 2 hours, mice were sacrificed using CO₂ asphyxiation and an incision in the skin from the abdomen to neck was immediately made to expose the lungs and trachea. A small cut in the upper trachea was then introduced for insertion of a blunted, 22-gauge needle, and a nylon string was tied around the trachea to seal the trachea around the needle. The trachea (containing the inserted needle), lungs and heart were then removed en masse by carefully cutting the connective tissue beneath the lungs, and the bronchus of left lung was clipped with a Dieffenbach vessel clip. The right lung was injected with PBS and aspirated 3 times using a 1 ml syringe, and the recovered lavage fluid was saved on ice.

Bronchoalveolar lavage fluid (BALF) was then analyzed to determine how the targeted TLR7 agonist works. BALF samples were centrifuged at 1500 rpm for 5 min at 4° C., and the supernatant was aliquoted and stored at −80° C., for cytokine/chemokine analyses. Cell pellets were resuspended and cultured in pre-warmed RPMI 1640 medium for 2 hours and then washed 3x with pre-warmed PBS prior to harvesting for qPCR assay. The right lung was then tied with a nylon string and used for subsequent analysis of hydroxyproline content. The left lung was inflated with 1 ml PBS using the inserted syringe and transferred to 10% formalin solution for subsequent histological analyses.

Lobes of the right lung collected above were weighed, placed in a pressure-tight vial (Supelco Inc., Bellefonte, PA; #27003), and hydrolyzed with 6N HCl (10 ml/g, v/w) in a sand bath at 120° C., for 3.5 hours. The hydrolyzed solution was cooled at 4° C., for 15 min and transferred to a 1.5 ml Eppendorf tube prior to centrifugation at 12,000 rcf for 15 min at 4° C. Supernatant was carefully collected, aliquoted and used for hydroxyproline (HYP) analysis.

For the subsequent HYP analysis, 10 μl of sample was transferred into a 96-well plate and neutralized with 10 μl of 5.3 M sodium hydroxide solution. Isopropanol (40 pl) was then added to each well followed by 20 μl of oxidation buffer and the mixture was incubated on a shaker at room temperature for 5 min. Analytical reagent (260 μl) was added, and the plate was incubated on a shaker at room temperature for 30 seconds and incubated immediately at 60° C., for 25 min. The absorbance was measured at 560 nm (A₅₆₀) within 15 min. All reagents were prepared according to a previously reported protocol.

For histological analysis of the lung sections, fixed lungs (see above) were embedded in paraffin, sectioned and stained with hematoxylin-eosin (H&E), Masson's trichrome or F3 (anti-mouse FRβ antibody). Tissue sections were examined in a blinded manner by a licensed pathologist. More than 90×10⁶ cells were quantified per section using Aperio-Image Scope (Leica Biosystems, Wetzlar, DE).

CCL18 and IL-1β were quantified in induced THP-1 cell supernatants using a human DuoSet ELISA Development System (R&D Systems Europe, Abingdon, UK: #DY394-05) and an IL-1 beta Human ELISA Kit (Thermo Fisher Scientific, Waltham, MA; #BMS224-2) as described by manufactures. BALF samples were analyzed for mouse IFN-γ using ELISA MAX™ Deluxe (Biolegend, San Diego, CA; #430804).

Finally, for the in vivo folate imaging studies, major organs (heart, lung, spleen, liver, small intestine, large intestine, and kidney) were resected and imaged using an AMI live imager (Spectral Instruments Imaging, Tucson, AZ). For in vivo folate receptor labeling studies, lungs of the mice were harvested immediately following euthanasia, digested with a lung dissociation kit (Miltenyi Biotec, Bergisch Gladbach, DE; #130-098-427) as described by gentleMACS Octo Dissociator with Heathers (Miltenyi Biotec, Bergisch Gladbach, DE; #130-096-427) as described by manual and filtered through a 70 μm cell strainer (Miltenyi Biotec, Bergisch Gladbach, DE: #130-098-462). Cells collected in the filtrate were depleted of erythrocytes by ammonium sulfate lysis, washed 2× in cold PBS and labeled for 30 min on ice with antibodies to desired macrophage markers (FITC-CD11b, Biolegend, San Diego, CA; #101205; FE-F4/80. Biolegend. San Diego, CA: #123109). Labeled macrophages were then washed twice in PBS, stained with 7AAD (viability stain) for 15 min and analyzed by flow cytometry using BD Accuri C6 Software (BD Biosciences, San Jose, CA).

As shown in FIG. 7A (top panel) untreated lungs (PBS control column) and BM-treated lungs on day 7 display a similar high density of alveoli interconnected by minimal extracellular matrix. In contrast, at day 14 post-BM instillation, the sizes and frequencies of air sacs were significantly decreased and the density of extracellular matrix visibly increased, suggesting the development of significant fibrosis in the treated mice. By day 21, the pathology in this model had already begun to spontaneously resolve, with many mice eventually recovering from the BM-induced trauma by day 35.

Evidence for development of inflammation by day 7 is seen from the infiltration of FRβ-expressing macrophages (see lower panel of FIG. 7A and quantitation in FIG. 7B) that are almost completely absent from the healthy lungs but continue to accumulate through day 14 in the BM-exposed lungs. Further, staining with F3 showed significant expression of FRβ in the IPF lung (majorly in the interstitial space) as previously reported in the literature (FIG. 7A). Expression of FRβ was restricted to the inflamed lung (either IPF patient or BM-induced PF, but not in healthy lung). Moreover, FRβ-expressing macrophages were observed in mouse lungs on day 7 after the administration of BM with a maximum expression on day 14 (FIG. 7B). These results corroborated with previously reported FRβ expression on the activated macrophages in the inflamed lung.

That these FRβ-expressing macrophages can be targeted with folate-linked molecules was then demonstrated by the accumulation of OTL38, a folate-targeted fluorescent dye, in the lungs of BM-treated but not healthy mice following tail vein injection. As shown in FIG. 7B. OTL38 fluorescence was only observed in the kidneys of healthy mice (i.e. its major site of excretion), with little or no uptake in other tissues.

FIGS. 7C and 7D show FRβ IHC staining of human IPF lung tissue (FIG. 7C) and healthy human lung tissue (FIG. 7D). Eight-week-old C57BL/6 male mice were placed on a folate deficient chow for 1 week prior to the BM or PBS instillation, 10 days after the instillation, mice were injected via tail vein with 10 nmol (for in vivo imaging) or 100 nmol (for in vivo labeling) of OTL38 with or without 200-fold excess of FA-glucosamine. After 2 hours, mice were sacrificed prior to analysis. For in vivo folate imaging studies, major organs (heart, lung, spleen, liver, small intestine, large intestine and kidney) were resected and imaged using an AMI live imager (Spectral Instruments Imaging, Tucson, AZ). For in vivo folate receptor labeling studies, lungs of the mice were harvested immediately following euthanasia, digested and then labeled with antibodies to desired macrophages markers (FITC-CD11b, PE-F4/80) and 7AAD (live/dead staining) and analyzed by flow cytometry.

FIG. 7E shows images of various mice tissues/organs taken from mice with (BM) or without (PBS control) BM-induced experimental fibrosis and imaged with a folate receptor-targeted fluorescent dye, OTL38, with healthy (column a) or BM-treated mice (columns b and c) tail vein injected with 10 nmol OTL38 in the absence (b) or presence (c) of 200-fold excess of a folate-targeted glucosamine (competitive reagent of FRβ, which blocks the binding of OTL38) on day 10 post induction of fibrosis and euthanized 2 h later for tissue resection and fluorescence imaging, supporting that the inventive FA-targeting compound of the present disclosure exhibit FRβ-specific binding without uptake in other healthy tissue.

Tail vein injection of OTL38 into BM-treated mice yielded not only the aforementioned fluorescence in the kidneys, but also pronounced accumulation in the fibrotic lungs (see FIG. 7E). That this lung uptake was largely mediated by folate receptors could be demonstrated by the nearly quantitative blockade of lung accumulation when the BM-treated mice were simultaneously injected with 200-fold excess folate-glucosamine (i.e. a competitive inhibitor of FRβ-binding (see FIG. 7E)). These data demonstrate that a folate-targeted molecule binds selectively to folate receptor expressing cells in fibrotic tissue without accumulating to any significant extent in other tissues of the body. In other words, the FRβ-expressing macrophages can in fact be targeted with folate-linked molecules and, in clinical application, localize almost exclusively to the fibrotic tissue. As such, when a targeted moiety is used in the compounds of the present disclosure, any TLR7 agonist that is not captured by the targeted fibrotic (or cancerous) tissue will be minimal.

Next, to determine what cell type is capturing the folate-dye compound in the lungs of BM-treated mice, lungs from the above animals were digested with collagenase and examined by flow cytometry for cell-specific dye uptake. FIG. 7F shows data from a FACS analysis resulting from the in vivo labeling of such mice experiencing BM-induced experimental fibrosis that were tail vein injected with PBS (row 1) or 100 nmol OTL38 in the absence (row 2) or presence (row 3) of 200-fold excess of the folate-targeted [glucosamine]. As shown in FIG. 7F, no macrophage-like cells displayed any fluorescence when isolated from BM-treated mice not injected with OTL38 (see row 1). In contrast, about 22% of the macrophage-like cells from OLT38-injected fibrotic mice showed significant folate-targeted dye retention (row 2), which supports that OTL38 targets the FRβ positive macrophages in the inflamed lung. Indeed, the dye uptake was specifically folate receptor-mediated, as demonstrated by the observation that concurrent tail vein injection of 200-fold excess folate-glucosamine blocked essentially all folate-dye retention, demonstrating that accumulation of the dye required unoccupied folate receptors. Importantly, this conclusion is further supported by data showing that FRβ expression was essentially nondetectable in untreated lungs (see FIG. 7A) but increased dramatically during the development of fibrosis in BM-treated lungs (see FIGS. 7A-7D). FRβ expression was also prominently expressed in the lungs of human IPF patients.

### Example 5

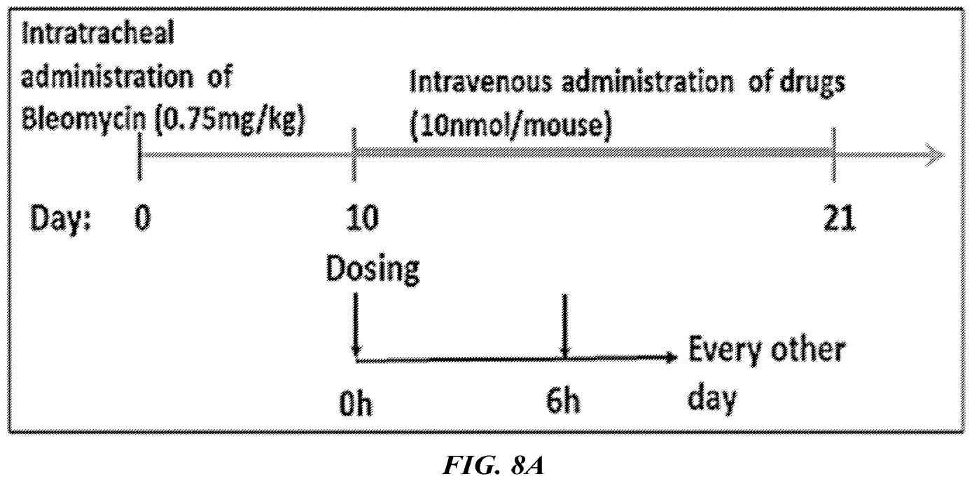

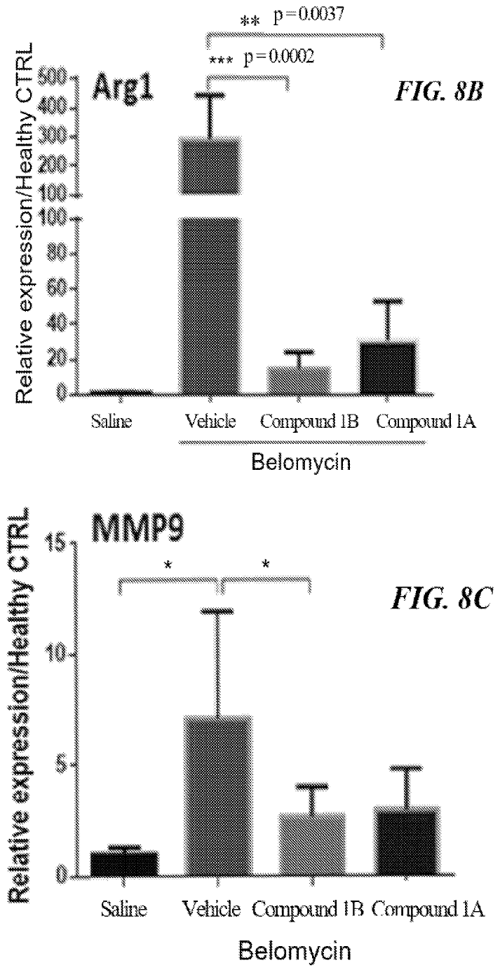

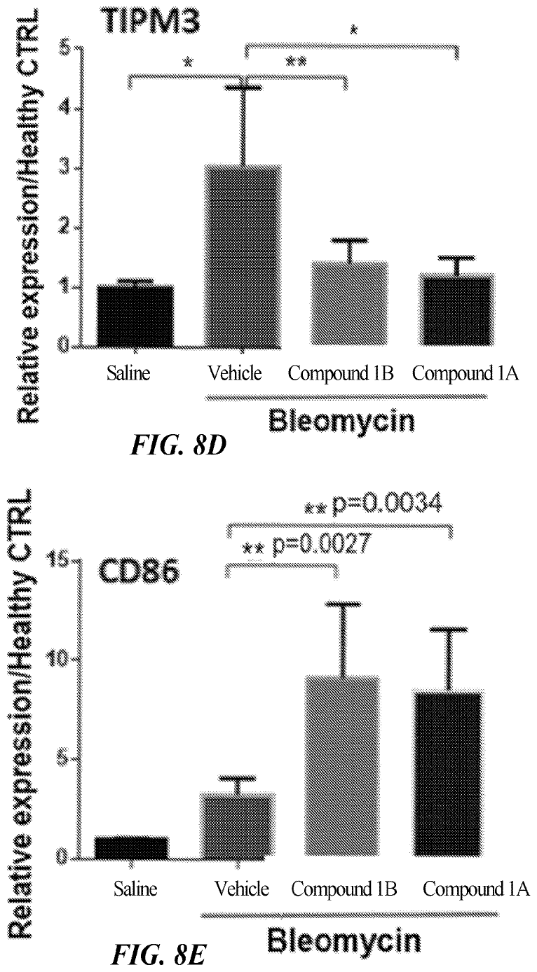

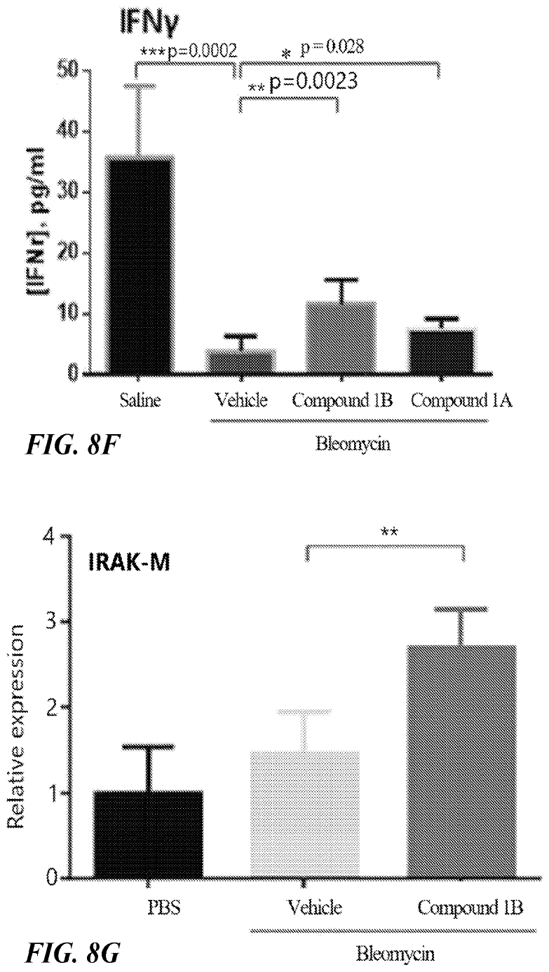

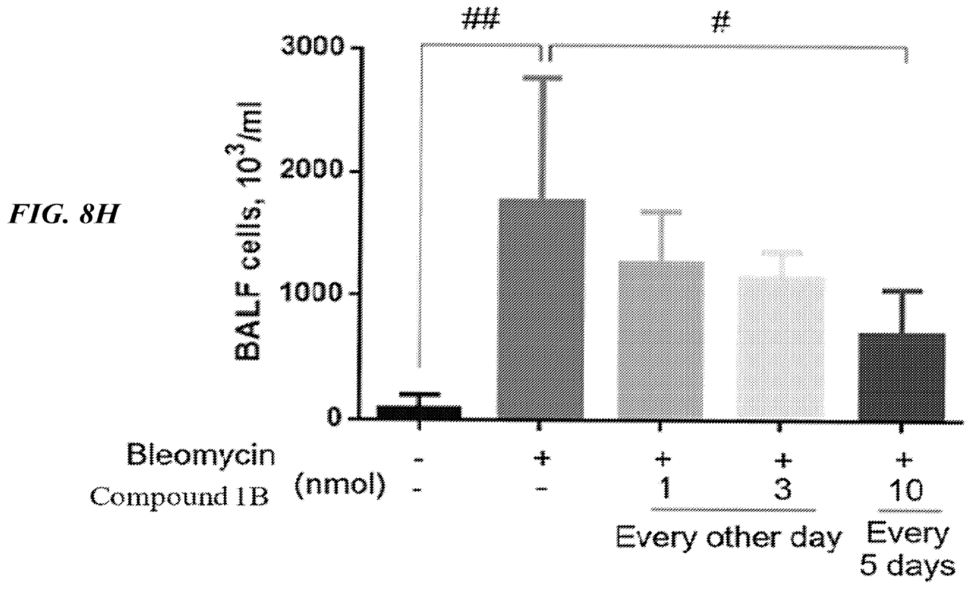

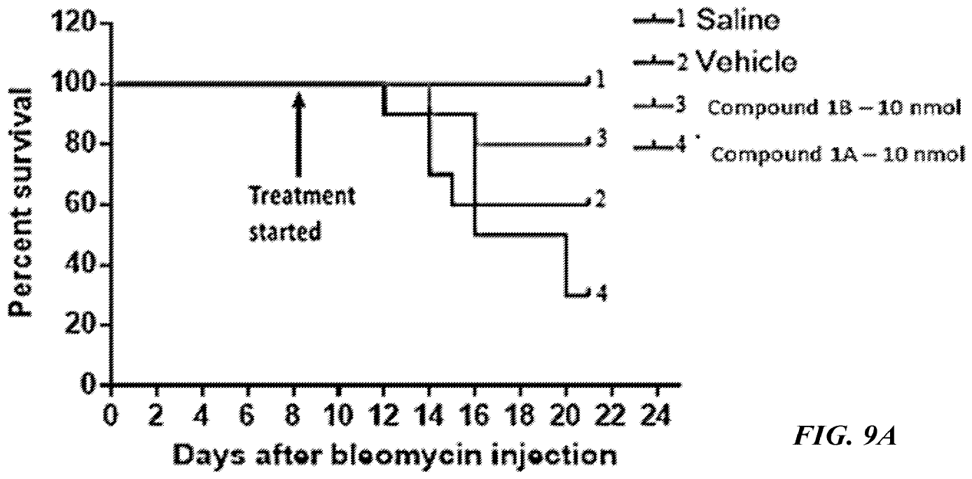

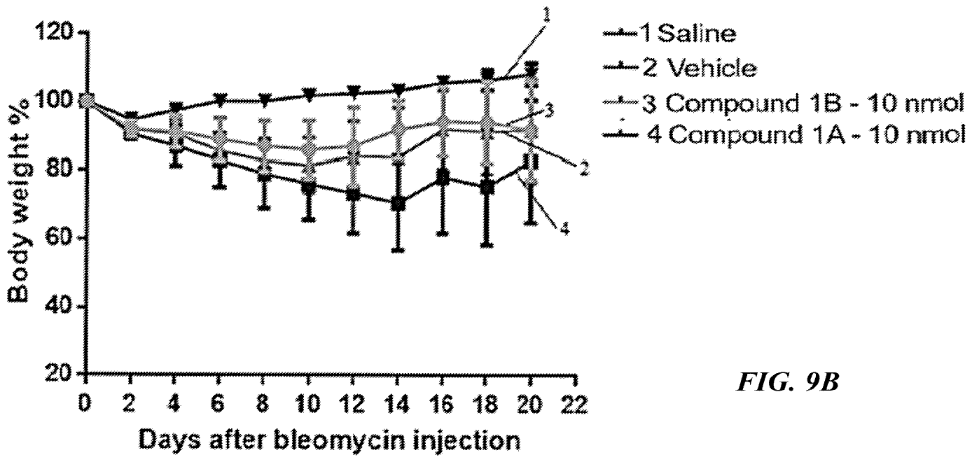

With an ability to target attached drugs to FRβ expressing fibrotic macrophages established, it was then investigated whether a folate targeted TLR7 agonist might be capable of suppressing the signs and symptoms of fibrosis in BM-treated mice. For this, BM-treated mice were intravenously injected every other day beginning on day 10 with either vehicle (3% DMSO in PBS) or Compound 1B (see FIG. 8A). Because the TLR7-54 agonist caused rapid body weight loss followed by death (see FIGS. 9A and 9B), Compound 1A could not be similarly evaluated in vivo. In BM-induced experimental pulmonary fibrosis in mice, inflammation is known to persist for about 9-10 days after BM installation. Because, inflammation to fibrosis switch happens in this model approximately day 9 to day 14, and anti-inflammatory markers start appearing at about day 10, dosing began on day 10 (FIG. 8A).

Two doses were given every other day till day 21. The individual doses on a day are separated by 6 hours to prevent any “tolerance” to TLR agonists. Mice were then sacrificed on day 21 and subjected immediately to bronchoalveolar lavage followed by resection of the lungs for immunohistochemistry and quantitation of collagen and hydroxyproline.

FIGS. 8B-8G show graphical data representative of various marker levels measured from mice treated with the BM model of FIG. 8A, with BALF collected on day 21 and centrifuged at 4° C., the resulting pellet resuspended in the medium and seeded into 96-well plates, cultured for 2 hours, washed with pre-warmed PBS 3 times, and cells harvested for qPCR; the data showing that Arg1 (FIG. 8B), MMP9 (FIG. 8C), TIMP 3 (FIG. 8D) (e.g., anti-inflammatory markers) were all downregulated. CD86 (FIG. 8E) and IFN-γ (FIG. 8F) (e.g., proinflammatory markers) were both upregulated. Further, the negative regulator of TLR7 signaling IRAK-4 was upregulated (FIG. 8G), as were the number of BALF cells present (FIG. 811). Indeed, the total number of mice BALF cells decreased in a dose-dependent manner following treatment with different doses of Compound 1B. Each value shown in FIGS. 8B-8G represents the mean±S.D, for each group; *P<0.05. **P<0.005, ***<0.0005; for the saline versus vehicle group, Compound 1A and Compound 1B-treated groups versus vehicle group calculated by Student's t test, except for the BALF cell count and protein concentration measurements, in which the Compound 1B treated and vehicle group were calculated by Dunnett's multiple comparison test; and vehicle=3% DMSO in PBS.

As shown in FIGS. 8B-8D, qPCR analysis of the anti-inflammatory markers in the macrophage subpopulation of bronchioalveolar lavage cells revealed that Arg1, MMP9, and tissue inhibitor of TIMP 3 were all elevated in BM-induced mice relative to the control mice. More importantly, parallel studies demonstrated that the same anti-inflammatory markers were all suppressed when BM-induced mice were treated with Compound 1B, yielding levels of the anti-inflammatory markers similar to those seen in healthy mice. Consistent with these data, quantitation of proinflammatory markers revealed that transcripts of CD86 (qPCR) and concentrations of IFN-γ (ELISA of lavage fluid) were both elevated following treatment with Compound 1B (see FIGS. 7E and 7F). Taken together with the observed upregulation of IRAK-4 (i.e. a marker of TLR activation; results shown in FIG. 7G) and the total number of BALF cells present being decreased in a dose-dependent manner after treatment with different doses of Compound 1B (FIG. 7H), these data demonstrate that administration of a folate-targeted TLR7 agonist can reprogram macrophages from an anti-inflammatory M2-like phenotype to an anti-fibrotic M1-like phenotype in the lungs of BM-treated mice in vivo.

### Example 6

An additional study was conducted to determine if the above-described reprogramming of fibrotic lung macrophages resulted in actual improvement of the fibrotic/anti-inflammatory condition in the fibrotic mice. Lung tissue from the above mice was embedded in paraffin, and sectioned and stained with H&E and Masson's trichrome for evaluation of tissue density and extracellular collagen deposition, respectively.

FIGS. 9A and 9B show survival curves (FIG. 9A) and body weight change (FIG. 9B) of mice having experimental pulmonary fibrosis treated with non-targeted and targeted TLR7 agonists. The data supports that administration of the compounds of the present disclosure (here, for example, Compound 1B) increases survival of BM-treated mice without causing significant body weight loss. Each value represents the mean±S.D, for each group.

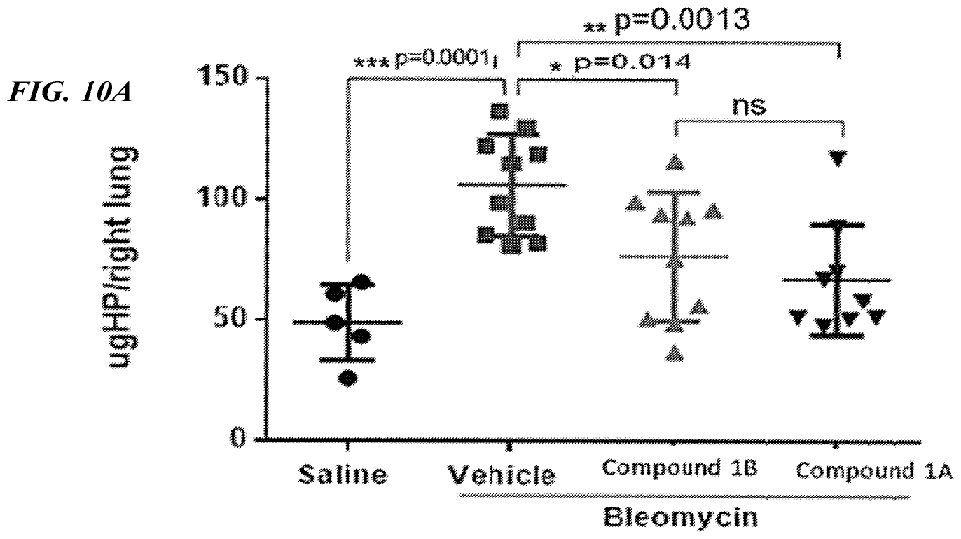

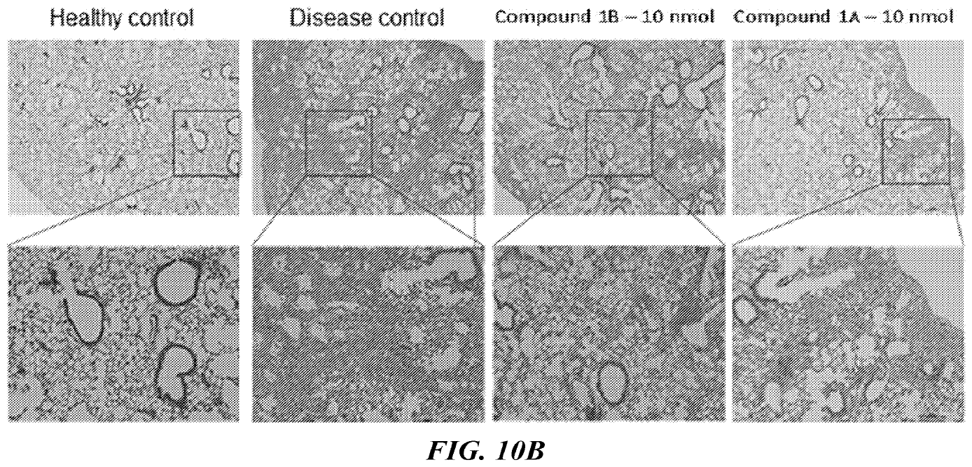

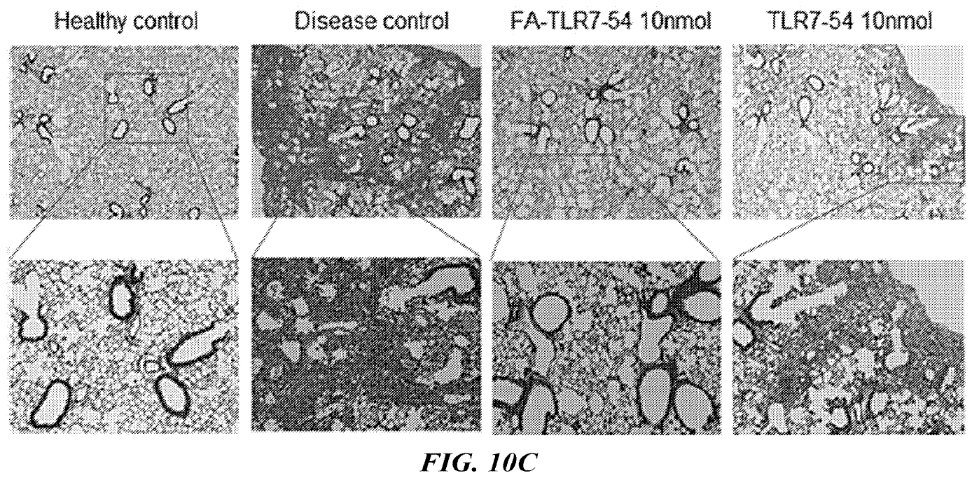

FIG. 10A shows the hydroxyproline content (pg/lung) of lung tissue to utilize collagen deposition as a measure of fibrosis. Tissue at day 21 for each of the following are shown: a healthy control (saline)(•), a disease control (vehicle)(▪), treated with free drug TLR7 agonist (Compound 1A)(▾), and treated with a folate-targeted TLR7 agonist (Compound 1B)(▴). BM-induced mice treated with 10 nmol of either Compound 1B (A) and Compound 1A (7) showed a significant decrease in the total hydroxyproline content per lung as compared with the vehicle control (▪). Each value shown in FIG. 9A represents the mean±S.D, for each group; *P<0.05, **P<0.005, ***<0.0005; saline versus vehicle group, Compound 1A and Compound 1B-treated groups versus vehicle group by Student's t test.

FIGS. 10B and 10C show stained images of the lung tissue represented in FIG. 10A with H&E staining (FIG. 10B) and Masson's trichrome (collagen) staining (FIG. 10C).

As shown in the H&E stains of the panel of FIG. 10B, healthy lungs contain an abundance of air sacs surrounded by thin reticular membranes. In contrast, BM-induced lungs display far fewer alveoli with pronounced deposition of extracellular matrix where air sacs once existed. Most importantly, BM-instilled mice treated beginning on day 10 with Compound 1B exhibited a lung architecture that resembles that of healthy mice (FIG. 10B), suggesting that targeting of Compound 1A to the fibrotic lung macrophages is effective to suppress the major hallmarks of pulmonary fibrosis. That this prevention of fibrosis indeed involves the blockade of collagen deposition is documented by Masson's trichome staining of parallel lunch sections (FIG. 10C), where the collagen stain is strongly suppressed in mice injected via tail vein with the Compound 1B (FIG. 10B). Accordingly, the data supports the IPF mice treated with at least Compound 1B (▴) demonstrate suppression of the IPF pathology (e.g., fibrosis).

Finally, to confirm that Compound 1B did indeed impact the production of collagen in vivo, hydroxyproline (a major component of collagen) was quantitated in total hydrolysates of the affected lungs. More specifically, lung tissue from the above mice was perfused with PBS, hydrolyzed with acid, and analyzed for hydroxyproline content. As shown in FIG. 10A, induction of fibrosis induces a large increase in the hydroxyproline content and this increase was suppressed upon treatment with Compound 1B. Accordingly, the data supports that treatment with the targeted TLR7 agonist compounds of the present disclosure reduces (and even counters) the deposition of collagen, and thus fibrosis, in vivo.

In sum, overall survival of mice injected with optimized BM dose (0.75 mg/kg) was significantly improved by treatment with Compound 1B, whereas there was no survival benefit with Compound 1A except for showing significant weight loss (>25%, FIG. 7). While free drug performed better in the reduction of hydroxyproline content, the poor survival seen can be attributed to overall toxicity (i.e. weight loss, see FIG. 7B). This was not surprising as the systemic administration of TLR7 agonists has been known to cause toxicity.

### Example 7

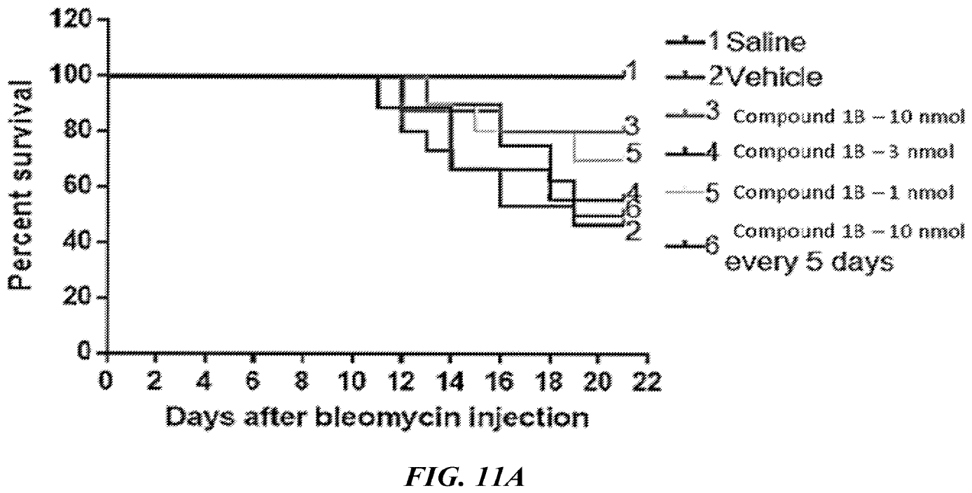

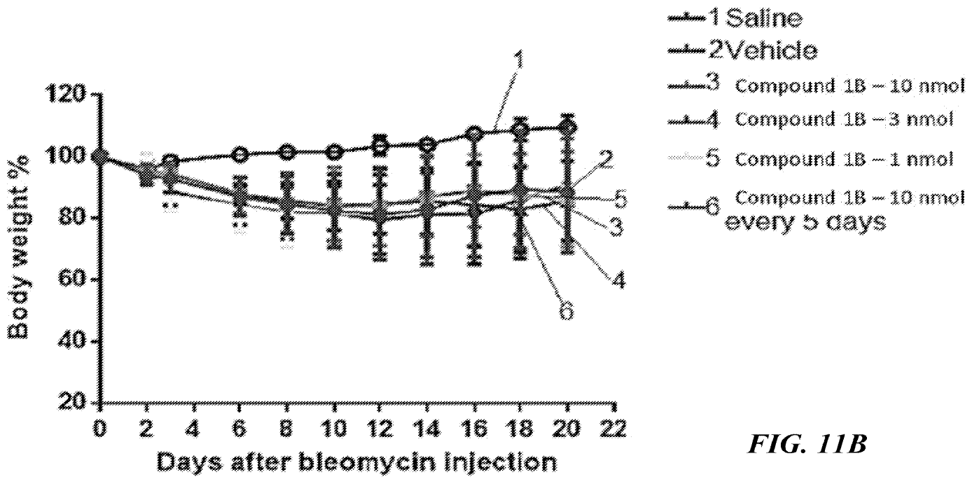

Because use of the nontargeted TLR7 agonist to treat IPF (or other fibrotic diseases) has been prevented by its systemic activation of the immune system and resulting toxicity, it was assessed whether any obvious toxicities might have accompanied systemic administration of Compound 1B in mice. To this end, BM-induced mice were treated every other day beginning on day 10 with 0, 1, 3, or 10 nmoles of Compound 1B and body weight, lung hydroxyproline content, and histological analyses were performed on day 21. Unlike conventional systemic administration, the targeted drug not only improved the survival, but also reduced the weight loss underlining the significance of targeting approach (FIGS. 11A and 11B).

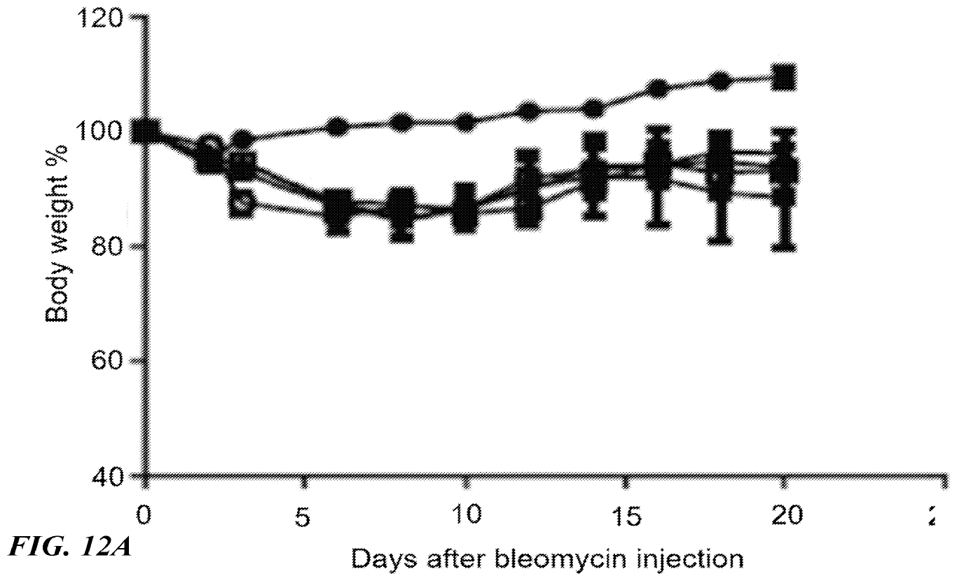

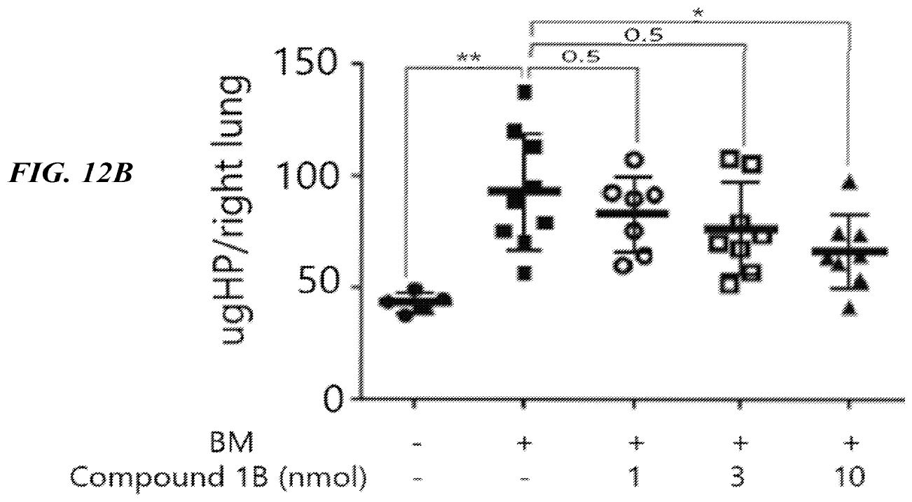

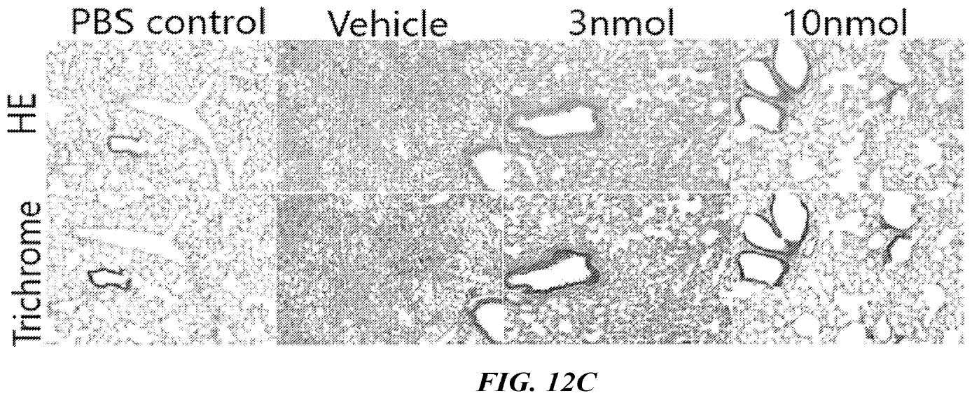

FIG. 12 shows data relating to the dose-dependent effect of a folate-targeted TLR7 agonist on the suppression of fibrosis in BM-induced mice, using collagen deposition as a measure of fibrosis. The data are represented by: healthy control (PBS, •). BM-induced mice with the treatment vehicle (▪), 1 nmol Compound 1B (∘), 3 nmol Compound 1B (□), or 10 nmol Compound 1B (▴)), with subpart A showing graphical data related to the body weight of the BM-induced mice over time, subpart B showing measurement of hydroxyproline content of the lung tissue (μg/lung) treated with different doses (10 nmol, 3nmol, or 1 nmol of the Compound 1B), and subpart C showing images for histological analysis of the right lung tissue with H&E staining and Trichrome staining.

As seen in FIG. 11B and subpart A of FIG. 12, no difference in weight loss was observed between mice treated with 0, 1, 3, or 10 nmoles of Compound 1B, suggesting that no gross toxicity was caused by repeated dosing with the compound. That these treatments were still having the anticipated effects on lung fibrosis could nevertheless be seen from comparison of the hydroxyproline contents of the various lung hydrolysates, where the order of efficacy was 10 nmol/mouse>3 nmol/mouse>1 nmol/mouse>0 nmol/mouse (subparts B and C of FIG. 12). More importantly, detailed analyses of the lung histology demonstrated that as the dose of Compound 1B increased, lung histology improved, which suggests that the tissue in which the TLR7 agonist was most strongly concentrated was in fact the tissue in which the microscopic morphology was most normal. Taken together, these data support that the targeting of the TLR7 agonist FRβ+ macrophages in fibrotic/anti-inflamed tissue can effectively prevent fibrosis (i.e. the M2-phenotype) without systemic activation of the immune system that otherwise limits TLR7 agonist use in humans.

Finally, to determine if this proinflammatory effect can be achieved with lower doses, a therapeutic study with two lower doses (3 nmol/kg and I nmol/kg) was undertaken (FIGS. 9 and 10). Interestingly, while the low doses showed significant reduction in hydroxyproline content and collagen deposition levels, the 10 nmol dose provided the best survival rates.

### Example 8

Other representative embodiments of the compounds hereof were examined in in vitro studies.

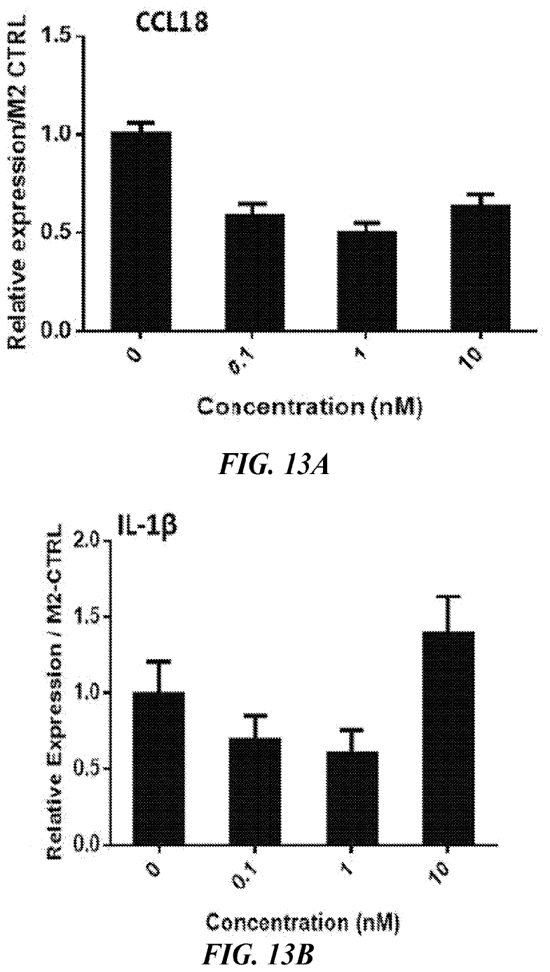

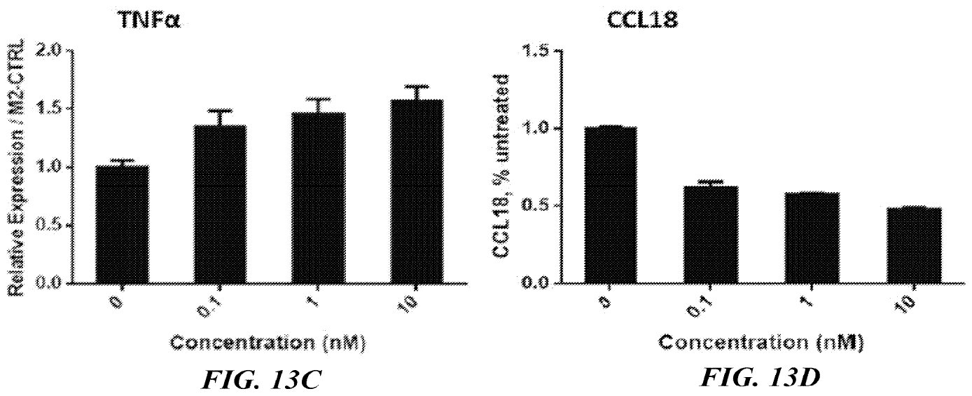

FIGS. 13A-13D show graphical data representative of various marker levels measured from human THP-1 cells that were induced to M2 macrophages with 20 ng/mL IL-4, 20 ng/mL IL-13, 5 ng/mL IL-6. The cells were subsequently reprogrammed with different nM concentrations of a TLR7 agonist having formula IV (e.g., Compound 2A) for 48 hours and harvested for gene analysis by qPCR, mRNA levels of the following markers relative to the expression of a M2-like macrophage control: CCL18 mRNA levels (FIG. 12A), IL-1β mRNA levels (FIG. 13B), and TNFα levels (FIG. 13C), and FIG. 13D show protein analysis results after cell supernatants were collected. Secreted CCL18 protein was detected by ELISA.

In FIGS. 13A-13D, an agonist compound of the present disclosure having formula IV (e.g., Compound 2A) was evaluated with respect to its ability to reprogram M2-like macrophages to M1-like macrophages.

Primarily, human monocytic (THP-1) cells were induced to the M2-like phenotype using the methods and materials previously described. In particular, THP-1 cells were seeded into 96-well plates at a density of 60,000 cells/well. Cells were differentiated into unpolarized macrophages by 48 h incubation with 200 nM PMA followed by 24 hours incubation in fresh RPMI medium. The resulting macrophages were polarized to an M2-like phenotype by incubation with 20 ng/nm IL-4, 20 ng/ml IL-1β, and 5 ng/mL IL-6 for 48h. Cultures were maintained at 37° C. in a humidified 5% CO₂ incubator.

To evaluate whether Compound 2A could reprogram the anti-inflammatory macrophages into a less fibrotic/proinflammatory phenotype, IL-4, IL-6 plus IL-13 stimulated THP-1 cells were incubated with different concentrations of Compound 2A, and the mRNA levels of several anti-inflammatory markers were examined using qPCR and ELISA—namely, CCL18, IL-1β, and TNFα.

As shown in FIGS. 13A and 13B, incubation with Compound 2A (free drug) for 48 hours induced a decrease in CCL18 and IL-1β expression, suggesting that the TLR7 agonist can indeed promote a shift in these anti-inflammatorily polarized THP-1 cells towards a less fibrotic/proinflammatory phenotype. (Note FIG. 13B shows a bell-shaped curve indicative of Compound 2A having an inhibitory response at lower concentrations and a stimulatory response at high concentrations, which is a common response curve with certain drugs.) Moreover, when expression of TNFα (a proinflammatory phenotype marker) was examined, an increase in its expression was observed (FIG. 13C), confirming that the THP-1 shift from anti- to pro-inflammatory properties occurred.

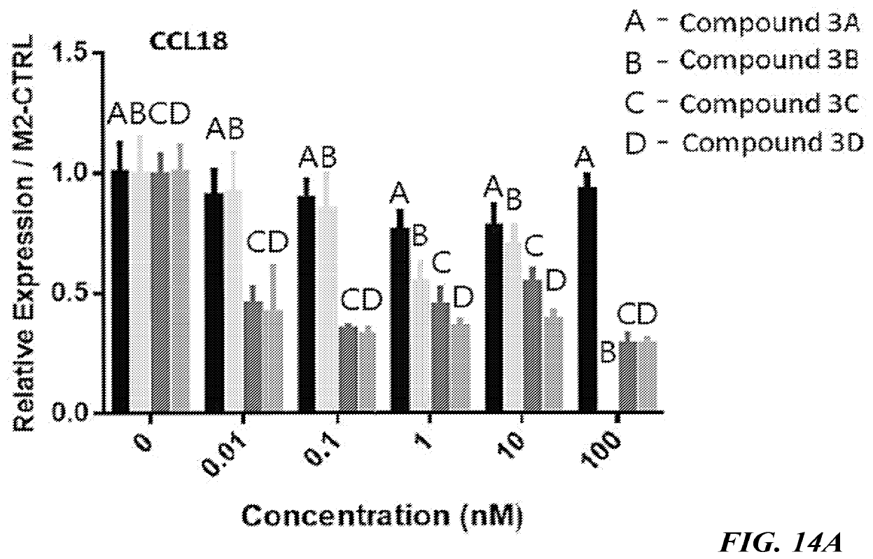

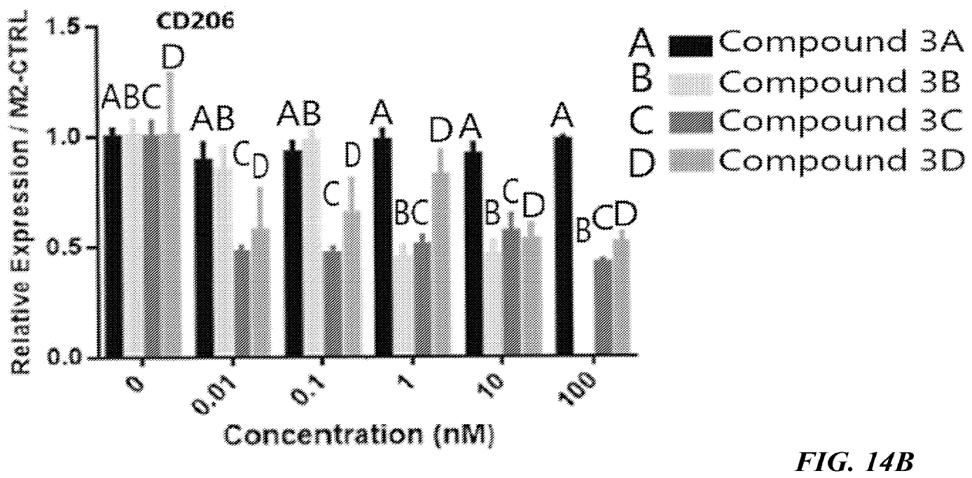

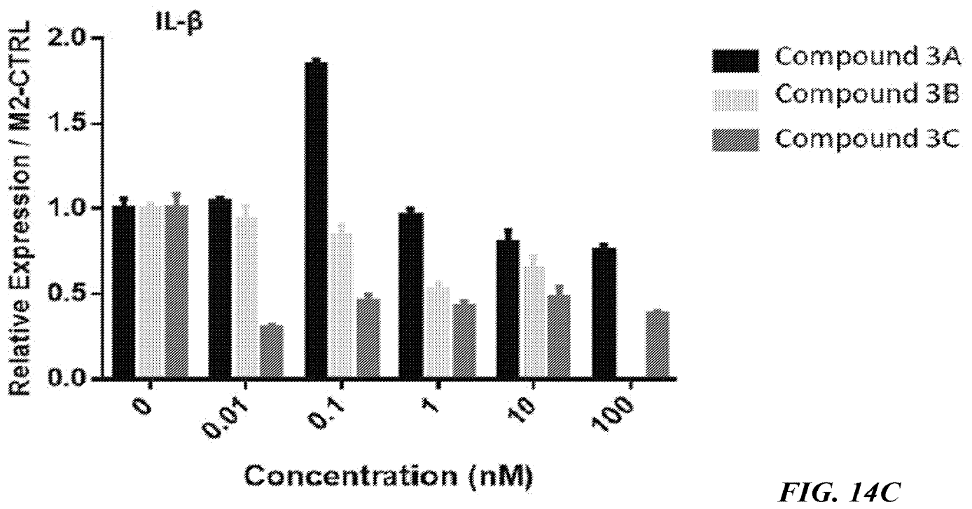

In addition to the nonconjugated TLR7 agonist, conjugated compounds of the present disclosure were likewise evaluated. Human THP-1 cells were induced to macrophages having the M2-like phenotype per the methods set forth herein (e.g., using 20 ng/mL IL-4, 20 ng/mL IL-13, 5 ng/mL IL-6), then reprogrammed with different nM concentrations of various compounds of the present disclosure for 2 hours; namely, a nonconjugated (free drug) TLR7 agonist compound having formula I and/or II (data shown collectively as Compound 3A), a folate-conjugated TLR7 agonist compound having formula XV (having a releasable linker) (e.g., Compound 3B), a folate-conjugated TLR7 agonist compound having formula XVII (having a non-releasable linker) (e.g., Compound 3C), and a folate-conjugated TLR7 agonist compound having formula XVI (having a non-releasable linker) (e.g., Compound 3D). The cells were subsequently harvested for gene analysis by qPCR and the relative expression of CCL18 (FIG. 14A), CD206 (FIG. 14B), and IL-1β (FIG. 14C) analyzed.

Expression of the various anti-inflammatory (M2 phenotype) markers CCL18, IL-1β, and CD206 markers were quantified. As shown in FIGS. 14A-14C, expression of each of these anti-inflammatory markers were reduced after administration of each of Compound 3B, Compound 3D, and Compound 3C, with Compound 3D and Compound 3C (both with non-releasable linkers) most effective relative to the other compounds.

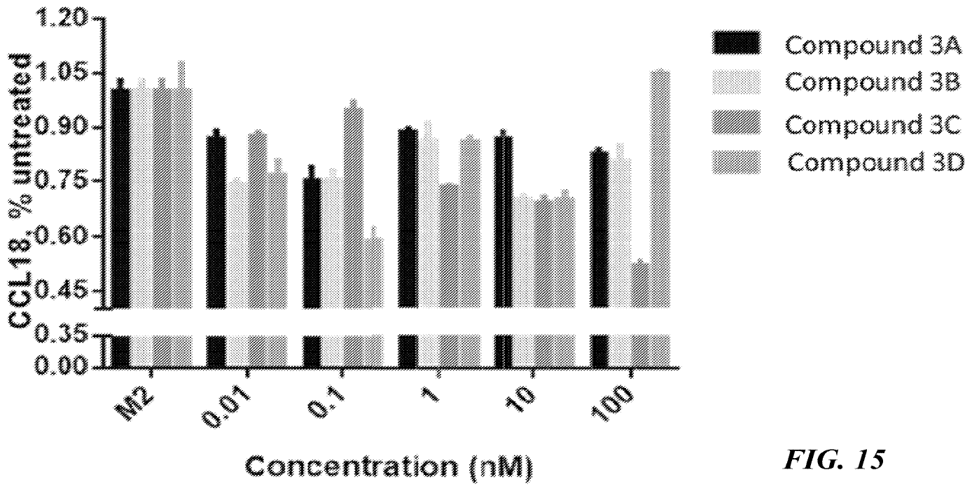

FIG. 15 shows secreted CCL18 protein levels in each of the groups of THP-1 cells of FIGS. 14A-14C after treatment with the Compound 3A, Compound 3B, Compound 3C, or Compound 3D. Compound 3A and the folate-targeted TLR7 compounds (e.g., Compound 3B, Compound 3C, and Compound 3D) downregulate the secretion of CCL18 at a low concentration range (0.1-10 nM).

Additionally, cell supernatants were collected and secreted CCL18 protein was detected by ELISA. FIG. 15 confirms that Compound 3A (free drug) and the folate-targeted compounds (Compound 3B, Compound 3C, and Compound 3D) all downregulated the secretion of CCL18 at a low concentration range (0.1-10 nM), further supporting that, akin to the examples described in connection with Compound 1A and Compound 1B, these compounds can similarly reprogram M2-like anti-inflammatory macrophages to Mi-like proinflammatory macrophages through like mechanisms.

### Example 9

Upon repeating the studies described above (see grey bars, FIGS. 3A-3F), the same qualitative changes were observed, only the magnitude of the impact of Compound 1B was somewhat reduced. This reduction in potency was expected because the nontargeted TLR7 agonist enters the cultured cells immediately, whereas its folate-targeted counterpart is designed to enter cells only after folate receptor-binding and receptor-mediated endocytosis. Because low molecular weight water-soluble drugs like Compound 1A and Compound 1B are often excreted from the body within 2 hours of injection, a more physiologically relevant in vitro model of drug exposure in vivo is to limit incubation of a cell with drug for only two hours and then examine drug efficacy after an additional 46 hours of incubation in the absence of the drug. As shown in FIGS. 4A-4E, when THP-1 cells were incubated with the TLR7 agonists for 2 hours prior to replacement of the drug-containing medium with drug-free medium, Compound 1B was observed to have superior potency relative to Compound 1A, especially in the case of TNFα induction where the folate-targeted compound was dramatically improved. This is most likely because the folate-targeted TLR7 agonist was captured by the folate receptor positive cells, whereas Compound 1A was not retained by the same cells.

These data support that Compound 1B should be more effective in reprogramming anti-inflammatory macrophages in vivo, with the added advantage that the folate-conjugated drug (e.g., Compound 1B) should also cause less systemic toxicity because it is concentrated in the FRβ-expressing macrophages and unable to enter folate receptor negative cells that predominate throughout the body (e.g., Compound 1B is designed to be impermeable to folate receptor negative cells).

Further, to ensure that the above mRNA analyses accurately reflected the levels of anti-inflammatory cytokines produced by IL-4, IL-6 plus IL-13 stimulated THP-1 cells, the concentrations of CCL18 and IL-1β polypeptides in the THP-1 supernatants were quantitated by ELISA assay. As shown in FIGS. 6A and 6B, both Compound 1A and Compound 1B induced reductions in CCL18 and IL-1β when incubated continuously with agonist for 48 hours; however, Compound 1B again was found to be superior when drug exposure was limited to only 2 hours (see FIGS. 6C and 6D).

### Example 10

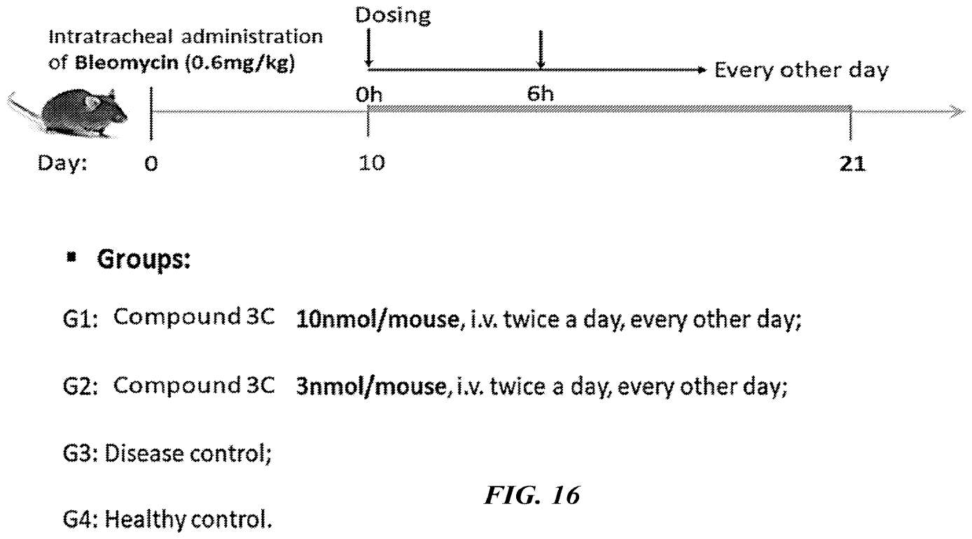

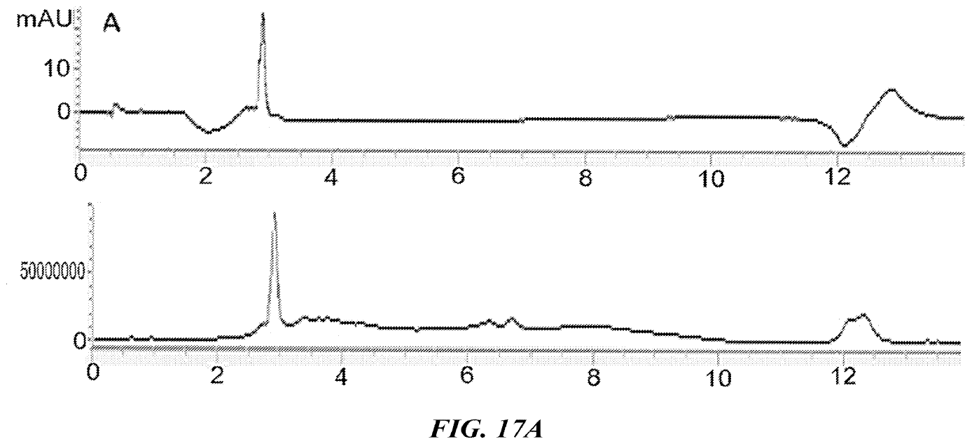

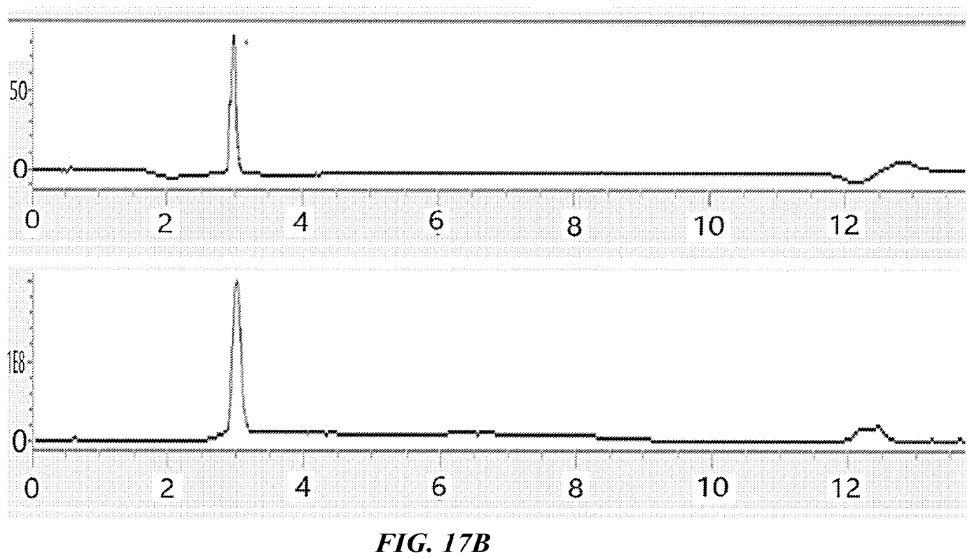

FIG. 16 illustrates the in vivo study methodology of at least one embodiment of a compound of the present disclosure in a BM murine model, the compound having formula XVII (e.g., Compound 3C). FIGS. 17A and 17B are the LC-MS spectrum of Compound 3C and support the high purity of the compound and no free drug was detected.

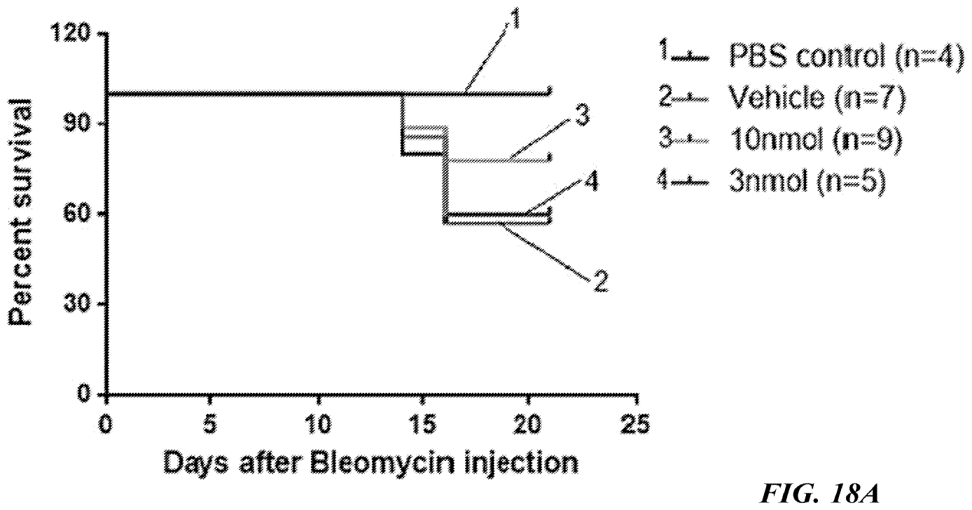

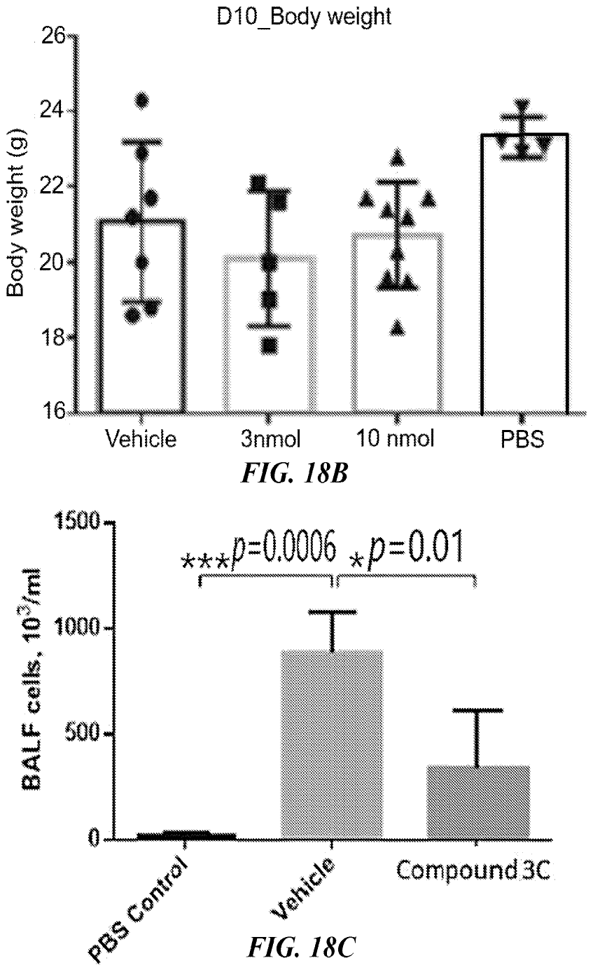

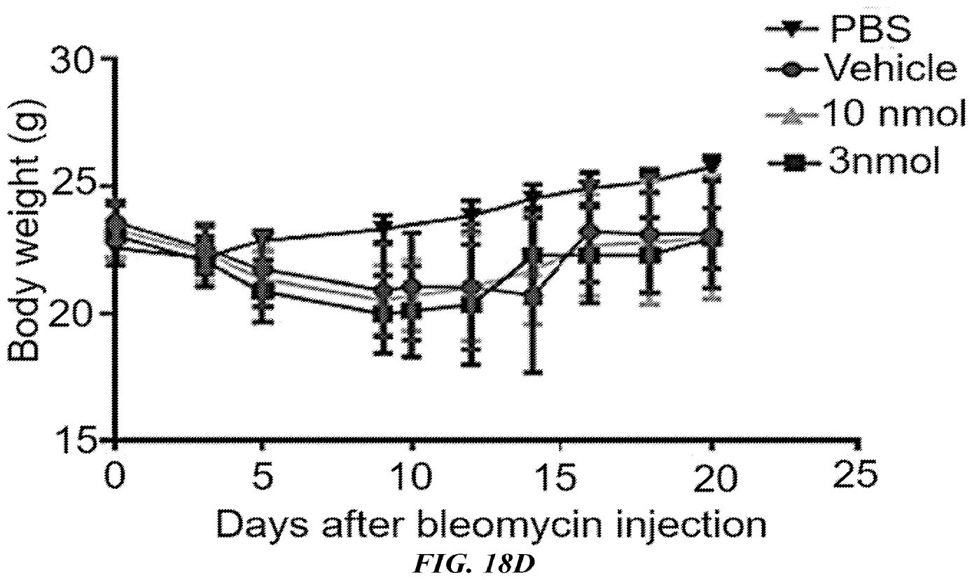

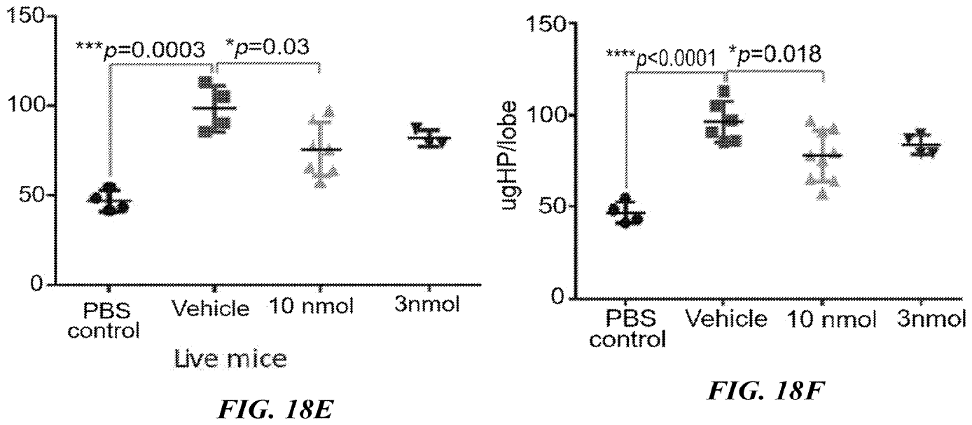

FIGS. 18A-18F shows results from the subject mice of the in vivo study methodology of FIG. 16, including survival curves (FIG. 18A), body weight changes (FIGS. 18B and 18D), concentration of cells with BALF (FIG. 17C), hydroxyproline concentration (μgHP/lobe) in live mice (FIG. 18E) and in all mice (e.g., inclusive of both live mice and those that died before day 21) (FIG. 18F). The 10 nmol concentration dosage of the compound having formula XVII (e.g., Compound 3C) increased the survival rates of the subject mice, while concurrently decreasing the HP and number of BALF cells. Also, the 3 nmol concentration dosage did not show measurable benefit to the subject mice.

### Example 11

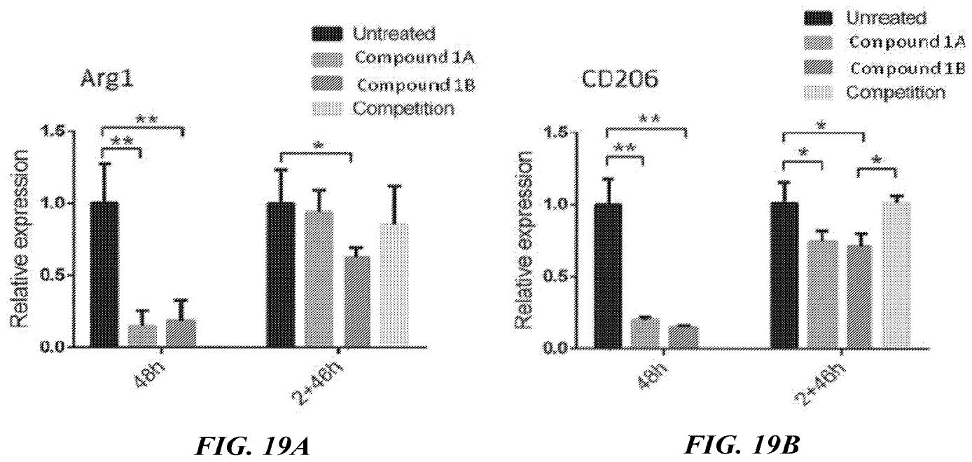

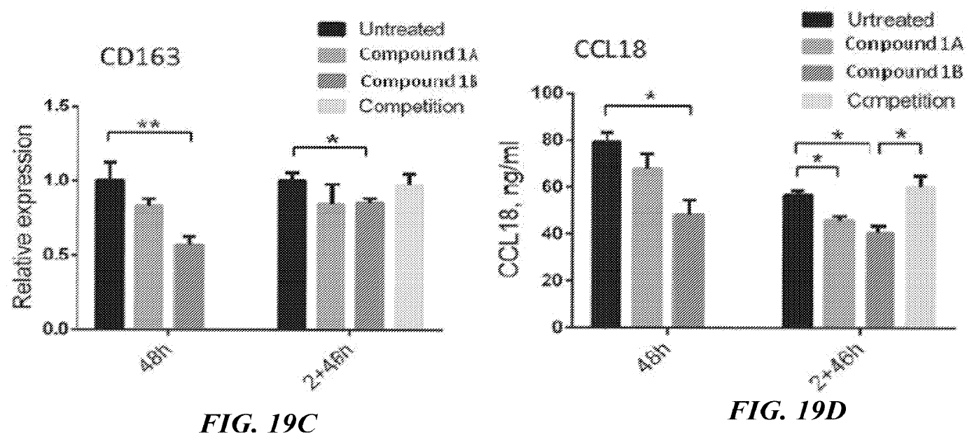

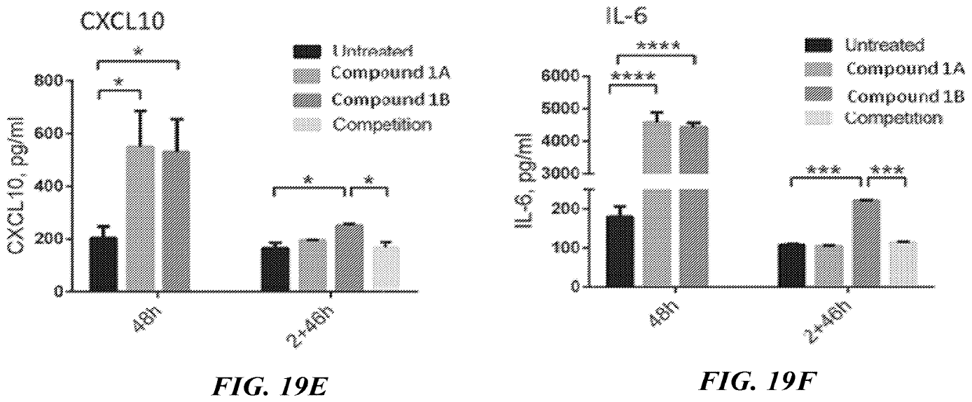

M2-induced human monocyte-derived macrophages were treated with 100 nM of Compound 1A or Compound 1B either continuously for 48 hours, or initially for 2 hours in the presence or absence of FA-glucosamine (competition) followed by 46 hours in the absence of drug (2+46 hours). As shown in FIG. 19, mRNA levels of anti-inflammatory markers. Arg1 (FIG. 19A), CD206 (FIG. 19B) and CD163 (FIG. 19C), and protein levels of secreted anti-inflammatory CCL18 (FIG. 19D) and proinflammatory cytokines, CXCL10 (FIG. 19E) and IL-6 (FIG. 19F) (n=3, technical replicates) were then determined. Changes in both sets of cytokines were inhibited by blockade of unoccupied folate receptors with excess FA-glucosamine (2+46 hours, competition). This data supports that Compound 1B binds to folate receptor since the downregulation of biomarkers was blocked with excess FA-glucosamine (competitor).

### Example 12

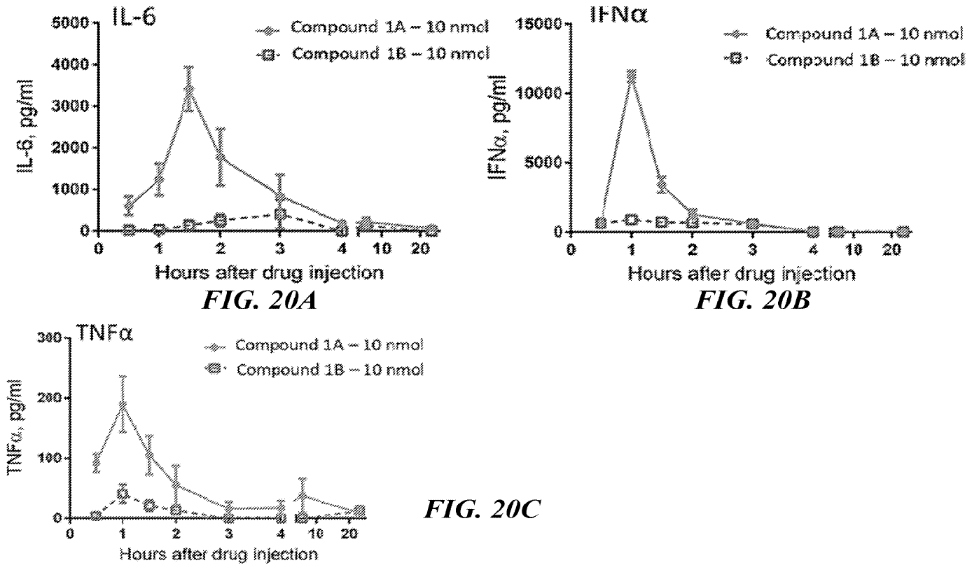

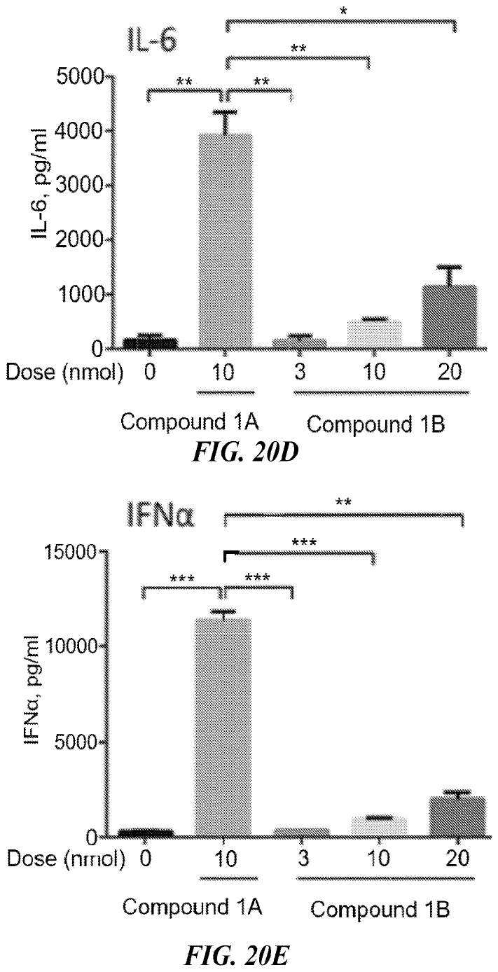

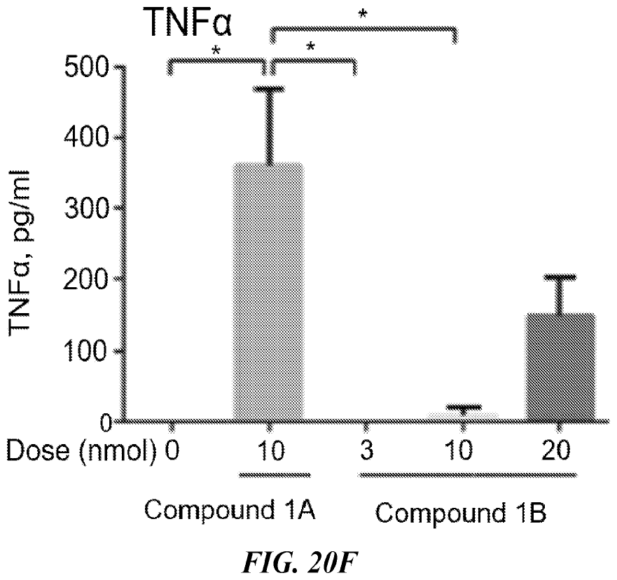

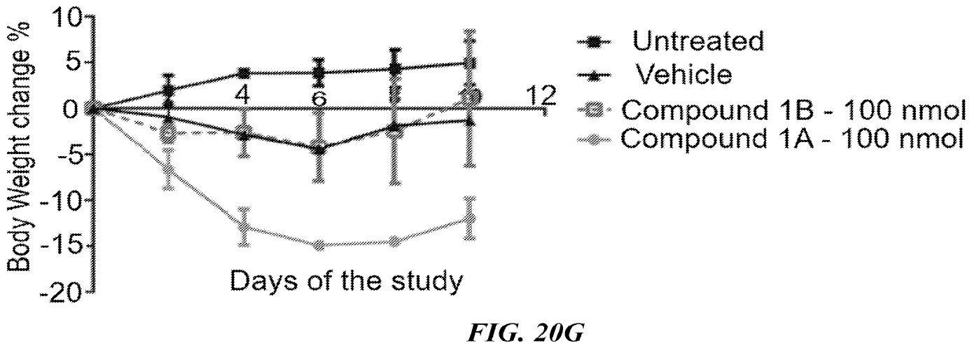

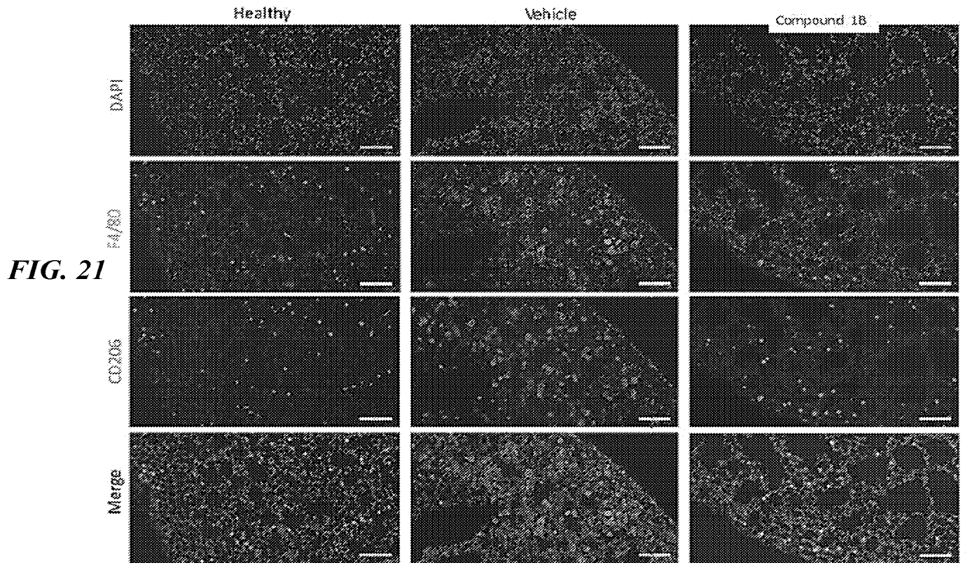

Healthy mice were tail vein injected with 10 nmol Compound 1A (circles) or Compound 1B (squares), and peripheral blood was collected at indicated time points after drug injection. (FIGS. 20A-C) Measurement of plasma IL-6 (FIG. 20A), IFNα (FIG. 20B) and TNFα (FIG. 20C) (n=3). (FIGS. 20D-F). The effect of drug concentration on plasma levels of IL-6 (FIG. 20D), IFNα (FIG. 20E), and TNFα (FIG. 20F) was determined at 1.5 hours, 1 hour, or 1 hour after treatment, respectively (n=2) (FIG. 20G). Compound 1A stimulates systemic cytokine release in healthy mice, while Compound 1B does not. Furthermore, Compound 1B stimulates less inflammatory cytokine release than half the dose of Compound 1A. These data suggest that TLR7 agonists can be safely employed to reprogram fibrotic lung macrophages to an anti-fibrotic/pro-inflammatory state if they are targeted to the pulmonary macrophages with a folate receptor targeting ligand.

### Example 13

Sections from the same healthy and fibrotic lungs described in FIG. 6 were stained with DAPI (nuclei; blue), anti-F4/80 (macrophages; red), and anti-CD206 (M2 macrophage marker: green), and images were obtained with a Leica Versa 8 whole-slide scanner as described in Methods (n=2). Scale bars, 100 μm. Differences between treated groups indicate that Compound 1B produces a robust anti-fibrotic and proinflammatory response in vivo.

### Example 14

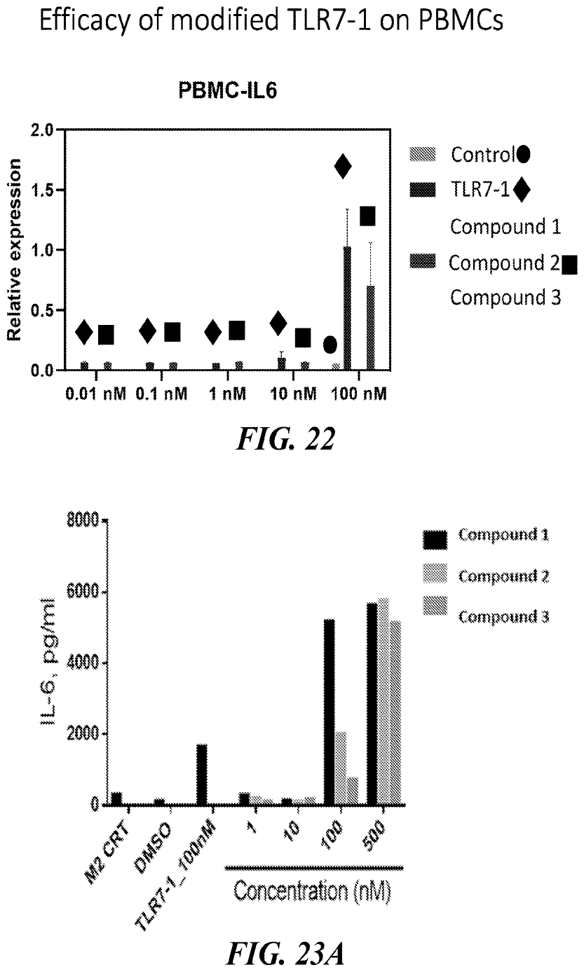

To evaluate the efficacy of TLR-7 agonists, Compound 1 (TLR7-TA), Compound 2 (TLR7-1B) and Compound 3 (TLR7-1C) were treated with peripheral blood mono nuclear cells (PBMCs) for 24 hours. TLR7-1 was used as control. Cell culture supernatant was isolated and tested for IL-6 using enzyme-linked immunosorbent assay (ELISA) (FIG. 22). As shown in FIG. 22, TLR7 agonists resulted in increased expression of IL-6 in PBMCs.

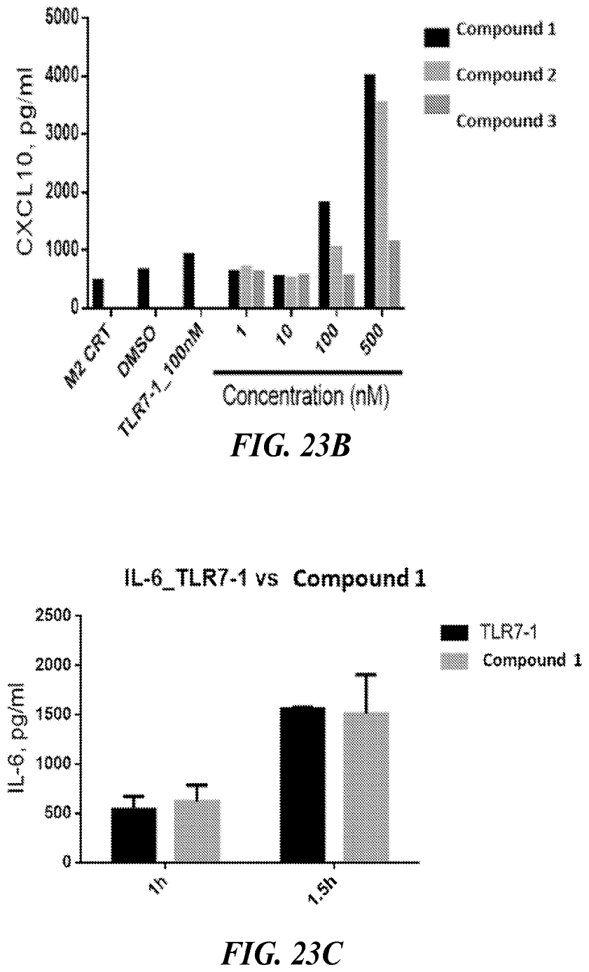

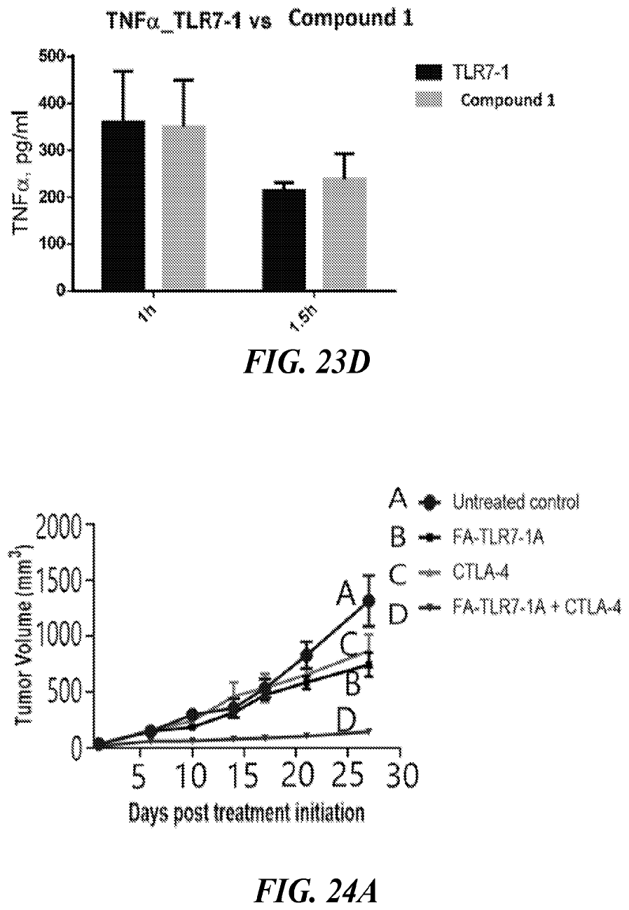

When these compounds were treated with human primary monocyte-derived M2-macrophages for 48 hours, they induced IL-6 and CXCL-10 more efficiently compared to the parent compound TLR7-1 (FIGS. 23A and 23B). Compounds 1, 2 and 3 polarize the M2 macrophages to M1 macrophages as shown by the increased M1 markers IL-6 (FIG. 3A) and CXCL10 (FIG. 2311).

### Example 15

Healthy mice were tail vein injected with 10 nmol of Compound A (TLR-1) or Compound 1 (TLR-1A), and peripheral blood was collected at indicated time points after drug injection. (FIGS. 23C and 23D). The effect of drug on plasma levels of IL-6 (FIG. 23C) and TNFα (FIG. 23D) was determined at 1 hour or 1.5 hours after treatment. Both compounds stimulated systemic cytokine release in healthy mice.

### Example 16

### Combination Therapy of Folate-TLR7-1A (Compound 1000) with Checkpoint Inhibitors in 4T1 Tumor Model

Six- to eight-week-old female BALB/c mice were purchased from Charles River Laboratories, housed in a sterile environment on a standard 12-hour light-dark cycle, and maintained on folate deficient diet. Upon arrival, the mice were transferred to a folic acid-deficient diet.

On day 14, mice were implanted orthotopically with 5.0×10⁴ of 4T1 cells and tumors were allowed to grow until they reach ~50 mm³. The 4T1 cell line was purchased from American Type Culture Collection (ATCC) and cultured as monolayers in 1640 RPMI supplemented with 10% heat-inactivated fetal bovine serum, and 1% of penicillin-streptomycin at 37° C., in a 5% CO₂/95% humidified-air atmosphere. The study was performed with 10 passages of thawing the cells from frozen stocks.

Mice were then randomized into groups based on their tumor volume and treated with folate-TLR7-1A (Compound 1000), folate-TLR7-1A (Compound 1000)+CKI and CKI. In all the experiments, folate-TLR7-1A (Compound 1000) was administrated through intravenous injection daily (100 μL, 3 nmole/mouse) for 5 days per week and the checkpoint inhibitor (CKI) antibodies, anti-PD-1 and anti-CTLA-4, were given intraperitoneal (i.p.) injection on days 3, 6, 10, 13, 17 and 20 at a dose of 200 pg/mouse. Control groups received PBS on the same schedule.

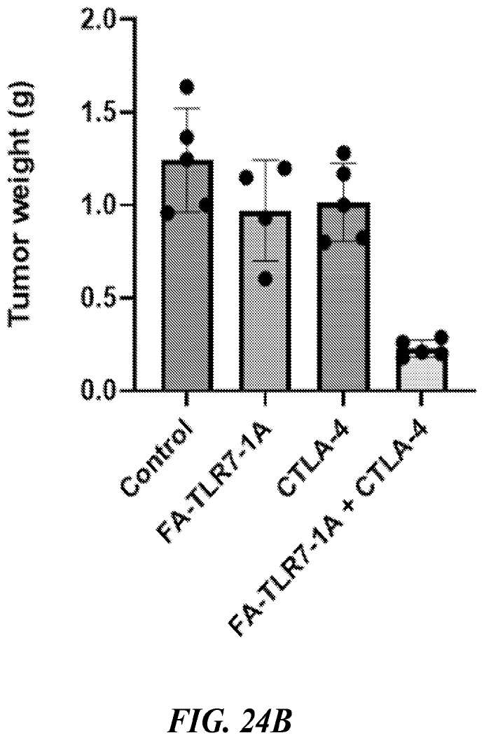

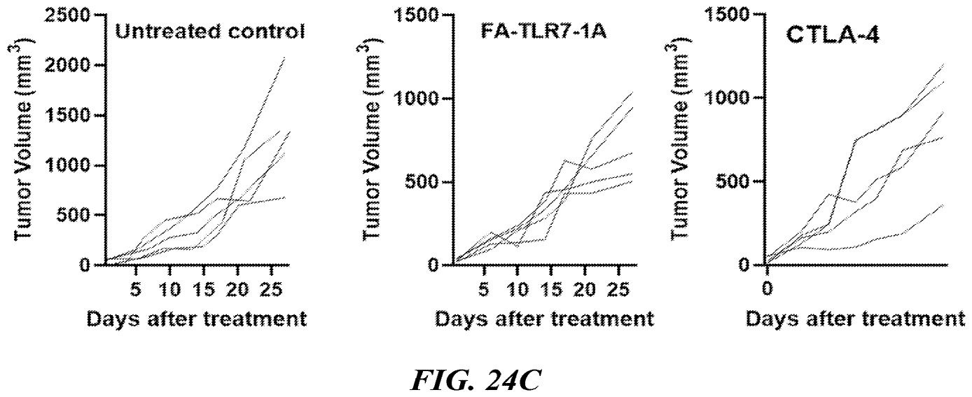

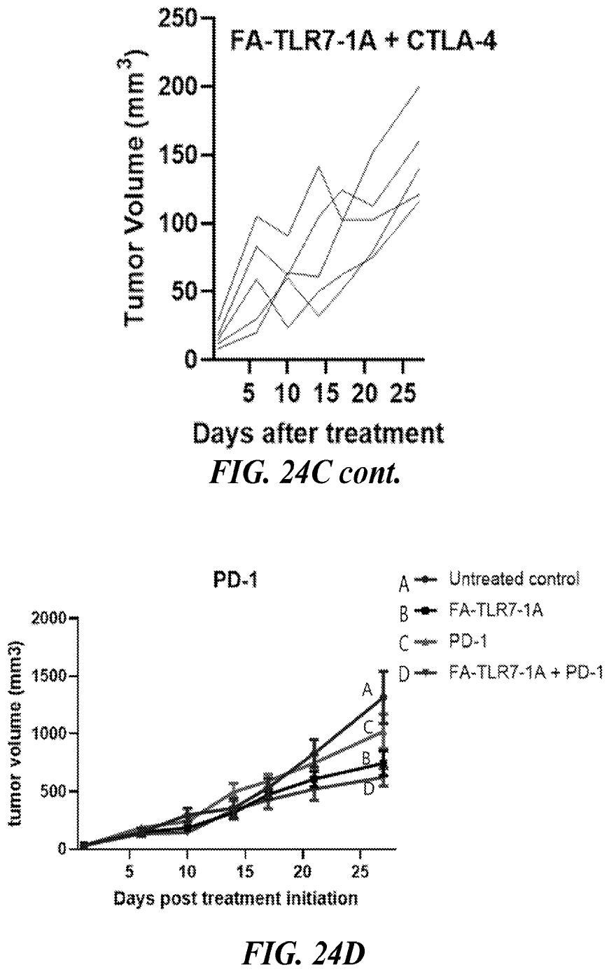

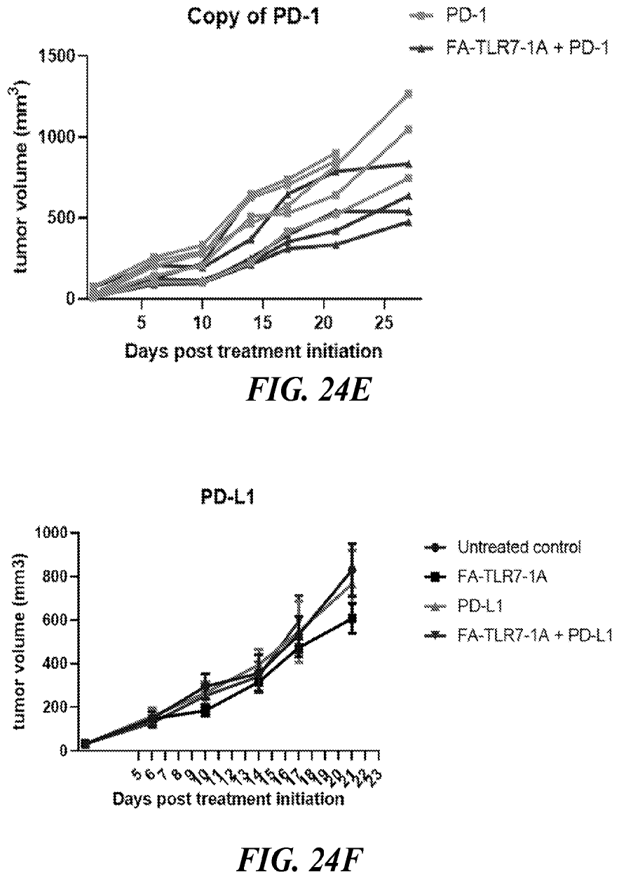

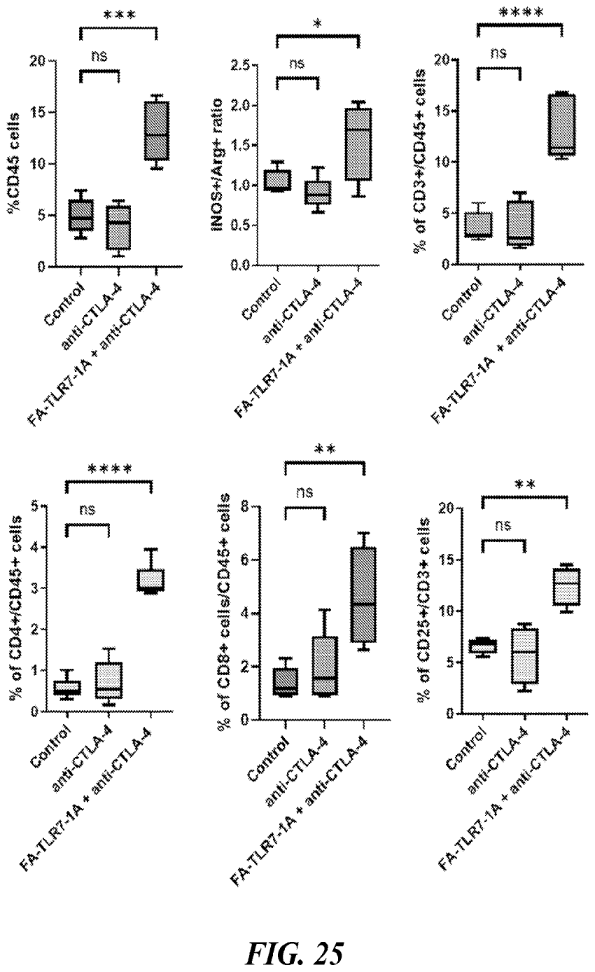

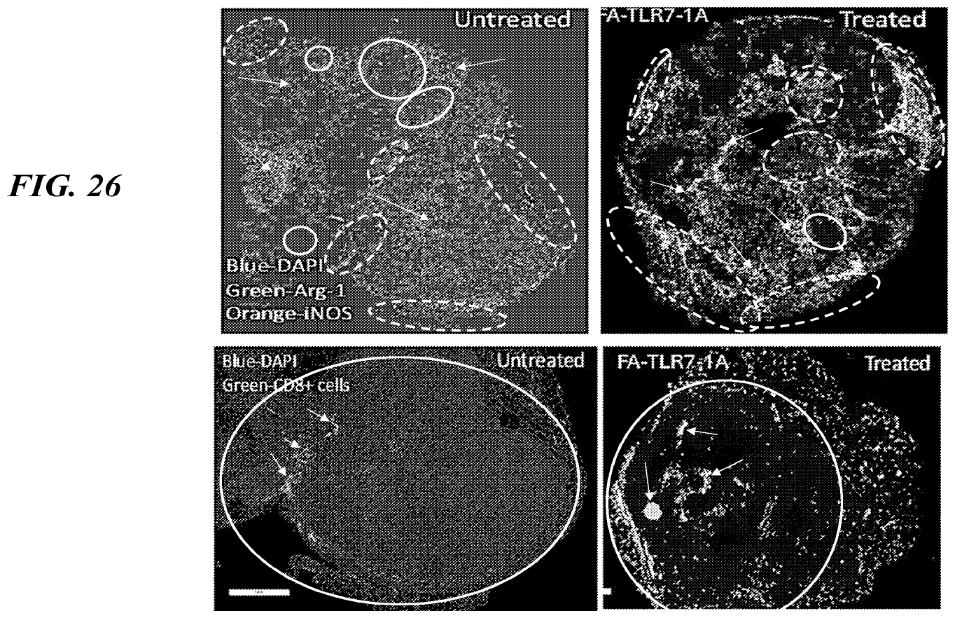

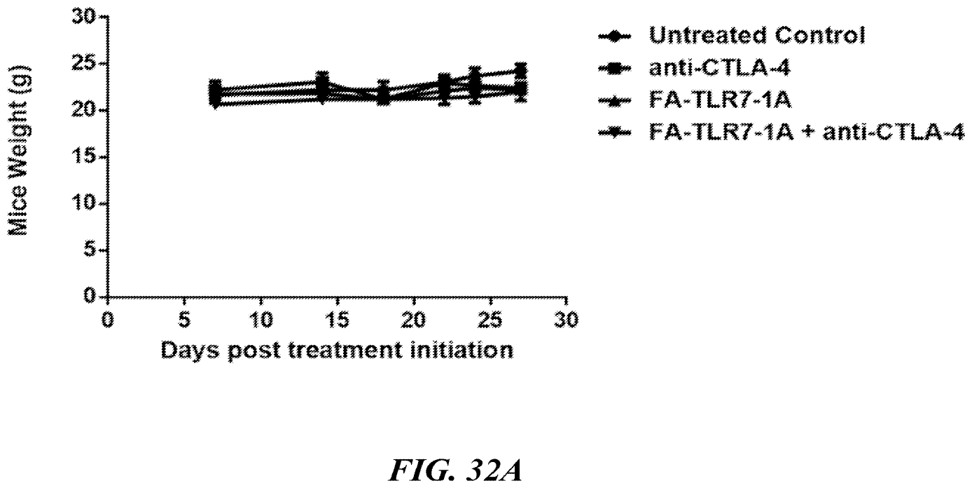

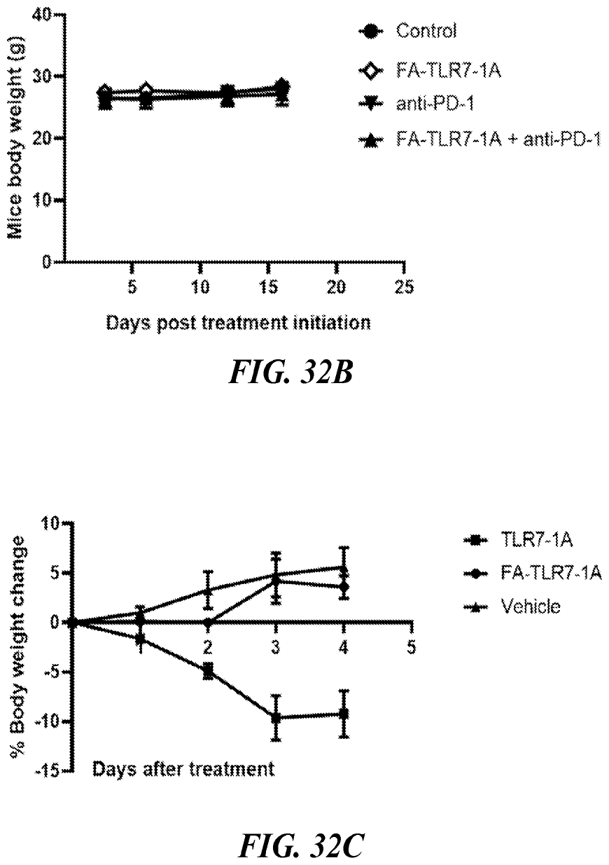

Tumor volume was concurrently measured with calipers using the formula (a×b²)/2 (a being the largest and b being the smallest diameter of the tumor) and the tumors were subsequently digested using mouse tumor dissociation kit (Cat #130-096-730, Miltenyi Biotech. Bergisch Gladbach, DE) according to manufacturer's protocol. Digested tumor cells were resuspended in 1×RBC lysis buffer (Cat #420301, BioLegend, San Diego, CA) for 10 minutes, washed and filtered through 70 mm filters, and resuspended in 2% FBS in PBS to obtain single-cell suspensions. The resulting single cell suspensions were stained for antibodies and the samples were then analyzed by flow cytometry. The results were plotted using GraphPad prism software and immunohistochemistry was performed on the 4T1 tumor tissues post-treatment (FIGS. 24A-24D-FIG. 26). In addition, change in mice body weight during the combination therapy treatment was monitored (FIG. 32A).

### Example 17

### Combination Therapy of Folate-TLR7-1A (Compound 1000) with Checkpoint Inhibitors in MC38 Tumor Model

Six- to eight-week-old male C57BL/6 mice (Jackson Laboratory. Bar Harbor, ME) were housed in a sterile environment on a standard 12-hour light-dark cycle and maintained on folate deficient diet. Upon arrival, the mice were transferred to a folic acid-deficient diet (TD.95247, Envigo Ltd., Indianapolis, IN).

A MC38 cell line was cultured in DMEM with sodium pyruvate and L-glutamine supplemented with 10% FBS, 1% non-essential amino acid (NEAA) cell culture supplement, 1% penicillin/streptomycin, gentamycin and 1% 4-(2-hydroxyethyl)-1-piperazineethanesulfonic acid (HEPES) at 37° C., in a humidified environment with 5% CO₂. The study was performed with 10 passages of thawing the cells from frozen stocks.

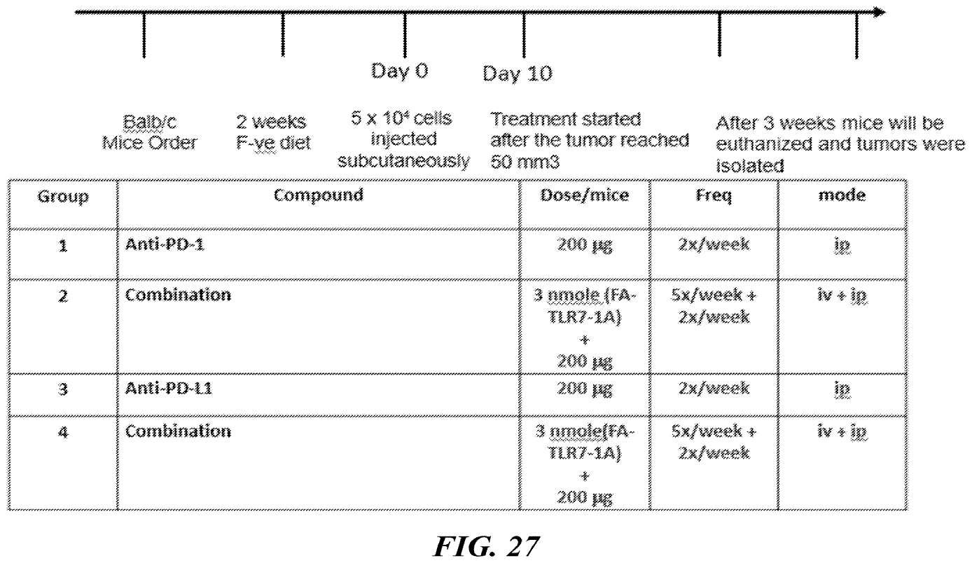

On day 14, 0.5 million MC38 cells were suspended in 100 pl PBS and injected subcutaneously into the right flank of the mice. When the tumor volume reached ~50 mm³, the mice were randomly grouped based on their tumor volume and treated with folate-TLR7-1A (Compound 1000), folate-TLR7-1A (Compound 1000)+anti-PD-1 and anti-PD-1 antibody. Anti-PD-1 antibody was given by intraperitoneal (i.p.) injection on days 3, 5, 10, and 12 at a dose of 100 g/mouse. Control groups received PBS on the same schedule. FIG. 27 outlines the study methodology.

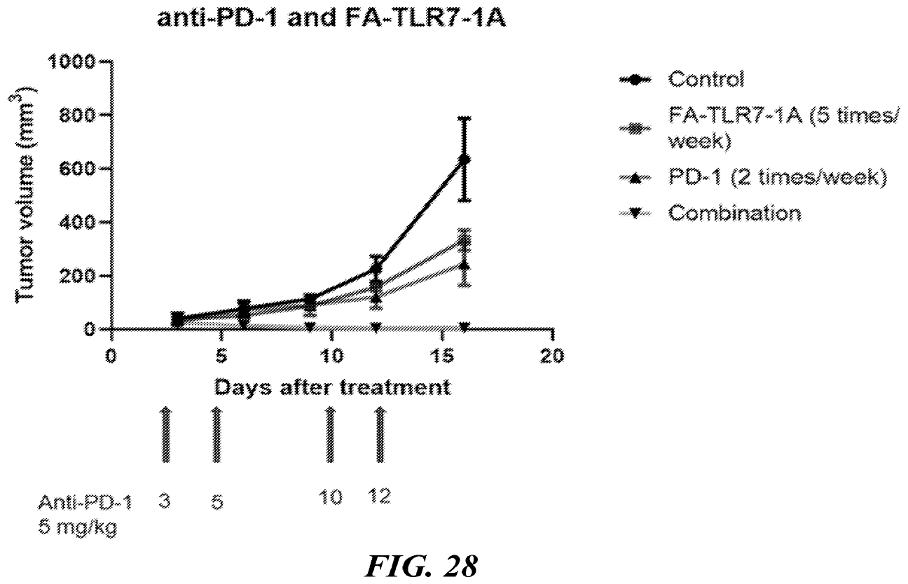

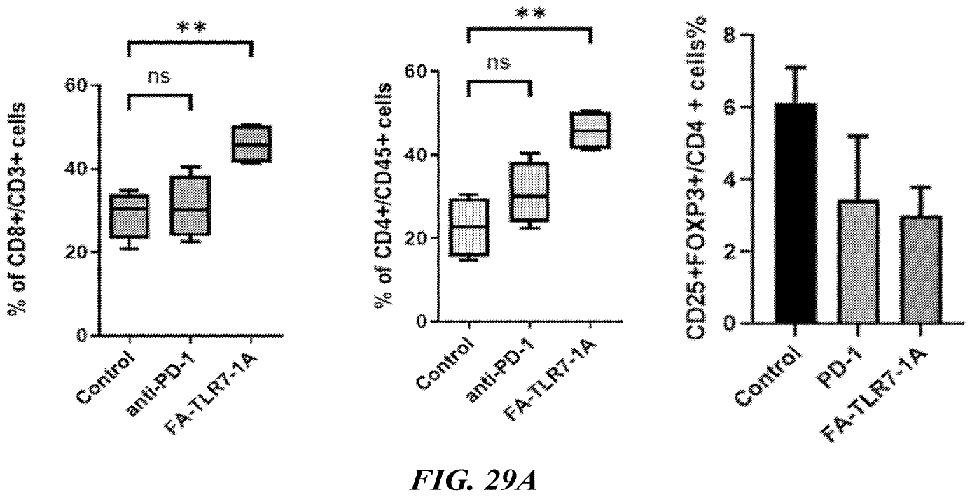

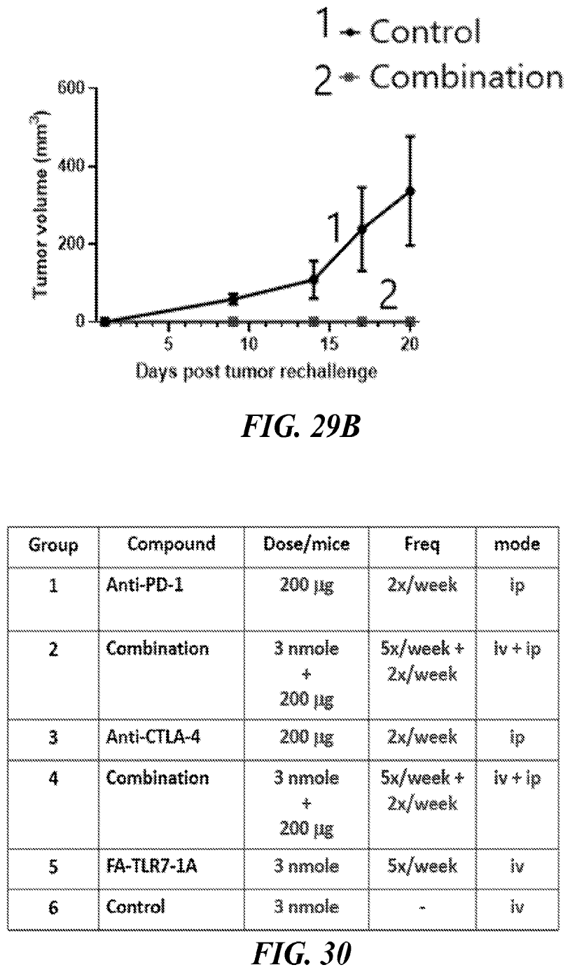

Tumor volume was concurrently measured with calipers using the formula (a×b²)/2 (a being the largest, and b being the smallest diameter of the tumor), and the tumors were subsequently digested using mouse tumor dissociation kit (Cat #130-096-730, Miltenyi Biotech, Bergisch Gladbach, DE) according to manufacturer's protocol. Digested tumor cells were resuspended in 1×RBC lysis buffer (Cat #420301, BioLegend, San Diego, CA) for 10 minutes, washed and filtered through 70 mm filters, and resuspended in 2% FBS in PBS to obtain single-cell suspensions. The resulting single cell suspensions were stained for antibodies and the samples were then analyzed by flow cytometry. The results were plotted using GraphPad prism software (FIG. 28 and FIG. 29A).

Thereafter, 0.5 million MC38 cells in 100 pl PBS were subcutaneously injected into the left flank of both the healthy and cured mice from the above-described experiment. Tumor volume was concurrently measured with calipers using the formula (a×b²)/2 (a being the largest, and b being the smallest diameter of the tumor) and plotted using GraphPad prism software (FIG. 29B). In addition, change in mice body weight during the combination therapy treatment was monitored (FIG. 32B).

### Example 18

### Combination Therapy of Folate-TLR7-1A with Checkpoint Inhibitors in LL/2 Lewis Lung Carcinoma Tumor Model

LL/2 (Lewis lung) carcinoma models were then assessed. The five-year lung cancer survival rate of Lewis lung cancer is only around 20%, which is considerably lower than other cancers. If lung cancer is detected early, the five-year survival rate climbs to about 56%, but only about 16% of lung cancers are detected early enough to achieve this increased survival rate. For later stage detection, the five-year survival rate can be as slow as 5%. As such, it would be extremely beneficial to identify a treatment for Lewis lung carcinoma that is effective and non-toxic.

Six- to eight-week-old male C57BL/6 mice (Jackson Laboratory, Bar Harbor, ME) were housed in a sterile environment on a standard 12-hour light-dark cycle and maintained on folate deficient diet. Upon arrival, the mice were transferred to a folic acid-deficient diet.

LL/2 (LLC1) Lewis lung carcinoma cells were cultured in DMEM with sodium pyruvate and L-glutamine supplemented with FBS, glucose, penicillin/streptomycin and sodium bicarbonate at 37° C., in a humidified environment with 5% CO₂. Cells were maintained in 75 cm² tissue culture flasks. The study was performed with 10 passages of thawing the cells from frozen stocks.

On day 14, 0.5 million LL/2 cells were suspended in 100 pl PBS and injected subcutaneously into the right flank of the mice. When the tumor volume reached ~50 mm, the mice were randomly grouped based on their tumor volume and treated with folate-TLR7-1A (Compound 1000), folate-TLR7-1A (Compound 1000)+anti-CTLA-4 and anti-CTLA-4 antibody. Anti-CTLA-4 antibody was given by intraperitoneal (i.p.) injection on days 3, 6, 10, 13, and 17 at a dose of 200 g/mouse. Control groups received PBS on the same schedule. FIG. 30 shows a summary of the study methodology.

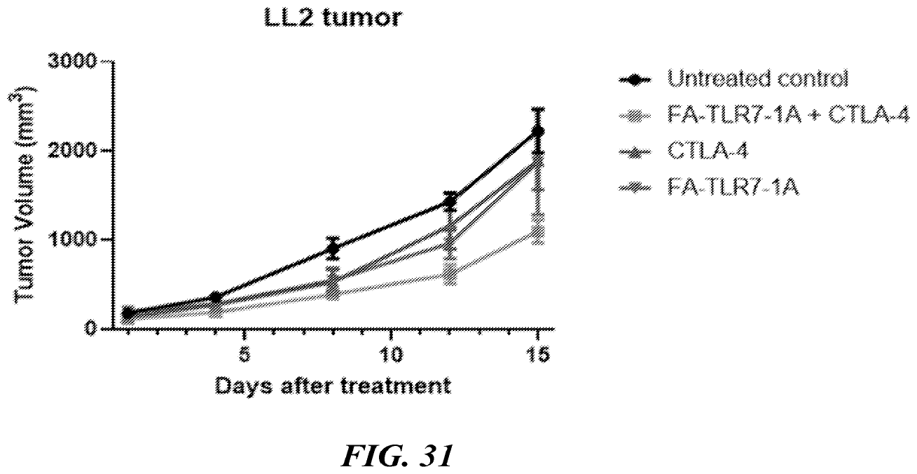

Tumor volume was concurrently measured with calipers using the formula (a×b²)/2 (a being the largest and b being the smallest diameter of the tumor) and the tumors were subsequently digested using mouse tumor dissociation kit (Cat #130-096-730, Miltenyi Biotech, Bergisch Gladbach, DE) according to manufacturer's protocol. Digested tumor cells were resuspended in 1×RBC lysis buffer (Cat #420301, BioLegend, San Diego, CA) for 10 minutes, washed and filtered through 70 mm filters, and resuspended in 2% FBS in PBS to obtain single-cell suspensions. The resulting single cell suspensions were stained for antibodies and the samples were then analyzed by flow cytometry. The results were plotted using GraphPad prism software (FIG. 31).

### Example 19

Healthy mice were tail vein injected with 60 nmoles/mice/day of Compound 1 (TLR7-1A) or Compound 1000 (FA-LTR7-1A), and the change in mice body weight was assessed each day for 4 days post-treatment as a measure of systemic toxicity following chronic administration of the compounds as described (FIG. 32C).

### Enumerated Embodiments of the Claims

Clause 1. A method of treating a subject with cancer, the method comprising:
administering a first therapy to a subject, the first therapy comprising:
   at least one compound or pharmaceutically acceptable salt or hydrate thereof comprising a radical of an immune modulator attached, via a linker, to a folate ligand or functional fragment or analog thereof, or
   a composition comprising the at least one compound and one or more pharmaceutically acceptable carriers, adjuvants, diluents, excipients, and/or vehicles, or combinations thereof; and
administering a second therapy to the subject, the second therapy comprising one or more immune checkpoint inhibitors.
Clause 2. The method of clause 1, wherein administering the second therapy to the subject further comprises administering a therapeutically effective amount of one or more immune checkpoint inhibitors.
Clause 3. The method of clause 1, wherein administering the at least one compound of the first therapy reprograms M2-type macrophages of the subject to M1-type macrophages and enhances a potency of the one or more immune checkpoint inhibitors of the second therapy relative to a baseline potency of the one or more immune checkpoint inhibitors.
Clause 4. The method of clause 1, wherein the radical of the immune modulator or pharmaceutically acceptable salt or hydrate thereof comprises a toll-like receptor (TLR) 3 agonist, a TLR 7 agonist, a TLR 8 agonist, a TLR9 agonist, or a TLR 7/8 agonist.
Clause 5. The method of clause 1, wherein the at least one compound is or comprises the formula:

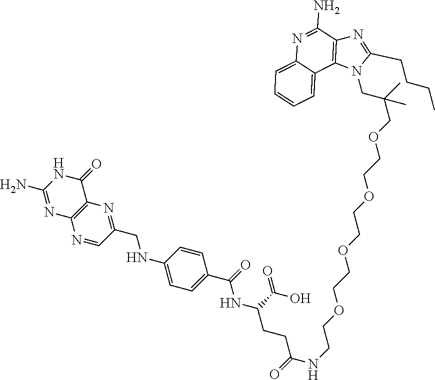

or is a pharmaceutically acceptable salt or hydrate thereof.
Clause 6. The method of clause 1, wherein the at least one compound has or comprises the following formula:

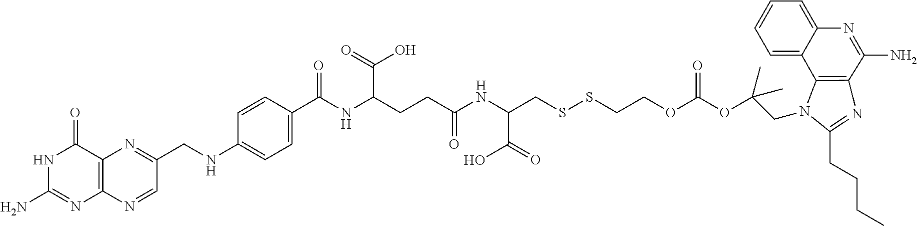 [SMILES: CCCCc1nc2c(N)nc3ccccc3c2n1CC(C)(C)OC(=O)OCCSSCC(NC(=O)CCC(NC(=O)c1ccc(NCc2cnc3nc(N)[nH]c(=O)c3n2)cc1)C(=O)O)C(=O)O]

or is a pharmaceutically acceptable salt or hydrate thereof
Clause 7. The method of any one of clauses 1-4, wherein the radical of the immune modulator is or comprises a TLR agonist of Formula X or XX, or is a pharmaceutically acceptable salt or hydrate of Formula X or XX:

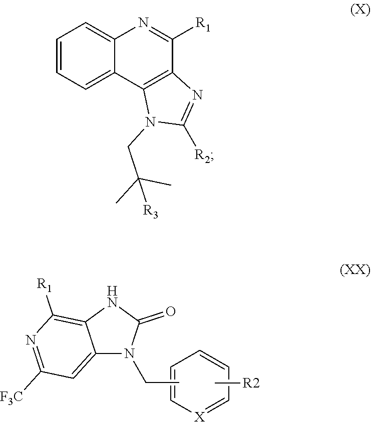

wherein, in Formulas X and XX:
R₁ is —NH₂ or —NH—R_{1X},
R₂ is an H, an alkyl, an alkenyl, an alkynyl, an alicyclic, an aryl, a biaryl, a heteroaryl, —NH—R_{2X}, —O—R_{2X}, —S—R_{2X},

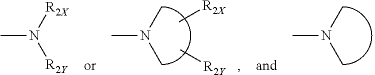

is a 3-10 membered N-containing non-aromatic mono- or bicyclic heterocycle, wherein:
in Formula X, R₃ is —OH, —SH, —NH₂ or —NH—R_{1X};
in Formula XX, X is a CH or an N; and
each of R_{1X}, R_{2X}, and R_{2Y} is independently selected from the group consisting of an H, an alkyl, an alkenyl, an alkynyl, an alicyclic, an aryl, a biaryl, and a heteroaryl.
Clause 8. The method of clause 1, wherein the at least one compound of the first therapy comprises:

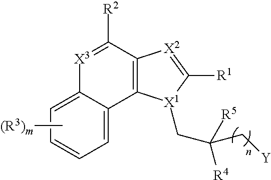

or is a pharmaceutically acceptable salt or hydrate thereof, wherein, in Formula 2-I:
R¹, R³, R⁴, and R⁵ are each independently a hydrogen (H), an alkyl, an alkoxyl, an alkenyl, an alkynyl, an alicyclic, an aryl, a biaryl, a halo, a heteroaryl, —COR^{2x},

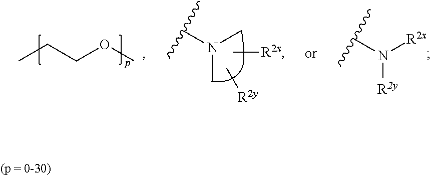

R² is a H, —OH, —NH₂, —NHR^{2x}, N₃, —NH—CH₂—NH₂, —CONH₂, —SO₂NH₂, —NH—CS—NH₂,

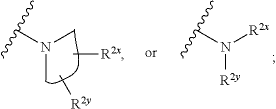

Y is a point of attachment to the linker and/or targeting ligand of the at least one compound and comprises a H, —OH, —NH₂, —NHR^{2x}, —O—R^{2X}, —SO—R^{2x}, —SH, —SO₃H, —N₃, —CHO, —COOH, —CONH₂, —COSH, —COR^{2x}, —SO₂NH₂, alkenyl, alkynyl, alkoxyl, —NH—CH₂—NH₂, —CONH₂, —SO₂NH₂, —NH—CS—NH₂,

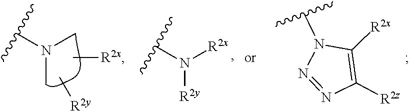

where:
   each of R^{2x} and R^{2y} is independently selected from the group consisting of H, —OH, —CH₂—OH, —NH₂, —CH₂—NH₂, —COOMe, —COOH, —CONH₂, —COCH₃, alkyl, alkenyl, alkynyl, alicyclic, aryl, biaryl, and heteroaryl, and each R^{2Z} is independently selected from the group consisting of —NH₂, —NR^{2q}R^{2q′}, —O—R^{2q}, —SO—R^{2q}, and —COR^{2q}; wherein each of R^{2q} and R^{2q′} is independently alkyl or H; and

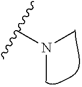 [SMILES: *N(C)C]

is a 3-10 membered N-containing heterocycle that is non-aromatic, mono- or bicyclic;
wherein, in Formula 2-I, each of X¹, X², and X³ is independently CR^{q} or N, and each R^{q} is independently H, halogen, or an optionally substituted alkyl; and
wherein, in Formula 2-I, n is 0-30, and m is 0-4.
Clause 9. The method of any one of the foregoing clauses, wherein the subject is experiencing, or at risk for experiencing, a cancer or a relapse of a cancer, and the step of administering the first therapy further comprises administering or applying to the subject a therapeutically effective amount of the at least one compound or pharmaceutically acceptable salt or hydrate thereof.
Clause 10. The method of any one of the foregoing clauses, wherein the first therapy is administered to the subject intravenously, orally, intramuscularly, intraperitoneally, topically or by inhalation.
Clause 11. The method of clause 3, wherein the M2-type macrophages of the subject are myeloid-derived suppressor cells (MDSCs), tumor-associated macrophages (TAMs), or both MDSCs and TAMs.
Clause 12. The method of any one of the foregoing clauses, wherein the subject has programmed death-ligand 1 (PD-L1), programmed death 1 (PD-1), or cytotoxic T-lymphocyte-associated antigen 4 (CTLA-4) positive tumors.
Clause 13. The method of any one of the foregoing clauses, performed to treat a relapse of a cancer in the subject or resistance to checkpoint blockade therapy in the subject.
Clause 14. The method of any one of the foregoing clauses, wherein subject is a human, a mouse, or any other mammal.
Clause 15. The method of clause 1, wherein the one or more immune checkpoint inhibitors of the second therapy each comprises a small molecule or other agent that disrupts an immune checkpoint of a cell of the subject.
Clause 16. The method of any one of clauses 1-14, wherein the one or more immune checkpoint inhibitors of the second therapy each comprises a small molecule or other agent that disrupts an immune checkpoint of a cell of the subject.
Clause 17. The method of any one of the foregoing clauses, wherein the one or more immune checkpoint inhibitors of the second therapy are each independently selected from the group consisting of pembrolizumab, nivolumab, ipilimumab, cemiplimab, atezolizumab, avelumab, durvalumab, pidilizumab, monoclonal antibody MEDI-0680, monoclonal antibody REGN2810, or fusion protein AMP-224 that targets PD-1, ociperlimab, islelizumab, a combination of ociperlimab and islelizumab, BMS-936559/MDX-1105, MPDL3280A/RG7446/atezolizumab, MSB0010718C/avelumab, or MED14736/durvalumab, tiragolumab, zimberelimab, tremelimumab, relatlimab, monoclonal antibody IMP321, nivolumab, etigilimab, domvanalimab, tiragolumab (RG6058), vibostolimab, avelumab, and durvalumab.
Clause 18. The method of any one of the foregoing clauses, wherein the radical of the immune modulator comprises a TLR agonist having the following formula:

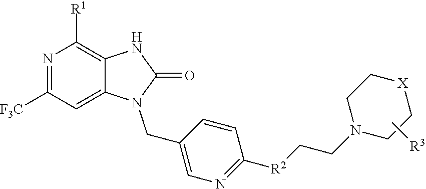

wherein:
R¹ is an amine group,
R² is a single bond —NH—,
R³ is an H, an alkyl, a hydroxy group, or any other substituted group thereof,
X is a CH₂, NH, O, or S, and
the linker is attached at R¹, R² or R³.
Clause 19. The method of any one of clauses 1-6, 8, 11, and 15, wherein the linker is a non-releasable linker.
Clause 20. The method of any one of clauses 1-6, 8, 11, and 15, wherein the linker is a releasable linker.
Clause 21. The method of any one of clauses 1-6, 8, 11, and 15, wherein the linker of the at least one compound or pharmaceutically acceptable salt or hydrate of the first therapy comprises a polyethylene glycol (PEG) linker or a PEG derivative linker and is a non-releasable linker.
Clause 22. The method of any one of the foregoing clauses, wherein the one or more immune checkpoint inhibitors of the second therapy inhibit an immune checkpoint of a cell selected from a group consisting of PD-1, PD-L1, CTLA-4, V-domain Ig suppressor of T cell activation (VISTA), lymphocyte activating 3 (LAG3), T cell immunoglobulin and mucin domain-containing protein 3 (TIM3), T cell immunoreceptor with Ig and ITIM domains (TIGIT), programmed death ligand 2 (PD-L2), indoleamine 2,3-deioxygenase (IDO), arginase-1 (AGR1), B7 family inhibitory ligand B7-H3 (B7-H3), B7 family inhibitory ligand B7-H4 (B7-H4), 2B4 (cluster of differentiation 244), B- and T-lymphocyte attenuator (BTLA), adenosine A2A receptor (A2aR), and/or a member of the family of killer cell immunoglobulin-like receptors (KIRs) such as KIRs and C-type lectin receptors, and signal transducer and activator of transcription (STAT3).
Clause 23. The method of any one of the foregoing clauses, wherein the first and second therapies are administered simultaneously or sequentially (in either order).
Clause 24. The method of any one of the foregoing clauses, wherein administering the at least one compound or pharmaceutically acceptable salt or hydrate of the first therapy activates anti-tumor cells or an anti-inflammatory signaling cascade in the subject.
Clause 25. A method of enhancing a potency of one or more immune checkpoint inhibitors administered to a subject comprising:
administering, to the subject, one or more compounds or pharmaceutically acceptable salts or hydrates thereof comprising a radical of an immune modulator attached, via a linker, to a folate ligand or functional fragment or analog thereof; and
contacting a targeted cell of the subject with the one or more compounds or pharmaceutically acceptable salts or hydrates to reprogram M2-type macrophages of the subject to M1-type macrophages.
Clause 26. The method of clause 25, wherein the radical of the immune modulator is a TLR 3 agonist, a TLR 7 agonist, a TLR 8 agonist, a TLR9 agonist, or a TLR 7/8 agonist.
Clause 27. The method of claim 25, wherein the radical of the immune modulator is or comprises a TLR7 agonist and the linker is a releasable linker.
Clause 28. The method of clause 25, wherein the radical of the immune modulator is or comprises a TLR7 agonist and the linker is a non-releasable linker.
Clause 29. The method of clause 25, wherein the one or more compounds or pharmaceutically acceptable salts or hydrates thereof is or comprises the formula:

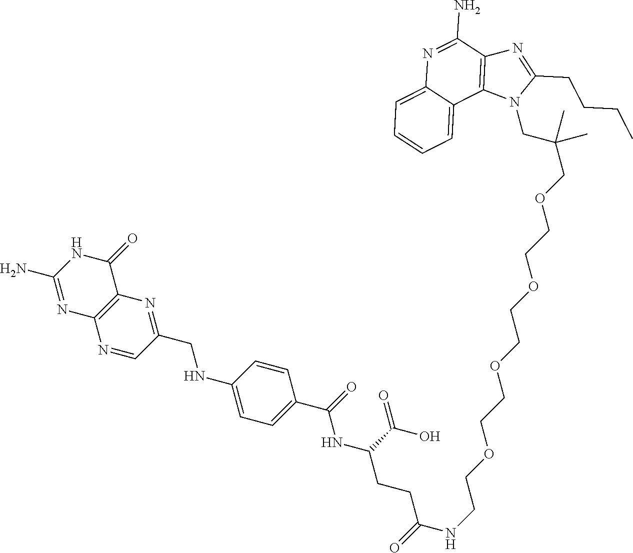

or is a pharmaceutically acceptable salt or hydrate thereof.
Clause 30. The method of clause 25, 16, or 29, wherein the linker is anon-releasable linker.
Clause 31. The method of any one of clauses 25-30, wherein the cancerous disease state comprises a cancer recurrence.
Clause 32. The method of clause 24, wherein the anti-tumor cells are T cells, macrophages, or both T cells and macrophages.
Clause 33. A combination for use in the treatment of cancer in a subject, the combination comprising:
a first medicament comprising:
   at least one compound or pharmaceutically acceptable salt or hydrate thereof comprising a radical of an immune modulator attached, via a linker, to a folate ligand or functional fragment or analog thereof, wherein the radical of the immune modulator targets
   a pattern recognition receptor or a damage associated molecular pattern (DAMP), or
   a composition comprising the at least one compound or pharmaceutically acceptable salt or hydrate thereof and one or more pharmaceutically acceptable carriers, adjuvants, diluents, excipients, and/or vehicles; and
a second medicament comprising one or more immune checkpoint inhibitors.
Clause 34. The combination of clause 33, wherein the radical of the immune modulator comprises a TLR 3 agonist, a TLR 7 agonist, a TLR 8 agonist, a TLR9 agonist, or a TLR 7/8 agonist.
Clause 35. The combination of clause 33, wherein at least one compound has or comprises the following formula:

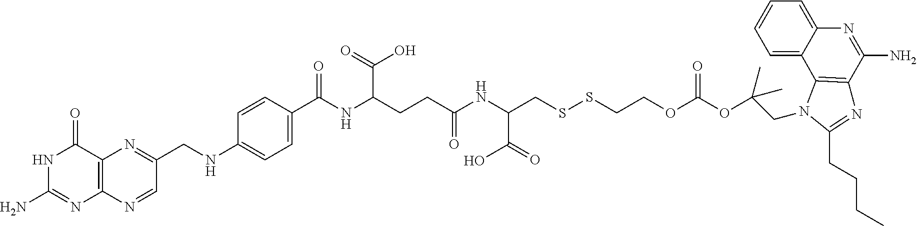 [SMILES: CCCCc1nc2c(N)nc3ccccc3c2n1CC(C)(C)OC(=O)OCCSSCC(NC(=O)CCC(NC(=O)c1ccc(NCc2cnc3nc(N)[nH]c(=O)c3n2)cc1)C(=O)O)C(=O)O]

or is a pharmaceutically acceptable salt or hydrate thereof.
Clause 36. The combination of clause 33, wherein the radical of the immune modulator comprises a TLR agonist of Formula X or XX, or is a pharmaceutically acceptable salt or hydrate of Formula X or XX:

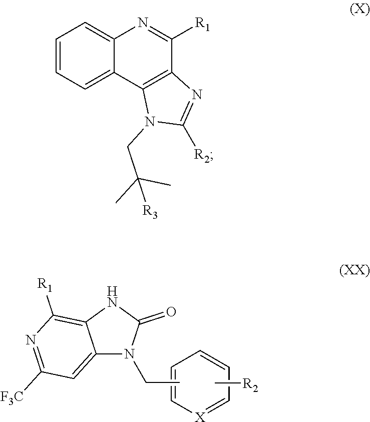

wherein, in Formulas X and XX:
R₁ is —NH₂ or —NH—R_{1X},
R₂ is an H, an alkyl, an alkenyl, an alkynyl, an alicyclic, an aryl, a biaryl, a heteroaryl, —NH—R_{2X}, —O—R_{2X}, —S—R_{2X},

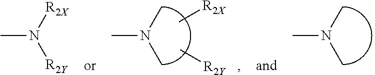

is a 3-10 membered N-containing non-aromatic mono- or bicyclic heterocycle, wherein:
in Formula X, R₃ is —OH, —SH, —NH₂ or —NH—R_{1X};
in Formula XX, X is a CH or an N; and
each of R_{1X}, R_{2X}, and R_{2Y} is independently selected from the group consisting of an H, an alkyl, an alkenyl, an alkynyl, an alicyclic, an aryl, a biaryl, and a heteroaryl.
Clause 37. The combination of clause 33, wherein the at least one compound of the first medicament comprises:

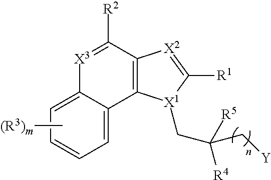

or is a pharmaceutically acceptable salt or hydrate thereof:
wherein, in Formula 2-I:
R¹, R³, R⁴, and R⁵ are each independently a hydrogen (H), an alkyl, an alkoxyl, an alkenyl, an alkynyl, an alicyclic, an aryl., a biaryl, a halo, a heteroaryl, —COR^{2x},

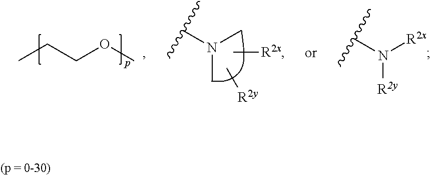

R² is a H, —OH, —NH₂, —NHR^{2x}, N₃, —NH—CH₂—NH₂, —CONH₂, —SO₂NH₂, —NH—CS—NH₂,

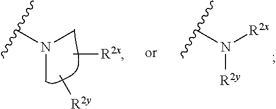

Y is a point of attachment to the linker and/or the targeting ligand of the at least one compound and comprises a H, —OH, —NH₂, —NHR^{2x}, —O—R^{2x}, —SO—R^{2x}, —SH, —SO₃H, —N₃, —CHO, —COOH, —CONH₂, —COSH, —COR^{2x}, —SO₂NH₂, alkenyl, alkynyl, alkoxyl, —NH—CH₂—NH₂, —CONH₂, —SO₂NH₂, —NH—CS—NH₂,

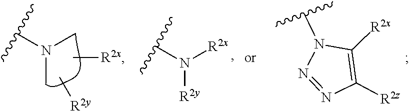

where:
   each of R^{2x} and R^{2y} is independently selected from the group consisting of H, —OH, —CH₂—OH, —NH₂, —CH₂—NH₂, —COOMe, —COOH, —CONH₂, —COCH₃, alkyl, alkenyl, alkynyl, alicyclic, aryl, biaryl, and heteroaryl, and each R^{2z} is independently selected from the group consisting of —NH₂, —NR^{2q}R^{2q′}, —O—R^{2q}, —SO—R^{2q}, and —COR^{2q}; wherein each of R^{2q} and R^{2q′} is independently alkyl or H; and

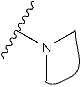 [SMILES: *N(C)C]

is a 3-10 membered N-containing heterocycle that is non-aromatic, mono- or bicyclic;
wherein, in Formula 2-I, each of X¹, X², and X³ is independently CR^{q} or N, and each R is independently H, halogen, or an optionally substituted alkyl; and wherein, in Formula 2-I, n is 0-30, and m is 0-4.
Clause 38. The combination of clause 33, wherein the radical of the immune modulator of the first medicament comprises a TLR agonist having the following formula or a pharmaceutically acceptable salt thereof:

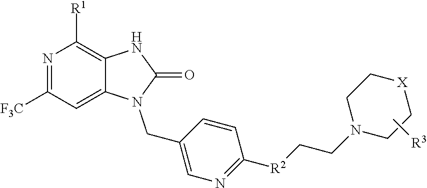

wherein:
R¹ is an amine group,
R² is a single bond —NH—.
R³ is an H, an alkyl, a hydroxy group, or any other substituted group thereof,
X is a CH₂, NH, O, or S, and
the linker is attached at R¹, R² or R³.
Clause 39. The combination of clause 33, wherein the at least one compound of the first medicament has or comprises the following formula:

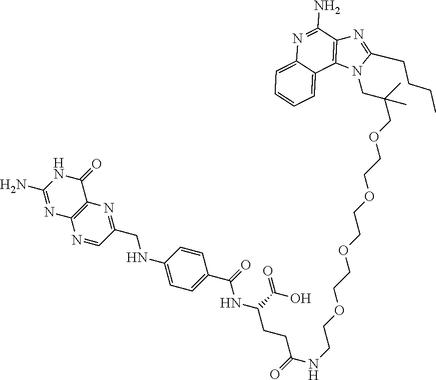

or is a pharmaceutically acceptable salt or hydrate thereof
Clause 40. The combination of clause 33, wherein the linker of the at least one compound or pharmaceutically acceptable salt or hydrate of the first medicament is a releasable linker.
Clause 41. The combination of clause 33, wherein the linker of the at least one compound or pharmaceutically acceptable salt or hydrate of the first medicament is a non-releasable linker.
Clause 42. The combination of clause 33, wherein the linker of the at least one compound or pharmaceutically acceptable salt or hydrate of the first medicament comprises a PEG linker or a PEG derivative linker and is a non-releasable linker.
Clause 43. The combination of any one of clauses 33-42, wherein the subject has a PD-L1, PD-1, or CTLA-4 positive tumor.
Clause 44. The combination of any one of clauses 33-43, wherein the one or more immune checkpoint inhibitors of the second medicament comprise a small molecule or other agent that disrupts an immune checkpoint of a cell of the subject.
Clause 45. The combination of any one of clauses 33-44, wherein the one or more checkpoint inhibitors of the second medicament are each independently selected from the group consisting of pembrolizumab, nivolumab, ipilimumab, cemiplimab, atezolizumab, avelumab, durvalumab, pidilizumab, monoclonal antibody MEDI-0680, monoclonal antibody REGN2810, or fusion protein AMP-224 that targets PD-1, ociperlimab, islelizumab, a combination of ociperlimab and islelizumab, BMS-936559/MDX-1105, MPDL3280A/RG7446/atezolizumab, MSB0010718C/avelumab, or MEDI4736/durvalumab, tiragolumab, zimberelimab, tremelimumab, relatlimab, monoclonal antibody IMP321, nivolumab, etigilimab, domvanalimab, tiragolumab (RG6058), vibostolimab, avelumab, and durvalumab.
Clause 46. The combination of any one of clauses 33-45, wherein the one or more immune checkpoint inhibitors of the second medicament are one or more agents that bind to or inhibit an immune checkpoint of a cell selected from a group consisting of PD-1, PD-L1, CTLA-4, VISTA, LAG3, TIM3, TIGIT, PD-L2, IDO, AGR1, B7-H3, B7-H4, 2B4, BTLA, A2aR, and/or a member of the family of KIRs such as KIRs and C-type lectin receptors, and STAT3.
Clause 47. The combination of any one of clauses 33-46, wherein the cancer comprises a cold tumor, a hot tumor, or an immune desert tumor.
Clause 48. The combination of any one of clauses 33-47 used for enhancing a potency of one or more immune checkpoint inhibitors administered to a subject with cancer and/or for treating a cancer.

1. A method of treating a subject with cancer, the method comprising:
administering a first therapy to a subject, the first therapy comprising:
at least one compound or pharmaceutically acceptable salt or hydrate thereof comprising a radical of an immune modulator attached, via a linker, to a folate ligand or functional fragment or analog thereof, or
a composition comprising the at least one compound and one or more pharmaceutically acceptable carriers, adjuvants, diluents, excipients, and/or vehicles, or combinations thereof; and
administering a second therapy to the subject, the second therapy comprising one or more immune checkpoint inhibitors.

2. The method of claim 1, wherein administering the second therapy to the subject further comprises administering a therapeutically effective amount of one or more immune checkpoint inhibitors.

3. The method of claim 1, wherein administering the at least one compound of the first therapy reprograms M2-type macrophages of the subject to M1-type macrophages and enhances a potency of the one or more immune checkpoint inhibitors of the second therapy relative to a baseline potency of the one or more immune checkpoint inhibitors.

4. The method of claim 1, wherein the radical of the immune modulator or pharmaceutically acceptable salt or hydrate thereof comprises a toll-like receptor (TLR) 3 agonist, a TLR 7 agonist, a TLR 8 agonist, a TLR9 agonist, or a TLR 7/8 agonist.

5. The method of claim 1, wherein the at least one compound has the formula: 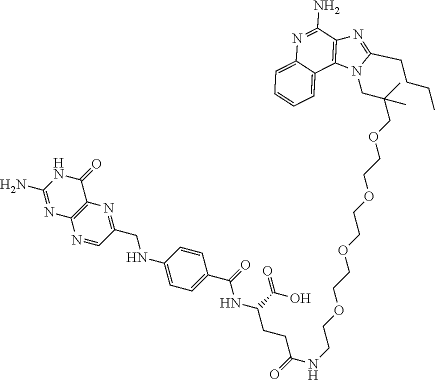
or is a pharmaceutically acceptable salt or hydrate thereof.

6. The method of claim 1, wherein the at least one compound has the following formula: 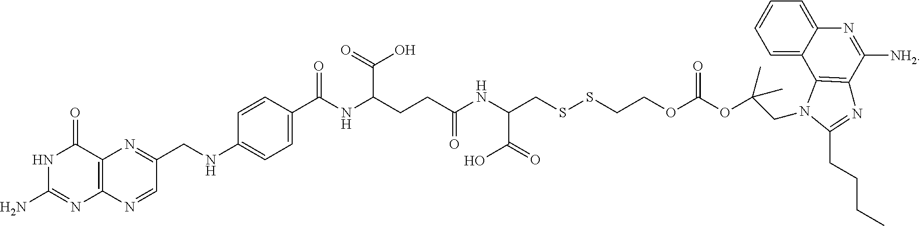 [SMILES: CCCCc1nc2c(N)nc3ccccc3c2n1CC(C)(C)OC(=O)OCCSSCC(NC(=O)CCC(NC(=O)c1ccc(NCc2cnc3nc(N)[nH]c(=O)c3n2)cc1)C(=O)O)C(=O)O]
or is a pharmaceutically acceptable salt or hydrate thereof.

7. The method of any one of claims 1-4, wherein the radical of the immune modulator is or comprises a TLR agonist of Formula X or XX, or is a pharmaceutically acceptable salt or hydrate of Formula X or XX: 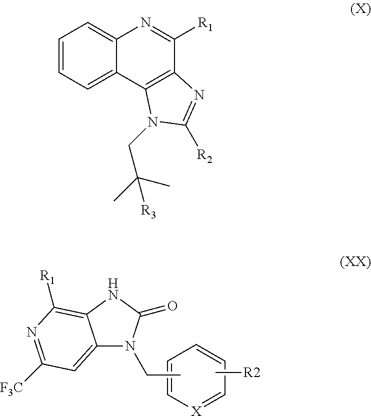
wherein, in Formulas X and XX:
R₁ is —NH₂ or —NH—R_{1X},
R₂ is an H, an alkyl, an alkenyl, an alkynyl, an alicyclic, an aryl, a biaryl, a heteroaryl, —NH—R_{2X}, —O—R_{2X}, —S—R_{2X}, 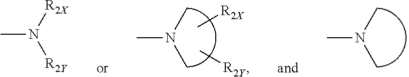
is a 3-10 membered, N-containing, non-aromatic, mono- or bicyclic heterocycle, wherein:
in Formula X, R₃ is —OH, —SH, —NH₂ or —NH—R_{1X};
in Formula XX, X is a CH or an N; and
each of R_{1X}, R_{2X}, and R_{2Y} is independently selected from the group consisting of an H, an alkyl, an alkenyl, an alkynyl, an alicyclic, an aryl, a biaryl, and a heteroaryl.

8. The method of claim 1, wherein the at least one compound of the first therapy comprises: 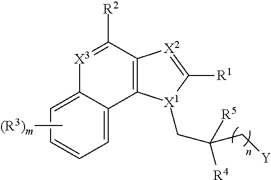
or is a pharmaceutically acceptable salt or hydrate thereof, wherein, in Formula 2-I:
R¹, R³, R⁴, and R⁵ are each independently a hydrogen (H), an alkyl, an alkoxyl, an alkenyl, an alkynyl, an alicyclic, an aryl, a biaryl, a halo, a heteroaryl, —COR^{2x}, 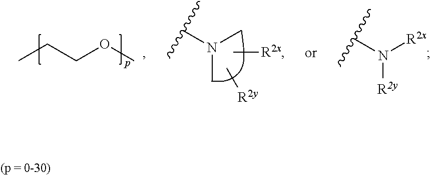
R² is a H, —OH, —NH₂, —NHR^{2x}, N₃, —NH—CH₂—NH₂, —CONH₂, —SO₂NH₂, —NH—CS—NH₂, 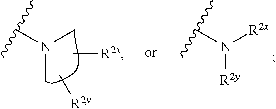
Y is a point of attachment to the linker and/or targeting ligand of the at least one compound and comprises a H, —OH, —NH₂, —NHR^{2x}, —O—R^{2x}, —SO—R^{2x}, —SH, —SO₃H, —N₃, —CHO, —COOH, —CONH₂, —COSH, —COR^{2x}, —SO₂NH₂, alkenyl, alkynyl, alkoxyl, —NH—CH₂—NH₂, —CONH₂, —SO₂NH₂, —NH—CS—NH₂, 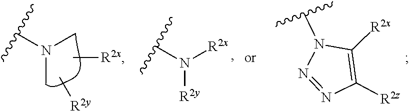
where:
each of R^{2x} and R^{2y} is independently selected from the group consisting of H, —OH, —CH₂—OH, —NH₂, —CH₂—NH₂, —COOMe, —COOH, —CONH₂, —COCH₃, alkyl, alkenyl, alkynyl, alicyclic, aryl, biaryl, and heteroaryl, and each R² is independently selected from the group consisting of —NH₂, —NR^{2q}R^{2q′}, —O—R^{2q}, —SO—R^{2q}, and —COR^{2q}; wherein each of R^{2q} and R^{2q′} is independently alkyl or H; and 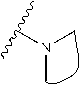 [SMILES: *N(C)C]
is a 3-10 membered N-containing heterocycle that is non-aromatic, mono- or bicyclic;
wherein, in Formula 2-I, each of X¹, X², and X³ is independently CR^{q} or N, and each R^{q} is independently H, halogen, or an optionally substituted alkyl; and
wherein, in Formula 2-I, n is 0-30, and m is 0-4.

9. The method of any one of claims 1-6 and 8, wherein the subject is experiencing, or at risk for experiencing, a cancer or a relapse of a cancer, and the step of administering the first therapy further comprises administering or applying to the subject a therapeutically effective amount of the at least one compound or pharmaceutically acceptable salt or hydrate thereof.

10. The method of any one of claims 1-6 and 8, wherein the first therapy is administered to the subject intravenously, orally, intramuscularly, intraperitoneally, topically or by inhalation.

11. The method of claim 3, wherein the M2-type macrophages of the subject are myeloid-derived suppressor cells (MDSCs), tumor-associated macrophages (TAMs), or both MDSCs and TAMs.

12. The method of any one of claims 1-6, 8, and 11, wherein the subject has programmed death-ligand 1 (PD-L1), programmed death 1 (PD-1), or cytotoxic T-lymphocyte-associated antigen 4 (CTLA-4) positive tumors.

13. The method of any one of claims 1-6, 8, and 11, performed to treat a relapse of a cancer in the subject or resistance to checkpoint blockade therapy in the subject.

14. The method of any one of claims 1-6, 8, and 11, wherein subject is a human, a mouse, or any other mammal.

15. The method of claim 1, wherein the one or more immune checkpoint inhibitors of the second therapy each comprises a small molecule or other agent that disrupts an immune checkpoint of a cell of the subject.

16. The method of any one of claims 1-6, 8, 11, and 15, wherein the one or more immune checkpoint inhibitors of the second therapy each comprises a small molecule or other agent that disrupts an immune checkpoint of a cell of the subject.

17. The method of any one of claims 1-6, 8, 11, and 15, wherein the one or more immune checkpoint inhibitors of the second therapy are each independently selected from the group consisting of pembrolizumab, nivolumab, ipilimumab, cemiplimab, atezolizumab, avelumab, durvalumab, pidilizumab, monoclonal antibody MEDI-0680, monoclonal antibody REGN2810, or fusion protein AMP-224 that targets PD-1, ociperlimab, islelizumab, a combination of ociperlimab and islelizumab, BMS-936559/MDX-1105, MPDL3280A/RG7446/atezolizumab, MSB0010718C/avelumab, or MEDI4736/durvalumab, tiragolumab, zimberelimab, tremelimumab, relatlimab, monoclonal antibody IMP321, nivolumab, etigilimab, domvanalimab, tiragolumab (RG6058), vibostolimab, avelumab, and durvalumab.

18. The method of any one of claims 1-6, 8, 11, and 15, wherein the radical of the immune modulator comprises a TLR agonist having the following formula: 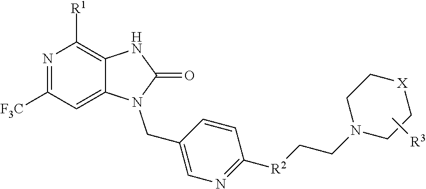
wherein:
R^{r} is an amine group,
R² is a single bond —NH—.
R³ is an H, an alkyl, a hydroxy group, or any other substituted group thereof,
X is a CH₂, NH, O, or S, and
the linker is attached at Rr, R² or R³.

19. The method of any one of claims 1-6, 8, 11, and 15, wherein the linker is a non-releasable linker.

20. The method of any one of claims 1-6, 8, 11, and 15, wherein the linker is a releasable linker.

21. The method of any one of claims 1-6, 8, 11, and 15, wherein the linker of the at least one compound or pharmaceutically acceptable salt or hydrate of the first therapy comprises a polyethylene glycol (PEG) linker or a PEG derivative linker and is a non-releasable linker.

22. The method of any one of claims 1-6, 8, 11, and 15, wherein the one or more immune checkpoint inhibitors of the second therapy inhibit an immune checkpoint of a cell selected from a group consisting of PD-1, PD-L1, CTLA-4, V-domain Ig suppressor of T cell activation (VISTA), lymphocyte activating 3 (LAG3), T cell immunoglobulin and mucin domain-containing protein 3 (TIM3), T cell immunoreceptor with Ig and ITIM domains (TIGIT), programmed death ligand 2 (PD-L2), indoleamine 2,3-deioxygenase (IDO), arginase-1 (AGR1), B7 family inhibitory ligand B7-H3 (B7-H3), B7 family inhibitory ligand B7-H4 (B7-H4), 2B4 (cluster of differentiation 244), B- and T-lymphocyte attenuator (BTLA), adenosine A2A receptor (A2aR), and/or a member of the family of killer cell immunoglobulin-like receptors (KIRs) such as KIRs and C-type lectin receptors, and signal transducer and activator of transcription (STAT3).

23. The method of any one of claims 1-6, 8, 11, and 15, wherein the first and second therapies are administered simultaneously or sequentially in either order.

24. The method of any one of claims 1-23, wherein administering the at least one compound or pharmaceutically acceptable salt or hydrate of the first therapy activates anti-tumor cells or an anti-inflammatory signaling cascade in the subject.

25. A method of enhancing a potency of one or more immune checkpoint inhibitors administered to a subject comprising:
administering, to the subject, one or more compounds or pharmaceutically acceptable salts or hydrates thereof comprising a radical of an immune modulator attached, via a linker, to a folate ligand or functional fragment or analog thereof; and
contacting a targeted cell of the subject with the one or more compounds or pharmaceutically acceptable salts or hydrates to reprogram M2-type macrophages of the subject to M1-type macrophages.

26. The method of claim 25, wherein the radical of the immune modulator is a toll-like receptor (TLR) 3 agonist, a TLR 7 agonist, a TLR 8 agonist, a TLR9 agonist, or a TLR 7/8 agonist.

27. The method of claim 25, wherein the radical of the immune modulator is or comprises a TLR7 agonist and the linker is a releasable linker.

28. The method of claim 25, wherein the radical of the immune modulator is or comprises a TLR7 agonist and the linker is a non-releasable linker.

29. The method of claim 25, wherein the one or more compounds is or comprises the formula: 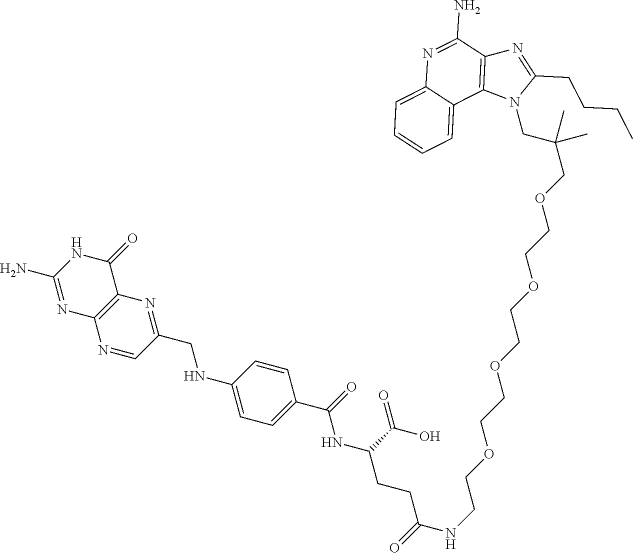
or is a pharmaceutically acceptable salt or hydrate thereof.

30. The method of claim 25 or claim 26, wherein the linker is a non-releasable linker.

31. The method of any one of claims 25-27 and 29, wherein the cancerous disease state comprises a cancer recurrence.

32. The method of claim 24, wherein the anti-tumor cells are T cells, macrophages, or both T cells and macrophages.

33. A combination for use in the treatment of cancer in a subject, the combination comprising:
a first medicament comprising:
at least one compound or pharmaceutically acceptable salt or hydrate thereof comprising a radical of an immune modulator attached, via a linker, to a folate ligand or functional fragment or analog thereof, wherein the radical of the immune modulator targets a pattern recognition receptor or a damage associated molecular pattern (DAMP), or
a composition comprising the at least one compound or pharmaceutically acceptable salt or hydrate thereof and one or more pharmaceutically acceptable carriers, adjuvants, diluents, excipients, and/or vehicles; and
a second medicament comprising one or more immune checkpoint inhibitors.

34. The combination of claim 33, wherein the radical of the immune modulator comprises a toll-like receptor (TLR) 3 agonist, a TLR 7 agonist, a TLR 8 agonist, a TLR9 agonist, or a TLR 7/8 agonist.

35. The combination of claim 33, wherein at least one compound has the following formula: 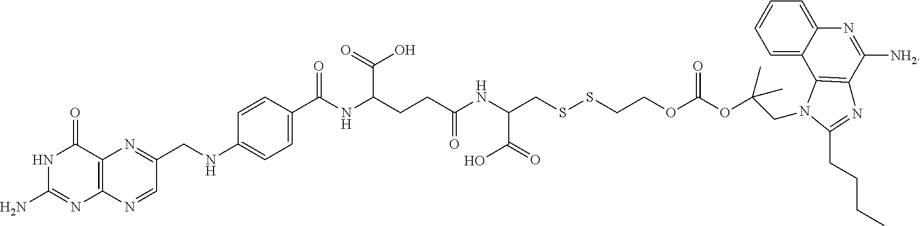 [SMILES: CCCCc1nc2c(N)nc3ccccc3c2n1CC(C)(C)OC(=O)OCCSSCC(NC(=O)CCC(NC(=O)c1ccc(NCc2cnc3nc(N)[nH]c(=O)c3n2)cc1)C(=O)O)C(=O)O]
or is a pharmaceutically acceptable salt or hydrate thereof.

36. The combination of claim 33, wherein the radical of the immune modulator comprises a TLR agonist of Formula X or XX, or is a pharmaceutically acceptable salt or hydrate of Formula X or XX: 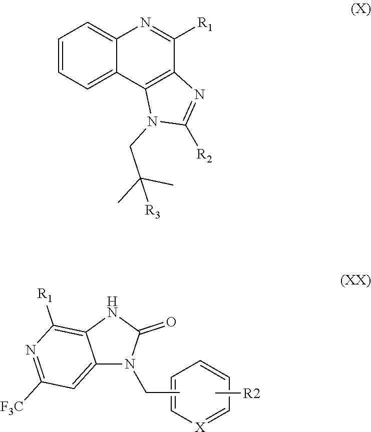
wherein, in Formulas X and XX:
R₁ is —NH₂ or —NH—R_{1X},
R₂ is an H, an alkyl, an alkenyl, an alkynyl, an alicyclic, an aryl, a biaryl, a heteroaryl, —NH—R_{2X}, —O—R_{2X}, —S—R_{2X}, 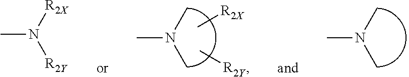
is a 3-10 membered N-containing non-aromatic mono- or bicyclic heterocycle, wherein: in Formula X, R₃ is —OH, —SH, —NH₂ or —NH—R_{1X};
in Formula XX, X is a CH or an N; and
each of R_{1X}, R_{2X}, and R_{2Y} is independently selected from the group consisting of an H, an alkyl, an alkenyl, an alkynyl, an alicyclic, an aryl, a biaryl, and a heteroaryl.

37. The combination of claim 33, wherein the at least one compound of the first medicament comprises: 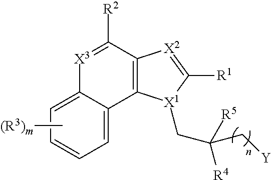
or is a pharmaceutically acceptable salt or hydrate thereof,
wherein, in Formula 2-I:
R¹, R³, R⁴, and R⁵ are each independently a hydrogen (H), an alkyl, an alkoxyl, an alkenyl, an alkynyl, an alicyclic, an aryl, a biaryl, a halo, a heteroaryl, —COR^{2x}, 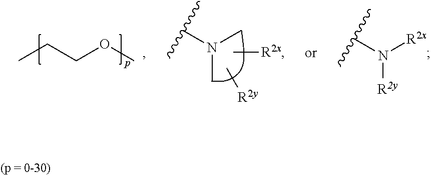
R² is a H, —OH, —NH₂, —NHR^{2x}, N₃, —NH—CH₂—NH₂, —CONH₂, —SO₂NH₂, —NH—CS—NH₂, 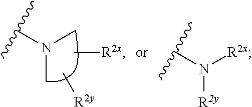
Y is a point of attachment to the linker and/or the targeting ligand of the at least one compound and comprises a H, —OH, —NH₂, —NHR^{2x}, —O—R^{2x}, —SO—R^{2x}, —SH, —SO₃H, —N₃, —CHO, —COOH, —CONH₂, —COSH, —COR^{2x}, —SO₂NH₂, alkenyl, alkynyl, alkoxyl, —NH—CH₂—NH₂, —CONH₂, —SO₂NH₂, —NH—CS—NH₂, 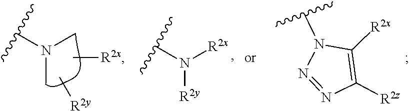
where:
each of R^{2x} and R^{2y} is independently selected from the group consisting of H, —OH, —CH₂—OH, —NH₂, —CH₂—NH₂, —COOMe, —COOH, —CONH₂, —COCH₃, alkyl, alkenyl, alknyl, alicyclic, aryl, biaryl, and heteroaryl, and each R^{2z} is independently selected from the group consisting of —NH₂, —NR^{2q}R^{2q′}, —O—R^{2q}, —SO—R^{2q}, and —COR^{2q}; wherein each of R^{2q} and R^{2q′} is independently alkyl or H; and 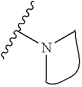 [SMILES: *N(C)C]
is a 3-10 membered N-containing heterocycle that is non-aromatic, mono- or bicyclic;
wherein, in Formula 2-I, each of X¹, X², and X³ is independently CR^{q} or N, and each R^{q} is independently H, halogen, or an optionally substituted alkyl; and
wherein, in Formula 2-I, n is 0-30, and m is 0-4.

38. The combination of claim 33, wherein the radical of the immune modulator of the first medicament comprises a TLR agonist having the following formula or a pharmaceutically acceptable salt thereof: 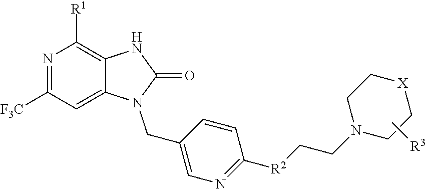
wherein:
R¹ is an amine group,
R² is a single bond —NH—.
R³ is an H, an alkyl, a hydroxy group, or any other substituted group thereof,
X is a CH₂, NH, O, or S, and
the linker is attached at R¹, R² or R³.

39. The combination of claim 33, wherein the at least one compound of the first medicament is or comprises the formula: 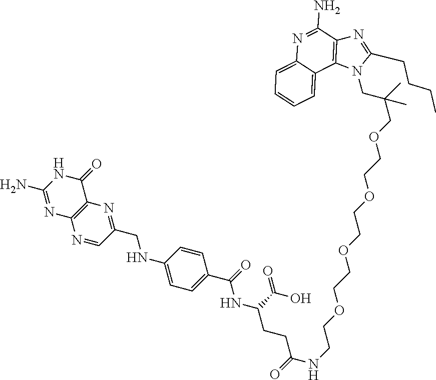
or is a pharmaceutically acceptable salt or hydrate thereof.

40. The combination of claim 33, wherein the linker of the at least one compound or pharmaceutically acceptable salt or hydrate of the first medicament is a releasable linker.

41. The combination of claim 33, wherein the linker of the at least one compound or pharmaceutically acceptable salt or hydrate of the first medicament is a non-releasable linker.

42. The combination of claim 33, wherein the linker of the at least one compound or pharmaceutically acceptable salt or hydrate of the first medicament comprises a polyethylene glycol (PEG) linker or a PEG derivative linker and is a non-releasable linker.

43. The combination of any one of claims 33-42, wherein the subject has a programmed death-ligand 1 (PD-L1), programmed death 1 (PD-1), or cytotoxic T-lymphocyte-associated antigen 4 (CTLA-4) positive tumor.

44. The combination of claim 33, wherein the one or more immune checkpoint inhibitors of the second medicament comprise a small molecule or other agent that disrupts an immune checkpoint of a cell of the subject.

45. The combination of any one of claims 33-42 and 44, wherein the one or more checkpoint inhibitors of the second medicament are each independently selected from the group consisting of pembrolizumab, nivolumab, ipilimumab, cemiplimab, atezolizumab, avelumab, durvalumab, pidilizumab, monoclonal antibody MEDI-0680, monoclonal antibody REGN2810, or fusion protein AMP-224 that targets PD-1, ociperlimab, islelizumab, a combination of ociperlimab and islelizumab, BMS-936559/MDX-1105, MPDL3280A/RG7446/atezolizumab, MSB0010718C/avelumab, or MEDI4736/durvalumab, tiragolumab, zimberelimab, tremelimumab, relatlimab, monoclonal antibody IMP321, nivolumab, etigilimab, domvanalimab, tiragolumab (RG6058), vibostolimab, avelumab, and durvalumab.

46. The combination of any one of claims 33-42 and 44, wherein the one or more immune checkpoint inhibitors of the second medicament are one or more agents that bind to or inhibit an immune checkpoint of a cell selected from a group consisting of PD-1, PD-L1, CTLA-4, V-domain Ig suppressor of T cell activation (VISTA), lymphocyte activating 3 (LAG3), T cell immunoglobulin and mucin domain-containing protein 3 (TIM3), T cell immunoreceptor with Ig and ITIM domains (TIGIT), programmed death ligand 2 (PD-L2), indoleamine 2,3-deioxygenase (IDO), arginase-1 (AGR1), B7 family inhibitory ligand B7-H3 (B7-H3), B7 family inhibitory ligand B7-H4 (B7-H4), 2B4 (cluster of differentiation 244), B- and T-lymphocyte attenuator (BTLA), adenosine A2A receptor (A2aR), and/or a member of the family of killer cell immunoglobulin-like receptors (KIRs) such as KIRs and C-type lectin receptors, and signal transducer and activator of transcription (STAT3).

47. The combination of any one of claims 33-42 and 44, wherein the cancer comprises a cold tumor, a hot tumor, or an immune desert tumor.

48. The combination of any one of claims 33-47 used for enhancing a potency of one or more immune checkpoint inhibitors administered to a subject with cancer and/or for treating a cancer.
